(19) United States
(12) Patent Application Publication
(10) Pub. No.: US 2025/0171556 A1
(43) Pub. Date: May 29, 2025

(54) MULTISPECIFIC ANTIBODIES FOR TREATING CD47-ASSOCIATED DISEASES

(71) Applicant: Virtuoso BINco, Inc., San Mateo, CA (US)

(72) Inventors: Xinhua WANG, San Mateo, CA (US); Xiaocheng CHEN, San Mateo, CA (US); Lei SHI, San Mateo, CA (US); Oi Kwan WONG, San Mateo, CA (US); Qi FEI, Shanghai (CN); Leonard POST, San Mateo, CA (US)

(21) Appl. No.: 18/704,831

(22) PCT Filed: Oct. 26, 2022
(86) PCT No.: PCT/CN2022/127745

§ 371 Date: Apr. 25, 2024

(30) Foreign Application Priority Data
Oct. 27, 2021 (WO) PCT/CN2021/126743

(51) Int. Cl. C07K 16/30; A61K 31/282; A61K 31/4745; A61K 31/513; A61K 39/00; A61K 39/395; A61P 35/00; C07K 16/28

(52) U.S. Cl. CPC C07K 16/30; A61K 31/282; A61K 31/4745; A61K 31/513; A61K 39/39558; A61P 35/00; C07K 16/2803; A61K 2039/505; C07K 2317/24; C07K 2317/31; C07K 2317/35; C07K 2317/73; C07K 2317/732; C07K 2317/76; C07K 2317/92

(57) ABSTRACT

The present disclosure describes multispecific antibodies comprising a first targeting moiety that specifically binds to CD47 and a second targeting moiety. The second targeting moiety can specifically bind to EpCAM or to EGFR. Also described herein include pharmaceutical compositions comprising the multispecific antibodies, and methods of use for killing CD47+ cells, such as CD47+ EpCAM+ cells or CD47+ EGFR+ cells, and for treating proliferative diseases.

### CROSS-REFERENCE

This application claims priority to PCT application no. PCT/CN2021/126743, filed on Oct. 27, 2021, the entirety of which is hereby incorporated by reference herein.

### SEQUENCE LISTING

The instant application contains a Sequence Listing which has been submitted electronically in ASCII format and is hereby incorporated by reference in its entirety. Said ASCII copy, created on Oct. 24, 2022, is named txt_55429-739_602_SL.txt and is 16 kilobytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

This disclosure provides multispecific antibodies comprising a CD47-binding domain and an EpCAM binding domain. In some embodiments, the multispecific antibody is bispecific, trispecific, or tetraspecific. In some embodiments, the multispecific antibody is bispecific. In some embodiments, the multispecific antibody is bivalent, trivalent, or tetravalent. In some embodiments, the multispecific antibody is bivalent.

In some embodiments of the multispecific antibody comprising the CD47-binding domain and the EpCAM binding domain, the multispecific antibody has a higher affinity for CD47 expressed on the surface of a tumor cell than for CD47 expressed on the surface of a normal cell, such as red blood cell, platelet, T cell or NK cell. In some embodiments, the tumor cell expresses EpCAM. In some embodiments, a concentration of the multispecific antibody required for half-maximal binding to a (e.g., human) red blood cell is greater than about 500 nanomolar (nM).

In some embodiments of the multispecific antibody comprising the CD47-binding domain and the EpCAM binding domain, the multispecific antibody binds to (e.g., human) CD47 with a dissociation constant (KD) of less than about 100 nanomolar (nM). The In some embodiments, the multispecific antibody binds to (e.g., human) CD47 with a dissociation constant (KD) of from about 1 nanomolar (nM) to about 100 nM, from about 1 nM to about 50 nM, or from about 5 nM to about 50 nM. In some embodiments, the multispecific antibody binds to EpCAM with a dissociation constant (KD) of less than about 500 nanomolar (nM). In some embodiments, the multispecific antibody binds to EpCAM with a dissociation constant (KD) of from about 0.2 nanomolar (nM) to about 500 nM, from about 1 nM to about 300 nM, from about 5 nM to about 200 nM, or from about 10 nM to about 150 nM. In some embodiments, the dissociation constant (KD) is determined by surface plasmon resonance.

In some embodiments of the multispecific antibody comprising the CD47-binding domain and the EpCAM binding domain, the EpCAM-binding domain is a human or engineered human EpCAM-binding domain. In some embodiments, the EpCAM binding domain comprises an antibody, or functional fragment or functional variant thereof, that binds specifically to EpCAM. In some embodiments, the antibody, or functional fragment or functional variant thereof that binds specifically to EpCAM comprises an anti-EpCAM heavy chain and an anti-EpCAM light chain. In some embodiments, the anti-EpCAM heavy chain comprises an anti-EpCAM heavy chain variable domain. In some embodiments, the anti-EpCAM heavy chain variable domain comprises a variable domain of an IgG1, IgG2, IgG3, or IgG4 heavy chain. In some embodiments, the anti-EpCAM light chain comprises an anti-EpCAM light chain variable domain. In some embodiments, the anti-EpCAM light chain variable domain comprises a Kappa or Lambda light chain. In some embodiments, the EpCAM binding domain comprises a single-chain variable fragment (scFv). In some embodiments, the EpCAM binding domain comprises an antigen-binding fragment (Fab).

In some embodiments of the multispecific antibody comprising the CD47-binding domain and the EpCAM binding domain, the EpCAM binding domain comprises a T cell receptor (TCR) constant region. In some embodiments, the TCR constant region of the EpCAM binding domain comprises a TCR alpha constant domain and a TCR beta constant domain. In some embodiments, the EpCAM binding domain comprises a VH-EpCAM domain and a VL-EpCAM domain; wherein the VH-EpCAM domain is fused to the TCR alpha constant domain; and the VL-EpCAM domain is fused to the TCR beta constant domain. In some embodiments, the EpCAM binding domain comprises a VH-EpCAM domain and a VL-EpCAM domain; wherein the VH-EpCAM domain is fused to the TCR beta constant domain; and the VL-EpCAM domain is fused to the TCR alpha constant domain. In some embodiments, the EpCAM binding domain comprises a VH-EpCAM domain and a VL-EpCAM domain; wherein the VH-EpCAM domain is fused to the CH1 IgG domain; and the VL-EpCAM domain is fused to the CL IgG domain.

In some embodiments of the multispecific antibody comprising the CD47-binding domain and the EpCAM binding domain, the EpCAM-binding domain comprises three heavy chain (HC) complementarity determining regions (CDRs): HC-CDR1, HC-CDR2, and HC-CDR3; wherein the HC-CDR1, the HC-CDR2, and the HC-CDR3 of the EpCAM binding domain comprises amino acid sequences selected from those set forth in Table 9. In some embodiments, the HC-CDR1 of the EpCAM binding domain comprises an amino acid sequence selected from SEQ ID NOs: 222, 225, 231, 234, 243, and 246. In some embodiments, the HC-CDR2 of the EpCAM binding domain comprises an amino acid sequence selected from SEQ ID NOs: 223, 226, 232, 235, 244, and 247. In some embodiments, the HC-CDR3 of the EpCAM binding domain comprises an amino acid sequence selected from SEQ ID NOs: 224, 227, 233, 236, 245, and 248.

In some embodiments of the multispecific antibody comprising the CD47-binding domain and the EpCAM binding domain, the EpCAM-binding domain comprises three light chain (LC) complementarity determining regions (CDRs): the LC-CDR1, the LC-CDR2, and the LC-CDR3; wherein the LC-CDR1, the LC-CDR2, and the LC-CDR3 of the EpCAM binding domain comprises amino acid sequences selected from those set forth in Table 10. In some embodiments, the LC-CDR1 of the EpCAM binding domain comprises an amino acid sequence selected from SEQ ID NOs: 276, 279, 285, 288, 297, and 300. In some embodiments, the LC-CDR2 of the EpCAM binding domain comprises an amino acid sequence selected from SEQ ID NOs: 277, 280, 286, 289, 298, and 301. In some embodiments, the LC-CDR3 of the EpCAM binding domain comprises an amino acid sequence selected from SEQ ID NOs: 278, 281, 287, 290, 299, and 302.

In some embodiments of the multispecific antibody comprising the CD47-binding domain and the EpCAM binding domain, the EpCAM-binding domain comprises a heavy chain framework region (HFR) comprising an amino acid sequence selected from those set forth in Table 11; and wherein the HFR comprises from 0-3 amino acid modification(s). In some embodiments, the EpCAM-binding domain comprises a heavy chain framework region (HFR) comprising an amino acid sequence selected from SEQ ID NOs: 331-334, 335-338, 343-346, 347-350, 355-358, and 359-362; and wherein the HFR comprises from 0-3 amino acid modification(s). In some embodiments, the EpCAM-binding domain comprises a heavy chain framework region (HFR) comprising a set of amino acid sequences of SEQ ID NOs: 331-334; and wherein at least one set of the amino acid sequences comprises from 0-3 amino acid modification(s). In some embodiments, the EpCAM-binding domain comprises a heavy chain framework region (HFR) comprising a set of amino acid sequences of SEQ ID NOs: 335-338 and wherein at least one set of the amino acid sequences comprises from 0-3 amino acid modification(s). In some embodiments, the EpCAM-binding domain comprises a heavy chain framework region (HFR) comprising a set of amino acid sequences of SEQ ID NOs: 343-346; and wherein at least one set of the amino acid sequences comprises from 0-3 amino acid modification(s). In some embodiments, the EpCAM-binding domain comprises a heavy chain framework region (HFR) comprising a set of amino acid sequences of SEQ ID NOs: 347-350; and wherein at least one set of the amino acid sequences comprises from 0-3 amino acid modification(s). In some embodiments, the EpCAM-binding domain comprises a heavy chain framework region (HFR) comprising a set of amino acid sequences of SEQ ID NOs: 355-358; and wherein at least one set of the amino acid sequences comprises from 0-3 amino acid modification(s). In some embodiments, the EpCAM-binding domain comprises a heavy chain framework region (HFR) comprising a set of amino acid sequences of SEQ ID NOs: 359-362; and wherein at least one set of the amino acid sequences comprises from 0-3 amino acid modification(s). In some embodiments, the HFR of the EpCAM-binding domain comprises from 0-2 amino acid modification(s). In some embodiments, the HFR of the EpCAM-binding domain comprises from 0-1 amino acid modification(s).

In some embodiments of the multispecific antibody comprising the CD47-binding domain and the EpCAM binding domain, the EpCAM-binding domain comprises a light chain framework region (LFR) comprising an amino acid sequence selected from those set forth in Table 12; and wherein the LFR comprises from 0-3 amino acid modification(s). In some embodiments, the EpCAM-binding domain comprises a light chain framework region (LFR) comprising an amino acid sequence selected from SEQ ID NOs: 367-370, 371-374, 379-382, 383-386, 391-394, and 395-398; and wherein the HFR comprises from 0-3 amino acid modification(s). In some embodiments, the EpCAM-binding domain comprises a light chain framework region (LFR) comprising a set of amino acid sequences of SEQ ID NOs: 367-370; and wherein at least one set of the amino acid sequences comprises from 0-3 amino acid modification(s). In some embodiments, the EpCAM-binding domain comprises a light chain framework region (LFR) comprising a set of amino acid sequences of SEQ ID NOs: 371-374; and wherein at least one set of the amino acid sequences comprises from 0-3 amino acid modification(s). In some embodiments, the EpCAM-binding domain comprises a light chain framework region (LFR) comprising a set of amino acid sequences of SEQ ID NOs: 379-382; and wherein at least one set of the amino acid sequences comprises from 0-3 amino acid modification(s). In some embodiments, the EpCAM-binding domain comprises a light chain framework region (LFR) comprising a set of amino acid sequences of SEQ ID NOs: 383-386; and wherein at least one set of the amino acid sequences comprises from 0-3 amino acid modification(s). In some embodiments, the EpCAM-binding domain comprises a light chain framework region (LFR) comprising a set of amino acid sequences of SEQ ID NOs: 391-394; and wherein at least one set of the amino acid sequences comprises from 0-3 amino acid modification(s). In some embodiments, the EpCAM-binding domain comprises a light chain framework region (LFR) comprising a set of amino acid sequences of SEQ ID NOs: 395-398; and wherein at least one set of the amino acid sequences comprises from 0-3 amino acid modification(s). In some embodiments, the LFR of the EpCAM-binding domain comprises from 0-2 amino acid modification(s). In some embodiments, the LFR of the EpCAM-binding domain comprises from 0-1 amino acid modification(s).

In some embodiments of the multispecific antibody comprising the CD47-binding domain and the EpCAM binding domain, the EpCAM-binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 13. In some embodiments, the EpCAM-binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 14. In some embodiments, the EpCAM-binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 15. In some embodiments, the EpCAM-binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 16. In some embodiments, the EpCAM binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 17.

In some embodiments of the multispecific antibody comprising the CD47-binding domain and the EpCAM binding domain, the EpCAM binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18. In some embodiments, the EpCAM binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18. In some embodiments, the EpCAM binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18.

In some embodiments of the multispecific antibody comprising the CD47-binding domain and the EpCAM binding domain, the CD47-binding domain is a human or engineered human CD47-binding domain. In some embodiments, the CD47 binding domain comprises an antibody, or functional fragment or functional variant thereof, that binds specifically to CD47. In some embodiments, the antibody, or functional fragment or functional variant thereof that binds specifically to CD47 comprises an anti-CD47 heavy chain and an anti-CD47 light chain. In some embodiments, the anti-CD47 heavy chain comprises an anti-CD47 heavy chain variable domain. In some embodiments, the anti-CD47 heavy chain variable domain comprises a variable domain of an IgG1, IgG2, IgG3, or IgG4 heavy chain. In some embodiments, the anti-CD47 light chain comprises an anti-CD47 light chain variable domain. In some embodiments, the anti-CD47 light chain variable domain comprises a variable domain of a Kappa or Lambda light chain. In some embodiments, the anti-CD47 heavy chain variable domain comprises the variable domain of an IgG1 heavy chain and the anti-CD47 light chain variable domain comprises a Kappa light chain. In some embodiments, the anti-CD47 heavy chain variable domain comprises the variable domain of an IgG1 heavy chain and the anti-CD47 light chain variable domain comprises a Lambda light chain. In some embodiments, the CD47 binding domain comprises a single-chain variable fragment (scFv). In some embodiments, the CD47 binding domain comprises an antigen-binding fragment (Fab).

In some embodiments of the multispecific antibody comprising the CD47-binding domain and the EpCAM binding domain, the CD47 binding domain comprises a T cell receptor (TCR) constant region. In some embodiments, the TCR constant region of the CD47 binding domain comprises a TCR alpha constant domain and a TCR beta constant domain. In some embodiments, the CD47 binding domain comprises a VH-CD47 domain and a VL-CD47 domain; wherein the VH-CD47 domain is fused to the TCR alpha constant domain; and the VL-CD47 domain is fused to the TCR beta constant domain. In some embodiments, the CD47 binding domain comprises a VH-CD47 domain and a VL-CD47 domain; wherein the VH-CD47 domain is fused to the TCR beta constant domain; and the VL-CD47 domain is fused to the TCR alpha constant domain. In some embodiments, the CD47 binding domain comprises a VH-CD47 domain and a VL-CD47 domain; wherein the VH-CD47 domain is fused to the CH1 IgG domain; and the VL-CD47 domain is fused to the CL IgG domain.

In some embodiments of the multispecific antibody comprising the CD47-binding domain and the EpCAM binding domain, the CD47-binding domain comprises three heavy chain (HC) complementarity determining regions (CDRs), HC-CDR1, HC-CDR2, and HC-CDR3; wherein the HC-CDR1, the HC-CDR2, and the HC-CDR3 of the CD47 binding domain comprises amino acid sequences selected from those set forth in Table 1; and wherein at least one of the HC-CDR1, the HC-CDR2, and the HC-CDR3 comprise from 0-3 amino acid modification(s). In some embodiments, at least one of the HC-CDR1, the HC-CDR2, and the HC-CDR3 of the CD47-binding domain comprise from 0-2 amino acid modification(s). In some embodiments, at least one of the HC-CDR1, the HC-CDR2, and the HC-CDR3 of the CD47-binding domain comprise from 0-1 amino acid modification(s).

In some embodiments of the multispecific antibody comprising the CD47-binding domain and the EpCAM binding domain, the CD47 binding domain comprises three light chain (LC) complementarity determining regions (CDRs), the LC-CDR1, the LC-CDR2, and the LC-CDR3; wherein the LC-CDR1, the LC-CDR2, and the LC-CDR3 of the CD47 binding domain comprises amino acid sequences selected from those set forth in Table 2; and wherein at least one of the LC-CDR1, the LC-CDR2, and the LC-CDR3 comprise from 0-3 amino acid modification(s). In some embodiments, at least one of the LC-CDR1, the LC-CDR2, and the LC-CDR3 of the CD47-binding domain comprise from 0-2 amino acid modification(s). In some embodiments, at least one of the LC-CDR1, the LC-CDR2, and the LC-CDR3 of the CD47-binding domain comprise from 0-1 amino acid modification(s).

In some embodiments of the multispecific antibody comprising the CD47-binding domain and the EpCAM binding domain, the CD47 binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 3. In some embodiments, the CD47 binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 4. In some embodiments, the CD47 binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 5. In some embodiments, the CD47 binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 6. In some embodiments, the CD47 binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 7.

In some embodiments of the multispecific antibody comprising the CD47-binding domain and the EpCAM binding domain, the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8. In some embodiments, the CD47 binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8. In some embodiments, the CD47 binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8.

In some embodiments of the multispecific antibody comprising the CD47-binding domain and the EpCAM binding domain, the multispecific antibody further comprising an Fc domain. In some embodiments, the Fc domain is a human Fc domain. In some embodiments, the isotype of the Fc domain is IgG1, IgG2, IgG3, or IgG4. In some embodiments, the Fc domain is a heterodimeric Fc domain, wherein the heterodimeric Fc region comprises a knob chain and a hole chain, forming a knob-into-hole (KiH) structure. In some embodiments, the knob chain comprises an IgG1, IgG2, IgG3, or IgG4 domain. In some embodiments, the knob chain comprises the mutation T366W and the hole chain comprises the mutations T366S, L368A, and Y407V, wherein amino acid position numbering is according to the EU index of Kabat et al. In some embodiments, the hole chain comprises an IgG1, IgG2, IgG3, or IgG4 domain. In some embodiments, the multispecific antibody has an asymmetric three-chain knob-into-hole structure.

In some embodiments of the multispecific antibody comprising the CD47-binding domain and the EpCAM binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 26.

In some embodiments of the multispecific antibody comprising the CD47-binding domain and the EpCAM binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 27.

In some embodiments of the multispecific antibody comprising the CD47-binding domain and the EpCAM binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 28.

In some embodiments of the multispecific antibody comprising the CD47-binding domain and the EpCAM binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 29.

In some embodiments of the multispecific antibody comprising the CD47-binding domain and the EpCAM binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 30.

In some embodiments of the multispecific antibody comprising the CD47-binding domain and the EpCAM binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 31.

In some embodiments of the multispecific antibody comprising the CD47-binding domain and the EpCAM binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 32.

In some embodiments of the multispecific antibody comprising the CD47-binding domain and the EpCAM binding domain, less than about 1 nanomolar (nM) or less than about 0.001 nM of the multispecific antibody increases a percentage of HCT-15 cells engulfed by macrophage by at least 4-fold compared to a nonspecific IgG1 antibody control. In some embodiments, a concentration of the antibody required to mediate antibody-dependent cellular phagocytosis of an EpCAM-positive, CD47-positive tumor cell by a macrophage is from about 0.001 nanomolar (nM) to about 3 nM. In some embodiments, the EpCAM-positive, CD47-positive tumor cell is an OVISE cell or an HCT15 cell. In some embodiments, the EpCAM-positive, CD47-positive tumor cell is selected from a ductal pancreatic adenocarcinoma cell, an ovarian clear cell adenocarcinoma cell, a colon adenocarcinoma cell, a lung adenocarcinoma cell, a lung squamous cell carcinoma cell, a lung small cell carcinoma cell, a hepatocellular carcinoma cell, a gastric adenocarcinoma cell, a cholangiocarcinoma cell, a head and neck squamous cell carcinoma cell, a prostate carcinoma cell, an endometrial carcinoma cell, an esophageal cancer cell, an ovarian adenocarcinoma cell, a vulvar squamous cell carcinoma cell.

In some embodiments of the multispecific antibody comprising the CD47-binding domain and the EpCAM binding domain, about 100 nanomolar (nM) of the multispecific antibody inhibits the binding of SIRPα to CD47 on the surface of a cell by at least about 30%. In some embodiments, the cell is a CD47+ EpCAM+ tumor cell. In some embodiments, the cell expresses at least as many EpCAM proteins on its surface as an HCC-44 cell. In some embodiments, the cell expresses at least 50,000, at least 100,000, at least 300,000, at least 600,000 or at least 1,500,000 EpCAM proteins on its surface. In some embodiments, about 1000 nanomolar (nM) of the multispecific antibody does not induce hemolysis of red blood cells in a hemagglutination assay.

This disclosure provides a complex comprising the multispecific antibody comprising the CD47-binding domain and the EpCAM binding domain (as described herein) and a CD47+ EpCAM+ target cell. In some embodiments, the target cell expresses at least as many EpCAM proteins on its surface as an HCC-44 cell. In some embodiments, the target cell expresses at least 50,000, at least 100,000, at least 300,000, at least 600,000 or at least 1,500,000 EpCAM proteins on its surface. In some embodiments, the CD47+ EpCAM+ target cell is a cancer cell. In some embodiments, the cancer cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

This disclosure provides a method of inducing phagocytosis of a CD47+ EpCAM+ target cell comprising administering the multispecific antibody comprising the CD47-binding domain and the EpCAM binding domain (as described herein). In some embodiments, the multispecific antibody is administered at a concentration of less than about 5 nanomolar (nM), less than about 2 nM, less than about 1 nM, less than about 0.5 nM, less than about 0.2 nM, less than about 0.1 nM, or less than about 0.05 nM.

This disclosure provides a method of killing a CD47+ EpCAM+ target cell comprising administering the multispecific antibody comprising the CD47-binding domain and the EpCAM binding domain (as described herein), wherein the multispecific antibody inhibits the binding of SIRPα to CD47 on the surface of the CD47+ EpCAM+ target cell. In some embodiments, the antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 to about 0.05 nM, or less than about 0.01 nM. In some embodiments, the CD47+ EpCAM+ target cell is selected from an HCT-15 cell, an A431 cell, an HCC-44 cell, a SKOV-3 cell, an OVISE cell, or a CFPAC-1 cell. In some embodiments, the CD47+ EpCAM+ target cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

This disclosure provides a method of killing a CD47+ EpCAM+ target cell comprising administering the multispecific antibody comprising the CD47-binding domain and the EpCAM binding domain (as described herein), wherein the multispecific antibody induces antibody-dependent cellular cytotoxicity that kills the CD47+ EpCAM+ target cell. In some embodiments, the antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 to about 0.05 nM, or less than about 0.01 nM. In some embodiments, the CD47+ EpCAM+ target cell is selected from an HCT-15 cell, an A431 cell, an HCC-44 cell, a SKOV-3 cell, an OVISE cell, or a CFPAC-1 cell. In some embodiments, the CD47+ EpCAM+ target cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

This disclosure provides a pharmaceutical composition comprising the multispecific antibody comprising the CD47-binding domain and the EpCAM binding domain (as described herein).

This disclosure provides a method of treating a subject with cancer comprising administering to the subject the pharmaceutical composition comprising the multispecific antibody comprising the CD47-binding domain and the EpCAM binding domain (as described herein). In some embodiments, the cancer is ductal pancreatic adenocarcinoma cell, ovarian clear cell adenocarcinoma cell, ovarian adenocarcinoma cell, colon adenocarcinoma cell, lung adenocarcinoma, rectum adenocarcinoma, esophageal cancer, gastric cancer, non-small cell lung cancer, small cell lung cancer, endometrial cancer, prostate adenocarcinoma, thyroid carcinoma, breast cancer, cervical carcinoma, renal cell carcinoma, testis cancer, or vulvar squamous cell carcinoma.

This disclosure provides multispecific antibodies comprising a CD47-binding domain and an EGFR binding domain. In some embodiments, the multispecific antibody is bispecific, trispecific, or tetraspecific. In some embodiments, the multispecific antibody is bispecific. In some embodiments, the multispecific antibody is bivalent, trivalent, or tetravalent. In some embodiments, the multispecific antibody is bivalent.

In some embodiments, the multispecific antibody has a higher affinity for CD47 expressed on the surface of a tumor cell than for CD47 expressed on the surface of a normal cell, such as red blood cell, platelet, T cell, B cell or NK cell. In some embodiments, the tumor cell expresses EGFR. In some embodiments, a concentration of the multispecific antibody required for half-maximal binding to a (e.g., human) red blood cell is greater than about 200 nanomolar (nM).

In some embodiments of the multispecific antibody comprising the CD47-binding domain and the EGFR binding domain, the multispecific antibody binds to (e.g., human) CD47 with a dissociation constant (KD) of less than about 100 nanomolar (nM). In some embodiments, the multispecific antibody binds to (e.g., human) CD47 with a dissociation constant (KD) of from about 1 nanomolar (nM) to about 100 nM, from about 1 nM to about 50 nM, or from about 5 nM to about 50 nM. In some embodiments, the multispecific antibody binds to EGFR with a dissociation constant (KD) of less than about 25 nanomolar (nM). In some embodiments, the multispecific antibody binds to EGFR with a dissociation constant (KD) of from about 0.2 nanomolar (nM) to about 25 nM, from about 0.2 nM to about 10 nM, from about 0.2 nM to about 2 nM, or from about 2 nM to about 10 nM. In some embodiments, the dissociation constant (KD) is determined by surface plasmon resonance.

In some embodiments of the multispecific antibody comprising the CD47-binding domain and the EGFR binding domain, the CD47-binding domain is a human or engineered human CD47-binding domain. In some embodiments, the CD47 binding domain comprises an antibody, or functional fragment or functional variant thereof, that binds specifically to CD47. In some embodiments, the antibody, or functional fragment or functional variant thereof that binds specifically to CD47 comprises an anti-CD47 heavy chain and an anti-CD47 light chain. In some embodiments, the anti-CD47 heavy chain comprises an anti-CD47 heavy chain variable domain. In some embodiments, the anti-CD47 heavy chain variable domain comprises a variable domain of an IgG1, IgG2, IgG3, or IgG4 heavy chain. In some embodiments, the anti-CD47 light chain comprises an anti-CD47 light chain variable domain. In some embodiments, the anti-CD47 light chain variable domain comprises a Kappa or Lambda light chain. In some embodiments, the anti-CD47 heavy chain variable domain comprises the variable domain of an IgG1 heavy chain and the anti-CD47 light chain variable domain comprises a Kappa light chain. In some embodiments, the anti-CD47 heavy chain variable domain comprises the variable domain of an IgG1 heavy chain and the anti-CD47 light chain variable domain comprises a Lambda light chain. In some embodiments, the CD47 binding domain comprises a single-chain variable fragment (scFv). In some embodiments, the CD47 binding domain comprises an antigen-binding fragment (Fab).

In some embodiments of the multispecific antibody comprising the CD47-binding domain and the EGFR binding domain, the CD47 binding domain comprises a T cell receptor (TCR) constant region. In some embodiments, the TCR constant region of the CD47 binding domain comprises a TCR alpha constant domain and a TCR beta constant domain. In some embodiments, the CD47 binding domain comprises a VH-CD47 domain and a VL-CD47 domain; wherein the VH-CD47 domain is fused to the TCR alpha constant domain; and the VL-CD47 domain is fused to the TCR beta constant domain. In some embodiments, the CD47 binding domain comprises a VH-CD47 domain and a VL-CD47 domain; wherein the VH-CD47 domain is fused to the TCR beta constant domain; and the VL-CD47 domain is fused to the TCR alpha constant domain. In some embodiments, the CD47 binding domain comprises a VH-CD47 domain and a VL-CD47 domain; wherein the VH-CD47 domain is fused to the CH1 IgG domain; and the VL-CD47 domain is fused to the CL IgG domain.

In some embodiments of the multispecific antibody comprising the CD47-binding domain and the EGFR binding domain, the CD47-binding domain comprises three heavy chain (HC) complementarity determining regions (CDRs), HC-CDR1, HC-CDR2, and HC-CDR3; wherein the HC-CDR1, the HC-CDR2, and the HC-CDR3 of the CD47 binding domain comprises amino acid sequences selected from those set forth in Table 1; and wherein at least one of the HC-CDR1, the HC-CDR2, and the HC-CDR3 comprise from 0-3 amino acid modification(s). In some embodiments, at least one of the HC-CDR1, the HC-CDR2, and the HC-CDR3 of the CD47-binding domain comprise from 0-2 amino acid modification(s). In some embodiments, at least one of the HC-CDR1, the HC-CDR2, and the HC-CDR3 of the CD47-binding domain comprise from 0-1 amino acid modification(s).

In some embodiments of the multispecific antibody comprising the CD47-binding domain and the EGFR binding domain, the CD47 binding domain comprises three light chain (LC) complementarity determining regions (CDRs), the LC-CDR1, the LC-CDR2, and the LC-CDR3; wherein the LC-CDR1, the LC-CDR2, and the LC-CDR3 of the CD47 binding domain comprises amino acid sequences selected from those set forth in Table 2; and wherein at least one of the LC-CDR1, the LC-CDR2, and the LC-CDR3 comprise from 0-3 amino acid modification(s). In some embodiments, at least one of the LC-CDR1, the LC-CDR2, and the LC-CDR3 of the CD47-binding domain comprise from 0-2 amino acid modification(s). In some embodiments, at least one of the LC-CDR1, the LC-CDR2, and the LC-CDR3 of the CD47-binding domain comprise from 0-1 amino acid modification(s).

In some embodiments of the multispecific antibody comprising the CD47-binding domain and the EGFR binding domain, the CD47 binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 3. In some embodiments, the CD47 binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 4. In some embodiments, the CD47 binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 5. In some embodiments, the CD47 binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 6. In some embodiments, the CD47 binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 7.

In some embodiments of the multispecific antibody comprising the CD47-binding domain and the EGFR binding domain, the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8. In some embodiments, the CD47 binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8. In some embodiments, the CD47 binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8.

In some embodiments of the multispecific antibody comprising the CD47-binding domain and the EGFR binding domain, the EGFR binding domain comprises a T cell receptor (TCR) constant region. In some embodiments, the TCR constant region of the EGFR binding domain comprises a TCR alpha constant domain and a TCR beta constant domain. In some embodiments, the EGFR binding domain comprises a VH-EGFR domain and a VL-EGFR domain; wherein the VH-EGFR domain is fused to the TCR alpha constant domain; and the VL-EGFR domain is fused to the TCR beta constant domain. In some embodiments, the EGFR binding domain comprises a VH-EGFR domain and a VL-EGFR domain; wherein the VH-EGFR domain is fused to the TCR beta constant domain; and the VL-EGFR domain is fused to the TCR alpha constant domain. In some embodiments, the EGFR binding domain comprises a VH-EGFR domain and a VL-EGFR domain; wherein the VH-EGFR domain is fused to the CH1 IgG domain; and the VL-EGFR domain is fused to the CL IgG domain.

In some embodiments of the multispecific antibody comprising the CD47-binding domain and the EGFR binding domain, the EGFR-binding domain comprises three heavy chain (HC) complementarity determining regions (CDRs), HC-CDR1, HC-CDR2, and HC-CDR3; wherein the HC-CDR1, the HC-CDR2, and the HC-CDR3 of the EGFR binding domain comprises amino acid sequences selected from those set forth in Table 19; and wherein at least one of the HC-CDR1, the HC-CDR2, and the HC-CDR3 comprise from 0-3 amino acid modification(s). In some embodiments, at least one of the HC-CDR1, the HC-CDR2, and the HC-CDR3 of the EGFR-binding domain comprise from 0-2 amino acid modification(s). In some embodiments, at least one of the HC-CDR1, the HC-CDR2, and the HC-CDR3 of the EGFR-binding domain comprise from 0-1 amino acid modification(s).

In some embodiments of the multispecific antibody comprising the CD47-binding domain and the EGFR binding domain, the EGFR binding domain comprises three light chain (LC) complementarity determining regions (CDRs), the LC-CDR1, the LC-CDR2, and the LC-CDR3; wherein the LC-CDR1, the LC-CDR2, and the LC-CDR3 of the EGFR binding domain comprises amino acid sequences selected from those set forth in Table 20; and wherein at least one of the LC-CDR1, the LC-CDR2, and the LC-CDR3 comprise from 0-3 amino acid modification(s). In some embodiments, at least one of the LC-CDR1, the LC-CDR2, and the LC-CDR3 of the EGFR-binding domain comprise from 0-2 amino acid modification(s). In some embodiments, at least one of the LC-CDR1, the LC-CDR2, and the LC-CDR3 of the EGFR-binding domain comprise from 0-1 amino acid modification(s).

In some embodiments of the multispecific antibody comprising the CD47-binding domain and the EGFR binding domain, the EGFR binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 21. In some embodiments, the EGFR binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 22. In some embodiments, the EGFR binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 23. In some embodiments, the EGFR binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 24.

In some embodiments of the multispecific antibody comprising the CD47-binding domain and the EGFR binding domain, the EGFR-binding domain comprises a heavy chain variable domain and a light chain variable domain. In some embodiments, the EGFR binding domain comprises a single-chain variable fragment (scFv). In some embodiments, the EGFR binding domain comprises an antigen-binding fragment (Fab). In some embodiments, the EGFR binding domain comprises a Fab domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 25.

In some embodiments of the multispecific antibody comprising the CD47-binding domain and the EGFR binding domain, the multispecific antibody further comprising an Fc domain. In some embodiments, the Fc domain is a human Fc domain. In some embodiments, the isotype of the Fc domain is IgG1, IgG2, IgG3, or IgG4. In some embodiments, the Fc domain is a heterodimeric Fc domain, wherein the heterodimeric Fc region comprises a knob chain and a hole chain, forming a knob-into-hole (KiH) structure. In some embodiments, the knob chain comprises an IgG1, IgG2, IgG3, or IgG4 domain. In some embodiments, the knob chain comprises the mutation T366W and the hole chain comprises the mutations T366S, L368A, and Y407V, wherein amino acid position numbering is according to the EU index of Kabat et al. In some embodiments, the hole chain comprises an IgG1, IgG2, IgG3, or IgG4 domain. In some embodiments, the multispecific antibody has an asymmetric three-chain knob-into-hole structure.

In some embodiments of the multispecific antibody comprising the CD47-binding domain and the EGFR binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 33.

In some embodiments of the multispecific antibody comprising the CD47-binding domain and the EGFR binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 34.

In some embodiments of the multispecific antibody comprising the CD47-binding domain and the EGFR binding domain, the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 35.

In some embodiments of the multispecific antibody comprising the CD47-binding domain and the EGFR binding domain, less than about 1 nanomolar (nM) or less than about 0.01 nM of the multispecific antibody increases a percentage of A431 cells engulfed by macrophage by at least 4-fold compared to a nonspecific IgG1 antibody control. In some embodiments, a concentration of the antibody required to mediate antibody-dependent cellular phagocytosis of an EGFR-positive, CD47-positive tumor cell by a macrophage is from about 0.01 nanomolar (nM) to about 3 nM. In some embodiments, the EGFR-positive, CD47-positive tumor cell is an OVISE cell or an A431 cell. In some embodiments, the EGFR-positive, CD47-positive tumor cell is selected from a ductal pancreatic adenocarcinoma cell, an ovarian clear cell adenocarcinoma cell, a colon adenocarcinoma cell, a lung adenocarcinoma cell, a lung squamous cell carcinoma cell, a lung small cell carcinoma cell, a hepatocellular carcinoma cell, a gastric adenocarcinoma cell, a cholangiocarcinoma cell, a head and neck squamous cell carcinoma cell, a prostate carcinoma cell, an endometrial carcinoma cell, an ovarian adenocarcinoma cell, a vulvar squamous cell carcinoma cell.

In some embodiments of the multispecific antibody comprising the CD47-binding domain and the EGFR binding domain, about 100 nanomolar (nM) of the multispecific antibody inhibits the binding of SIRPα to CD47 on the surface of a cell by at least about 30%. In some embodiments, the cell is a CD47+ EGFR+ tumor cell. In some embodiments, the cell expresses at least as many EGFR proteins on its surface as an HCC-44 cell. In some embodiments, the cell expresses at least 50,000, at least 100,000, at least 300,000, at least 600,000 or at least 1,500,000 EGFR proteins on its surface. In some embodiments, about 400 nanomolar (nM) of the multispecific antibody does not induce hemolysis of red blood cells in a hemagglutination assay.

This disclosure provides a complex comprising a multispecific antibody comprising the CD47-binding domain and the EGFR binding domain (as described herein) and a CD47+ EGFR+ target cell. In some embodiments, the target cell expresses at least as many EGFR proteins on its surface as an HCC-44 cell. In some embodiments, the target cell expresses at least 50,000, at least 100,000, at least 300,000, at least 600,000 or at least 1,500,000 EGFR proteins on its surface. In some embodiments, the CD47+ EGFR+ target cell is a cancer cell. In some embodiments, the cancer cell is epidermoid carcinoma cell, colorectal adenocarcinoma cell, head and neck squamous cell carcinoma cell, bladder adenocarcinoma cell, pancreas adenocarcinoma cell, lung squamous cell carcinoma, gastric carcinoma cell, ductal pancreatic adenocarcinoma cell, ovarian clear cell adenocarcinoma cell, ovarian adenocarcinoma cell, colon adenocarcinoma cell, lung adenocarcinoma, rectum adenocarcinoma, esophageal cancer, gastric cancer, non-small cell lung cancer, small cell lung cancer, endometrial cancer, prostate adenocarcinoma, thyroid carcinoma, breast cancer, cervical carcinoma, renal cell carcinoma, testis cancer, or vulvar squamous cell carcinoma.

This disclosure provides a method of inducing phagocytosis of a CD47+ EGFR+ target cell comprising administering a multispecific antibody comprising the CD47-binding domain and the EGFR binding domain (as described herein). In some embodiments, the multispecific antibody is administered at a concentration of less than about 5 nanomolar (nM), less than about 2 nM, less than about 1 nM, less than about 0.5 nM, less than about 0.2 nM, less than about 0.1 nM, or less than about 0.05 nM.

This disclosure provides a method of killing a CD47+ EGFR+ target cell comprising administering a multispecific antibody comprising the CD47-binding domain and the EGFR binding domain (as described herein), wherein the multispecific antibody inhibits the binding of SIRPα to CD47 on the surface of the CD47+ EGFR+ target cell. In some embodiments, the antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 nM to about 0.05 nM, or less than about 0.01 nM. In some embodiments, the CD47+ EGFR+ target cell is selected from an A431 cell, a NCI-H747 cell, an ASPC-1 cell, an EBC-1 cell, or a SNU-5 cell. In some embodiments, the CD47+ EGFR+ target cell is selected from an epidermoid carcinoma cell, a colorectal adenocarcinoma cell, a head and neck squamous cell carcinoma cell, a bladder carcinoma cell, a pancreas adenocarcinoma cell, a lung squamous cell carcinoma cell, a lung adenocarcinoma cell, a gastric carcinoma cell, a ductal pancreatic adenocarcinoma cell, an ovarian clear cell adenocarcinoma cell, an ovarian adenocarcinoma cell, a colon adenocarcinoma cell, a rectum adenocarcinoma cell, a esophageal cancer cell, a gastric cancer cell, a non-small cell lung cancer cell, a small cell lung cancer cell, an endometrial cancer cell, a prostate adenocarcinoma cell, a thyroid carcinoma cell, a breast cancer cell, a cervical carcinoma cell, a renal cell carcinoma cell, a testis cancer cell, and a vulvar squamous cell carcinoma cell.

This disclosure provides a method of killing a CD47+ EGFR+ target cell comprising administering a multispecific antibody comprising the CD47-binding domain and the EGFR binding domain (as described herein), wherein the multispecific antibody induces antibody-dependent cellular cytotoxicity that kills the CD47+ EGFR+ target cell. In some embodiments, the antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 nM to about 0.05 nM, or less than about 0.01 nM. In some embodiments, the CD47+ EGFR+ target cell is selected from an A431 cell, a NCI-H747 cell, an ASPC-1 cell, an EBC-1 cell, or a SNU-5 cell. In some embodiments, the CD47+ EGFR+ target cell is selected from an epidermoid carcinoma cell, a colorectal adenocarcinoma cell, a head and neck squamous cell carcinoma cell, a bladder carcinoma cell, a pancreas adenocarcinoma cell, a lung squamous cell carcinoma cell, a lung adenocarcinoma cell, a gastric carcinoma cell, a ductal pancreatic adenocarcinoma cell, an ovarian clear cell adenocarcinoma cell, an ovarian adenocarcinoma cell, a colon adenocarcinoma cell, a rectum adenocarcinoma cell, a esophageal cancer cell, a gastric cancer cell, a non-small cell lung cancer cell, a small cell lung cancer cell, an endometrial cancer cell, a prostate adenocarcinoma cell, a thyroid carcinoma cell, a breast cancer cell, a cervical carcinoma cell, a renal cell carcinoma cell, a testis cancer cell, and a vulvar squamous cell carcinoma cell.

This disclosure provides a pharmaceutical composition comprising a multispecific antibody comprising the CD47-binding domain and the EGFR binding domain (as described herein).

This disclosure provides a method of treating a subject with cancer comprising administering to the subject a pharmaceutical composition comprising a multispecific antibody comprising the CD47-binding domain and the EGFR binding domain (as described herein). In some embodiments, the cancer is epidermoid carcinoma, colorectal adenocarcinoma, head and neck squamous cell carcinoma, bladder carcinoma, pancreas adenocarcinom, lung squamous cell carcinoma, lung adenocarcinoma, gastric carcinoma, ductal pancreatic adenocarcinoma, ovarian clear cell adenocarcinoma, ovarian adenocarcinoma, colon adenocarcinoma, rectum adenocarcinoma, esophageal cancer, gastric cancer, non-small cell lung cancer, small cell lung cancer, endometrial cancer, prostate adenocarcinoma, thyroid carcinoma, breast cancer, cervical carcinoma, renal cell carcinoma, testis cancer, or vulvar squamous cell carcinoma.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A illustrates an example three-chain, knob-into-hole, CD47×EpCAM or CD47×EGFR bispecific antibody.

FIG. 1B illustrates an example four-chain, knob-into-hole, CD47×EpCAM or CD47×EGFR bispecific antibody in a TCR-chimera antibody format.

FIG. 2 illustrates the ELISA binding of an example CD47×EpCAM bispecific antibody on human CD47 (FIG. 2A) and human EpCAM recombinant proteins (FIG. 2B). Both arms of the antibody were shown to bind to their respective target antigen robustly.

FIGS. 3A-3B illustrate an example CD47×EpCAM bispecific antibody (“CD47×EpCAM_v5”) with very weak SIRPα blocking activity on CD47+ only Raji tumor cells (FIG. 3A) but potent blocking activity on EpCAM+/CD47+ HCT15 tumor cells (FIG. 3B).

FIG. 4 illustrates an example CD47×EpCAM bispecific antibody (“CD47×EpCAM_v5”) with potent antibody dependent cellular phagocytosis (ADCP) activity on EpCAM+/CD47+ tumor cells.

FIG. 5 illustrates an example CD47×EpCAM bispecific antibody (“CD47×EpCAM_v5”) with potent antibody-dependent cellular cytotoxicity (ADCC) activity on EpCAM+/CD47+ tumor cells.

FIGS. 6A-6B illustrate reduced red blood cells (RBCs) binding of an example CD47×EpCAM bispecific antibody (CD47×EpCAM_v5) as compared to that of a reference antibody “BMK-1” (such as described herein) as measured by mean fluorescence intensity MFI (FIG. 6A) and % of selective binding (FIG. 6B). No hemagglutination was observed with the treatment of the “CD47×EpCAM_v5” bispecific antibody as shown in FIG. 6C.

FIG. 7 illustrates less T cell binding of an example CD47×EpCAM bispecific antibody (“CD47×EpCAM_v5”) as compared to the reference “BMK-1” (such as described herein) and “VIR47.V8.”

FIG. 8A illustrates complete tumor inhibition in an OE-19 model by an example CD47×EpCAM bispecific antibody (“CD47×EpCAM_v5”), superior over the reference antibodies “BMK-1” and “BMK-2” (such as described herein) even at a lower dose. FIG. 8B illustrates the “CD47×EpCAM_v5” bispecific antibody induced complete tumor regression in a KATOIII model and showed superior efficacy over reference antibodies: monospecific EpCAM mAb (Mab002-11), CD47 mAb (“VIR47.V8”), “BMK-1” and “BMK-2” (such as described herein). All mice in the bispecific antibody group were tumor free even 10 days after the last dosing.

FIGS. 9A-9B illustrate ELISA binding of an example CD47×EGFR bispecific antibody “Panitumumab×VIR47.V8” on human EGFR (FIG. 9A) and human CD47 (FIG. 9B) recombinant proteins.

FIGS. 10A-10C illustrate the anti-tumor activities of CD47×EpCAM_v5 bispecific antibody as a single agent and in combination with oxaliplatin (FIG. 10A), 5-fluorouracil (5-FU, FIG. 10B) or irinotecan (FIG. 10C) in a colorectal cancer xenograft model, DLD-1.

FIGS. 11A-11B illustrate the anti-tumor activities of combining irinotecan with benchmark CD47 antibody (BMK-1), anti-CD47 bivalent antibody (VIR47.V8) and two CD47×EpCAM bispecific antibodies CD47×EpCAM_v5 and CD47×EpCAM_v6 in a colorectal cancer xenograft model, DLD-1.

FIG. 12 illustrates the anti-tumor activities of irinotecan and CD47×EpCAM bispecific antibody CD47×EpCAM_v5 as single agents or in combination in a hepatocellular carcinoma xenograft model, Hep3B2.1-7.

FIG. 13 illustrates the ELISA binding of CD47×EpCAM bispecific antibodies v9, v10, v11 on human EpCAM (FIG. 13A) and human CD47 recombinant proteins (FIG. 13B). Both arms of the antibodies were shown to bind to their respective target antigen robustly.

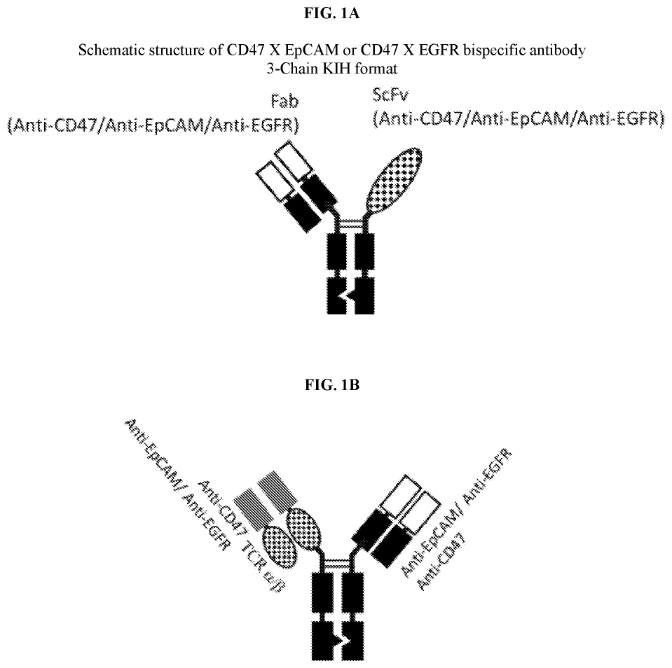

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Cluster of differentiation 47 (CD47), also known as integrin-associated protein (IAP), is a ˜50 kDa immunoglobulin superfamily membrane glycoprotein that is overexpressed in numerous blood cancers and solid tumors. High CD47 expression often correlates with more aggressive diseases and poorer clinical outcomes.

CD47 on the surface of CD47+ cells interacts with signal regulatory protein alpha (SIRPα) expressed on cells of the innate and adaptive immune systems, such as macrophages and dendritic cells. This interaction sends a “don't eat me” signal that inhibits phagocytosis, thereby allowing CD47+ cells to evade immune surveillance.

These data suggest that CD47 may serve as an immune checkpoint and that blocking the CD47-SIRPα interaction could have therapeutic value by switching off the “don't eat me” signal. Thus, blocking CD47 has emerged as a promising therapeutic strategy with numerous studies showing that interrupting the CD47-SIRPα signaling pathway promotes anti-tumor activity against human cancers in vitro and in vivo.

Several anti-CD47 monoclonal antibodies (mAbs) have been shown to increase phagocytosis of acute myeloid leukemia cells, non-Hodgkin's lymphoma cells, breast cancer cells, and ovarian cancer cells. In clinical studies, CD47 mAbs enhanced the anti-tumor activity of other therapeutic antibodies. At least six anti-CD47 mAbs and three SIRPα fusion proteins are in active phase I or II clinical trials for the treatment of human hematological malignancies and solid tumors.

The efficacy of anti-CD47 mAbs is limited by their interactions with red blood cells (RBCs), which also express CD47. RBCs act as a sink to sequester anti-CD47 antibodies, thereby preventing them from binding to malignant CD47-expressing (CD47+) cells. Furthermore, anti-CD47 mAb binding to RBCs leads to hemagglutination and lysis of the RBCs, resulting in anemia. Thus, there is a need for improved methods of treating malignant diseases mediated by CD47+ cells with reduced off-tumor effects.

Epithelial cell adhesion molecule (EpCAM), also known as CD236, is a ˜35 kDa type I transmembrane glycoprotein which functions as a homophilic Ca2+-independent cell-cell adhesion molecule. EpCAM is also involved in cell signaling, proliferation, differentiation, formation and maintenance of organ morphology. Its overexpression is found on many metastasizing epithelial cancers, such as breast, prostate, ovarian, lung, colon, renal and gastric cancers, highlighting its potential as an ideal target for immunotherapies (Spizzo et. al, Breast Cancer Res Treat 86:207-213).

Epidermal growth factor receptor (EGFR), also known as ErbB-1 or HER1, is a transmembrane protein that is a receptor for members of the epidermal growth factor family of extracellular protein ligands. EGFR is a member of the ErbB family of receptors, a subfamily of four closely related receptor tyrosine kinases: EGFR, HER2/neu, Her 3, and Her4. Overexpression of EGFR has been associated with a number of cancers, including adenocarcinoma of the lung, colorectal cancers, glioblastoma, and epithelial tumors of the head and neck.

In some embodiments, disclosed herein are anti-CD47 antibodies, anti-EpCAM antibodies, and multi-specific antibodies which comprise a targeting moiety to CD47 and a targeting moiety to EpCAM, or a combination thereof. In some embodiments, also described herein are multispecific (e.g., bispecific) antibodies which comprise a first targeting moiety to CD47 and a second targeting moiety to EpCAM. Additional embodiments, further described herein, are methods of treating a cancer with an anti-CD47 antibody, an anti-EpCAM antibody, and a multi-specific antibody (e.g. a bispecific CD47/EpCAM antibody).

In some embodiments, disclosed herein are anti-CD47 antibodies, anti-EGFR antibodies, and multi-specific antibodies which comprise a targeting moiety to CD47 and a targeting moiety to EGFR or a combination thereof. In some embodiments, also described herein are multispecific (e.g., bispecific) antibodies which comprise a first targeting moiety to CD47 and a second targeting moiety to EGFR. Additional embodiments, further described herein, are methods of treating a cancer with an anti-CD47 antibody, an anti-EGFR antibody, and a multi-specific antibody (e.g. a bispecific CD47/EGFR antibody).

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described. In this application, the use of the singular includes the plural unless specifically stated otherwise. It is noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error. Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification and attached claims are approximations that can vary depending upon the desired properties sought to be obtained by the present application. Generally the term “about,” as used herein when referring to a measurable value such as an amount of weight, time, dose, etc. is meant to encompass in one example variations of ±20% or ±10%, in another example ±5%, in another example ±1%, and in yet another example ±0.1% from the specified amount, as such variations are appropriate to perform the disclosed method.

The section headings used herein are for organizational purposes only and are not to be construed as limiting the subject matter described.

“Antibodies” and “immunoglobulins” (Igs) are glycoproteins having the same structural characteristics. The terms are used synonymously. In some instances, the antigen specificity of the immunoglobulin is known.

The term “antibody” is used in the broadest sense and covers fully assembled antibodies, antibody fragments that can bind antigen (e.g., Fab, F(ab′)2, Fv, single chain antibodies, diabodies, antibody chimeras, hybrid antibodies, bispecific antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

“Native antibodies” and “native immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular cytotoxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

The CDR sequence(s) for the antibodies disclosed herein, or the anti-CD47 or anti-ICAM1 binding domain sequences disclosed herein, may be defined or determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215 (1): 175-82; and U.S. Pat. No. 7,709,226); or (iii) the ImMunoGeneTics (IMGT) numbering system, for example, as described in Lefranc, M.-P., 1999, The Immunologist, 7:132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The term “chimeric” antibody refers to an antibody in which a portion of the heavy and/or light chain is derived from a particular source or species, while the remainder of the heavy and/or light chain is derived from a different source or species.

The term “human antibody” or “humanized antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). In some instances, human antibodies are also produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al, Proc. Natl. Acad. Sci. USA 90 (1993) 2551-2555; Jakobovits, A., et al, Nature 362 (1993)255-258; Bruggemann, M., et al, Year Immunol. 7 (1993) 33-40). In additional instances, human antibodies are also produced in phage display libraries (Hoogenboom, H. R., and Winter, G., J. Mol. Biol. 227 (1992) 381-388; Marks, J. D., et al, J. Mol. Biol. 222 (1991) 581-597). The techniques of Cole et al. and Boerner et al. are also available for the preparation of human monoclonal antibodies (Cole, et al., Monoclonal Antibodies and Cancer Therapy, Alan R. Liss, p. 77 (1985); and Boerner, P., et al, J. Immunol. 147 (1991) 86-95).

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The term “valent” as used herein denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule. The bispecific antibodies according to the invention are at least “bivalent” and may be “trivalent” or “multivalent” (e.g. “tetravalent” or “hexavalent”). In a particular aspect, the antibodies of the present invention have two or more binding sites and are bispecific. That is, the antibodies may be bispecific even in cases where there are more than two binding sites (i.e. that the antibody is trivalent or multivalent). In particular, the invention relates to bispecific bivalent antibodies, having one binding site for each antigen they specifically bind to.

The term “bispecific” means that the antibody is able to specifically bind to two distinct antigenic determinants, for example two binding sites each formed by a pair of an antibody heavy chain variable domain (VH) and an antibody light chain variable domain (VL) binding to different antigens. Such a bispecific antibody is an 1+1 format. Other bispecific antibody formats are 2+1 formats (comprising two binding sites for a first antigen or epitope and one binding site for a second antigen or epitope) or 2+2 formats (comprising two binding sites for a first antigen or epitope and two binding sites for a second antigen or epitope). Typically, a bispecific antibody comprises two antigen binding sites, each of which is specific for a different antigenic determinant. The term “multispecific” means that the antibody is able to specifically bind to two or more distinct antigenic determinants for example at least two binding sites each formed by a pair of an antibody heavy chain variable domain (VH) and an antibody light chain variable domain (VL) binding to different antigens.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The terms “cancer” and “tumor” are used interchangeably herein, encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

As used herein, the terms “Antibody-dependent cellular cytotoxicity” and “ADCC” refer to a cell-mediated reaction in which non-specific cytotoxic cells (e.g., Natural Killer (NK) cells, neutrophils, and macrophages) recognize bound antibody on a target cell and subsequently cause lysis of the target cell.

### Anti-CD47 Binding Domains

In certain embodiments, disclosed herein is an anti-CD47 binding domain. In some embodiments, the CD47-binding domain is a human or engineered human CD47-binding domain. In some embodiments, the CD47 binding domain comprises an antibody, or functional fragment or functional variant thereof, that binds specifically to CD47. In some embodiments, the antibody, or functional fragment or functional variant thereof that binds specifically to CD47 comprises an anti-CD47 heavy chain and an anti-CD47 light chain. In some embodiments, the anti-CD47 heavy chain comprises an anti-CD47 heavy chain variable domain. In some embodiments, the anti-CD47 heavy chain variable domain comprises a variable domain of an IgG1, IgG2, IgG3, or IgG4 heavy chain. In some embodiments, the anti-CD47 light chain comprises an anti-CD47 light chain variable domain. In some embodiments, the anti-CD47 light chain variable domain comprises a Kappa or Lambda light chain. In some embodiments, the anti-CD47 heavy chain variable domain comprises the variable domain of an IgG1 heavy chain and the anti-CD47 light chain variable domain comprises a Kappa light chain. In some embodiments, the anti-CD47 heavy chain variable domain comprises the variable domain of an IgG1 heavy chain and the anti-CD47 light chain variable domain comprises a Lambda light chain. In some embodiments, the CD47 binding domain comprises a single-chain variable fragment (scFv). In some embodiments, the CD47 binding domain comprises an antigen-binding fragment (Fab).

In some embodiments, the CD47-binding domain comprises three heavy chain (HC) complementarity determining regions (CDRs), HC-CDR1, HC-CDR2, and HC-CDR3; wherein the HC-CDR1, the HC-CDR2, and the HC-CDR3 of the CD47 binding domain comprises amino acid sequences selected from those set forth in Table 1; and wherein at least one of the HC-CDR1, the HC-CDR2, and the HC-CDR3 comprise from 0-3 amino acid modification(s). In some embodiments, at least one of the HC-CDR1, the HC-CDR2, and the HC-CDR3 of the CD47-binding domain comprise from 0-2 amino acid modification(s). In some embodiments, at least one of the HC-CDR1, the HC-CDR2, and the HC-CDR3 of the CD47-binding domain comprise from 0-1 amino acid modification(s).

In some embodiments, the HC-CDR1, the HC-CDR2, and the HC-CDR3 of the CD47 binding domain comprise the amino acid sequences of SEQ ID NOs: 1-3; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 4-6; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 7-9; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 10-12; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 13-15; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 16-18; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 19-21; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 22-24; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 25-27; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 28-30; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 31-33; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 34-36; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 37-39; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 40-42; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 43-45; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 46-48; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 49-51; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 52-54; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 55-57; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 58-60; and wherein at least one of the HC-CDR1, the HC-CDR2, and the HC-CDR3 comprise from 0-3 (e.g., 0-2, or 0-1) amino acid modification(s).

TABLE 1
Example CDR sequences of the anti-CD47 heavy chain binding
domains, as determined according to Kabat nomenclature.

| | SEQ ID | | SEQ ID | | SEQ ID | |
|---|---|---|---|---|---|---|
| Construct | NO | CDR1 | NO | CDR2 | NO | CDR3 |
| VIR47 | 1 | NFAMS | 2 | TISASGGRTFYADS | 3 | EGSFGEGVDP |
| | | | | VKG | | |
| M.1D15E11 | 4 | SYVMH | 5 | YIDPYNDGTKYNE | 6 | GGMARRSFDY |
| | | | | KFKG | | |
| M.20B10E9 | 7 | DTYMH | 8 | RIDPANGKTKSDP | 9 | GDGYYVGGLDY |
| | | | | KFQG | | |
| M.69H1H9 | 10 | EYTIH | 11 | SINPNNGLFRYNQ | 12 | DYGFGYSDY |
| | | | | KFKG | | |
| M.30A5C6 | 13 | DTYMH | 14 | RIDPANGNTKYDP | 15 | GDGHYVGGLNY |
| | | | | KFQG | | |
| M.95D4A11 | 16 | SYWMN | 17 | SIDPYDSETHYNQ | 18 | GRGYAMDY |
| | | | | KFKD | | |
| R.1H3 | 19 | SSGYYMC | 20 | CIYMRSGDTYLA | 21 | GGTSYALNL |
| | | | | SWAKG | | |
| R.9B11 | 22 | SYWMA | 23 | CIYIGSGSGTTYY | 24 | WAGSDGHFTL |
| | | | | ASWAK | | |
| R.13A2 | 25 | SYYYVC | 26 | CIYAGSSGDTYY | 27 | ARGYTGYSYQFN |
| | | | | ASWAKG | | F |
| R.14G4 | 28 | SYHMC | 29 | CIYPNNGFTWYA | 30 | GSGNSYNQLDL |
| | | | | SWAKG | | |
| R.15G10 | 31 | SYHMN | 32 | IIYVGSGNADYAS | 33 | GPTNTYPTYFNL |
| | | | | WAKG | | |
| R.18B9 | 34 | SGYMC | 35 | CIYAGSSGKTYY | 36 | DDGGTYYTDYL |
| | | | | ASWATG | | YL |
| R.19H7 | 37 | SYHMC | 38 | CIYPNNDFTWYA | 39 | ASGNAYNLLDL |
| | | | | TWAKG | | |
| R.20H1 | 40 | SGYYMC | 41 | CIYVRSDSTYYAS | 42 | GVTGDSYRFNF |
| | | | | WAKG | | |
| R.1B11 | 43 | SYHMS | 44 | IIYADTGNADYA | 45 | GPTNTYPTYFAL |
| | | | | SWTKG | | |
| Hu1D15E11- | 46 | SYVMH | 47 | YIDPYNEGTKYN | 48 | GGMARRSFDY |
| V1 | | | | EKFKG | | |
| Hu1D15E11- | 49 | SYVMH | 50 | YIDPYNEGTKYN | 51 | GGMARRSFDY |
| V2 | | | | EKFKG | | |
| hu18B9.3i- | 52 | SGYMS | 53 | SIYAGSSGKTYY | 54 | DSGGTYYTDYLY |
| V1 | | | | ADWAKG | | A |
| hu18B9.3i- | 55 | SGYMS | 56 | SIYAGSSGKTYY | 57 | DSGGTYYTDYLY |
| V2 | | | | ADWAKG | | L |
| hu18B9.3i- | 58 | SGYMS | 59 | SIYAGSSGKTYY | 60 | DSGGTYYTDYLY |
| V3 | | | | ADWAKG | | L |

In some embodiments, the CD47 binding domain comprises three light chain (LC) complementarity determining regions (CDRs), the LC-CDR1, the LC-CDR2, and the LC-CDR3; wherein the LC-CDR1, the LC-CDR2, and the LC-CDR3 of the CD47 binding domain comprises amino acid sequences selected from those set forth in Table 2; and wherein at least one of the LC-CDR1, the LC-CDR2, and the LC-CDR3 comprise from 0-3 amino acid modification(s). In some embodiments, at least one of the LC-CDR1, the LC-CDR2, and the LC-CDR3 of the CD47-binding domain comprise from 0-2 amino acid modification(s). In some embodiments, at least one of the LC-CDR1, the LC-CDR2, and the LC-CDR3 of the CD47-binding domain comprise from 0-1 amino acid modification(s).

In some embodiments, the LC-CDR1, the LC-CDR2, and the LC-CDR3 of the CD47 binding domain comprise the amino acid sequences of SEQ ID NOs: 61-63; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 64-66; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 67-69; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 70-72; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 73-75; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 76-78; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 79-81; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 82-84; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 85-87; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 88-90; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 91-93; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 94-96; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 97-99; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 100-102; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 103-105; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 106-108; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 109-111; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 112-114; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 115-117; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 118-120; and wherein at least one of the LC-CDR1, the LC-CDR2, and the LC-CDR3 comprise from 0-3 (e.g., 0-2, or 0-1) amino acid modification(s).

TABLE 2
Example CDR sequences of the anti-CD47 light chain binding
domains, as determined according to Kabat nomenclature.

| | SEQ ID | | SEQ ID | | SEQ ID | |
|---|---|---|---|---|---|---|
| Construct | NO | CDR1 | NO | CDR2 | NO | CDR3 |
| VIR47 | 61 | SGDALPKKYAY | 62 | EDNKRPS | 63 | YSTDISGNHWV |
| M.1D15E11 | 64 | LASQTIGTWLT | 65 | AATTLAD | 66 | QQLYSTPWT |
| M.20B10E9 | 67 | HASQNIHVWLS | 68 | KASNLHT | 69 | QQGQSYPYT |
| M.69H1H9 | 70 | RASQGIRGNLD | 71 | STSNLNS | 72 | LQRNAFPWT |
| M.30A5C6 | 73 | HASQNINVWLS | 74 | KASNLHT | 75 | QQGQNYPYT |
| M.95D4A11 | 76 | RASGNIHNYLA | 77 | NAKTLAD | 78 | QHFWSTPPT |
| R.1H3 | 79 | QASQSISNECS | 80 | RASTLES | 81 | QSTYYGTTGNT |
| R.9B11 | 82 | QASQSINNLLA | 83 | DASDLAS | 84 | QGYFYGSSVDLV |
| R.13A2 | 85 | QASQSISNELS | 86 | RASTLES | 87 | QCTYITYGNA |
| R.14G4 | 88 | QSSESVYRDNCL | 89 | KASTLAS | 90 | AGYKSTITDGTA |
| | | G | | | | |
| R.15G10 | 91 | QASRSITSYLA | 92 | AASNVAS | 93 | QSYYAISTNT |
| R.18B9 | 94 | QASEGISSYLS | 95 | KASTLTS | 96 | QCTYGSSGSSGY |
| | | | | | | GAA |
| R.19H7 | 97 | QSSESVYRDNCL | 98 | RASTLAS | 99 | AGYKSTITDGTA |
| | | G | | | | |
| R.20H1 | 100 | QASQSISNELS | 101 | RASTLES | 102 | QSTYYGTSYGNA |
| R.1B11 | 103 | QASQSITTYLA | 104 | GASNLES | 105 | QSYYAISKSA |
| Hu1D15E11- | 106 | LASQTIGTWLT | 107 | AATTLAE | 108 | QQLYSTPWT |
| V1 | | | | | | |
| Hu1D15E11- | 109 | LASQTIGTWLT | 110 | AATTLAE | 111 | QQLYSTPWT |
| V2 | | | | | | |
| hu18B9.3i- | 112 | RASEGISSYLS | 113 | KASTLTS | 114 | QSTYGSSGSSGY |
| V1 | | | | | | GAA |
| hu18B9.3i- | 115 | RASEGISSYLS | 116 | KASTLTS | 117 | QSAYGSSGSSGY |
| V2 | | | | | | GAA |
| hu18B9.3i- | 118 | RASEGISSYLS | 119 | KASTLTS | 120 | QSTYGSSGSSGY |
| V3 | | | | | | AAA |

In some embodiments, the HC-CDR1, the HC-CDR2, and the HC-CDR3 of the CD47 binding domain comprise the amino acid sequences of SEQ ID NOs: 1-3; and the LC-CDR1, the LC-CDR2, and the LC-CDR3 of the CD47 binding domain comprise the amino acid sequences of SEQ ID NOs: 61-63; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 4-6; and the LC-CDR1, the LC-CDR2, and the LC-CDR3 of the CD47 binding domain comprise the amino acid sequences of SEQ ID NOs: 64-66; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 7-9; and the LC-CDR1, the LC-CDR2, and the LC-CDR3 of the CD47 binding domain comprise the amino acid sequences of SEQ ID NOs: 67-69; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 10-12; and the LC-CDR1, the LC-CDR2, and the LC-CDR3 of the CD47 binding domain comprise the amino acid sequences of SEQ ID NOs: 70-72; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 13-15; and the LC-CDR1, the LC-CDR2, and the LC-CDR3 of the CD47 binding domain comprise the amino acid sequences of SEQ ID NOs: 73-75; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 16-18; and the LC-CDR1, the LC-CDR2, and the LC-CDR3 of the CD47 binding domain comprise the amino acid sequences of SEQ ID NOs: 76-78; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 19-21; and the LC-CDR1, the LC-CDR2, and the LC-CDR3 of the CD47 binding domain comprise the amino acid sequences of SEQ ID NOs: 79-81; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 22-24; and the LC-CDR1, the LC-CDR2, and the LC-CDR3 of the CD47 binding domain comprise the amino acid sequences of SEQ ID NOs: 82-84; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 25-27; and the LC-CDR1, the LC-CDR2, and the LC-CDR3 of the CD47 binding domain comprise the amino acid sequences of SEQ ID NOs: 85-87; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 28-30; and the LC-CDR1, the LC-CDR2, and the LC-CDR3 of the CD47 binding domain comprise the amino acid sequences of SEQ ID NOs: 88-90; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 31-33; and the LC-CDR1, the LC-CDR2, and the LC-CDR3 of the CD47 binding domain comprise the amino acid sequences of SEQ ID NOs: 91-93; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 34-36; and the LC-CDR1, the LC-CDR2, and the LC-CDR3 of the CD47 binding domain comprise the amino acid sequences of SEQ ID NOs: 94-96; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 37-39; and the LC-CDR1, the LC-CDR2, and the LC-CDR3 of the CD47 binding domain comprise the amino acid sequences of SEQ ID NOs: 97-99; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 40-42; and the LC-CDR1, the LC-CDR2, and the LC-CDR3 of the CD47 binding domain comprise the amino acid sequences of SEQ ID NOs: 100-102; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 43-45; and the LC-CDR1, the LC-CDR2, and the LC-CDR3 of the CD47 binding domain comprise the amino acid sequences of SEQ ID NOs: 103-105; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 46-48; and the LC-CDR1, the LC-CDR2, and the LC-CDR3 of the CD47 binding domain comprise the amino acid sequences of SEQ ID NOs: 106-108; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 49-51; and the LC-CDR1, the LC-CDR2, and the LC-CDR3 of the CD47 binding domain comprise the amino acid sequences of SEQ ID NOs: 109-111; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 52-54; and the LC-CDR1, the LC-CDR2, and the LC-CDR3 of the CD47 binding domain comprise the amino acid sequences of SEQ ID NOs: 112-114; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 55-57; and the LC-CDR1, the LC-CDR2, and the LC-CDR3 of the CD47 binding domain comprise the amino acid sequences of SEQ ID NOs: 115-117; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 58-60; and the LC-CDR1, the LC-CDR2, and the LC-CDR3 of the CD47 binding domain comprise the amino acid sequences of SEQ ID NOs: 118-120; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the CD47 binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 3.

In some embodiments, the CD47 binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 121-143.

In some embodiments, the CD47 binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 4.

In some embodiments, the CD47 binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 144-164.

In some embodiments, the CD47 binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 3; and a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 4.

In some embodiments, the CD47 binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 121-143; and a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 144-164.

In some embodiments, the CD47 binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 5.

In some embodiments, the CD47 binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 165-188.

In some embodiments, the CD47 binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 6.

In some embodiments, the CD47 binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 189-212.

In some embodiments, the CD47 binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 5; and a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 6.

In some embodiments, the CD47 binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 165-188; and a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 189-212.

In some embodiments, the CD47 binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 7.

In some embodiments, the CD47 binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 213.

TABLE 3
Example variable domain sequences of the anti-CD47
heavy chain binding domains.

| Construct | Amino Acid Sequence | SEQ |
|---|---|---|
| Description | (N to C) | ID NO |
| VIR47 | EVQLLESGGGLVQPGGSLRLSCAASGFTFSNFAMSWVRQAPGKGLEW | 121 |
| hIgG4-SPL | VSTISASGGRTFYADSVKGRITISRDNSKNTLFLQMNGLRAEDTAVYY | |
| VH | CAKEGSFGEGVDPWGQGTLVTVSS | |
| VIR47.V7 | EVQLLESGGGLVQPGGSLRLSCAASGFTFSNFAMSWVRQAPGKGLEW | 122 |
| hIgG1 | VSTISASGGRTFYADSVKGRITISRDNSKNTLYLQMNSLRAEDTAVYY | |
| VH | CAKEGSFGEGVDPWGQGTLVTVSS | |
| VIR47.V8 | EVQLLESGGGLVQPGGSLRLSCAASGFTFSNFAMSWVRQAPGKGLEW | 123 |
| hIgG1 | VSTISASGGRTFYADSVKGRFTISRDNSKNTLYLQMNSLRAEDTAVY | |
| VH | YCAKEGSFGEGVDPWGQGTLVTVSS | |
| M.1D15E11-VH | EVQLQQSGPELVKPGAPVKMSCKASGYTFTSYVMHWVKQKPGQGLE | 124 |
| | WIGYIDPYNDGTKYNEKFKGKATLTSDKSSSTAYMELSSLTSEDSAV | |
| | YYCSRGGMARRSFDYWGQGTTLTVSS | |
| M.20B10E9-VH | EVQLQQSGAELVRPGASVKLSCTASGFNIEDTYMHWVKQRPEQGLEW | 125 |
| | IGRIDPANGKTKSDPKFQGKATITTDTSSNTAYLQLSSLTSEDTAVYY | |
| | CAKGDGYYVGGLDYWGQGTTLTVSS | |
| M.69H1H9-VH | EVQLQQSGPELVKPGASVKMSCKTSGYTFTEYTIHWVKQSHEKSLEW | 126 |
| | IGSINPNNGLFRYNQKFKGKATLTVDKSSSTAYMELRSLTSEDSAVYY | |
| | CARDYGFGYSDYWGQGTTLTVSS | |
| M.30A5C6-VH | EVQLQQSGAELVKPGASVKLSCTASGFNIEDTYMHWVKQRPEQGLE | 127 |
| | WIGRIDPANGNTKYDPKFQGKATVTADTSSNTAYLQLSSLTSEDTAV | |
| | YCCARGDGHYVGGLNYWGQGTSLTVSS | |
| M.95D4A11-VH | QVQLQQPGAELVRPGASVKLSCKASGYTFTSYWMNWVKQRPEQGLE | 128 |
| | WIGSIDPYDSETHYNQKFKDKAILTVDKSSSTAYMQLSSLTSEDSAVY | |
| | YCARGRGYAMDYWGQGTSVTVSS | |
| R.1H3-VH | QSLEESGGDLVKPGASLTLTCTASGFSFSSGYYMCWVRQAPGKGLEW | 129 |
| | IACIYMRSGDTYLASWAKGQFTISKTSSTTVTLQMTSLTAADTATYFC | |
| | GRGGTSYALNLWGQGTLVTVSS | |
| R.9B11-VH | QEQLEESGGDLVKPEGSLTLTCTASGFSFSSYWMAWVRQAPGKGLEW | 130 |
| | IACIYIGSGSGTTYYASWAKGRFTISKTSSTTVTLQMTSLTAADTAIY | |
| | FCARWAGSDGHFTLWGPGTLVTVS | |
| R.13A2-VH | QSLEESGGDLVKPGASLTVTCTASGFSFSSYYYVCWVRQAPGKGLEWI | 131 |
| | ACIYAGSSGDTYYASWAKGRFTISKTSSTTVTLQMTSLTAADTATYFC | |
| | ARGYTGYSYQFNFWGPGTLVTVSS | |
| R.14G4-VH | QEQLKESGGGLVTPGGTLTLTCTASGFTITSYHMCWVRQAPGKGLEW | 132 |
| | IGCIYPNNGFTWYASWAKGRFTISKTSSTTVDLKMTSPTTEEMATYFC | |
| | ARGSGNSYNQLDLWGQGTLVTVSS | |
| R.15G10-VH | QSLEESGGRLVTPGTPLTLTCTASGFTISSYHMNWVRQAPGEGLEWIG | 133 |
| | IIYVGSGNADYASWAKGRFTISKASTTVDLKITSPTTEDTATYFCARG | |
| | PTNTYPTYFNLWGPGTLVTVSS | |
| R.18B9-VH | QSLEESGGDLVKPGASLTLTCKASGFSFNSGYMCWVRQAPGKGLEWI | 134 |
| | ACIYAGSSGKTYYASWATGRFTISKTSSTTVTLQMTSLTVADTATYFC | |
| | ARDDGGTYYTDYLYLWGPGTLVTVSS | |
| R.19H7-VH | QEQLKESGGGLVTPGGTLTLTCTASGFTISSYHMCWVRQAPGKGLEWI | 135 |
| | GCIYPNNDFTWYATWAKGRFTISKTSSTTVDLRMTTPTTEETATYFC | |
| | ARASGNAYNLLDLWGQGTLVTVSS | |
| R.20H1-VH | QEQLVESGGGLVKPGASLTLTCKASGFSFSSGYYMCWVRQAPGKGLE | 136 |
| | WIGCIYVRSDSTYYASWAKGRFTISKASSTTVTLQMTSLTAADTATYF | |
| | CARGVTGDSYRFNFWGPGTLVTVSS | |
| R.1B11-VH | QSLEESGGRLVTPGTPLTLTCTASGFSLNSYHMSWVRQAPGEGLEWIG | 137 |
| | IIYADTGNADYASWTKGRFTISKASTTVELRITSPTSEDTATYFCARG | |
| | PTNTYPTYFALWGPGTLVTVSS | |
| Hu1D15E11-V1 VH | EVQLVQSGAEVKKPGASVKVSCKASGYTFTSYVMHWVRQAPGQRLE | 138 |
| | WMGYIDPYNEGTKYNEKFKGRVTITRDTSASTAYMELSSLRSEDTAV | |
| | YYCSRGGMARRSFDYWGQGTLVTVSS | |
| Hu1D15E11-V2 VH | EVQLVQSGAEVKKPGASVKVSCKASGYTFTSYVMHWVRQAPGQRLE | 139 |
| | WMGYIDPYNEGTKYNEKFKGRVTITSDTSASTAYMELSSLRSEDTAV | |
| | YYCSRGGMARRSFDYWGQGTLVTVSS | |
| hu18B9.3i-V1 VH | EVQLVESGGGLVQPGGSLRLSCAASGFSFNSGYMSWVRQAPGKGLEW | 140 |
| | IASIYAGSSGKTYYADWAKGRFTISKTSSKTTVYLQMNSLRAEDTAV | |
| | YYCARDSGGTYYTDYLYAWGQGTLVTVSS | |
| hu18B9.3i-V2 VH | EVQLVESGGGLVQPGGSLRLSCAASGFSFNSGYMSWVRQAPGKGLEW | 141 |
| | IASIYAGSSGKTYYADWAKGRFTISKTSSKTTVYLQMNSLRAEDTAV | |
| | YYCARDSGGTYYTDYLYLWGQGTLVTVSS | |
| hu18B9.3i-V3 VH | EVQLVESGGGLVQPGGSLRLSCAASGFSFNSGYMSWVRQAPGKGLEW | 142 |
| | IASIYAGSSGKTYYADWAKGRFTISKTSSKTTVYLQMNSLRAEDTAV | |
| | YYCARDSGGTYYTDYLYLWGQGTLVTVSS | |
| VIR47.V8 HCV | EVQLLESGGGLVQPGGSLRLSCAASGFTFSNFAMSWVRQAPGKGLEW | 143 |
| | VSTISASGGRTFYADSVKGRFTISRDNSKNTLYLQMNSLRAEDTAVY | |
| | YCAKEGSFGEGVDPWGQGTLVTVL | |

CDRs, as determined according to Kabat nomenclature, are underlined and bolded.

TABLE 4
Example variable domain sequences of the anti-CD47
light chain binding domains.

| Construct | Amino Acid Sequence | SEQ |
|---|---|---|
| Description | (N to C) | ID NO |
| VIR47.V8-VL | SYELTQPPSVSVSPGQTARITCSGDALPKKYAYWYQQKSGQAPVLVIY | 144 |
| | EDNKRPSGIPERFSGSSSGTMATLTISGAQVEDEADYYCYSTDISGNH | |
| | WVFGGGTKLTVL | |
| M.1D15E11-VL | DIQMTQSPASQSASLGESVTITCLASQTIGTWLTWYQQKPGKSPQLLI | 145 |
| | YAATTLADGVPSRFSGSGSGTKFSFKISSLQAEDFVSYYCQQLYSTPW | |
| | TFGGGTKLEIK | |
| M.20B10E9-VL | DIQMNQSPSSLSASLGDTITITCHASQNIHVWLSWYQQKPGNIPKLLIY | 146 |
| | KASNLHTGVPSRFSGSGSGTGFTLTISSLQPEDIATYYCQQGQSYPYTF | |
| | GGGTKLEIK | |
| M.69H1H9-VL | DIQMIQSPSSMFASLGDRVSLSCRASQGIRGNLDWYQQKPGGTIKLLI | 147 |
| | YSTSNLNSGVPSRFSGSGSGSDYSLTISSLESEDFADYYCLQRNAFPWT | |
| | FGGGTKLEIK | |
| M.30A5C6-VL | DIQMNQSPSSLSASLGDTITITCHASQNINVWLSWYQQKPGNIPKLLIY | 148 |
| | KASNLHTGVPSRFSGSGSGTGFTLTISSLQPEDIATYYCQQGQNYPYTF | |
| | GGGTKLEIK | |
| M.95D4A11-VL | DIQMTQSPASLSASVGETVTITCRASGNIHNYLAWYQQKQGKSPQLLV | 149 |
| | YNAKTLADGVPSRFSGSGSGTQYSLKINSLQPEDFGSYYCQHFWSTPP | |
| | TFGGGTKLEIK | |
| R.1H3-VL | DVVMTQTPASVSEPVGGTVTINCQASQSISNECSWYQQKPGQPPKLLI | 150 |
| | YRASTLESGVPSRFSGSGSGTEFTLTISDLESADAATYYCQSTYYGTT | |
| | GNTFGGGTEVVVK | |
| R.9B11-VL | ALVMTQTPASVEAAVGGTVTINCQASQSINNLLA WYQQKPGQRPKLL | 151 |
| | IYDASDLASGVSSRFKGSGSGTEYTLTISGVQSADAATYYCQGYFYGS | |
| | SVDLVFGGGTEVVVK | |
| R.13A2-VL | DVVMTQTPASVEAAVGGTVTIKCQASQSISNELSWYQQKPGQPPKLLI | 152 |
| | YRASTLESGVPSRFKGSGSGTEFTLTISDLESADAATYYCQCTYITYG | |
| | NAFGGGTEVVVK | |
| R.14G4-VL | AIVMTQTPASVSATLGGSVTINCQSSESVYRDNCLGWFQQKPGQPPKL | 153 |
| | LIYKASTLASGVPSRFKGSGSGTQFTLTISDVVSDDAATYYCAGYKSTI | |
| | TDGTAFGGGTEVVVK | |
| R.15G10-VL | AFELTQTPSSVEAAVGGTVTIKCQASRSITSYLAWYQHKPGQPPTLLIY | 154 |
| | AASNVASGVSSRFKGSRSGTEYTLTISDLESADAATYYCQSYYAISTNT | |
| | FGGGTEVVVK | |
| R. 18B9-VL | DVVMTQTPASVEAAVGGTVTIKCQASEGISSYLSWYQQKPGQPPKLLI | 155 |
| | YKASTLTSGVPSRFKGSGSGTDFTLTISDLESADAATYYCQCTYGSSG | |
| | SSGYGAAFGGGTEVVVK | |
| R.19H7-VL | AIVMTQTPASVSVAVGGSVTINCQSSESVYRDNCLGWFQQKPGQPPK | 156 |
| | LLIYRASTLASGVPSRFKGSGSGTQFTLTISDVVSDDAATYYCAGYKS | |
| | TITDGTAFGGGTEVVVK | |
| R.20H1-VL | DVVMTQTPASVEAAVGGTVTIKCQASQSISNELSWYQQKSGQPPKLLI | 157 |
| | YRASTLESGVPSRFKGSGSGTEFTLTISDLESADAATYYCQSTYYGTS | |
| | YGNAFGGGTEVVVK | |
| R. 1B11-VL | AFELTQTPSSVEAAVGGTVTIKCQASQSITTYLAWYQQKPGQPPKLLI | 158 |
| | YGASNLESGVPSRFKGSRSGTEYTLTISDLESADAATYYCQSYYAISKS | |
| | AFGGGTEVVVK | |
| Hu1D15E11-V1 | DIQMTQSPSSLSASVGDRVTITCLASQTIGTWLTWYQQKPGKAPKLLI | 159 |
| VL | YAATTLAEGVPSRFSGSGSGTDFTLTISSLQPEDFATYYCQQLYSTPW | |
| | TFGGGTKVEIK | |
| Hu1D15E11-V2 | DIQMTQSPSSLSASVGDRVTITCLASQTIGTWLTWYQQKPGKAPKLLI | 160 |
| VL | YAATTLAEGVPSRFSGSGSGTDFTLTISSLQPEDFATYYCQQLYSTPW | |
| | TFGGGTKVEIK | |
| hu18B9.3i-V1 | DIQMTQSPSSLSASVGDRVTITCRASEGISSYLSWYQQKPGKAPKLLIY | 161 |
| VL | KASTLTSGVPSRFSGSGSGTDFTLTISSLQPEDFATYYCQSTYGSSGSS | |
| | GYGAAFGGGTKVEIK | |
| hu18B9.3i-V2 | DIQMTQSPSSLSASVGDRVTITCRASEGISSYLSWYQQKPGKAPKLLIY | 162 |
| VL | KASTLTSGVPSRFSGSGSGTDFTLTISSLQPEDFATYYCQSAYGSSGSS | |
| | GYGAAFGGGTKVEIK | |
| hu18B9.3i-V3 | DIQMTQSPSSLSASVGDRVTITCRASEGISSYLSWYQQKPGKAPKLLIY | 163 |
| VL | KASTLTSGVPSRFSGSGSGTDFTLTISSLQPEDFATYYCQSTYGSSGSS | |
| | GYAAAFGGGTKVEIK | |
| VIR47.V8 LCV | SYELTQPPSVSVSPGQTARITCSGDALPKKYAYWYQQKSGQAPVLVIY | 164 |
| | EDNKRPSGIPERFSGSSSGTMATLTISGAQVEDEADYYCYSTDISGNH | |
| | WVFGGGTKLTVL | |

CDRs, as determined according to Kabat nomenclature, are underlined and bolded.

TABLE 5
Example full-length heavy chain sequences of the
anti-CD47 binding domains.

| Construct | Amino Acid Sequence | SEQ |
|---|---|---|
| Description | (N to C) | ID NO |
| VIR47 | EVQLLESGGGLVQPGGSLRLSCAASGFTFSNFAMSWVRQAPGKGLEWVSTIS | 165 |
| hIgG4-SPL | ASGGRTFYADSVKGRITISRDNSKNTLFLQMNGLRAEDTAVYYCAKEGSFGE | |
| HC | GVDPWGQGTLVTVSSASTKGPSVFPLAPCSRSTSESTAALGCLVKDYFPEPVT | |
| | VSWNSGALTSGVHTFPAVLQSSGLYSLSSVVTVPSSSLGTKTYTCNVDHKPSN | |
| | TKVDKRVESKYGPPCPPCPAPEFLGGPSVFLFPPKPKDTLMISRTPEVTCVVVD | |
| | VSQEDPEVQFNWYVDGVEVHNAKTKPREEQFNSTYRVVSVLTVLHQDWLNG | |
| | KEYKCKVSNKGLPSSIEKTISKAKGQPREPQVYTLPPSQEEMTKNQVSLTCLV | |
| | KGFYPSDIAVEWESNGQPENNYKTTPPVLDSDGSFFLYSRLTVDKSR WQEGN | |
| | VFSCSVMHEALHNHYTQKSLSLSLGK | |
| VIR47 | EVQLLESGGGLVQPGGSLRLSCAASGFTFSNFAMSWVRQAPGKGLEWVSTIS | 166 |
| hIgG1 | ASGGRTFYADSVKGRITISRDNSKNTLFLQMNGLRAEDTAVYYCAKEGSFGE | |
| HC | GVDPWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMISRTPEVTCVV | |
| | VDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVDKSRWQQ | |
| | GNVFSCSVMHEALHNHYTQKSLSLSPGK | |
| VIR47.V7 | EVQLLESGGGLVQPGGSLRLSCAASGFTFSNFAMSWVRQAPGKGLEWVSTIS | 167 |
| hIgG1 | ASGGRTFYADSVKGRITISRDNSKNTLYLQMNSLRAEDTAVYYCAKEGSFGE | |
| HC | GVDPWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMISRTPEVTCVV | |
| | VDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVDKSRWQQ | |
| | GNVFSCSVMHEALHNHYTQKSLSLSPGK | |
| VIR47.V8 | EVQLLESGGGLVQPGGSLRLSCAASGFTFSNFAMSWVRQAPGKGLEWVSTIS | 168 |
| hIgG1 | ASGGRTFYADSVKGRFTISRDNSKNTLYLQMNSLRAEDTAVYYCAKEGSFG | |
| HC | EGVDPWGQGTLVTVSSASTKGPSVFPLAPSSKSTSGGTAALGCLVKDYFPEPV | |
| | TVSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| M.1D15E11_ | EVQLQQSGPELVKPGAPVKMSCKASGYTFTSYVMHWVKQKPGQGLEWIGYI | 169 |
| HC | DPYNDGTKYNEKFKGKATLTSDKSSSTAYMELSSLTSEDSAVYYCSRGGMA | |
| | RRSFDYWGQGTTLTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| M.20B10E9_ | EVQLQQSGAELVRPGASVKLSCTASGFNIEDTYMHWVKQRPEQGLEWIGRID | 170 |
| HC | PANGKTKSDPKFQGKATITTDTSSNTAYLQLSSLTSEDTAVYYCAKGDGYY | |
| | VGGLDYWGQGTTLTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| M.69H1H9_ | EVQLQQSGPELVKPGASVKMSCKTSGYTFTEYTIHWVKQSHEKSLEWIGSINP | 171 |
| HC | NNGLFRYNQKFKGKATLTVDKSSSTAYMELRSLTSEDSAVYYCARDYGFGY | |
| | SDYWGQGTTLTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKKVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWL | |
| | NGKEYKCKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKSRWQQG | |
| | NVFSCSVMHEALHNHYTQKSLSLSPGK | |
| M.30A5C6_ | EVQLQQSGAELVKPGASVKLSCTASGFNIEDTYMHWVKQRPEQGLEWIGRID | 172 |
| HC | PANGNTKYDPKFQGKATVTADTSSNTAYLQLSSLTSEDTAVYCCARGDGHY | |
| | VGGLNYWGQGTSLTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| M.95D4A11_ | QVQLQQPGAELVRPGASVKLSCKASGYTFTSYWMNWVKQRPEQGLEWIGSI | 173 |
| HC | DPYDSETHYNQKFKDKAILTVDKSSSTAYMQLSSLTSEDSAVYYCARGRGY | |
| | AMDYWGQGTSVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMISRTPEVTCVV | |
| | VDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVDKSRWQQ | |
| | GNVFSCSVMHEALHNHYTQKSLSLSPGK | |
| R.1H3_HC | QSLEESGGDLVKPGASLTLTCTASGFSFSSGYYMCWVRQAPGKGLEWIACIY | 174 |
| | MRSGDTYLASWAKGQFTISKTSSTTVTLQMTSLTAADTATYFCGRGGTSYA | |
| | LNLWGQGTLVTVSSASTKGPSVFPLAPSSKSTSGGTAALGCLVKDYFPEPVTV | |
| | SWNSGALTSGVHTFPAVLQSSGLYSLSSVVTVPSSSLGTQTYICNVNHKPSNTK | |
| | VDKKVEPKSCDKTHTCPPCPAPELLGGPSVFLFPPKPKDTLMISRTPEVTCVVV | |
| | DVSHEDPEVKFNWYVDGVEVHNAKTKPREEQYNSTYRVVSVLTVLHQDWL | |
| | NGKEYKCKVSNKALPAPIEKTISKAKGQPREPQVYTLPPSRDELTKNQVSLTCL | |
| | VKGFYPSDIAVEWESNGQPENNYKTTPPVLDSDGSFFLYSKLTVDKSRWQQG | |
| | NVFSCSVMHEALHNHYTQKSLSLSPGK | |
| R.9B11_HC | QEQLEESGGDLVKPEGSLTLTCTASGFSFSSYWMAWVRQAPGKGLEWIACIY | 175 |
| | IGSGSGTTYYASWAKGRFTISKTSSTTVTLQMTSLTAADTAIYFCARWAGSD | |
| | GHFTLWGPGTLVTVSLASTKGPSVFPLAPSSKSTSGGTAALGCLVKDYFPEPV | |
| | TVSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| R.13A2_HC | QSLEESGGDLVKPGASLTVTCTASGFSFSSYYYVCWVRQAPGKGLEWIACIY | 176 |
| | AGSSGDTYYASWAKGRFTISKTSSTTVTLQMTSLTAADTATYFCARGYTGYS | |
| | YQFNFWGPGTLVTVSSASTKGPSVFPLAPSSKSTSGGTAALGCLVKDYFPEPV | |
| | TVSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| R.14G4_HC | QEQLKESGGGLVTPGGTLTLTCTASGFTITSYHMCWVRQAPGKGLEWIGCIY | 177 |
| | PNNGFTWYASWAKGRFTISKTSSTTVDLKMTSPTTEEMATYFCARGSGNSY | |
| | NQLDLWGQGTLVTVSSASTKGPSVFPLAPSSKSTSGGTAALGCLVKDYFPEPV | |
| | TVSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| R.15G10_HC | QSLEESGGRLVTPGTPLTLTCTASGFTISSYHMNWVRQAPGEGLEWIGIIYVGS | 178 |
| | GNADYASWAKGRFTISKASTTVDLKITSPTTEDTATYFCARGPTNTYPTYFN | |
| | LWGPGTLVTVSSASTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVDV | |
| | SHEDPEVKFNWYVDGVEVHNAKTKPREEQYNSTYRVVSVLTVLHQDWLNG | |
| | KEYKCKVSNKALPAPIEKTISKAKGQPREPQVYTLPPSRDELTKNQVSLTCL VK | |
| | GFYPSDIAVEWESNGQPENNYKTTPPVLDSDGSFFLYSKLTVDKSRWQQGNV | |
| | FSCSVMHEALHNHYTQKSLSLSPGK | |
| R.18B9_HC | QSLEESGGDLVKPGASLTLTCKASGFSFNSGYMCWVRQAPGKGLEWIACIYA | 179 |
| | GSSGKTYYASWATGRFTISKTSSTTVTLQMTSLTVADTATYFCARDDGGTYY | |
| | TDYLYLWGPGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| R.19H7_HC | QEQLKESGGGLVTPGGTLTLTCTASGFTISSYHMCWVRQAPGKGLEWIGCIY | 180 |
| | PNNDFTWYATWAKGRFTISKTSSTTVDLRMTTPTTEETATYFCARASGNAYN | |
| | LLDLWGQGTLVTVSSASTKGPSVFPLAPSSKSTSGGTAALGCLVKDYFPEPVT | |
| | VSWNSGALTSGVHTFPAVLQSSGLYSLSSVVTVPSSSLGTQTYICNVNHKPSNT | |
| | KVDKKVEPKSCDKTHTCPPCPAPELLGGPSVFLFPPKPKDTLMISRTPEVTCVV | |
| | VDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVDKSRWQQ | |
| | GNVFSCSVMHEALHNHYTQKSLSLSPGK | |
| R.20H1_HC | QEQLVESGGGLVKPGASLTLTCKASGFSFSSGYYMCWVRQAPGKGLEWIGCI | 181 |
| | YVRSDSTYYASWAKGRFTISKASSTTVTLQMTSLTAADTATYFCARGVTGDS | |
| | YRFNFWGPGTLVTVSSASTKGPSVFPLAPSSKSTSGGTAALGCLVKDYFPEPV | |
| | TVSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| R.1B11_HC | QSLEESGGRLVTPGTPLTLTCTASGFSLNSYHMSWVRQAPGEGLEWIGIIYAD | 182 |
| | TGNADYASWTKGRFTISKASTTVELRITSPTSEDTATYFCARGPTNTYPTYFA | |
| | LWGPGTLVTVSSASTKGPSVFPLAPSSKSTSGGTAALGCLVKDYFPEPVTVSW | |
| | NSGALTSGVHTFPAVLQSSGLYSLSSVVTVPSSSLGTQTYICNVNHKPSNTKVD | |
| | KKVEPKSCDKTHTCPPCPAPELLGGPSVFLFPPKPKDTLMISRTPEVTCVVVDV | |
| | SHEDPEVKFNWYVDGVEVHNAKTKPREEQYNSTYRVVSVLTVLHQDWLNG | |
| | KEYKCKVSNKALPAPIEKTISKAKGQPREPQVYTLPPSRDELTKNQVSLTCLVK | |
| | GFYPSDIAVEWESNGQPENNYKTTPPVLDSDGSFFLYSKLTVDKSRWQQGNV | |
| | FSCSVMHEALHNHYTQKSLSLSPGK | |
| Hu1D15E11- | EVQLVQSGAEVKKPGASVKVSCKASGYTFTSYVMHWVRQAPGQRLEWMGY | 183 |
| V1_HC | IDPYNEGTKYNEKFKGRVTITRDTSASTAYMELSSLRSEDTAVYYCSRGGM | |
| | ARRSFDYWGQGTLVTVSSASTKGPSVFPLAPSSKSTSGGTAALGCLVKDYFPE | |
| | PVTVSWNSGALTSGVHTFPAVLQSSGLYSLSSVVTVPSSSLGTQTYICNVNHKP | |
| | SNTKVDKKVEPKSCDKTHTCPPCPAPELLGGPSVFLFPPKPKDTLMISRTPEVT | |
| | CVVVDVSHEDPEVKFNWYVDGVEVHNAKTKPREEQYNSTYRVVSVLTVLHQ | |
| | DWLNGKEYKCKVSNKALPAPIEKTISKAKGQPREPQVYTLPPSRDELTKNQVS | |
| | LTCLVKGFYPSDIAVEWESNGQPENNYKTTPPVLDSDGSFFLYSKLTVDKSRW | |
| | QQGNVFSCSVMHEALHNHYTQKSLSLSPGK | |
| Hu1D15E11- | EVQLVQSGAEVKKPGASVKVSCKASGYTFTSYVMHWVRQAPGQRLEWMGY | 184 |
| V2_HC | IDPYNEGTKYNEKFKGRVTITSDTSASTAYMELSSLRSEDTAVYYCSRGGMA | |
| | RRSFDYWGQGTLVTVSSASTKGPSVFPLAPSSKSTSGGTAALGCLVKDYFPEP | |
| | VTVSWNSGALTSGVHTFPAVLQSSGLYSLSSVVTVPSSSLGTQTYICNVNHKPS | |
| | NTKVDKKVEPKSCDKTHTCPPCPAPELLGGPSVFLFPPKPKDTLMISRTPEVTC | |
| | VVVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | TCLVKGFYPSDIAVEWESNGQPENNYKTTPPVLDSDGSFFLYSKLTVDKSRWQ | |
| | QGNVFSCSVMHEALHNHYTQKSLSLSPGK | |
| hu18B9.3i-V1 | EVQLVESGGGLVQPGGSLRLSCAASGFSFNSGYMSWVRQAPGKGLEWIASIY | 185 |
| HC | AGSSGKTYYADWAKGRFTISKTSSKTTVYLQMNSLRAEDTAVYYCARDSGG | |
| | TYYTDYLYAWGQGTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYICNVN | |
| | HKPSNTKVDKKVEPKSCDKTHTCPPCPAPELLGGPSVFLFPPKPKDTLMISRTP | |
| | EVTCVVVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSRDELTKN | |
| | QVSLTCLVKGFYPSDIAVEWESNGQPENNYKTTPPVLDSDGSFFLYSKLTVDK | |
| | SRWQQGNVFSCSVMHEALHNHYTQKSLSLSPGK | |
| hu18B9.3i-V2 | EVQLVESGGGLVQPGGSLRLSCAASGFSFNSGYMSWVRQAPGKGLEWIASIY | 186 |
| HC | AGSSGKTYYADWAKGRFTISKTSSKTTVYLQMNSLRAEDTAVYYCARDSGG | |
| | TYYTDYLYLWGQGTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYICNVN | |
| | HKPSNTKVDKKVEPKSCDKTHTCPPCPAPELLGGPSVFLFPPKPKDTLMISRTP | |
| | EVTCVVVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSRDELTKN | |
| | QVSLTCLVKGFYPSDIAVEWESNGQPENNYKTTPPVLDSDGSFFLYSKLTVDK | |
| | SRWQQGNVFSCSVMHEALHNHYTQKSLSLSPGK | |
| hu18B9.3i-V3 | EVQLVESGGGLVQPGGSLRLSCAASGFSFNSGYMSWVRQAPGKGLEWIASIY | 187 |
| HC | AGSSGKTYYADWAKGRFTISKTSSKTTVYLQMNSLRAEDTAVYYCARDSGG | |
| | TYYTDYLYLWGQGTLVTVSSASTKGPSVFPLAPSSKSTSGGTAALGCLVKDY | |
| | FPEPVTVSWNSGALTSGVHTFPAVLQSSGLYSLSSVVTVPSSSLGTQTYICNVN | |
| | HKPSNTKVDKKVEPKSCDKTHTCPPCPAPELLGGPSVFLFPPKPKDTLMISRTP | |
| | EVTCVVVDVSHEDPEVKFNWYVDGVEVHNAKTKPREEQYNSTYRVVSVLTV | |
| | LHQDWLNGKEYKCKVSNKALPAPIEKTISKAKGQPREPQVYTLPPSRDELTKN | |
| | QVSLTCLVKGFYPSDIAVEWESNGQPENNYKTTPPVLDSDGSFFLYSKLTVDK | |
| | SRWQQGNVFSCSVMHEALHNHYTQKSLSLSPGK | |
| CD47. | EVQLLESGGGLVQPGGSLRLSCAASGFTFSNFAMSWVRQAPGKGLEWVSTIS | 188 |
| VIR47.V8. | ASGGRTFYADSVKGRFTISRDNSKNTLYLQMNSLRAEDTAVYYCAKEGSFG | |
| hulgG1.knob. | EGVDPWGQGTLVTVSSASTKGPSVFPLAPSSKSTSGGTAALGCLVKDYFPEPV | |
| Flag | TVSWNSGALTSGVHTFPAVLQSSGLYSLSSVVTVPSSSLGTQTYICNVNHKPSN | |
| | TKVDKKVEPKSCDKTHTCPPCPAPELLGGPSVFLFPPKPKDTLMISRTPEVTCV | |
| | VVDVSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKNQVSL | |
| | WCLVKGFYPSDIAVEWESNGQPENNYKTTPPVLDSDGSFFLYSKLTVDKSRW | |
| | QQGNVFSCSVMHEALHNHYTQKSLSLSPGGGGSDYKDDDDK | |

CDRs, as determined according to Kabat nomenclature, are underlined and bolded.

TABLE 6
Example full-length light chain sequences of the anti-CD47 binding domains.
CDRs, as determined according to Kabat nomenclature,
are underlined and bolded.

| Construct | Amino Acid Sequence | SEQ |
|---|---|---|
| Description | (N to C) | ID NO |
| VIR47 | SYEMTQPPSVSVSPGQTARITCSGDALPKKYAYWYQQKSGQAPVLVIYEDNK | 189 |
| LC | RPSGIPERFSGSSSGTMATLTISGAQVEDEADYYCYSTDISGNHWVFGGGTEL | |
| | TVLGQPKAAPSVTLFPPSSEELQANKATLVCLISDFYPGAVTVAWKADSSPVK | |
| | AGVETTTPSKQSNNKYAASSYLSLTPEQWKSHRSYSCQVTHEGSTVEKTVAPT | |
| | ECS | |
| VIR47.V7 | SYEMTQPPSVSVSPGQTARITCSGDALPKKYAYWYQQKSGQAPVLVIYEDNK | 190 |
| LC | RPSGIPERFSGSSSGTMATLTISGAQVEDEADYYCYSTDISGNHWVFGGGTKL | |
| | TVLGQPKAAPSVTLFPPSSEELQANKATLVCLISDFYPGAVTVAWKADSSPVK | |
| | AGVETTTPSKQSNNKYAASSYLSLTPEQWKSHRSYSCQVTHEGSTVEKTVAPT | |
| | ECS | |
| VIR47.V8 | SYELTQPPSVSVSPGQTARITCSGDALPKKYAYWYQQKSGQAPVLVIYEDNK | 191 |
| LC | RPSGIPERFSGSSSGTMATLTISGAQVEDEADYYCYSTDISGNHWVFGGGTKL | |
| | TVLGQPKAAPSVTLFPPSSEELQANKATLVCLISDFYPGAVTVAWKADSSPVK | |
| | AGVETTTPSKQSNNKYAASSYLSLTPEQWKSHRSYSCQVTHEGSTVEKTVAPT | |
| | ECS | |
| M.1D15E11_ | DIQMTQSPASQSASLGESVTITCLASQTIGTWLTWYQQKPGKSPQLLIYAATT | 192 |
| LC | LADGVPSRFSGSGSGTKFSFKISSLQAEDFVSYYCQQLYSTPWTFGGGTKLEI | |
| | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| M.20B10E9_ | DIQMNQSPSSLSASLGDTITITCHASQNIHVWLSWYQQKPGNIPKLLIYKASNL | 193 |
| LC | HTGVPSRFSGSGSGTGFTLTISSLQPEDIATYYCQQGQSYPYTFGGGTKLEIKR | |
| | TVAAPSVFIFPPSDEQLKSGTASVVCLLNNFYPREAKVQWKVDNALQSGNSQE | |
| | SVTEQDSKDSTYSLSSTLTLSKADYEKHKVYACEVTHQGLSSPVTKSFNRGEC | |
| M.69H1H9_ | DIQMIQSPSSMFASLGDRVSLSCRASQGIRGNLDWYQQKPGGTIKLLIYSTSN | 194 |
| LC | LNSGVPSRFSGSGSGSDYSLTISSLESEDFADYYCLQRNAFPWTFGGGTKLEIK | |
| | RTVAAPSVFIFPPSDEQLKSGTASVVCLLNNFYPREAKVQWKVDNALQSGNSQ | |
| | ESVTEQDSKDSTYSLSSTLTLSKADYEKHKVYACEVTHQGLSSPVTKSFNRGE | |
| | C | |
| M.30A5C6_ | DIQMNQSPSSLSASLGDTITITCHASQNINVWLSWYQQKPGNIPKLLIYKASNL | 195 |
| LC | HTGVPSRFSGSGSGTGFTLTISSLQPEDIATYYCQQGQNYPYTFGGGTKLEIKR | |
| | TVAAPSVFIFPPSDEQLKSGTASVVCLLNNFYPREAKVQWKVDNALQSGNSQE | |
| | SVTEQDSKDSTYSLSSTLTLSKADYEKHKVYACEVTHQGLSSPVTKSFNRGEC | |
| M.95D4A11_ | DIQMTQSPASLSASVGETVTITCRASGNIHNYLAWYQQKQGKSPQLLVYNAK | 196 |
| LC | TLADGVPSRFSGSGSGTQYSLKINSLQPEDFGSYYCQHFWSTPPTFGGGTKLE | |
| | IKRTVAAPSVFIFPPSDEQLKSGTASVVCLLNNFYPREAKVQWKVDNALQSGN | |
| | SQESVTEQDSKDSTYSLSSTLTLSKADYEKHKVYACEVTHQGLSSPVTKSFNR | |
| | GEC | |
| R.1H3_LC | DVVMTQTPASVSEPVGGTVTINCQASQSISNECSWYQQKPGQPPKLLIYRAST | 197 |
| | LESGVPSRFSGSGSGTEFTLTISDLESADAATYYCQSTYYGTTGNTFGGGTEV | |
| | VVKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKSF | |
| | NRGEC | |
| R.9B11_LC | ALVMTQTPASVEAAVGGTVTINCQASQSINNLLAWYQQKPGQRPKLLIYDAS | 198 |
| | DLASGVSSRFKGSGSGTEYTLTISGVQSADAATYYCQGYFYGSSVDLVFGGG | |
| | TEVVVKRTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQGLSSPVT | |
| | KSFNRGEC | |
| R.13A2_LC | DVVMTQTPASVEAAVGGTVTIKCQASQSISNELSWYQQKPGQPPKLLIYRAS | 199 |
| | TLESGVPSRFKGSGSGTEFTLTISDLESADAATYYCQCTYITYGNAFGGGTEV | |
| | VVKRTVAAPSVFIFPPSDEQLKSGTASVVCLLNNFYPREAKVQWKVDNALQS | |
| | GNSQESVTEQDSKDSTYSLSSTLTLSKADYEKHKVYACEVTHQGLSSPVTKSF | |
| | NRGEC | |
| R.14G4_LC | AIVMTQTPASVSATLGGSVTINCQSSESVYRDNCLGWFQQKPGQPPKLLIYKA | 200 |
| | STLASGVPSRFKGSGSGTQFTLTISDVVSDDAATYYCAGYKSTITDGTAFGGG | |
| | TEVVVKRTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQGLSSPVT | |
| | KSFNRGEC | |
| R.15G10_LC | AFELTQTPSSVEAAVGGTVTIKCQASRSITSYLAWYQHKPGQPPTLLIYAASN | 201 |
| | VASGVSSRFKGSRSGTEYTLTISDLESADAATYYCQSYYAISTNTFGGGTEVV | |
| | VKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSFN | |
| | RGEC | |
| R.18B9_LC | DVVMTQTPASVEAAVGGTVTIKCQASEGISSYLSWYQQKPGQPPKLLIYKAS | 202 |
| | TLTSGVPSRFKGSGSGTDFTLTISDLESADAATYYCQCTYGSSGSSGYGAAFG | |
| | GGTEVVVKRTVAAPSVFIFPPSDEQLKSGTASVVCLLNNFYPREAKVQWKVD | |
| | NALQSGNSQESVTEQDSKDSTYSLSSTLTLSKADYEKHKVYACEVTHQGLSSP | |
| | VTKSFNRGEC | |
| R.19H7_LC | AIVMTQTPASVSVAVGGSVTINCQSSESVYRDNCLGWFQQKPGQPPKLLIYR | 203 |
| | ASTLASGVPSRFKGSGSGTQFTLTISDVVSDDAATYYCAGYKSTITDGTAFGG | |
| | GTEVVVKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSSPV | |
| | TKSFNRGEC | |
| R.20H1_LC | DVVMTQTPASVEAAVGGTVTIKCQASQSISNELSWYQQKSGQPPKLLIYRAS | 204 |
| | TLESGVPSRFKGSGSGTEFTLTISDLESADAATYYCQSTYYGTSYGNAFGGGT | |
| | EVVVKRTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGLSSPVTK | |
| | SFNRGEC | |
| R.1B11_LC | AFELTQTPSSVEAAVGGTVTIKCQASQSITTYLAWYQQKPGQPPKLLIYGASN | 205 |
| | LESGVPSRFKGSRSGTEYTLTISDLESADAATYYCQSYYAISKSAFGGGTEVV | |
| | VKRTVAAPSVFIFPPSDEQLKSGTASVVCLLNNFYPREAKVQWKVDNALQSG | |
| | NSQESVTEQDSKDSTYSLSSTLTLSKADYEKHKVYACEVTHQGLSSPVTKSFN | |
| | RGEC | |
| Hu1D15E11- | DIQMTQSPSSLSASVGDRVTITCLASQTIGTWLTWYQQKPGKAPKLLIYAATT | 206 |
| V1 LC | LAEGVPSRFSGSGSGTDFTLTISSLQPEDFATYYCQQLYSTPWTFGGGTKVEI | |
| | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| Hu1D15E11- | DIQMTQSPSSLSASVGDRVTITCLASQTIGTWLTWYQQKPGKAPKLLIYAATT | 207 |
| V2 LC | LAEGVPSRFSGSGSGTDFTLTISSLQPEDFATYYCQQLYSTPWTFGGGTKVEI | |
| | KRTVAAPSVFIFPPSDEQLKSGTASVVCLLNNFYPREAKVQWKVDNALQSGNS | |
| | QESVTEQDSKDSTYSLSSTLTLSKADYEKHKVYACEVTHQGLSSPVTKSFNRG | |
| | EC | |
| hu18B9.3i-V1 | DIQMTQSPSSLSASVGDRVTITCRASEGISSYLSWYQQKPGKAPKLLIYKASTL | 208 |
| LC | TSGVPSRFSGSGSGTDFTLTISSLQPEDFATYYCQSTYGSSGSSGYGAAFGGGT | |
| | KVEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTKS | |
| | FNRGEC | |
| hu18B9.3i-V2 | DIQMTQSPSSLSASVGDRVTITCRASEGISSYLSWYQQKPGKAPKLLIYKASTL | 209 |
| LC | TSGVPSRFSGSGSGTDFTLTISSLQPEDFATYYCQSAYGSSGSSGYGAAFGGGT | |
| | KVEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTKS | |
| | FNRGEC | |
| hu18B9.3i-V3 | DIQMTQSPSSLSASVGDRVTITCRASEGISSYLSWYQQKPGKAPKLLIYKASTL | 210 |
| LC | TSGVPSRFSGSGSGTDFTLTISSLQPEDFATYYCQSTYGSSGSSGYAAAFGGGT | |
| | KVEIKRTVAAPSVFIFPPSDEQLKSGTASVVCLLNNFYPREAKVQWKVDNALQ | |
| | SGNSQESVTEQDSKDSTYSLSSTLTLSKADYEKHKVYACEVTHQGLSSPVTKS | |
| | FNRGEC | |
| VIR47.V8. | SYELTQPPSVSVSPGQTARITCSGDALPKKYAYWYQQKSGQAPVLVIYEDNK | 211 |
| Lambda LC | RPSGIPERFSGSSSGTMATLTISGAQVEDEADYYCYSTDISGNHWVFGGGTKL | |
| | TVLGQPKAAPSVTLFPPSSEELQANKATLVCLISDFYPGAVTVAWKADSSPVK | |
| | AGVETTTPSKQSNNKYAASSYLSLTPEQWKSHRSYSCQVTHEGSTVEKTVAPT | |
| | ECS | |
| VIR47.V8 LC | SYELTQPPSVSVSPGQTARITCSGDALPKKYAYWYQQKSGQAPVLVIYEDNK | 212 |
| Cetuximab | RPSGIPERFSGSSSGTMATLTISGAQVEDEADYYCYSTDISGNHWVFGGGTKL | |
| xVIR47.V8_V | TVLPDIQNPDPAVYQLRDSKSSDKSVCLFTDFDSQTQVSQSKDSDVYITDKCV | |
| 2 | LDMRSMDFKSNSAVAWSQKSDFACANAFQNSIIPEDTFFCS | |

TABLE 7
Example single-chain variable fragment (scFv)
sequence(s) of the anti-CD47 binding domains.

| Construct | Amino Acid Sequence | SEQ |
|---|---|---|
| Description | (N to C) | ID NO |
| VIR47.V8 | EVQLLESGGGLVQPGGSLRLSCAASGFTFS | 213 |
| scFv Hole | NFAMSWVRQAPGKGLEWVSTISASGGRTFY | |
| | ADSVKGRFTISRDNSKNTLYLQMNSLRAED | |
| | TAVYYCAKEGSFGEGVDPWGQGTLVTVSSG | |
| | GGGSGGGGSGGGGSSYELTQPPSVSVSPGQ | |
| | TARITCSGDALPKKYAYWYQQKSGQAPVLV | |
| | IYEDNKRPSGIPERFSGSSSGTMATLTISG | |
| | AQVEDEADYYCYSTDISGNHWVFGGGTKLT | |
| | VLGGSGGSGGASKTHTCPPCPAPELLGGPS | |
| | VFLFPPKPKDTLMISRTPEVTCVVVDVSHE | |
| | DPEVKFNWYVDGVEVHNAKTKPREEQYNST | |
| | YRVVSVLTVLHQDWLNGKEYKCKVSNKALP | |
| | APIEKTISKAKGQPREPQVYTLPPSRDELT | |
| | KNQVSLSCAVKGFYPSDIAVEWESNGQPEN | |
| | NYKTTPPVLDSDGSFFLVSKLTVDKSRWQQ | |
| | GNVFSCSVMHEALHNHYTQKSLSLSPGK | |

TABLE 8
Example TCR-chimera format sequence(s) of the anti-CD47 binding domains.

| Construct | Amino Acid Sequence | SEQ |
|---|---|---|
| Description | (N to C) | ID NO |
| CD47 TCRα | PDIQNPDPAVYQLRDSKSSDKSVCLFTDFDSQTQVSQSKDSDVYITDKCVLD | 214 |
| | MRSMDFKSNSAVAWSQKSDFACANAFQNSIIPEDTFFCS | |
| CD47 TCRβ | LEDLKNVFPPEVAVFEPSECEISHTQKATLVCLATGFYPDHVELSWWVNGK | 215 |
| | EVHSGVCTDPQPLKEQPALQDSRYALSSRLRVSATFWQNPRNHFRCQVQFY | |
| | GLSENDEWTQDRAKPVTQIVSAEAWGRAS | |
| Fc-knob | DKTHTCPPCPAPELLGGPSVFLFPPKPKDTLMISRTPEVTCVVVDVSHEDP | 216 |
| | EVKFNWYVDGVEVHNAKTKPREEQYNSTYRVVSVLTVLHQDWLNGKEYKCK | |
| | VSNKALPAPIEKTISKAKGQPREPQVYTLPPCREEMTKNQVSLWCLVKGFY | |
| | PSDIAVEWESNGQPENNYKTTPPVLDSDGSFFLYSKLTVDKSRWQQGNVFS | |
| | CSVMHEALHNHYTQKSLSLSPGK | |
| VIR47.V8 | EVQLLESGGGLVQPGGSLRLSCAASGFTFSNFAMSWVRQAPGKGLEWVSTI | 217 |
| TCRβ.HC.Fc | SASGGRTFYADSVKGRFTISRDNSKNTLYLQMNSLRAEDTAVYYCAKEGSF | |
| knob | GEGVDPWGQGTLVTVLEDLKNVFPPEVAVFEPSECEISHTQKATLVCLAT | |
| | GFYPDHVELSWWVNGKEVHSGVCTDPQPLKEQPALQDSRYALSSRLRVSA | |
| | TFWQNPRNHFRCQVQFYGLSENDEWTQDRAKPVTQIVSAEAWGRASDKTH | |
| | TCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVS | |
| | NKALPAPIEKTISKAKGQPREPQVYTLPPCREEMTKNQVSLWCLVKGFYP | |
| | SDIAVEWESNGQPENNYKTTPPVLDSDGSFFLYSKLTVDKSRWQQGNVFS | |
| | CSVMHEALHNHYTQKSLSLSPGK | |
| VIR47.V8 | SYELTQPPSVSVSPGQTARITCSGDALPKKYAYWYQQKSGQAPVLVIYEDN | 218 |
| TCRα.LC | KRPSGIPERFSGSSSGTMATLTISGAQVEDEADYYCYSTDISGNHWVFGGG | |
| | TKLTVLPDIQNPDPAVYQLRDSKSSDKSVCLFTDFDSQTQVSQSKDSDVYI | |
| | TDKCVLDMRSMDFKSNSAVAWSQKSDFACANAFQNSIIPEDTFFCS | |

In some embodiments of an anti-CD47 binding domain described herein, a heavy chain variable fragment (VH) of the anti-CD47 binding domain is fused to a first T-cell receptor (TCR) constant region, and a light chain variable domain (VL) of the anti-CD47 binding domain is fused to a second TCR constant region, wherein the first and second TCR constant regions are capable of forming a dimer. In some embodiments, the TCR constant regions are the TCR alpha and TCR beta constant regions. In some embodiments, the VH-TCR fusion polypeptide is fused to a human IgG (e.g., IgG1) Fc domain. See WO 2019/057122.

In some embodiments, an anti-CD47 binding domain described herein comprises a T cell receptor (TCR) constant region. In some embodiments, the TCR constant region comprises a TCR alpha constant domain and a TCR beta constant domain. In some embodiments, the anti-CD47 binding domain comprises a VH-CD47 domain and a VL-CD47 domain, wherein the VH-CD47 domain is fused to the TCR beta constant domain; and the VL-CD47 domain is fused to the TCR alpha constant domain. In some embodiments, the anti-CD47 binding domain comprises a VH-CD47 domain and a VL-CD47 domain, wherein the VH-CD47 domain is fused to the first constant domain of heavy chain (CH1) IgG domain; and the VL-CD47 domain is fused to the constant domain of light chain (CL) IgG domain.

In some embodiments, an anti-CD47 binding domain described herein comprises a T cell receptor (TCR) constant region. In some embodiments, the TCR constant region comprises a TCR alpha constant domain and a TCR beta constant domain. In some embodiments, an anti-CD47 binding domain described herein comprises a VH-CD47 domain and a VL-CD47 domain, wherein the VH-CD47 domain is fused to the TCR alpha constant domain; and the VL-CD47 domain is fused to the TCR beta constant domain. In some embodiments, the anti-CD47 binding domain comprises a VH-CD47 domain and a VL-CD47 domain, wherein the VH-CD47 domain is fused to the first constant domain of heavy chain (CH1) IgG domain; and the VL-CD47 domain is fused to the constant domain of light chain (CL) IgG domain.

In some embodiments, the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8. In some embodiments, the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 214-218. In some embodiments, the CD47 binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8. In some embodiments, the CD47 binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 214. In some embodiments, the CD47 binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8. In some embodiments, the CD47 binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 215.

In some embodiments, the CD47 binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8; and a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8.

In some embodiments, the CD47 binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 214; and a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 215.

In some embodiments, the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 216.

In some embodiments, the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 217.

In some embodiments, the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 218.

In some embodiments, the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 217; and a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 218.

In some embodiments, an anti-CD47 binding domain comprises a full-length antibody. In other embodiments, the anti-CD47 binding domain comprises a binding fragment. In some instances, the anti-CD47 binding domain comprises an antibody or binding fragment thereof, a monoclonal antibody or binding fragment thereof, a chimeric antibody or binding fragment thereof, a humanized antibody or binding fragment thereof, or an engineered antibody or binding fragment thereof. In some cases, the anti-CD47 binding domain comprises a monovalent Fab, a bivalent Fab′2, a single-chain variable fragment (scFv), or binding fragment thereof.

In some embodiments, an anti-CD47 binding domain described herein has an EC50 of from about 0.02 nM to about 2.27 nM. In some embodiments, an anti-CD47 binding domain described herein has an EC50 of from at least about 0.02 nM. In some embodiments, an anti-CD47 binding domain described herein has an EC50 of from at most about 2.27 nM. In some embodiments, an anti-CD47 binding domain described herein has an EC50 of from about 0.02 nM to about 0.16 nM, about 0.02 nM to about 0.21 nM, about 0.02 nM to about 1.03 nM, about 0.02 nM to about 1.2 nM, about 0.02 nM to about 2.27 nM, about 0.16 nM to about 0.21 nM, about 0.16 nM to about 1.03 nM, about 0.16 nM to about 1.2 nM, about 0.16 nM to about 2.27 nM, about 0.21 nM to about 1.03 nM, about 0.21 nM to about 1.2 nM, about 0.21 nM to about 2.27 nM, about 1.03 nM to about 1.2 nM, about 1.03 nM to about 2.27 nM, or about 1.2 nM to about 2.27 nM, in an in vitro phagocytosis assay to determine ADCP activity, for instance, using macrophages and targeting cancer cells such as metastatic cancer cells from a colorectal cancer, for example, HCT-15 cells or from esophageal, for example, KYSE-150 cells.

In some embodiments, an anti-CD47 binding domain described herein has an EC50 of from about 0.002 nM to about 0.138 nM. In some embodiments, an anti-CD47 binding domain described herein has an EC50 of from at least about 0.002 nM. In some embodiments, an anti-CD47 binding domain described herein has an EC50 of from at most about 0.138 nM. In some embodiments, an anti-CD47 binding domain described herein has an EC50 of from about 0.002 nM to about 0.012 nM, about 0.002 nM to about 0.053 nM, about 0.002 nM to about 0.108 nM, about 0.002 nM to about 0.138 nM, about 0.012 nM to about 0.053 nM, about 0.012 nM to about 0.108 nM, about 0.012 nM to about 0.138 nM, about 0.053 nM to about 0.108 nM, about 0.053 nM to about 0.138 nM, or about 0.108 nM to about 0.138 nM in an in vitro cytotoxicity assay to determine ADCC activity, for instance, using NK92/CD16a176V or PBMCs effector cells and targeting cancer cells such as metastatic cancer cells from a colorectal cancer, for example, HCT-15 cells or from esophageal, for example, KYSE-150 cells.

In some embodiments, an anti-CD47 binding domain described herein has decreased red blood cell (RBC) binding compared to CD47 BMK-1 (such as described herein). In some cases, the decreased RBC binding is a decrease of at least 10%, 20%, 30%, 40%, 50%, 60%, 70%, 80%, 90%, 95%, or 100% compared to reference antibody CD47 BMK-1.

In some embodiments, an anti-CD47 antibody described herein has an improved serum half-life compared to reference antibody CD47 BMK-2 or CD47 BMK-1 (such as described herein). In some embodiments, an anti-CD47 antibody described herein has an improved serum half-life compared to reference antibody CD47 BMK-2 or CD47 BMK-1. In some instances, the improved serum half-life is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than reference antibody CD47 BMK-2 or CD47 BMK-1. In some instances, the improved serum half-life is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than reference antibody CD47 BMK-2 or CD47 BMK-1.

In some cases, the serum half-life of an anti-CD47 binding domain described herein is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer. In some cases, the serum half-life of an anti-CD47 binding domain described herein is about 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer.

### Anti-EpCAM Binding Domains

In certain embodiments, disclosed herein is an anti-EpCAM-binding domain. In some embodiments, the EpCAM-binding domain is a human or engineered human EpCAM-binding domain. In some embodiments, the EpCAM binding domain comprises an antibody, or functional fragment or functional variant thereof, that binds specifically to EpCAM. In some embodiments, the antibody, or functional fragment or functional variant thereof that binds specifically to EpCAM comprises an anti-EpCAM heavy chain and an anti-EpCAM light chain. In some embodiments, the anti-EpCAM heavy chain comprises an anti-EpCAM heavy chain variable domain. In some embodiments, the anti-EpCAM heavy chain variable domain comprises a variable domain of an IgG1, IgG2, IgG3, or IgG4 heavy chain. In some embodiments, the anti-EpCAM light chain comprises an anti-EpCAM light chain variable domain. In some embodiments, the anti-EpCAM light chain variable domain comprises a Kappa or Lambda light chain. In some embodiments, the anti-EpCAM light chain variable domain comprises a Kappa light chain. In some embodiments, the anti-EpCAM light chain variable domain comprises a Lambda light chain. In some embodiments, the EpCAM binding domain comprises a single-chain variable fragment (scFv). In some embodiments, wherein the EpCAM binding domain comprises an antigen-binding fragment (Fab).

In some embodiments, the EpCAM-binding domain comprises three heavy chain (HC) complementarity determining regions (CDRs): HC-CDR1, HC-CDR2, and HC-CDR3; wherein the HC-CDR1, the HC-CDR2, and the HC-CDR3 of the EpCAM binding domain comprises amino acid sequences selected from those set forth in Table 9. In some embodiments, the HC-CDR1 of the EpCAM binding domain comprises an amino acid sequence selected from SEQ ID NOs: 222, 225, 231, 234, 243, and 246. In some embodiments, the the HC-CDR2 of the EpCAM binding domain comprises an amino acid sequence selected from SEQ ID NOs: 223, 226, 232, 235, 244, and 247. In some embodiments, the HC-CDR3 of the EpCAM binding domain comprises an amino acid sequence selected from SEQ ID NOs: 224, 227, 233, 236, 245, and 248. In some embodiments, at least one of the HC-CDR1, the HC-CDR2, and the HC-CDR3 of the CD47-binding domain comprise from 0-2 amino acid modification(s). In some embodiments, at least one of the HC-CDR1, the HC-CDR2, and the HC-CDR3 of the CD47-binding domain comprise from 0-1 amino acid modification(s).

In some embodiments, the HC-CDR1, the HC-CDR2, and the HC-CDR3 of the CD47 binding domain comprise the amino acid sequences of SEQ ID NOs: 219-221; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 222-224; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 225-227; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 228-230; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 231-233; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 234-236; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 237-239; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 240-242; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 243-245; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 246-248; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 249-251; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 252-254; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 255-257; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 258-260; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 261-263; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 264-266; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 267-269; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 270-272; and wherein at least one of the HC-CDR1, the HC-CDR2, and the HC-CDR3 comprise from 0-3 (e.g., 0-2, or 0-1) amino acid modification(s).

In some embodiments, the EpCAM-binding domain comprises three light chain (LC) complementarity determining regions (CDRs): the LC-CDR1, the LC-CDR2, and the LC-CDR3; wherein the LC-CDR1, the LC-CDR2, and the LC-CDR3 of the EpCAM binding domain comprises amino acid sequences selected from those set forth in Table 10. In some embodiments, the LC-CDR1 of the EpCAM binding domain comprises an amino acid sequence selected from SEQ ID NOs: 276, 279, 285, 288, 297, and 300. In some embodiments, the LC-CDR2 of the EpCAM binding domain comprises an amino acid sequence selected from SEQ ID NOs: 277, 280, 286, 289, 298, and 301. In some embodiments, the LC-CDR3 of the EpCAM binding domain comprises an amino acid sequence selected from SEQ ID NOs: 278, 281, 287, 290, 299, and 302. In some embodiments, at least one of the HC-CDR1, the HC-CDR2, and the HC-CDR3 of the CD47-binding domain comprise from 0-2 amino acid modification(s). In some embodiments, at least one of the HC-CDR1, the HC-CDR2, and the HC-CDR3 of the CD47-binding domain comprise from 0-1 amino acid modification(s).

In some embodiments, the LC-CDR1, the LC-CDR2, and the LC-CDR3 of the CD47 binding domain comprise the amino acid sequences of SEQ ID NOs: 273-275; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 276-278; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 279-281; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 282-284; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 285-287; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 288-290; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 291-293; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 294-296; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 297-299; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 300-302; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 303-305; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 306-308; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 309-311; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 312-314; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 315-317; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 318-320; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 321-323; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 324-326; and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 219-221; and the LC-CDR1, the LC-CDR2, and the LC-CDR3 of the CD47 binding domain comprise the amino acid sequences of SEQ ID NOs: 273-275; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 222-224; and the LC-CDR1, the LC-CDR2, and the LC-CDR3 of the CD47 binding domain comprise the amino acid sequences of SEQ ID NOs: 276-278; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 225-227; and the LC-CDR1, the LC-CDR2, and the LC-CDR3 of the CD47 binding domain comprise the amino acid sequences of SEQ ID NOs: 279-281; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 228-230; and the LC-CDR1, the LC-CDR2, and the LC-CDR3 of the CD47 binding domain comprise the amino acid sequences of SEQ ID NOs: 282-284; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 231-233; and the LC-CDR1, the LC-CDR2, and the LC-CDR3 of the CD47 binding domain comprise the amino acid sequences of SEQ ID NOs: 285-287; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 234-236; and the LC-CDR1, the LC-CDR2, and the LC-CDR3 of the CD47 binding domain comprise the amino acid sequences of SEQ ID NOs: 288-290; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 237-239; and the LC-CDR1, the LC-CDR2, and the LC-CDR3 of the CD47 binding domain comprise the amino acid sequences of SEQ ID NOs: 291-293; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 240-242; and the LC-CDR1, the LC-CDR2, and the LC-CDR3 of the CD47 binding domain comprise the amino acid sequences of SEQ ID NOs: 294-296; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 243-245; and the LC-CDR1, the LC-CDR2, and the LC-CDR3 of the CD47 binding domain comprise the amino acid sequences of SEQ ID NOs: 297-299; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 246-248; and the LC-CDR1, the LC-CDR2, and the LC-CDR3 of the CD47 binding domain comprise the amino acid sequences of SEQ ID NOs: 300-302; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 249-251; and the LC-CDR1, the LC-CDR2, and the LC-CDR3 of the CD47 binding domain comprise the amino acid sequences of SEQ ID NOs: 303-305; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 252-254; and the LC-CDR1, the LC-CDR2, and the LC-CDR3 of the CD47 binding domain comprise the amino acid sequences of SEQ ID NOs: 306-308; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 255-257; and the LC-CDR1, the LC-CDR2, and the LC-CDR3 of the CD47 binding domain comprise the amino acid sequences of SEQ ID NOs: 309-311; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 258-260; and the LC-CDR1, the LC-CDR2, and the LC-CDR3 of the CD47 binding domain comprise the amino acid sequences of SEQ ID NOs: 312-314; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 261-263; and the LC-CDR1, the LC-CDR2, and the LC-CDR3 of the CD47 binding domain comprise the amino acid sequences of SEQ ID NOs: 315-317; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 264-266; and the LC-CDR1, the LC-CDR2, and the LC-CDR3 of the CD47 binding domain comprise the amino acid sequences of SEQ ID NOs: 318-320; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 267-269; and the LC-CDR1, the LC-CDR2, and the LC-CDR3 of the CD47 binding domain comprise the amino acid sequences of SEQ ID NOs: 321-323; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 270-272; and the LC-CDR1, the LC-CDR2, and the LC-CDR3 of the CD47 binding domain comprise the amino acid sequences of SEQ ID NOs: 324-326; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the EpCAM-binding domain comprises a heavy chain framework region (HFR) comprising an amino acid sequence selected from those set forth in Table 11; and wherein the HFR comprises from 0-3 amino acid modification(s). In some embodiments, the EpCAM-binding domain comprises a heavy chain framework region (HFR) comprising an amino acid sequence selected from SEQ ID NOs: 331-334, 335-338, 343-346, 347-350, 355-358, and 359-362; and wherein the HFR comprises from 0-3 amino acid modification(s). In some embodiments, the HFR of the EpCAM-binding domain comprises from 0-2 amino acid modification(s). In some embodiments, the HFR of the EpCAM-binding domain comprises from 0-1 amino acid modification(s).

In some embodiments, the heavy chain framework region (HFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 327-330; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the heavy chain framework region (HFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 331-334; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the heavy chain framework region (HFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 335-338; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the heavy chain framework region (HFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 339-342; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the heavy chain framework region (HFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 343-346; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the heavy chain framework region (HFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 347-350; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the heavy chain framework region (HFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 351-354; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the heavy chain framework region (HFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 355-358; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the heavy chain framework region (HFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 359-362; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the EpCAM-binding domain comprises a light chain framework region (LFR) comprising an amino acid sequence selected from those set forth in Table 12; and wherein the LFR comprises from 0-3 amino acid modification(s). In some embodiments, the EpCAM-binding domain comprises a light chain framework region (LFR) comprising an amino acid sequence selected from SEQ ID NOs: 367-370, 371-374, 379-382, 383-386, 391-394, and 395-398; and wherein the HFR comprises from 0-3 amino acid modification(s). In some embodiments, the LFR of the EpCAM-binding domain comprises from 0-2 amino acid modification(s). In some embodiments, the LFR of the EpCAM-binding domain comprises from 0-1 amino acid modification(s).

In some embodiments, the light chain framework region (LFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 363-366; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the light chain framework region (LFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 367-370; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the light chain framework region (LFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 371-374; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the light chain framework region (LFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 375-378; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the light chain framework region (LFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 379-382; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the light chain framework region (LFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 383-386; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the light chain framework region (LFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 387-390; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the light chain framework region (LFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 391-394; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the light chain framework region (LFR) of the EpCAM-binding domain comprises a pre-CDR1 region, an inter-CDR1/2 region, an inter-CDR2/3 region, and a post-CDR3 region. In some embodiments, the pre-CDR1 region, the inter-CDR1/2 region, the inter-CDR2/3 region, and the post-CDR3 region comprise the amino acid sequences of SEQ ID NOs: 395-398; wherein at least one of the pre-CDR1 region, the inter-CDR1/2 region, the inter-CDR2/3 region, and the post-CDR3 region comprise from 0-5 amino acid modification(s). In some embodiments, at least one of the pre-CDR1 region, the inter-CDR1/2 region, the inter-CDR2/3 region, and the post-CDR3 region comprise from 0-4 amino acid modification(s). In some embodiments, at least one of the pre-CDR1 region, the inter-CDR1/2 region, the inter-CDR2/3 region, and the post-CDR3 region comprise from 0-3 amino acid modification(s). In some embodiments, at least one of the pre-CDR1 region, the inter-CDR1/2 region, the inter-CDR2/3 region, and the post-CDR3 region comprise from 0-2 amino acid modification(s). In some embodiments, at least one of the pre-CDR1 region, the inter-CDR1/2 region, the inter-CDR2/3 region, and the post-CDR3 region comprise from 0-1 amino acid modification(s).

In some embodiments, the pre-CDR1 region, the inter-CDR1/2 region, the inter-CDR2/3 region, and the post-CDR3 region of the heavy chain framework region (HFR) of the EpCAM-binding domain comprise the amino acid sequences of SEQ ID NOs: 327-330; and the pre-CDR1 region, the inter-CDR1/2 region, the inter-CDR2/3 region, and the post-CDR3 region of the light chain framework region (LFR) of the EpCAM-binding domain comprise the amino acid sequences of SEQ ID NOs: 363-366; wherein at least one of the pre-CDR1 region, the inter-CDR1/2 region, the inter-CDR2/3 region, and the post-CDR3 region of the heavy chain framework region (HFR) of the EpCAM-binding domain comprise from 0-5 (e.g., 0-4, 0-3, 0-2, or 0-1) amino acid modification(s); and wherein at least one of the pre-CDR1 region, the inter-CDR1/2 region, the inter-CDR2/3 region, and the post-CDR3 region of the light chain framework region (LFR) of the EpCAM-binding domain comprise from 0-5 (e.g., 0-4, 0-3, 0-2, or 0-1) amino acid modification(s).

In some embodiments, the pre-CDR1 region, the inter-CDR1/2 region, the inter-CDR2/3 region, and the post-CDR3 region of the heavy chain framework region (HFR) of the EpCAM-binding domain comprise the amino acid sequences of SEQ ID NOs: 331-334; and the pre-CDR1 region, the inter-CDR1/2 region, the inter-CDR2/3 region, and the post-CDR3 region of the light chain framework region (LFR) of the EpCAM-binding domain comprise the amino acid sequences of SEQ ID NOs: 367-370; wherein at least one of the pre-CDR1 region, the inter-CDR1/2 region, the inter-CDR2/3 region, and the post-CDR3 region of the heavy chain framework region (HFR) of the EpCAM-binding domain comprise from 0-5 (e.g., 0-4, 0-3, 0-2, or 0-1) amino acid modification(s); and wherein at least one of the pre-CDR1 region, the inter-CDR1/2 region, the inter-CDR2/3 region, and the post-CDR3 region of the light chain framework region (LFR) of the EpCAM-binding domain comprise from 0-5 (e.g., 0-4, 0-3, 0-2, or 0-1) amino acid modification(s).

In some embodiments, the pre-CDR1 region, the inter-CDR1/2 region, the inter-CDR2/3 region, and the post-CDR3 region of the heavy chain framework region (HFR) of the EpCAM-binding domain comprise the amino acid sequences of SEQ ID NOs: 335-338; and the pre-CDR1 region, the inter-CDR1/2 region, the inter-CDR2/3 region, and the post-CDR3 region of the light chain framework region (LFR) of the EpCAM-binding domain comprise the amino acid sequences of SEQ ID NOs: 371-374; wherein at least one of the pre-CDR1 region, the inter-CDR1/2 region, the inter-CDR2/3 region, and the post-CDR3 region of the heavy chain framework region (HFR) of the EpCAM-binding domain comprise from 0-5 (e.g., 0-4, 0-3, 0-2, or 0-1) amino acid modification(s); and wherein at least one of the pre-CDR1 region, the inter-CDR1/2 region, the inter-CDR2/3 region, and the post-CDR3 region of the light chain framework region (LFR) of the EpCAM-binding domain comprise from 0-5 (e.g., 0-4, 0-3, 0-2, or 0-1) amino acid modification(s).

In some embodiments, the pre-CDR1 region, the inter-CDR1/2 region, the inter-CDR2/3 region, and the post-CDR3 region of the heavy chain framework region (HFR) of the EpCAM-binding domain comprise the amino acid sequences of SEQ ID NOs: 339-342; and the pre-CDR1 region, the inter-CDR1/2 region, the inter-CDR2/3 region, and the post-CDR3 region of the light chain framework region (LFR) of the EpCAM-binding domain comprise the amino acid sequences of SEQ ID NOs: 375-378; wherein at least one of the pre-CDR1 region, the inter-CDR1/2 region, the inter-CDR2/3 region, and the post-CDR3 region of the heavy chain framework region (HFR) of the EpCAM-binding domain comprise from 0-5 (e.g., 0-4, 0-3, 0-2, or 0-1) amino acid modification(s); and wherein at least one of the pre-CDR1 region, the inter-CDR1/2 region, the inter-CDR2/3 region, and the post-CDR3 region of the light chain framework region (LFR) of the EpCAM-binding domain comprise from 0-5 (e.g., 0-4, 0-3, 0-2, or 0-1) amino acid modification(s).

In some embodiments, the pre-CDR1 region, the inter-CDR1/2 region, the inter-CDR2/3 region, and the post-CDR3 region of the heavy chain framework region (HFR) of the EpCAM-binding domain comprise the amino acid sequences of SEQ ID NOs: 343-346; and the pre-CDR1 region, the inter-CDR1/2 region, the inter-CDR2/3 region, and the post-CDR3 region of the light chain framework region (LFR) of the EpCAM-binding domain comprise the amino acid sequences of SEQ ID NOs: 379-382; wherein at least one of the pre-CDR1 region, the inter-CDR1/2 region, the inter-CDR2/3 region, and the post-CDR3 region of the heavy chain framework region (HFR) of the EpCAM-binding domain comprise from 0-5 (e.g., 0-4, 0-3, 0-2, or 0-1) amino acid modification(s); and wherein at least one of the pre-CDR1 region, the inter-CDR1/2 region, the inter-CDR2/3 region, and the post-CDR3 region of the light chain framework region (LFR) of the EpCAM-binding domain comprise from 0-5 (e.g., 0-4, 0-3, 0-2, or 0-1) amino acid modification(s).

In some embodiments, the pre-CDR1 region, the inter-CDR1/2 region, the inter-CDR2/3 region, and the post-CDR3 region of the heavy chain framework region (HFR) of the EpCAM-binding domain comprise the amino acid sequences of SEQ ID NOs: 347-350; and the pre-CDR1 region, the inter-CDR1/2 region, the inter-CDR2/3 region, and the post-CDR3 region of the light chain framework region (LFR) of the EpCAM-binding domain comprise the amino acid sequences of SEQ ID NOs: 383-386; wherein at least one of the pre-CDR1 region, the inter-CDR1/2 region, the inter-CDR2/3 region, and the post-CDR3 region of the heavy chain framework region (HFR) of the EpCAM-binding domain comprise from 0-5 (e.g., 0-4, 0-3, 0-2, or 0-1) amino acid modification(s); and wherein at least one of the pre-CDR1 region, the inter-CDR1/2 region, the inter-CDR2/3 region, and the post-CDR3 region of the light chain framework region (LFR) of the EpCAM-binding domain comprise from 0-5 (e.g., 0-4, 0-3, 0-2, or 0-1) amino acid modification(s).

In some embodiments, the pre-CDR1 region, the inter-CDR1/2 region, the inter-CDR2/3 region, and the post-CDR3 region of the heavy chain framework region (HFR) of the EpCAM-binding domain comprise the amino acid sequences of SEQ ID NOs: 351-354; and the pre-CDR1 region, the inter-CDR1/2 region, the inter-CDR2/3 region, and the post-CDR3 region of the light chain framework region (LFR) of the EpCAM-binding domain comprise the amino acid sequences of SEQ ID NOs: 387-390; wherein at least one of the pre-CDR1 region, the inter-CDR1/2 region, the inter-CDR2/3 region, and the post-CDR3 region of the heavy chain framework region (HFR) of the EpCAM-binding domain comprise from 0-5 (e.g., 0-4, 0-3, 0-2, or 0-1) amino acid modification(s); and wherein at least one of the pre-CDR1 region, the inter-CDR1/2 region, the inter-CDR2/3 region, and the post-CDR3 region of the light chain framework region (LFR) of the EpCAM-binding domain comprise from 0-5 (e.g., 0-4, 0-3, 0-2, or 0-1) amino acid modification(s).

In some embodiments, the pre-CDR1 region, the inter-CDR1/2 region, the inter-CDR2/3 region, and the post-CDR3 region of the heavy chain framework region (HFR) of the EpCAM-binding domain comprise the amino acid sequences of SEQ ID NOs: 355-358; and the pre-CDR1 region, the inter-CDR1/2 region, the inter-CDR2/3 region, and the post-CDR3 region of the light chain framework region (LFR) of the EpCAM-binding domain comprise the amino acid sequences of SEQ ID NOs: 391-394; wherein at least one of the pre-CDR1 region, the inter-CDR1/2 region, the inter-CDR2/3 region, and the post-CDR3 region of the heavy chain framework region (HFR) of the EpCAM-binding domain comprise from 0-5 (e.g., 0-4, 0-3, 0-2, or 0-1) amino acid modification(s); and wherein at least one of the pre-CDR1 region, the inter-CDR1/2 region, the inter-CDR2/3 region, and the post-CDR3 region of the light chain framework region (LFR) of the EpCAM-binding domain comprise from 0-5 (e.g., 0-4, 0-3, 0-2, or 0-1) amino acid modification(s).

In some embodiments, the pre-CDR1 region, the inter-CDR1/2 region, the inter-CDR2/3 region, and the post-CDR3 region of the heavy chain framework region (HFR) of the EpCAM-binding domain comprise the amino acid sequences of SEQ ID NOs: 359-362; and the pre-CDR1 region, the inter-CDR1/2 region, the inter-CDR2/3 region, and the post-CDR3 region of the light chain framework region (LFR) of the EpCAM-binding domain comprise the amino acid sequences of SEQ ID NOs: 395-398; wherein at least one of the pre-CDR1 region, the inter-CDR1/2 region, the inter-CDR2/3 region, and the post-CDR3 region of the heavy chain framework region (HFR) of the EpCAM-binding domain comprise from 0-5 (e.g., 0-4, 0-3, 0-2, or 0-1) amino acid modification(s); and wherein at least one of the pre-CDR1 region, the inter-CDR1/2 region, the inter-CDR2/3 region, and the post-CDR3 region of the light chain framework region (LFR) of the EpCAM-binding domain comprise from 0-5 (e.g., 0-4, 0-3, 0-2, or 0-1) amino acid modification(s).

In some embodiments, the EpCAM-binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 13.

In some embodiments, the EpCAM-binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 399-416.

In some embodiments, the EpCAM-binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 14.

In some embodiments, the EpCAM-binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 417-434.

In some embodiments, the EpCAM-binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 13; and a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 14.

In some embodiments, the EpCAM-binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 399-416; and a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 417-434 or SEQ ID NOs: 513-514.

In some embodiments, the EpCAM-binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 15.

In some embodiments, the EpCAM-binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 435-443 or SEQ ID NOs: 515-516.

In some embodiments, the EpCAM-binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 16.

In some embodiments, the EpCAM-binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 444-452 or SEQ ID NOs: 517-518.

In some embodiments, the EpCAM-binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 15; and a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 16.

In some embodiments, the EpCAM-binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 435-443; and a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 444-452.

In some embodiments, the EpCAM binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 17.

In some embodiments, the EpCAM binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence selected from SEQ ID NOs: 453-457.

In some embodiments of an anti-EpCAM binding domain described herein, a heavy chain variable fragment (VH) of the anti-EpCAM binding domain is fused to a first T-cell receptor (TCR) constant region, and a light chain variable domain (VL) of the anti-EpCAM binding domain is fused to a second TCR constant region, wherein the first and second TCR constant regions are capable of forming a dimer. In some embodiments, the TCR constant regions are the TCR alpha and TCR beta constant regions. In some embodiments, the VH-TCR fusion polypeptide is fused to a human IgG (e.g., IgG1) Fc domain. See WO 2019/057122.

In some embodiments, an anti-EpCAM binding domain described herein comprises a T cell receptor (TCR) constant region. In some embodiments, the TCR constant region comprises a TCR alpha constant domain and a TCR beta constant domain. In some embodiments, the anti-EpCAM binding domain comprises a VH-EpCAM domain and a VL-EpCAM domain, wherein the VH-EpCAM domain is fused to the TCR beta constant domain; and the VL-EpCAM domain is fused to the TCR alpha constant domain. In some embodiments, the anti-EpCAM binding domain comprises a VH-EpCAM domain and a VL-EpCAM domain, wherein the VH-EpCAM domain is fused to the first constant domain of heavy chain (CH1) IgG domain; and the VL-EpCAM domain is fused to the constant domain of light chain (CL) IgG domain.

In some embodiments, an anti-EpCAM binding domain described herein comprises a T cell receptor (TCR) constant region. In some embodiments, the TCR constant region comprises a TCR alpha constant domain and a TCR beta constant domain. In some embodiments, an anti-EpCAM binding domain described herein comprises a VH-EpCAM domain and a VL-EpCAM domain, wherein the VH-EpCAM domain is fused to the TCR alpha constant domain; and the VL-EpCAM domain is fused to the TCR beta constant domain. In some embodiments, the anti-EpCAM binding domain comprises a VH-EpCAM domain and a VL-EpCAM domain, wherein the VH-EpCAM domain is fused to the first constant domain of heavy chain (CH1) IgG domain; and the VL-EpCAM domain is fused to the constant domain of light chain (CL) IgG domain.

In some embodiments, the EpCAM binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18. In some embodiments, the EpCAM binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18.

In some embodiments, the EpCAM binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18; and a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18.

In some embodiments, the EpCAM binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 458.

In some embodiments, the EpCAM binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 459.

In some embodiments, the EpCAM binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 458; and a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 459.

TABLE 9
Example CDR sequences of the anti-EpCAM heavy chain binding domains,
as determined according to Kabat nomenclature.

| | SEQ ID | | SEQ ID | | SEQ ID | |
|---|---|---|---|---|---|---|
| Construct | NO | CDR1 | NO | CDR2 | NO | CDR3 |
| mAb-1 | 219 | SYDIN | 220 | WIYPRDGSTKYNE | 221 | DPYYYGTIYGYFD |
| | | | | RFKG | | V |
| 001-11 | 222 | SYDIN | 223 | WIYPRDGSTKYA | 224 | DPYYYGTIYGYF |
| | | | | QKFQG | | DV |
| 001-12 | 222 | SYDIN | 223 | WIYPRDGSTKYA | 224 | DPYYYGTIYGYF |
| | | | | QKFQG | | DV |
| 001-18 | 225 | SYDIN | 226 | WIYPREGSTKYA | 227 | DPYYYGTIYGYF |
| | | | | QKFQG | | DV |
| mAb-2 | 228 | TYAMH | 229 | RIRSKTYNYATFY | 230 | GSSYAMDY |
| | | | | ADSVKD | | |
| 002-7/8 | 231 | TYAMH | 232 | RIRSKTYNYATFY | 233 | GSSYAMDY |
| | | | | ADSVKG | | |
| 002-8 | 231 | TYAMH | 232 | RIRSKTYNYATFY | 233 | GSSYAMDY |
| | | | | ADSVKG | | |
| 002-11 | 234 | TYAMH | 235 | RIRSKTYNYATFY | 236 | GSSYAMDY |
| | | | | ADSVKG | | |
| mAb-4 | 237 | SYWMH | 238 | VIDPSNSYSNYNQ | 239 | PYNHGRDAMDY |
| | | | | KFKG | | |
| mAb-8 | 240 | SYGIS | 241 | EIYPRSDNTYYNE | 242 | RAGMWFAY |
| | | | | KFKG | | |
| 008-1 | 243 | SYGIS | 244 | EIYPRSDNTYYAQ | 245 | RAGMWFAY |
| | | | | KFQG | | |
| mAb8-1 | 246 | SYGIS | 247 | EIYPRSDNTYYAQ | 248 | RAGMWFAY |
| | | | | KFQG | | |
| mAb-21 | 249 | SDFAWN | 250 | YITYSGTTSYNPSL | 251 | SFHFFAYGFAY |
| | | | | KS | | |
| mAb-28 | 252 | SYGIS | 253 | EIYPTRDYTYYNE | 254 | DGPPFAY |
| | | | | NFKG | | |
| mAb-30 | 255 | DYYMS | 256 | EINPDNGDTSHNQ | 257 | YFSY |
| | | | | KFKG | | |
| mAb-33 | 258 | NTYIH | 259 | RIGPANGNTKYAP | 260 | AYYRIY |
| | | | | KFQG | | |
| mAb-35 | 261 | NTYMH | 262 | RIAPANGNTKYAP | 263 | IYQLPY |
| | | | | KFQD | | |
| mAb-36 | 264 | DYYIT | 265 | WIFPGRGISFYNEN | 266 | WGGTWFAY |
| | | | | FKD | | |
| mAb-37 | 267 | NTYMH | 268 | RIAPANGNTKYDP | 269 | IYQLPY |
| | | | | KFQD | | |
| mAb-39 | 270 | SYGVT | 271 | WINTYSGVPTYAD | 272 | SHSNYDF |
| | | | | DFKG | | |

TABLE 10
Example CDR sequences of the anti-EpCAM light chain binding
domains, as determined according to Kabat nomenclature.

| | SEQ ID | | SEQ ID | | SEQ ID | |
|---|---|---|---|---|---|---|
| Construct | NO | CDR1 | NO | CDR2 | NO | CDR3 |
| mAb-1 | 273 | RASQDIGNSLN | 274 | VTSRLDS | 275 | LQYASSPFT |
| 001-11 | 276 | RASQDIGNSLN | 277 | VTSRLDS | 278 | LQYASSPFT |
| 001-12 | 276 | RASQDIGNSLN | 277 | VTSRLDS | 275 | LQYASSPFT |
| 001-18 | 279 | RASQDIGNSLN | 280 | VTSRLDS | 281 | LQYASSPFT |
| mAb-2 | 282 | RASQSIGTSLH | 283 | YASESIS | 284 | QQSNSWPYT |
| 002-7/8 | 285 | RASQSIGTSLH | 286 | YASESIS | 287 | QQSNSWPYT |
| 002-8 | 285 | RASQSIGTSLH | 286 | YASESIS | 287 | QQSNSWPYT |
| 002-11 | 288 | RASQSIGTSLH | 289 | YASESIS | 290 | QQSNSWPYT |
| mAb-4 | 291 | RSSQSFVHING | 292 | KVSNRFS | 293 | FQGSH |
| | | NTYLE | | | | |
| mAb-8 | 294 | SASSSVSDMH | 295 | EISKLAS | 296 | QHWSYPLMT |
| 008-1 | 297 | RASSSVSDMH | 298 | EISKLAS | 299 | QHWSYPLMT |
| mAb8-1 | 300 | RASSSVSDMH | 301 | EISKLAS | 302 | QHWSYPLMT |
| mAb-21 | 303 | KSSQSLLDSDG | 304 | LVSKLDS | 305 | WQGTH |
| | | KTYLN | | | | |
| mAb-28 | 306 | RSSKSLLHSNG | 307 | QMSSLAS | 308 | AQNLELPRT |
| | | FTYLH | | | | |
| mAb-30 | 309 | RSSQSIVDSDG | 310 | KVSNRFS | 311 | FQGSH |
| | | STYLE | | | | |
| mAb-33 | 312 | RASESVDNSGI | 313 | RASNLES | 314 | QQSYENPWT |
| | | SFMH | | | | |
| mAb-35 | 315 | RASESVDNYGI | 316 | RASSLES | 317 | QQSYKD |
| | | SFMH | | | | |
| mAb-36 | 318 | SASSSVSYIH | 319 | EISKLAS | 320 | QQWNYPLIT |
| mAb-37 | 321 | RASESVDNYGI | 322 | RASSLES | 323 | QQSYKD |
| | | SFMH | | | | |
| mAb-39 | 324 | QASQNVGTDVG | 325 | SASNRYT | 326 | QQYSSYPRT |

TABLE 11
Example frame region sequences of the anti-EpCAM heavy
chain binding domains (HFR(s)).

| Construct | | SEQ | Amino Acid Sequence |
|---|---|---|---|
| Description | | ID NO | (N to C) |
| mAb-1 | HC pre-CDR1 | 327 | QVQLQQSGPELVKPGTSVKLSCRASGYTFT |
| | HC inter-CDR1/2 | 328 | WVKQRPGQGLE |
| | HC inter-CDR2/3 | 329 | KATLTVDTSSSTAYMEFHTLTSEDSAVYFCAR |
| | HC post-CDR3 | 330 | WGTGTTVTVSS |
| 001-11 | HC pre-CDR1 | 331 | QVQLVQSGAEVKKPGASVKVSCKASGYTFT |
| | HC inter-CDR1/2 | 332 | WVRQAPGQGLEWMG |
| | HC inter-CDR2/3 | 333 | RVTMTVDTSTSTAYMELSSLRSEDTAVYYCAR |
| | HC post-CDR3 | 334 | WGQGTTVTVSS |
| 001-18 | HC pre-CDR1 | 335 | QVQLVQSGAEVKKPGASVKVSCKASGYTFT |
| | HC inter-CDR1/2 | 336 | WVRQAPGQGLEWMG |
| | HC inter-CDR2/3 | 337 | RVTMTVDTSTSTAYMELSSLRSEDTAVYYCAR |
| | HC post-CDR3 | 338 | WGQGTTVTVSS |
| mAb-2 | HC pre-CDR1 | 339 | EVQLVESGGGLVQPKGSLKLSCAASGFTFN |
| | HC inter-CDR1/2 | 340 | WVRQAPGKGLEWVA |
| | HC inter-CDR2/3 | 341 | RFTISRDDSQSMLYLQMNNLKTEDTAMFYCVT |
| | HC post-CDR3 | 342 | WGQGTSVTVSS |
| 002-7/8 | HC pre-CDR1 | 343 | EVQLVESGGGLVQPGGSLRLSCAASGFTFN |
| | HC inter-CDR1/2 | 344 | WVRQAPGKGLEWVA |
| | HC inter-CDR2/3 | 345 | RFTISRDNSKNTLYLQMNSLRAEDTAVYYCVT |
| | HC post-CDR3 | 346 | WGQGTTVTVSS |
| 002-11 | HC pre-CDR1 | 347 | EVQLVESGGGLVQPGGSLRLSCAAS |
| | HC inter-CDR1/2 | 348 | WVRQAPGKGLEWVA |
| | HC inter-CDR2/3 | 349 | YADSVKGRFTISRDDSKNTLYLQMNSLRAEDTAVYYCVT |
| | HC post-CDR3 | 350 | WGQGTTVTVSS |
| mAb-8 | HC pre-CDR1 | 351 | QVQLQQSGAELARPGASVQLSCKASGYTFT |
| | HC inter-CDR1/2 | 352 | WLKQRAGQGLEWIG |
| | HC inter-CDR2/3 | 353 | KATLTADKSSSTAYMELRSLTSEDSAVYFCAR |
| | HC post-CDR3 | 354 | WGQGTLVTVSA |
| 008-1 | HC pre-CDR1 | 355 | QVQLVQSGAEVKKPGSSVKVSCKASGYTFT |
| | HC inter-CDR1/2 | 356 | WVRQAPGQGLEWMG |
| | HC inter-CDR2/3 | 357 | RVTITADKSTSTAYMELSSLRSEDTAVYYCAR |
| | HC post-CDR3 | 358 | WGQGTLVTVSS |
| mAb8-1 | HC pre-CDR1 | 359 | QVQLVQSGAEVKKPGSSVKVSCKASGYTFT |
| | HC inter-CDR1/2 | 360 | WVRQAPGQGLEWMG |
| | HC inter-CDR2/3 | 361 | RVTITADKSTSTAYMELSSLRSEDTAVYYCAR |
| | HC post-CDR3 | 362 | WGQGTLVTVSS |

TABLE 12
Example frame region sequences of the anti-EpCAM
light chain binding domains (LFR(s)).

| Construct | | SEQ | Amino Acid Sequence |
|---|---|---|---|
| Description | | ID NO | (N to C) |
| mAb-1 | LC pre-CDR1 | 363 | DIQMTQSPSSLSASLGERVSLTC |
| | LC inter-CDR1/2 | 364 | WLQQKPDGTIKRLIY |
| | LC inter-CDR2/3 | 365 | GVPKRFSGSRSGSDYFFTISSLESEDFADYYC |
| | LC post-CDR3 | 366 | FGSGTKLEIK |
| 001-11 | LC pre-CDR1 | 367 | DIQMTQSPSSLSASVGDRVTLTC |
| | LC inter-CDR1/2 | 368 | WLQQKPGKAPKRLIY |
| | LC inter-CDR2/3 | 369 | GVPSRFSGSGSGTDYTFTISSLQPEDFATYYC |
| | LC post-CDR3 | 370 | FGQGTKLEIK |
| 001-18 | LC pre-CDR1 | 371 | DIQMTQSPSSLSASVGDRVTLTC |
| | LC inter-CDR1/2 | 372 | WLQQKPGKAPKRLIY |
| | LC inter-CDR2/3 | 373 | GVPSRFSGSRSGTDYTFTISSLQPEDFATYYC |
| | LC post-CDR3 | 374 | FGQGTKLEIK |
| mAb-2 | LC pre-CDR1 | 375 | DILLTQSPAILSVSPGERVSFSC |
| | LC inter-CDR1/2 | 376 | WYQQRTNGSPRLLIK |
| | LC inter-CDR2/3 | 377 | GIPSRFSGSGSGTDFTLSINSVESEDIADYYC |
| | LC post-CDR3 | 378 | FGGGTKLEIK |
| 002-7/8 | LC pre-CDR1 | 379 | DIQLTQSPSSLSASVGDRVTFTC |
| | LC inter-CDR1/2 | 380 | WYQQKPGKAPKLLIK |
| | LC inter-CDR2/3 | 381 | GIPSRFSGSGSGTDFTLTISSVQPEDIATYYC |
| | LC post-CDR3 | 382 | FGQGTKLEIK |
| 002-11 | LC pre-CDR1 | 383 | DIQLTQSPSSLSASVGDRVTFTC |
| | LC inter-CDR1/2 | 384 | WYQQKPGKAPKLLIK |
| | LC inter-CDR2/3 | 385 | GIPSRFSGSGSGTDFTLTISSVQPEDFATYYC |
| | LC post-CDR3 | 386 | FGQGTKLEIK |
| mAb-8 | LC pre-CDR1 | 387 | EIVLTQSPAITAASLGQKVTITC |
| | LC inter-CDR1/2 | 388 | WYQQKSGTSPKPWIY |
| | LC inter-CDR2/3 | 389 | GVPTRFSGSGSGTSYSLTISSMEAEDAAIYYC |
| | LC post-CDR3 | 390 | FGGGTKLEIK |
| 008-1 | LC pre-CDR1 | 391 | DIQMTQSPSSLSASVGDRVTITC |
| | LC inter-CDR1/2 | 392 | WYQQKPGKAPKLLIY |
| | LC inter-CDR2/3 | 393 | GVPSRFSGSGSGTDFTLTISSLQPEDFATYYC |
| | LC post-CDR3 | 394 | FGQGTKLEIK |
| mAb8-1 | LC pre-CDR1 | 395 | DIQMTQSPSSLSASVGDRVTITC |
| | LC inter-CDR1/2 | 396 | WYQQKPGKAPKLLIY |
| | LC inter-CDR2/3 | 397 | GVPSRFSGSGSGTDFTLTISSLQPEDFATYYC |
| | LC post-CDR3 | 398 | FGQGTKLEIK |

TABLE 13
Example variable domain sequences of the anti-EpCAM heavy chain binding domains. CDRs,
as determined according to Kabat nomenclature, are underlined and bolded.

| Construct | SEQ | Amino Acid Sequence |
|---|---|---|
| Description | ID NO | (N to C) |
| mAb-1 | 399 | QVQLQQSGPELVKPGTSVKLSCRASGYTFTSYDINWVKQRPGQGLEWIGWIYP |
| | | RDGSTKYNERFKGKATLTVDTSSSTAYMEFHTLTSEDSAVYFCARDPYYYGTI |
| | | YGYFDVWGTGTTVTVSS |
| 001-11 | 400 | QVQLVQSGAEVKKPGASVKVSCKASGYTFTSYDINWVRQAPGQGLEWMGWIY |
| | | PRDGSTKYAQKFQGRVTMTVDTSTSTAYMELSSLRSEDTAVYYCARDPYYYG |
| | | TIYGYFDVWGQGTTVTVSS |
| 001-12 | 511 | QVQLVQSGAEVKKPGASVKVSCKASGYTFTSYDINWVRQAPGQGLEWMGWIY |
| | | PRDGSTKYAQKFQGRVTMTVDTSTSTAYMELSSLRSEDTAVYYCARDPYYYG |
| | | TIYGYFDVWGQGTTVTVSS |
| 001-18 | 401 | QVQLVQSGAEVKKPGASVKVSCKASGYTFTSYDINWVRQAPGQGLEWMGWIY |
| | | PREGSTKYAQKFQGRVTMTVDTSTSTAYMELSSLRSEDTAVYYCARDPYYYG |
| | | TIYGYFDVWGQGTTVTVSS |
| mAb-2 | 402 | EVQLVESGGGLVQPKGSLKLSCAASGFTFNTYAMHWVRQAPGKGLEWVARIR |
| | | SKTYNYATFYADSVKDRFTISRDDSQSMLYLQMNNLKTEDTAMFYCVTGSSYA |
| | | MDYWGQGTSVTVSS |
| 002-7/8 | 403 | EVQLVESGGGLVQPGGSLRLSCAASGFTFNTYAMHWVRQAPGKGLEWVARIRS |
| | | KTYNYATFYADSVKGRFTISRDNSKNTLYLQMNSLRAEDTAVYYCVTGSSYA |
| | | MDYWGQGTTVTVSS |
| 002-8 | 512 | EVQLVESGGGLVQPGGSLRLSCAASGFTFNTYAMHWVRQAPGKGLEWVARIRS |
| | | KTYNYATFYADSVKGRFTISRDNSKNTLYLQMNSLRAEDTAVYYCVTGSSYA |
| | | MDYWGQGTTVTVSS |
| 002-11 | 404 | EVQLVESGGGLVQPGGSLRLSCAASGFTFNTYAMHWVRQAPGKGLEWVARIRS |
| | | KTYNYATFYADSVKGRFTISRDDSKNTLYLQMNSLRAEDTAVYYCVTGSSYAM |
| | | DYWGQGTTVTVSS |
| mAb-4 | 405 | QVQLQQPGAELVRPGTSVKLSCKASGYTFTSYWMHWIKQRPGQGLEWIGVIDP |
| | | SNSYSNYNQKFKGRATLTVDTYSSTAYMQLSSLTSEDSAVYYCARPYNHGRDA |
| | | MDYWGQGTSVTVSS |
| mAb-8 | 406 | QVQLQQSGAELARPGASVQLSCKASGYTFTSYGISWLKQRAGQGLEWIGEIYPR |
| | | SDNTYYNEKFKGKATLTADKSSSTAYMELRSLTSEDSAVYFCARRAGMWFAY |
| | | WGQGTLVTVSA |
| 008-1 | 407 | QVQLVQSGAEVKKPGSSVKVSCKASGYTFTSYGISWVRQAPGQGLEWMGEIYP |
| | | RSDNTYYAQKFQGRVTITADKSTSTAYMELSSLRSEDTAVYYCARRAGMWFA |
| | | YWGQGTLVTVSS |
| mAb8-1 | 408 | QVQLVQSGAEVKKPGSSVKVSCKASGYTFTSYGISWVRQAPGQGLEWMGEIYP |
| | | RSDNTYYAQKFQGRVTITADKSTSTAYMELSSLRSEDTAVYYCARRAGMWFA |
| | | YWGQGTLVTVSS |
| mAb-21 | 409 | DVQLQESGPGLVKPSQSLSLTCTVTGYSITSDFAWNWIRQFPGNKLEWVGYITY |
| | | SGTTSYNPSLKSRISITRDTSKNQFFLQLSSVTTEDTATYYCARSFHFFAYGFAY |
| | | WGQGTLVTVSA |
| mAb-28 | 410 | QVQLQQSGAELARPGASVKVSCKASGYTFTSYGISWVKQRTGQGLEWIGEIYPT |
| | | RDYTYYNENFKGKATLTADKSSTTAYMELRSLTSEDSAVYFCARDGPPFAYW |
| | | GQGTLVTVSA |
| mAb-30 | 411 | EVQLQQSGPELVKPGASVKISCKASGYTFTDYYMSWVKQSHGRSLEWIGEINPD |
| | | NGDTSHNQKFKGKATMTVDKSSGTGNMEIRSLTSEDAGIYYCTSYFSYWGQGT |
| | | LVTVSA |
| mAb-33 | 412 | EVLLQQSVADLVRPGASVKLSCTASGLNIKNTYIHWVKQMPEQGLEWIGRIGPA |
| | | NGNTKYAPKFQGKATITADTSSDTAYLQLSSLTSEDTAIYYCALAYYRIYWGQ |
| | | GTTLTVSS |
| mAb-35 | 413 | EVQLQQSVAELVRPGASVKLSCTASGFNIKNTYMHWVKQRPEQGLEWIGRIAP |
| | | ANGNTKYAPKFQDKATITANTSSNTAYLQLGSLTSEDTAIYYCAFIYQLPYWGQ |
| | | GTTLTVSS |
| mAb-36 | 414 | QVQVQQSGPELVKPGTSVKISCKTSGYTFTDYYITWVKQRPGQGLEWIGWIFPG |
| | | RGISFYNENFKDKATLTVDKSSNTAFMLLSSLTSEDSAVYFCARWGGTWFAY |
| | | WGQGTLITVSA |
| mAb-37 | 415 | EVQLQQSVAELVRPGASVKLSCTASGFNIKNTYMHWVKQRPEQGLEWIGRIAP |
| | | ANGNTKYDPKFQDKATITADSSSNTAYLHLSSLTSEDTAIYYCAFIYQLPYWGQ |
| | | GSTLTVSS |
| mAb-39 | 416 | QIQLVQSGPELKKPGETVKISCKASGYTFTSYGVTWVKQAPGKGLKWMGWINT |
| | | YSGVPTYADDFKGRFAFSLETSASTAYLQINNLKNEDTATYFCARSHSNYDFW |
| | | GQGTTLTVSS |

TABLE 14
Example variable domain sequences of the anti-EpCAM light chain binding domains. CDRs,
as determined according to Kabat nomenclature, are underlined and bolded.

| Construct | SEQ | Amino Acid Sequence |
|---|---|---|
| Description | ID NO | (N to C) |
| mAb-1 | 417 | DIQMTQSPSSLSASLGERVSLTCRASQDIGNSLNWLQQKPDGTIKRLIYVTSRLD |
| | | SGVPKRFSGSRSGSDYFFTISSLESEDFADYYCLQYASSPFTFGSGTKLEIK |
| 001-11 | 418 | DIQMTQSPSSLSASVGDRVTLTCRASQDIGNSLNWLQQKPGKAPKRLIYVTSRL |
| | | DSGVPSRFSGSGSGTDYTFTISSLQPEDFATYYCLQYASSPFTFGQGTKLEIK |
| 001-12 | 513 | DIQMTQSPSSLSASVGDRVTLTCRASQDIGNSLNWLQQKPGKAPKRLIYVTSRLD |
| | | SGVPSRFSGSRSGTDYTFTISSLQPEDFATYYCLQYASSPFTFGQGTKLEIK |
| 001-18 | 419 | DIQMTQSPSSLSASVGDRVTLTCRASQDIGNSLNWLQQKPGKAPKRLIYVTSRL |
| | | DSGVPSRFSGSRSGTDYTFTISSLQPEDFATYYCLQYASSPFTFGQGTKLEIK |
| mAb-2 | 420 | DILLTQSPAILSVSPGERVSFSCRASQSIGTSLHWYQQRTNGSPRLLIKYASESISG |
| | | IPSRFSGSGSGTDFTLSINSVESEDIADYYCQQSNSWPYTFGGGTKLEIK |
| 002-7/8 | 421 | DIQLTQSPSSLSASVGDRVTFTCRASQSIGTSLHWYQQKPGKAPKLLIKYASESIS |
| | | GIPSRFSGSGSGTDFTLTISSVQPEDIATYYCQQSNSWPYTFGQGTKLEIK |
| 002-8 | 514 | DIQLTQSPSSLSASVGDRVTFTCRASQSIGTSLHWYQQKPGKAPKLLIKYASESIS |
| | | GIPSRFSGSGSGTDFTLTISSVQPEDIATYYCQQSNSWPYTFGQGTKLEIK |
| 002-11 | 422 | DIQLTQSPSSLSASVGDRVTFTCRASQSIGTSLHWYQQKPGKAPKLLIKYASESIS |
| | | GIPSRFSGSGSGTDFTLTISSVQPEDFATYYCQQSNSWPYTFGQGTKLEIK |
| mAb-4 | 423 | DVLLTQTPLSLSVSLGDQASISCRSSQSFVHINGNTYLEWFLQKPGLSPKLLIYK |
| | | VSNRFSGVPDRFSGSGSGTDFTLKISRVEAEDLGIYYCFQGSHVPLTFGAGTKLE |
| | | LK |
| mAb-8 | 424 | EIVLTQSPAITAASLGQKVTITCSASSSVSDMHWYQQKSGTSPKPWIYEISKLASG |
| | | VPTRFSGSGSGTSYSLTISSMEAEDAAIYYCQHWSYPLMTFGGGTKLEIK |
| 008-1 | 425 | DIQMTQSPSSLSASVGDRVTITCRASSSVSDMHWYQQKPGKAPKLLIYEISKLAS |
| | | GVPSRFSGSGSGTDFTLTISSLQPEDFATYYCQHWSYPLMTFGQGTKLEIK |
| mAb8-1 | 426 | DIQMTQSPSSLSASVGDRVTITCRASSSVSDMHWYQQKPGKAPKLLIYEISKLAS |
| | | GVPSRFSGSGSGTDFTLTISSLQPEDFATYYCQHWSYPLMTFGQGTKLEIK |
| mAb-21 | 427 | DVVMTQTPLTLSVTIGQPASISCKSSQSLLDSDGKTYLNWLLQRPGQSPKRLIYL |
| | | VSKLDSGVPDRFTGSGSGTDFTLKISRVEAEDLGVYYCWQGTHFPQTFGGGTKL |
| | | EIK |
| mAb-28 | 428 | DIVMTQAAFSNPVTLGTSASISCRSSKSLLHSNGFTYLHWYLQKPGQSPQLLIYQ |
| | | MSSLASGVPDRESSSGSGTDFTLRISRVEAEDVGVYYCAQNLELPRTFGGGTKL |
| | | EIK |
| mAb-30 | 429 | DVLMTQSPLSLPVSLGDQASISCRSSQSIVDSDGSTYLEWYLQKPGQSPKLLIYK |
| | | VSNRFSGVPDRFSGSGSGTDFTLKISGVEAEDLGVYYCFQGSHVPYTFGGGTKL |
| | | EIK |
| mAb-33 | 430 | DIVLTQSPASLAVSLGQRATISCRASESVDNSGISFMHWYQQKPGQSPKLLIYRA |
| | | SNLESGIPARFSGSGSRTDFTLTINPVETDDVATYYCQQSYENPWTFGGGTKLEI |
| | | K |
| mAb-35 | 431 | DIVLTQSPASLAVSLGQRATISCRASESVDNYGISFMHWFQQKPGQPPKLLIFRA |
| | | SSLESGIPARFSGSGSRTDFTLTINPVETDDVATYYCQQSYKDPWTFGGATKLEI |
| | | K |
| mAb-36 | 432 | EIVLTQSPAITAAFLGQKVTITCSASSSVSYIHWYQQKSDTSPKPWIYEISKLASG |
| | | VPARFSGSGSGTSYSLTISSMEAEDAAIYYCQQWNYPLITFGGGTKLEIK |
| mAb-37 | 433 | DIVLTQSPTSLAVSLGQRATISCRASESVDNYGISFMHWYQQKPGQPPKLLIYRA |
| | | SSLESGIPARFSGSGSRTDFTLTINPVESDDVATYYCQQSYKDPWTFGGATKLEIK |
| mAb-39 | 434 | DIVMTQSQKFMSTTVGDRVSITCQASQNVGTDVGWYQQKPGQSPKLLIYSASN |
| | | RYTGVPDRFTGSGSGTDFTLTISNMQSEDLADYFCQQYSSYPRTFGGGTKLEIK |

TABLE 15
Example full-length heavy chain sequences of the anti-EpCAM binding domains. CDRs, as
determined according to Kabat nomenclature, are underlined and bolded.

| Construct | SEQ | Amino Acid Sequence |
|---|---|---|
| Description | ID NO | (N to C) |
| mAb-1 | 435 | QVQLQQSGPELVKPGTSVKLSCRASGYTFTSYDINWVKQRPGQGLEWIGWIYPR |
| | | DGSTKYNERFKGKATLTVDTSSSTAYMEFHTLTSEDSAVYFCARDPYYYGTIY |
| | | GYFDVWGTGTTVTVSSASTKGPSVFPLAPSSKSTSGGTAALGCLVKDYFPEPVT |
| | | VSWNSGALTSGVHTFPAVLQSSGLYSLSSVVTVPSSSLGTQTYICNVNHKPSNT |
| | | KVDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVFSC |
| | | SVMHEALHNHYTQKSLSLSPGK |
| 001-11 | 436 | QVQLVQSGAEVKKPGASVKVSCKASGYTFTSYDINWVRQAPGQGLEWMGWIYP |
| | | RDGSTKYAQKFQGRVTMTVDTSTSTAYMELSSLRSEDTAVYYCARDPYYYGTI |
| | | YGYFDVWGQGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQ |
| | | QGNVFSCSVMHEALHNHYTQKSLSLSPGK |
| 001-12 | 515 | QVQLVQSGAEVKKPGASVKVSCKASGYTFTSYDINWVRQAPGQGLEWMGWIYP |
| | | RDGSTKYAQKFQGRVTMTVDTSTSTAYMELSSLRSEDTAVYYCARDPYYYGTI |
| | | YGYFDVWGQGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQ |
| | | QGNVFSCSVMHEALHNHYTQKSLSLSPGK |
| 001-18 | 437 | QVQLVQSGAEVKKPGASVKVSCKASGYTFTSYDINWVRQAPGQGLEWMGWIYP |
| | | REGSTKYAQKFQGRVTMTVDTSTSTAYMELSSLRSEDTAVYYCARDPYYYGTI |
| | | YGYFDVWGQGTTVTVSSASTKGPSVFPLAPSSKSTSGGTAALGCLVKDYFPEP |
| | | VTVSWNSGALTSGVHTFPAVLQSSGLYSLSSVVTVPSSSLGTQTYICNVNHKP |
| | | SNTKVDKKVEPKSCDKTHTCPPCPAPELLGGPSVFLFPPKPKDTLMISRTPEV |
| | | TCVVVDVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQ |
| | | QGNVFSCSVMHEALHNHYTQKSLSLSPGK |
| mAb-2 | 438 | EVQLVESGGGLVQPKGSLKLSCAASGFTFNTYAMHWVRQAPGKGLEWVARIRS |
| | | KTYNYATFYADSVKDRFTISRDDSQSMLYLQMNNLKTEDTAMFYCVTGSSYAM |
| | | DYWGQGTSVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLNG |
| | | KEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNV |
| | | FSCSVMHEALHNHYTQKSLSLSPGK |
| 002-7/8 | 439 | EVQLVESGGGLVQPGGSLRLSCAASGFTFNTYAMHWVRQAPGKGLEWVARIRS |
| | | KTYNYATFYADSVKGRFTISRDNSKNTLYLQMNSLRAEDTAVYYCVTGSSYAM |
| | | DYWGQGTTVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLNG |
| | | KEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNV |
| | | FSCSVMHEALHNHYTQKSLSLSPGK |
| 002-8 | 516 | EVQLVESGGGLVQPGGSLRLSCAASGFTFNTYAMHWVRQAPGKGLEWVARIRS |
| | | KTYNYATFYADSVKGRFTISRDNSKNTLYLQMNSLRAEDTAVYYCVTGSSYAM |
| | | DYWGQGTTVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLNG |
| | | KEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNV |
| | | FSCSVMHEALHNHYTQKSLSLSPGK |
| 002- | 440 | EVQLVESGGGLVQPGGSLRLSCAASGFTFNTYAMHWVRQAPGKGLEWVARIRS |
| 11.huIgG1 | | KTYNYATFYADSVKGRFTISRDDSKNTLYLQMNSLRAEDTAVYYCVTGSSYAM |
| | | DYWGQGTTVTVSSASTKGPSVFPLAPSSKSTSGGTAALGCLVKDYFPEPVTVS |
| | | WNSGALTSGVHTFPAVLQSSGLYSLSSVVTVPSSSLGTQTYICNVNHKPSNTK |
| | | VDKKVEPKSCDKTHTCPPCPAPELLGGPSVFLFPPKPKDTLMISRTPEVTCVV |
| | | VDVSHEDPEVKFNWYVDGVEVHNAKTKPREEQYNSTYRVVSVLTVLHQDWLNG |
| | | KEYKCKVSNKALPAPIEKTISKAKGQPREPQVYTLPPSREEMTKNQVSLTCLV |
| | | KGFYPSDIAVEWESNGQPENNYKTTPPVLDSDGSFFLYSKLTVDKSRWQQGNV |
| | | FSCSVMHEALHNHYTQKSLSLSPGK |
| mAb-8 | 441 | QVQLQQSGAELARPGASVQLSCKASGYTFTSYGISWLKQRAGQGLEWIGEIYPR |
| | | SDNTYYNEKFKGKATLTADKSSSTAYMELRSLTSEDSAVYFCARRAGMWFAYWG |
| | | QGTLVTVSAASTKGPSVFPLAPSSKSTSGGTAALGCLVKDYFPEPVTVSWNSGA |
| | | LTSGVHTFPAVLQSSGLYSLSSVVTVPSSSLGTQTYICNVNHKPSNTKVDKKVE |
| | | PKSCDKTHTCPPCPAPELLGGPSVFLFPPKPKDTLMISRTPEVTCVVVDVSHED |
| | | PEVKFNWYVDGVEVHNAKTKPREEQYNSTYRVVSVLTVLHQDWLNGKEYKCKVS |
| | | NKALPAPIEKTISKAKGQPREPQVYTLPPSREEMTKNQVSLTCLVKGFYPSDIA |
| | | VEWESNGQPENNYKTTPPVLDSDGSFFLYSKLTVDKSRWQQGNVFSCSVMHEAL |
| | | HNHYTQKSLSLSPGK |
| 008-1.huIgG1 | 442 | QVQLVQSGAEVKKPGSSVKVSCKASGYTFTSYGISWVRQAPGQGLEWMGEIYP |
| | | RSDNTYYAQKFQGRVTITADKSTSTAYMELSSLRSEDTAVYYCARRAGMWFAY |
| | | WGQGTLVTVSSASTKGPSVFPLAPSSKSTSGGTAALGCLVKDYFPEPVTVSWN |
| | | SGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGNVFS |
| | | CSVMHEALHNHYTQKSLSLSPGK |
| mAb8- | 443 | QVQLVQSGAEVKKPGSSVKVSCKASGYTFTSYGISWVRQAPGQGLEWMGEIYP |
| 1.huIgG1.knob | | RSDNTYYAQKFQGRVTITADKSTSTAYMELSSLRSEDTAVYYCARRAGMWFAS |
| | | YWGQGTLVTVSSASTKGPSVFPLAPSSKSTSGGTAALGCLVKDYFPEPVTVSW |
| | | NGALTSGVHTFPAVLQSSGLYSLSSVVTVPSSSLGTQTYICNVNHKPSNTKVD |
| | | KKVEPKSCDKTHTCPPCPAPELLGGPSVFLFPPKPKDTLMISRTPEVTCVVVD |
| | | VSHEDPEVKFNWYVDGVEVHNAKTKPREEQYNSTYRVVSVLTVLHQDWLNGKE |
| | | YKCKVSNKALPAPIEKTISKAKGQPREPQVYTLPPSRDELTKNQVSLWCLVKG |
| | | FYPSDIAVEWESNGQPENNYKTTPPVLDSDGSFFLYSKLTVDKSRWQQGNVFS |
| | | CSVMHEALHNHYTQKSLSLSPGK |

TABLE 16
Example full-length light chain sequences of the anti-EpCAM binding domains. CDRs, as
determined according to Kabat nomenclature, are underlined and bolded.

| Construct | SEQ | Amino Acid Sequence |
|---|---|---|
| Description | ID NO | (N to C) |
| mAb-1 | 444 | DIQMTQSPSSLSASLGERVSLTCRASQDIGNSLNWLQQKPDGTIKRLIYVTSRLD |
| | | SGVPKRFSGSRSGSDYFFTISSLESEDFADYYCLQYASSPFTFGSGTKLEIKRTVA |
| | | APSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| 001-11 | 445 | DIQMTQSPSSLSASVGDRVTLTCRASQDIGNSLNWLQQKPGKAPKRLIYVTSRL |
| | | DSGVPSRFSGSGSGTDYTFTISSLQPEDFATYYCLQYASSPFTFGQGTKLEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| 001-12 | 517 | DIQMTQSPSSLSASVGDRVTLTCRASQDIGNSLNWLQQKPGKAPKRLIYVTSRL |
| | | DSGVPSRFSGSRSGTDYTFTISSLQPEDFATYYCLQYASSPFTFGQGTKLEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| 001-18 | 446 | DIQMTQSPSSLSASVGDRVTLTCRASQDIGNSLNWLQQKPGKAPKRLIYVTSRL |
| | | DSGVPSRFSGSRSGTDYTFTISSLQPEDFATYYCLQYASSPFTFGQGTKLEIKRTV |
| | | AAPSVFIFPPSDEQLKSGTASVVCLLNNFYPREAKVQWKVDNALQSGNSQESVT |
| | | EQDSKDSTYSLSSTLTLSKADYEKHKVYACEVTHQGLSSPVTKSFNRGEC |
| mAb-2 | 447 | DILLTQSPAILSVSPGERVSFSCRASQSIGTSLHWYQQRTNGSPRLLIKYASESISG |
| | | IPSRFSGSGSGTDFTLSINSVESEDIADYYCQQSNSWPYTFGGGTKLEIKRTVAAP |
| | | SVFIFPPSDEQLKSGTASVVCLLNNFYPREAKVQWKVDNALQSGNSQESVTEQD |
| | | SKDSTYSLSSTLTLSKADYEKHKVYACEVTHQGLSSPVTKSFNRGEC |
| 002-7/8 | 448 | DIQLTQSPSSLSASVGDRVTFTCRASQSIGTSLHWYQQKPGKAPKLLIKYASESIS |
| | | GIPSRFSGSGSGTDFTLTISSVQPEDIATYYCQQSNSWPYTFGQGTKLEIKRTVAA |
| | | PSVFIFPPSDEQLKSGTASVVCLLNNFYPREAKVQWKVDNALQSGNSQESVTEQ |
| | | DSKDSTYSLSSTLTLSKADYEKHKVYACEVTHQGLSSPVTKSFNRGEC |
| 002-8 | 518 | DIQLTQSPSSLSASVGDRVTFTCRASQSIGTSLHWYQQKPGKAPKLLIKYASESIS |
| | | GIPSRFSGSGSGTDFTLTISSVQPEDIATYYCQQSNSWPYTFGQGTKLEIKRTVAA |
| | | PSVFIFPPSDEQLKSGTASVVCLLNNFYPREAKVQWKVDNALQSGNSQESVTEQ |
| | | DSKDSTYSLSSTLTLSKADYEKHKVYACEVTHQGLSSPVTKSFNRGEC |
| 002-11 | 449 | DIQLTQSPSSLSASVGDRVTFTCRASQSIGTSLHWYQQKPGKAPKLLIKYASESIS |
| | | GIPSRFSGSGSGTDFTLTISSVQPEDFATYYCQQSNSWPYTFGQGTKLEIKRTVAA |
| | | PSVFIFPPSDEQLKSGTASVVCLLNNFYPREAKVQWKVDNALQSGNSQESVTEQ |
| | | DSKDSTYSLSSTLTLSKADYEKHKVYACEVTHQGLSSPVTKSFNRGEC |
| mAb-8 | 450 | EIVLTQSPAITAASLGQKVTITCSASSSVSDMHWYQQKSGTSPKPWIYEISKLASG |
| | | VPTRFSGSGSGTSYSLTISSMEAEDAAIYYCQHWSYPLMTFGGGTKLEIKRTVA |
| | | APSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| 008-1 | 451 | DIQMTQSPSSLSASVGDRVTITCRASSSVSDMHWYQQKPGKAPKLLIYEISKLAS |
| | | GVPSRFSGSGSGTDFTLTISSLQPEDFATYYCQHWSYPLMTFGQGTKLEIKRTVA |
| | | APSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |
| mAb8-1 | 452 | DIQMTQSPSSLSASVGDRVTITCRASSSVSDMHWYQQKPGKAPKLLIYEISKLAS |
| | | GVPSRFSGSGSGTDFTLTISSLQPEDFATYYCQHWSYPLMTFGQGTKLEIKRTVA |
| | | APSVFIFPPSDEQLKSGTASVVCLLNNFYPREAKVQWKVDNALQSGNSQESVTE |
| | | QDSKDSTYSLSSTLTLSKADYEKHKVYACEVTHQGLSSPVTKSFNRGEC |

TABLE 17
Example single-chain variable fragment (scFv) sequence(s) of the anti-EpCAM binding
domains.

| Construct | Amino Acid Sequence | SEQ |
|---|---|---|
| Description | (N to C) | ID NO |
| EpCAM. | DIQMTQSPSSLSASVGDRVTITCRASSSVSDMHWYQQKPGKAPKLLIYEISKLASG | 453 |
| Mab008-1. | VPSRFSGSGSGTDFTLTISSLQPEDFATYYCQHWSYPLMTFGQGTKLEIKGGGGSG | |
| scFv hole. | GGGSGGGGSQVQLVQSGAEVKKPGSSVKVSCKASGYTFTSYGISWVRQAPGQGL | |
| His | EWMGEIYPRSDNTYYAQKFQGRVTITADKSTSTAYMELSSLRSEDTAVYYCARR | |
| | AGMWFAYWGQGTLVTVSSGGSGGSGGASKTHTCPPCPAPELLGGPSVFLFPPKP | |
| | KDTLMISRTPEVTCVVVDVSHEDPEVKFNWYVDGVEVHNAKTKPREEQYNSTY | |
| | RVVSVLTVLHQDWLNGKEYKCKVSNKALPAPIEKTISKAKGQPREPQVYTLPPSR | |
| | DELTKNQVSLSCAVKGFYPSDIAVEWESNGQPENNYKTTPPVLDSDGSFFLVSKL | |
| | TVDKSRWQQGNVFSCSVMHEALHNHYTQKSLSLSPGGGGSHHHHHHHH | |
| EpCAM. | DIQMTQSPSSLSASVGDRVTLTCRASQDIGNSLNWLQQKPGKAPKRLIYVTSRLD | 454 |
| Mab001-11. | SGVPSRFSGSGSGTDYTFTISSLQPEDFATYYCLQYASSPFTFGQGTKLEIKGGGGS | |
| scFv hole. | GGGGSGGGGSQVQLVQSGAEVKKPGASVKVSCKASGYTFTSYDINWVRQAPGQ | |
| His | GLEWMGWIYPRDGSTKYAQKFQGRVTMTVDTSTSTAYMELSSLRSEDTAVYYC | |
| | ARDPYYYGTIYGYFDVWGQGTTVTVSSGGSGGSGGASKTHTCPPCPAPELLGGPS | |
| | VFLFPPKPKDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKAKGQPREPQ | |
| | VYTLPPSRDELTKNQVSLSCAVKGFYPSDIAVEWESNGQPENNYKTTPPVLDSDG | |
| | SFFLVSKLTVDKSRWQQGNVFSCSVMHEALHNHYTQKSLSLSPGGGGSHHHHHH | |
| | HH | |
| EpCAM. | DIQLTQSPSSLSASVGDRVTFTCRASQSIGTSLHWYQQKPGKAPKLLIKYASESISG | 455 |
| Mab002-7/8. | IPSRFSGSGSGTDFTLTISSVQPEDIATYYCQQSNSWPYTFGQGTKLEIKGGGGSGG | |
| scFv hole. | GGSGGGGSEVQLVESGGGLVQPGGSLRLSCAASGFTFNTYAMHWVRQAPGKGL | |
| His | EWVARIRSKTYNYATFYADSVKGRFTISRDNSKNTLYLQMNSLRAEDTAVYYCV | |
| | TGSSYAMDYWGQGTTVTVSSGGSGGSGGASKTHTCPPCPAPELLGGPSVFLFPPK | |
| | PKDTLMISRTPEVTCVVVDVSHEDPEVKFNWYVDGVEVHNAKTKPREEQYNSTY | |
| | RVVSVLTVLHQDWLNGKEYKCKVSNKALPAPIEKTISKAKGQPREPQVYTLPPSR | |
| | DELTKNQVSLSCAVKGFYPSDIAVEWESNGQPENNYKTTPPVLDSDGSFFLVSKL | |
| | TVDKSRWQQGNVFSCSVMHEALHNHYTQKSLSLSPGGGGSHHHHHHHH | |
| EpCAM. | DIQMTQSPSSLSASVGDRVTLTCRASQDIGNSLNWLQQKPGKAPKRLIYVTSRLD | 456 |
| Mab001-18. | SGVPSRFSGSRSGTDYTFTISSLQPEDFATYYCLQYASSPFTFGQGTKLEIKGGGGS | |
| scFv hole. | GGGGSGGGGSQVQLVQSGAEVKKPGASVKVSCKASGYTFTSYDINWVRQAPGQ | |
| His | GLEWMGWIYPREGSTKYAQKFQGRVTMTVDTSTSTAYMELSSLRSEDTAVYYC | |
| | ARDPYYYGTIYGYFDVWGQGTTVTVSSGGSGGSGGASKTHTCPPCPAPELLGGPS | |
| | VFLFPPKPKDTLMISRTPEVTCVVVDVSHEDPEVKFNWYVDGVEVHNAKTKPRE | |
| | EQYNSTYRVVSVLTVLHQDWLNGKEYKCKVSNKALPAPIEKTISKAKGQPREPQ | |
| | VYTLPPSRDELTKNQVSLSCAVKGFYPSDIAVEWESNGQPENNYKTTPPVLDSDG | |
| | SFFLVSKLTVDKSRWQQGNVFSCSVMHEALHNHYTQKSLSLSPGGGGSHHHHHH | |
| | HH | |
| EpCAM. | DIQLTQSPSSLSASVGDRVTFTCRASQSIGTSLHWYQQKPGKAPKLLIKYASESISG | 457 |
| Mab002-11. | IPSRFSGSGSGTDFTLTISSVQPEDFATYYCQQSNSWPYTFGQGTKLEIKGGGGSG | |
| scFv hole | GGGSGGGGSEVQLVESGGGLVQPGGSLRLSCAASGFTFNTYAMHWVRQAPGKG | |
| | LEWVARIRSKTYNYATFYADSVKGRFTISRDDSKNTLYLQMNSLRAEDTAVYYC | |
| | VTGSSYAMDYWGQGTTVTVSSGGSGGSGGASKTHTCPPCPAPELLGGPSVFLFPP | |
| | KPKDTLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVYTLPPS | |
| | RDELTKNQVSLSCAVKGFYPSDIAVEWESNGQPENNYKTTPPVLDSDGSFFLVSK | |
| | LTVDKSRWQQGNVFSCSVMHEALHNHYTQKSLSLSPGK | |

TABLE 18
Example TCR-chimera format sequence(s)
of the anti-EpCAM binding domains.

| Construct | Amino Acid Sequence | SEQ |
|---|---|---|
| Description | (N to C) | ID NO |
| Anti-EpCAM | DIQLTQSPSSLSASVGDRVT | 458 |
| TCRα LC | FTCRASQSIGTSLHWYQQKP | |
| | GKAPKLLIKYASESISGIPS | |
| | RFSGSGSGTDFTLTISSVQP | |
| | EDFATYYCQQSNSWPYTFGQ | |
| | GTKLEIKPDIQNPDPAVYQL | |
| | RDSKSSDKSVCLFTDFDSQT | |
| | QVSQSKDSDVYITDKCVLDM | |
| | RSMDFKSNSAVAWSQKSDFA | |
| | CANAFQNSIIPEDTFFPSPE | |
| | SS | |
| Anti-EpCAM | EVQLVESGGGLVQPGGSLRL | 459 |
| TCRβ HC | SCAASGFTFNTYAMHWVRQA | |
| | PGKGLEWVARIRSKTYNYAT | |
| | FYADSVKGRFTISRDDSKNT | |
| | LYLQMNSLRAEDTAVYYCVT | |
| | GSSYAMDYWGQGTTVTVLED | |
| | LKNVFPPEVAVFEPSEAEIS | |
| | HTQKATLVCLATGFYPDHVE | |
| | LSWWVNGKEVHSGVCTDPQP | |
| | LKEQPALQDSRYALSSRLRV | |
| | SATFWQNPRNHFRCQVQFYG | |
| | LSENDEWTQDRAKPVTQIVS | |
| | AEAWGRASDKTHTCPPCPAP | |
| | ELLGGPSVFLFPPKPKDTLM | |
| | ISRTPEVTCVVVDVSHEDPE | |
| | VKFNWYVDGVEVHNAKTKPR | |
| | EEQYNSTYRVVSVLTVLHQD | |
| | WLNGKEYKCKVSNKALPAPI | |
| | EKTISKAKGQPREPQVYTLP | |
| | PCREEMTKNQVSLWCLVKGF | |
| | YPSDIAVEWESNGQPENNYK | |
| | TTPPVLDSDGSFFLYSKLTV | |
| | DKSRWQQGNVFSCSVMHEAL | |
| | HNHYTQKSLSLSPG | |

### Anti-EGFR Binding Domains

In certain embodiments, disclosed herein is an anti-EGFR binding domain. In some embodiments, the EGFR-binding domain comprises a heavy chain variable domain and a light chain variable domain. In some embodiments, the EGFR binding domain comprises a single-chain variable fragment (scFv). In some embodiments, the EGFR binding domain comprises an antigen-binding fragment (Fab).

In some embodiments, the EGFR-binding domain comprises three heavy chain (HC) complementarity determining regions (CDRs), HC-CDR1, HC-CDR2, and HC-CDR3; wherein the HC-CDR1, the HC-CDR2, and the HC-CDR3 of the EGFR binding domain comprises amino acid sequences selected from those set forth in Table 19; and wherein at least one of the HC-CDR1, the HC-CDR2, and the HC-CDR3 comprise from 0-3 amino acid modification(s). In some embodiments, at least one of the HC-CDR1, the HC-CDR2, and the HC-CDR3 of the EGFR-binding domain comprise from 0-2 amino acid modification(s). In some embodiments, at least one of the HC-CDR1, the HC-CDR2, and the HC-CDR3 of the EGFR-binding domain comprise from 0-1 amino acid modification(s).

In some embodiments, the HC-CDR1, the HC-CDR2, and the HC-CDR3 of the CD47 binding domain comprise the amino acid sequences of SEQ ID NOs: 460-462; and wherein at least one of the HC-CDR1, the HC-CDR2, and the HC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 463-465; and wherein at least one of the HC-CDR1, the HC-CDR2, and the HC-CDR3 comprise from 0-3 (e.g., 0-2, or 0-1) amino acid modification(s).

In some embodiments, the EGFR binding domain comprises three light chain (LC) complementarity determining regions (CDRs), the LC-CDR1, the LC-CDR2, and the LC-CDR3; wherein the LC-CDR1, the LC-CDR2, and the LC-CDR3 of the EGFR binding domain comprises amino acid sequences selected from those set forth in Table 20; and wherein at least one of the LC-CDR1, the LC-CDR2, and the LC-CDR3 comprise from 0-3 amino acid modification(s). In some embodiments, at least one of the LC-CDR1, the LC-CDR2, and the LC-CDR3 of the EGFR-binding domain comprise from 0-2 amino acid modification(s). In some embodiments, at least one of the LC-CDR1, the LC-CDR2, and the LC-CDR3 of the EGFR-binding domain comprise from 0-1 amino acid modification(s).

In some embodiments, the LC-CDR1, the LC-CDR2, and the LC-CDR3 of the CD47 binding domain comprise the amino acid sequences of SEQ ID NOs: 466-468; and wherein at least one of the LC-CDR1, the LC-CDR2, and the LC-CDR3 comprise from 0-3 (e.g., 0-2, or 0-1) amino acid modification(s).

In some embodiments, the LC-CDR1, the LC-CDR2, and the LC-CDR3 of the CD47 binding domain comprise the amino acid sequences of SEQ ID NOs: 469-471; and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 460-462; and the LC-CDR1, the LC-CDR2, and the LC-CDR3 of the CD47 binding domain comprise the amino acid sequences of SEQ ID NOs: 466-468; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the HC-CDR1, the HC-CDR2, and the HC-CDR3 of the CD47 binding domain comprise the amino acid sequences of SEQ ID NOs: 463-465; and the LC-CDR1, the LC-CDR2, and the LC-CDR3 of the CD47 binding domain comprise the amino acid sequences of SEQ ID NOs: 469-471; wherein at least one of the HC-CDR1, the HC-CDR2, and the HC-CDR3 comprise from 0-3 (e.g., 0-2, or 0-1) amino acid modification(s); and wherein at least one of the LC-CDR1, the LC-CDR2, and the LC-CDR3 comprise from 0-3 (e.g., 0-2, or 0-1) amino acid modification(s).

In some embodiments, the EGFR binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 21.

In some embodiments, the EGFR binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 472 or 473.

In some embodiments, the EGFR binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 22.

In some embodiments, the EGFR binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 474 or 475.

In some embodiments, the EGFR binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 21; and a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 22.

In some embodiments, the EGFR binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 472 or 473; and a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 474 or 475.

In some embodiments, the EGFR binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 23.

In some embodiments, the EGFR binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 476 or 477.

In some embodiments, the EGFR binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 24.

In some embodiments, the EGFR binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 478 or 479.

In some embodiments, the EGFR binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 23; and a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 24.

In some embodiments, the EGFR binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 476 or 477; and a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 478 or 479.

In some embodiments, the EGFR binding domain comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 25.

In some embodiments, the EGFR binding domain comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 480 or 481.

In some embodiments of an anti-EGFR binding domain described herein, a heavy chain variable fragment (VH) of the anti-EGFR binding domain is fused to a first T-cell receptor (TCR) constant region, and a light chain variable domain (VL) of the anti-EGFR binding domain is fused to a second TCR constant region, wherein the first and second TCR constant regions are capable of forming a dimer. In some embodiments, the TCR constant regions are the TCR alpha and TCR beta constant regions. In some embodiments, the VH-TCR fusion polypeptide is fused to a human IgG (e.g., IgG1) Fc domain. See WO 2019/057122.

In some embodiments, an anti-EGFR binding domain described herein comprises a T cell receptor (TCR) constant region. In some embodiments, the TCR constant region comprises a TCR alpha constant domain and a TCR beta constant domain. In some embodiments, the anti-EGFR binding domain comprises a VH-EGFR domain and a VL-EGFR domain, wherein the VH-EGFR domain is fused to the TCR beta constant domain; and the VL-EGFR domain is fused to the TCR alpha constant domain. In some embodiments, the anti-EGFR binding domain comprises a VH-EGFR domain and a VL-EGFR domain, wherein the VH-EGFR domain is fused to the first constant domain of heavy chain (CH1) IgG domain; and the VL-EGFR domain is fused to the constant domain of light chain (CL) IgG domain.

In some embodiments, an anti-EGFR binding domain described herein comprises a T cell receptor (TCR) constant region. In some embodiments, the TCR constant region comprises a TCR alpha constant domain and a TCR beta constant domain. In some embodiments, an anti-EGFR binding domain described herein comprises a VH-EGFR domain and a VL-EGFR domain, wherein the VH-EGFR domain is fused to the TCR alpha constant domain; and the VL-EGFR domain is fused to the TCR beta constant domain. In some embodiments, the anti-EGFR binding domain comprises a VH-EGFR domain and a VL-EGFR domain, wherein the VH-EGFR domain is fused to the first constant domain of heavy chain (CH1) IgG domain; and the VL-EGFR domain is fused to the constant domain of light chain (CL) IgG domain.

In some embodiments, an anti-EGFR binding domain described herein has enhanced binding to human EGFR protein compared to a reference antibody. In some cases, the enhance binding is a decrease in KD of at least 10%, 20%, 30%, 40%, 50%, 60%, 70%, 80%, 90%, 100% compared to reference antibody.

In some embodiments, an anti-EGFR binding domain described herein has an EC50 of from about 0.023 nM to about 3.07 nM. In some embodiments, an anti-EGFR binding domain described herein has an EC50 of from at least about 0.023 nM. In some embodiments, an anti-EGFR binding domain described herein has an EC50 of from at most about 3.07 nM. In some embodiments, an anti-EGFR binding domain described herein has an EC50 of from about 0.023 nM to about 0.024 nM, about 0.023 nM to about 0.036 nM, about 0.023 nM to about 0.038 nM, about 0.023 nM to about 0.04 nM, about 0.023 nM to about 0.059 nM, about 0.023 nM to about 0.069 nM, about 0.023 nM to about 0.086 nM, about 0.023 nM to about 3.07 nM, about 0.024 nM to about 0.036 nM, about 0.024 nM to about 0.038 nM, about 0.024 nM to about 0.04 nM, about 0.024 nM to about 0.059 nM, about 0.024 nM to about 0.069 nM, about 0.024 nM to about 0.086 nM, about 0.024 nM to about 3.07 nM, about 0.036 nM to about 0.038 nM, about 0.036 nM to about 0.04 nM, about 0.036 nM to about 0.059 nM, about 0.036 nM to about 0.069 nM, about 0.036 nM to about 0.086 nM, about 0.036 nM to about 3.07 nM, about 0.038 nM to about 0.04 nM, about 0.038 nM to about 0.059 nM, about 0.038 nM to about 0.069 nM, about 0.038 nM to about 0.086 nM, about 0.038 nM to about 3.07 nM, about 0.04 nM to about 0.059 nM, about 0.04 nM to about 0.069 nM, about 0.04 nM to about 0.086 nM, about 0.04 nM to about 3.07 nM, about 0.059 nM to about 0.069 nM, about 0.059 nM to about 0.086 nM, about 0.059 nM to about 3.07 nM, about 0.069 nM to about 0.086 nM, about 0.069 nM to about 3.07 nM, or about 0.086 nM to about 3.07 nM in an in vitro cytotoxicity assay to determine ADCC activity, for instance, using NK92/CD16a176V effector cells and targeting cancer cells such as metastatic cancer cells from a vulvar squamous cell carcinoma, for example, A431 cells.

TABLE 19
Example CDR sequences of the anti-EGFR
heavy chain binding domains, as determined
according to Kabat nomenclature.

| | SEQ | | SEQ | | SEQ | |
|---|---|---|---|---|---|---|
| | ID | | ID | | ID | |
| Construct | NO | CDR1 | NO | CDR2 | NO | CDR3 |
| Cetuximab | 460 | NYGVH | 461 | VIWSGG | 462 | ALTYY |
| | | | | NTDYNT | | DYEFA |
| | | | | PFTS | | Y |
| Panitumumab | 463 | SGDY | 464 | HIYYSGN | 465 | DRVTG |
| | | YWT | | TNYNPS | | AFDI |
| | | | | LKS | | |

TABLE 20
Example CDR sequences of the anti-EGFR
light chain binding domains, as determined
according to Kabat nomenclature.

| Construct | SEQ ID | | SEQ ID | | SEQ ID | |
|---|---|---|---|---|---|---|
| | NO | CDR1 | NO | CDR2 | NO | CDR3 |
| Cetuximab | 466 | RASQSI | 467 | YASE | 468 | QQNN |
| | | GTNIH | | SIS | | NWPT |
| | | | | | | T |
| Panitumumab | 469 | QASQDI | 470 | DASN | 471 | QHFD |
| | | SNYLN | | LET | | HLPL |
| | | | | | | A |

TABLE 21
Example variable domain sequences of the
anti-EGFR heavy chain binding domains.
CDRs, as determined according to
Kabat nomenclature, are underlined
and bolded.

| Construct | Amino Acid Sequence | SEQ |
|---|---|---|
| Description | (N to C) | ID NO |
| Cetuximab VH | QVQLKQSGPGLVQPSQSLSITCTVSG | 472 |
| | FSLTNYGVHWVRQSPGKGLEWLGVIW | |
| | SGGNTDYNTPFTSRLSINKDNSKSQV | |
| | FFKMNSLQSNDTAIYYCARALTYYDY | |
| | EFAYWGQGTLVTVSA | |
| Panitumumab VH | QVQLQESGPGLVKPSETLSLTCTVSG | 473 |
| | GSVSSGDYYWTWIRQSPGKGLEWIGH | |
| | IYYSGNTNYNPSLKSRLTISIDTSKT | |
| | QFSLKLSSVTAADTAIYYCVRDRVTG | |
| | AFDIWGQGTMVTVSS | |

TABLE 22
Example variable domain sequences of the
anti-EGFR light chain binding domains.
CDRs, as determined according to Kabat
nomenclature, are underlined and bolded.

| Construct | Amino Acid Sequence | SEQ |
|---|---|---|
| Description | (N to C) | ID NO |
| Cetuximab VL | DILLTQSPVILSVSPGERVSFSCRA | 474 |
| | SQSIGTNIHWYQQRINGSPRLLIKY | |
| | ASESISGIPSRFSGSGSGTDFTLSI | |
| | NSVESEDIADYYCQQNNNWPTTFGA | |
| | GTKLELK | |
| Panitumumab VL | DIQMTQSPSSLSASVGDRVTITCQA | 475 |
| | SQDISNYLNWYQQKPGKAPKLLIYD | |
| | ASNLETGVPSRFSGSGSGTDFTFTI | |
| | SSLQPEDIATYFCQHFDHLPLAFGG | |
| | GTKVEIK | |

TABLE 23
Example full-length heavy chain sequences
of the anti-EGFR binding domains. CDRs, as
determined according to Kabat
nomenclature, are underlined and bolded.

| Construct | Amino Acid Sequence | SEQ |
|---|---|---|
| Description | (N to C) | ID NO |
| Cetuximab | QVQLKQSGPGLVQPSQSLSITCTVS | 476 |
| Knob HC | GFSLTNYGVHWVRQSPGKGLEWLGV | |
| | IWSGGNTDYNTPFTSRLSINKDNSK | |
| | SQVFFKMNSLQSNDTAIYYCARALT | |
| | YYDYEFAYWGQGTLVTVSAASTKGP | |
| | SVFPLAPSSKSTSGGTAALGCLVKD | |
| | YFPEPVTVSWNSGALTSGVHTFPAV | |
| | LQSSGLYSLSSVVTVPSSSLGTQTY | |
| | ICNVNHKPSNTKVDKKVEPKSCDKT | |
| | HTCPPCPAPELLGGPSVFLFPPKPK | |
| | DTLMISRTPEVTCVVVDVSHEDPEV | |
| | KFNWYVDGVEVHNAKTKPREEQYNS | |
| | TYRVVSVLTVLHQDWLNGKEYKCKV | |
| | SNKALPAPIEKTISKAKGQPREPQV | |
| | YTLPPSRDELTKNQVSLWCLVKGFY | |
| | PSDIAVEWESNGQPENNYKTTPPVL | |
| | DSDGSFFLYSKLTVDKSRWQQGNVF | |
| | SCSVMHEALHNHYTQKSLSLSPGK | |
| Panitumumab. | QVQLQESGPGLVKPSETLSLTCTVS | 477 |
| knob.HC | GGSVSSGDYYWTWIRQSPGKGLEWI | |
| | GHIYYSGNTNYNPSLKSRLTISIDT | |
| | SKTQFSLKLSSVTAADTAIYYCVRD | |
| | RVTGAFDIWGQGTMVTVSSASTKGP | |
| | SVFPLAPSSKSTSGGTAALGCLVKD | |
| | YFPEPVTVSWNSGALTSGVHTFPAV | |
| | LQSSGLYSLSSVVTVPSSSLGTQTY | |
| | ICNVNHKPSNTKVDKKVEPKSCDKT | |
| | HTCPPCPAPELLGGPSVFLFPPKPK | |
| | DTLMISRTPEVTCVVVDVSHEDPEV | |
| | KFNWYVDGVEVHNAKTKPREEQYNS | |
| | TYRVVSVLTVLHQDWLNGKEYKCKV | |
| | SNKALPAPIEKTISKAKGQPREPQV | |
| | YTLPPSRDELTKNQVSLWCLVKGFY | |
| | PSDIAVEWESNGQPENNYKTTPPVL | |
| | DSDGSFFLYSKLTVDKSRWQQGNVF | |
| | SCSVMHEALHNHYTQKSLSLSPGK | |

TABLE 24
Example full-length light chain sequences of
the anti-EGFR binding domains. CDRs, as
determined according to Kabat
nomenclature, are underlined and bolded.

| Construct | Amino Acid Sequence | SEQ |
|---|---|---|
| Description | (N to C) | ID NO |
| Cetuximab LC | DILLTQSPVILSVSPGERVSFSCRA | 478 |
| | SQSIGTNIHWYQQRINGSPRLLIKY | |
| | ASESISGIPSRFSGSGSGTDFTLSI | |
| | NSVESEDIADYYCQQNNNWPTTFGA | |
| | GTKLELKRTVAAPSVFIFPPSDEQL | |
| | KSGTASVVCLLNNFYPREAKVQWK | |
| | VDNALQSGNSQESVTEQDSKDSTY | |
| | SLSSTLTLSKADYEKHKVYACEVT | |
| | HQGLSSPVTKSFNRGEC | |
| Panitumumab LC | DIQMTQSPSSLSASVGDRVTITCQ | 479 |
| | ASQDISNYLNWYQQKPGKAPKLLI | |
| | YDASNLETGVPSRFSGSGSGTDFT | |
| | FTISSLQPEDIATYFCQHFDHLPL | |
| | AFGGGTKVEIKRTVAAPSVFIFPP | |
| | SDEQLKSGTASVVCLLNNFYPREA | |
| | KVQWKVDNALQSGNSQESVTEQDS | |
| | KDSTYSLSSTLTLSKADYEKHKVY | |
| | ACEVTHQGLSSPVTKSFNRGEC | |

TABLE 25
Example of Fc-hole sequence and
anti-EGFR cetuximab.HC.hole sequence

| Construct | Amino Acid Sequence | SEQ |
|---|---|---|
| Description | (N to C) | ID NO |
| Fc-hole | EPKSCDKTHTCPPCPAPELLGGPSV | 480 |
| | FLFPPKPKDTLMISRTPEVTCVVVD | |
| | VSHEDPEVKFNWYVDGVEVHNAKTK | |
| | PREEQYNSTYRVVSVLTVLHQDWLN | |
| | GKEYKCKVSNKALPAPIEKTISKAK | |
| | GQPREPQVCTLPPSREEMTKNQVSL | |
| | SCAVKGFYPSDIAVEWESNGQPENN | |
| | YKTTPPVLDSDGSFFLVSKLTVDKS | |
| | RWQQGNVFSCSVMHEALHNHYTQKS | |
| | LSLSPGK | |
| Cetuximab.Fab | QVQLKQSGPGLVQPSQSLSITCTVS | 481 |
| HC | GFSLTNYGVHWVRQSPGKGLEWLGV | |
| | IWSGGNTDYNTPFTSRLSINKDNSK | |
| | SQVFFKMNSLQSNDTAIYYCARALT | |
| | YYDYEFAYWGQGTLVTVSAASTKGP | |
| | SVFPLAPSSKSTSGGTAALGCLVKD | |
| | YFPEPVTVSWNSGALTSGVHTFPAV | |
| | LQSSGLYSLSSVVTVPSSSLGTQTY | |
| | ICNVNHKPSNTKVDKRVEPKSCDKT | |
| | HTCPPCPAPELLGGPSVFLFPPKPK | |
| | DTLMISRTPEVTCVVVDVSHEDPEV | |
| | KFNWYVDGVEVHNAKTKPREEQYNS | |
| | TYRVVSVLTVLHQDWLNGKEYKCKV | |
| | SNKALPAPIEKTISKAKGQPREPQV | |
| | CTLPPSREEMTKNQVSLSCAVKGFY | |
| | PSDIAVEWESNGQPENNYKTTPPVL | |
| | DSDGSFFLVSKLTVDKSRWQQGNVF | |
| | SCSVMHEALHNHYTQKSLSLSPGK | |

### Multispecific Antibodies

In certain embodiments, described herein is a multispecific anti-CD47 and anti-EpCAM antibody, or binding fragment thereof. In some instances, the multispecific anti-CD47 and anti-EpCAM antibody comprises at least one targeting moiety that specifically binds to CD47 and at least one targeting moiety that specifically binds to EpCAM. In some embodiments, the multispecific antibody comprises a CD47-binding domain as described herein. In some embodiments, the multispecific antibody comprises an EpCAM binding domain as described herein. In some embodiments, the multispecific antibody comprises a CD47-binding domain as described herein and an EpCAM binding domain as described herein. In some embodiments, the multispecific antibody is bispecific, trispecific, or tetraspecific. In some embodiments, the multispecific antibody is bispecific. In some embodiments, the multispecific antibody is bivalent, trivalent, or tetravalent. In some embodiments, the multispecific antibody is bivalent. In some instances, the multispecific antibody has more than one binding site that binds to CD47. In some instances, the multispecific antibody has more than one binding site that binds to EpCAM. In some instances, the multispecific antibody or binding fragment thereof is a multispecific antibody conjugate, a hybrid multispecific IgG, a variable domain only multispecific antibody, a CH1/CL fusion protein, a Fab fusion protein, a non-immunoglobulin fusion protein, a Fc-modified IgG, an appended & Fc-modified IgG, a modified Fc and CH3 fusion protein, an appended IgG-HC fusion, a Fc fusion, a CH3 fusion, an IgE/IgM CH2 fusion, or a F(ab′)2 fusion. In some embodiments, the multispecific antibody further comprises an Fc domain. In some embodiments, the Fc domain is a human Fc domain. In some embodiments, the isotype of the Fc domain is IgG1, IgG2, IgG3, or IgG4. In some embodiments, the Fc domain is a heterodimeric Fc domain, wherein the heterodimeric Fc region comprises a knob chain and a hole chain, forming a knob-into-hole (KiH) structure. In some embodiments, the knob chain comprises an IgG1, IgG2, IgG3, or IgG4 domain. In some embodiments, the knob chain comprises the mutation T366W and the hole chain comprises the mutations T366S, L368A, and Y407V, wherein amino acid position numbering is according to the EU index of Kabat et al. In some embodiments, the hole chain comprises an IgG1, IgG2, IgG3, or IgG4 domain. In some embodiments, the multispecific antibody has an asymmetric three-chain knob-into-hole structure.

In certain embodiments, described herein is a multispecific anti-CD47 and anti-EGFR antibody, or binding fragment thereof. In some instances, the multispecific anti-CD47 and anti-EGFR antibody comprises at least one targeting moiety that specifically binds to CD47 and at least one targeting moiety that specifically binds to EGFR. In some embodiments, the multispecific antibody comprises a CD47-binding domain as described herein. In some embodiments, the multispecific antibody comprises an EGFR binding domain as described herein. In some embodiments, the multispecific antibody comprises a CD47-binding domain as described herein and an EGFR binding domain as described herein. In some embodiments, the multispecific antibody is bispecific, trispecific, or tetraspecific. In some embodiments, the multispecific antibody is bispecific. In some embodiments, the multispecific antibody is bivalent, trivalent, or tetravalent. In some embodiments, the multispecific antibody is bivalent. In some instances, the multispecific antibody has more than one binding site that binds to CD47. In some instances, the multispecific antibody has more than one binding site that binds to EGFR. In some instances, the multispecific antibody or binding fragment thereof is a multispecific antibody conjugate, a hybrid multispecific IgG, a variable domain only multispecific antibody, a CH1/CL fusion protein, a Fab fusion protein, a non-immunoglobulin fusion protein, a Fc-modified IgG, an appended & Fc-modified IgG, a modified Fc and CH3 fusion protein, an appended IgG-HC fusion, a Fc fusion, a CH3 fusion, an IgE/IgM CH2 fusion, or a F(ab′)2 fusion. In some embodiments, the multispecific antibody further comprises an Fc domain. In some embodiments, the Fc domain is a human Fc domain. In some embodiments, the isotype of the Fc domain is IgG1, IgG2, IgG3, or IgG4. In some embodiments, the Fc domain is a heterodimeric Fc domain, wherein the heterodimeric Fc region comprises a knob chain and a hole chain, forming a knob-into-hole (KiH) structure. In some embodiments, the knob chain comprises an IgG1, IgG2, IgG3, or IgG4 domain. In some embodiments, the knob chain comprises the mutation T366W and the hole chain comprises the mutations T366S, L368A, and Y407V, wherein amino acid position numbering is according to the EU index of Kabat et al. In some embodiments, the hole chain comprises an IgG1, IgG2, IgG3, or IgG4 domain. In some embodiments, the multispecific antibody has an asymmetric three-chain knob-into-hole structure.

In some cases, the multispecific antibody further comprises one or more mutations in a framework region, e.g., in the CH1 domain, CH2 domain, CH3 domain, hinge region, or a combination thereof. In some instances, the one or more mutations are to stabilize the antibody and/or to increase half-life. In some instances, the one or more mutations are to modulate Fc receptor interactions, to increase ADCC or antibody-dependent cellular phagocytosis (ADCP). In other instances, the one or more mutations are to reduce or eliminate Fc effector functions such as FcγR-binding, ADCC or ADCP. In additional instances, the one or more mutations are to modulate glycosylation. In some cases, the one or more mutations enhance stability, increase half-life, decrease glycosylation, and/or modulate Fc receptor interactions, e.g., to increase or decrease ADCC and/or ADCP.

In some cases, the multispecific antibody comprises an IgG1 framework. In some embodiments, the constant region of the anti-CD47 or anti-EpCAM or EGFR antibody is modified at one or more amino acid positions to alter Fc receptor interaction. Exemplary residues that modulate or alter Fc receptor interaction include, but are not limited to, G236, S239, T250, M252, S254, T256, K326, A330, 1332, E333A, M428, H433, or N434 (Kabat numbering; EU index of Kabat et al 1991 Sequences of Proteins of Immunological Interest). In some instances, the mutation comprises G236A, S239D, T250Q, M252Y, S254T, T256E, K326W, A330L, 1332E, E333A, E333S, M428L, H433K, or N434F.

In some embodiments, the modification at one or more amino acid positions in the IgG1 constant region to alter Fc receptor interaction leads to increased half-life. In some instances, the modification at one or more amino acid positions comprise T250, M252, S254, T256, M428, H433, N434, or a combination thereof; e.g., comprising T250Q/M428L or M252Y/S254T/T256E and H433K/N434F.

In some embodiments, a multispecific antibody described above comprises a knobs-into-holes (KIH) format. In some cases, the KIH is located in the Fc region, in which the residues within the CH3 domain are optionally modified based on the disclosure of WO96/027011; Ridgway, et. al., Protein Eng. 9 (1996) 617-621; or Merchant, et. al., Nat. Biotechnol. 16 (1998) 677-681. In some cases, one of the CH3 domain pair is the “knob” chain while the other is the “hole” chain and additional disulfide bridges are optionally introduced to further stabilize the antibody and/or to increase yield.

In some instances, the multispecific antibody is an IgG1, and the CH3 domain of the “knob” chain comprises a T366W mutation and the CH3 domain of the “hole” chain comprises mutations T366S, L368A, and Y407V. In some cases, the CH3 domain of the “knob” chain further comprises a Y349C mutation which forms an interchain disulfide bridge with either E356C or S354C in the CH3 domain of the “hole” chain.

In some instances, the CH3 domain of the “knob” chain comprises R409D and K370E mutations and the CH3 domain of the “hole” chain comprises D399K and E357K. In some cases, the CH3 domain of the “knob” chain further comprises a T366W mutation and the CH3 domain of the “hole” chain further comprises mutations T366S, L368A, and Y407V.

In some embodiments, modification at one or more amino acid positions in the IgG1 constant region to alter Fc receptor interaction leads to increased ADCC and/or ADCP. In some instances, the modification at one or more amino acid positions comprises S239, K326, A330, 1332, E333, or a combination thereof. In some instances, the modification at one or more amino acid positions for increased ADCC and/or ADCP comprises, e.g., E333A, S239D/A330L/1332E, or K326W/E333S. In some cases, the modification at one or more amino acid positions for increased ADCC comprises S239D/A330L/1332E. In some cases, the modification at one or more amino acid positions for increased ADCP comprises K326W/E333S.

In some embodiments, the IgG1 constant region is afucosylated. In other embodiments, the IgG1 is expressed in cells incapable of fucosylation. In some embodiments, the cell is a mammalian cell such as a Chinese Hamster Ovary cell line. In some embodiments, the cell is unable to express fucosyltransferase 8 (FUT8).

In some embodiments, the multispecific antibody comprises an IgG2 framework. In some instance, one or more amino acid positions in the IgG2 framework are modified to alter Fc receptor interaction, e.g., to increase ADCC and/or CDC. In some cases, one or more amino acid positions in the IgG2 framework are modified to stabilize the antibody and/or to increase half-life. In some instances, one or more amino acid positions in the IgG2 framework are modified to modulate glycosylation. In some cases, the IgG2 constant region is afucosylated. In some embodiments, the IgG2 constant region is expressed in cells incapable of fucosylation. In some embodiments, the cell is a mammalian cell such as a Chinese Hamster Ovary cell line. In some embodiments, the cell is unable to express fucosyltransferase 8 (FUT8).

In some embodiments, the multispecific antibody comprises an IgG3 framework. In some instance, one or more amino acid positions in the IgG3 framework are modified to alter Fc receptor interaction, e.g., to increase ADCC and/or ADCP. In some cases, one or more amino acid positions in the IgG3 framework are modified to stabilize the antibody and/or to increase half-life. In some instances, one or more amino acid positions in the IgG3 framework are modified to modulate glycosylation. In some cases, the constant region of the antibody is modified at amino acid R435 to extend the half-life, e.g., R435H (Kabat numbering). In some instances, the constant region is deglycosylated at residue N297. In some instances, the IgG3 constant region is expressed in cells incapable of fucosylation. In some embodiments, the cell is a mammalian cell such as a Chinese Hamster Ovary cell line. In some embodiments, the cell is unable to express fucosyltransferase 8 (FUT8).

In some embodiments, the multispecific antibody comprises an IgG4 framework. In some instance, one or more amino acid positions in the IgG4 framework are modified to alter Fc receptor interaction, e.g., to increase ADCC and/or ADCP. For example, mutations to increase ADCC comprises, in some embodiments, S239D, I332E, and A330L (amino acid numbering is according to the EU index in Kabat et al), such as described in U.S. Pat. No. 8,093,359. In some cases, one or more amino acid positions in the IgG4 framework are modified to stabilize the antibody and/or to increase half-life. In some instances, one or more amino acid positions in the IgG4 framework are modified to modulate glycosylation. In some cases, the constant region is modified at a hinge region to prevent or reduce strand exchange. In some instances, the amino acid that is modified is S228 (e.g., S228P). In some instances, the IgG4 constant region is expressed in cells incapable of fucosylation. In some embodiments, the cell is a mammalian cell such as a Chinese Hamster Ovary cell line. In some embodiments, the cell is unable to express fucosyltransferase 8 (FUT8).

In some embodiments, the human IgG constant region is modified to alter its ADCC and/or ADCP activity, e.g., with an amino acid modification described in Natsume et al., 2008 Cancer Res, 68 (10): 3863-72; Idusogie et al., 2001 J Immunol, 166 (4): 2571-5; Moore et al., 2010 mAbs, 2 (2): 181-189; Lazar et al., 2006 PNAS, 103 (11): 4005-4010, Shields et al., 2001 JBC, 276 (9): 6591-6604; Stavenhagen et al., 2007 Cancer Res, 67 (18): 8882-8890; Stavenhagen et al., 2008 Advan. Enzyme Regul., 48:152-164; Alegre et al, 1992 J Immunol, 148:3461-3468; Reviewed in Kaneko and Niwa, 2011 Biodrugs, 25 (1): 1-11.

In some embodiments, the human IgG constant region is modified to induce heterodimerization. For example, having an amino acid modification within the CH3 domain at Thr366, which when replaced with a more bulky amino acid, e.g., Trp (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). In some cases, heterodimerization via CH3 modifications is further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248:7-15).

In some instances, a multispecific antibody described herein has reduced or lacks glycosylation but is not modified at amino acid Asn297 (Kabat numbering). In these instances, the glycosylation is, for example, eliminated by production of the antibody in a host cell that lacks a post-translational glycosylation capacity, for example a bacterial or yeast derived system or a modified mammalian cell expression system. In some embodiments, the cell is a mammalian cell such as a Chinese Hamster Ovary cell line. In some embodiments, the cell is unable to express fucosyltransferase 8 (FUT8). In certain aspects, such a system is a cell-free expression system.

In some embodiments, the multispecific protein comprising a CD47-binding first component and an EpCAM-binding second component have different affinities (KD) for their respective target antigens as measured by surface plasmon resonance.

In some embodiments related to the multispecific antibody comprising the CD47-binding domain and the EpCAM binding domain, the multispecific antibody has a higher affinity for CD47 expressed on the surface of a tumor cell than for CD47 expressed on the surface of a normal cell, such as red blood cell, platelet, T cell or NK cell. In some embodiments, the tumor cell expresses EpCAM. In some embodiments, a concentration of the multispecific antibody required for half-maximal binding to a (e.g., human) red blood cell is greater than about 500 nanomolar (nM). In some embodiments, the multispecific antibody binds to (e.g., human) CD47 with a dissociation constant (KD) of less than about 100 nanomolar (nM). In some embodiments, the multispecific antibody binds to (e.g., human) CD47 with a dissociation constant (KD) of from about 1 nanomolar (nM) to about 100 nM, from about 1 nM to about 50 nM, or from about 5 nM to about 50 nM. In some embodiments, the multispecific antibody binds to EpCAM with a dissociation constant (KD) of less than about 500 nanomolar (nM). In some embodiments, the multispecific antibody binds to EpCAM with a dissociation constant (KD) of from about 0.2 nanomolar (nM) to about 500 nM, from about 1 nM to about 300 nM, from about 5 nM to about 200 nM, or from about 10 nM to about 150 nM. In some embodiments, the dissociation constant (KD) is determined by surface plasmon resonance. In some embodiments, less than about 1 nanomolar (nM) or less than about 0.01 nM of the multispecific antibody increases a percentage of A431 cells engulfed by macrophage by at least 4-fold compared to a nonspecific IgG1 antibody control.

In some embodiments, the multispecific antibody comprising the CD47-binding domain and the EpCAM binding domain is characterized by a ratio of the KD of the CD47-binding domain to the KD of the EpCAM binding domain of at least 2:1, at least 5:1, at least 10:1, at least 20:1, or at least 100:1.

In some embodiments related to the multispecific antibody comprising the CD47-binding domain and the EpCAM binding domain, a concentration of the antibody required to mediate antibody-dependent cellular phagocytosis of an EpCAM-positive, CD47-positive tumor cell by a macrophage is from about 0.01 nanomolar (nM) to about 3 nM. In some embodiments, the EpCAM-positive, CD47-positive tumor cell is an OVISE cell or an A431 cell. In some embodiments, the EpCAM-positive, CD47-positive tumor cell is selected from a ductal pancreatic adenocarcinoma cell, an ovarian clear cell adenocarcinoma cell, a colon adenocarcinoma cell, a lung adenocarcinoma cell, a lung squamous cell carcinoma cell, a lung small cell carcinoma cell, a hepatocellular carcinoma cell, a gastric adenocarcinoma cell, a cholangiocarcinoma cell, a head and neck squamous cell carcinoma cell, a prostate carcinoma cell, an endometrial carcinoma cell, an esophageal cancer cell, an ovarian adenocarcinoma cell, a vulvar squamous cell carcinoma cell. In some embodiments, about 100 nanomolar (nM) of the multispecific antibody inhibits the binding of SIRPα to CD47 on the surface of a cell by at least about 30%. In some embodiments, the cell is a CD47+ EpCAM+ tumor cell. In some embodiments, the cell expresses at least as many EpCAM proteins on its surface as an HCC-44 cell. In some embodiments, the cell expresses at least 50,000, at least 100,000, at least 300,000, at least 600,000 or at least 1,500,000 EpCAM proteins on its surface. In some embodiments, about 400 nanomolar (nM) of the multispecific antibody does not induce hemolysis of red blood cells in a hemagglutination assay.

In some embodiments related to the multispecific antibody comprising a CD47-binding domain and an EpCAM binding domain, the multispecific antibody comprises one or more sequences (e.g., two or more, or three) each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 26.

In some embodiments related to the multispecific antibody comprising a CD47-binding domain and an EpCAM binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 482; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 483; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 484.

In some embodiments related to the multispecific antibody comprising a CD47-binding domain and an EpCAM binding domain, the multispecific antibody comprises one or more sequences (e.g., two or more, or three) each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 27.

In some embodiments related to the multispecific antibody comprising a CD47-binding domain and an EpCAM binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 485; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 486; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 487.

TABLE 26
Polypeptide sequences of an example three-
chain, anti-CD47 × anti-EpCAM bispecific
antibody “CD47xEpCAM_v5.”

| Construct | SEQ ID | Amino Acid Sequence |
|---|---|---|
| Description | NO | (N to C) |
| CD47. | 482 | EVQLLESGGGLVQPGGSLRLSCAAS |
| VIR47.V8. | | GFTFSNFAMSWVRQAPGKGLEWVST |
| huIgG1.knob | | ISASGGRTFYADSVKGRFTISRDNS |
| | | KNTLYLQMNSLRAEDTAVYYCAKEG |
| | | SFGEGVDPWGQGTLVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKKVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLWCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGK |
| VIR47.V8. | 483 | SYELTQPPSVSVSPGQTARITCSGD |
| Lambda LC | | ALPKKYAYWYQQKSGQAPVLVIYED |
| | | NKRPSGIPERFSGSSSGTMATLTIS |
| | | GAQVEDEADYYCYSTDISGNHWVFG |
| | | GGTKLTVLGQPKAAPSVTLFPPSSE |
| | | ELQANKATLVCLISDFYPGAVTVAW |
| | | KADSSPVKAGVETTTPSKQSNNKYA |
| | | ASSYLSLTPEQWKSHRSYSCQVTHE |
| | | GSTVEKTVAPTECS |
| EpCAM. | 484 | DIQLTQSPSSLSASVGDRVTFTCRA |
| Mab002-11. | | SQSIGTSLHWYQQKPGKAPKLLIKY |
| scFv hole | | ASESISGIPSRFSGSGSGTDFTLTI |
| | | SSVQPEDFATYYCQQSNSWPYTFGQ |
| | | GTKLEIKGGGGSGGGGSGGGGSEVQ |
| | | LVESGGGLVQPGGSLRLSCAASGFT |
| | | FNTYAMHWVRQAPGKGLEWVARIRS |
| | | KTYNYATFYADSVKGRFTISRDDSK |
| | | NTLYLQMNSLRAEDTAVYYCVTGSS |
| | | YAMDYWGQGTTVTVSSGGSGGSGGA |
| | | SKTHTCPPCPAPELLGGPSVFLFPP |
| | | KPKDTLMISRTPEVTCVVVDVSHED |
| | | PEVKFNWYVDGVEVHNAKTKPREEQ |
| | | YNSTYRVVSVLTVLHQDWLNGKEYK |
| | | CKVSNKALPAPIEKTISKAKGQPRE |
| | | PQVYTLPPSRDELTKNQVSLSCAVK |
| | | GFYPSDIAVEWESNGQPENNYKTTP |
| | | PVLDSDGSFFLVSKLTVDKSRWQQG |
| | | NVFSCSVMHEALHNHYTQKSLSLSP |
| | | GK |

TABLE 27
Polypeptide sequences of an example three-
chain, anti-CD47 × anti-EpCAM bispecific
antibody “CD47xEpCAM_v6.”

| Construct | SEQ ID | Amino Acid Sequence |
|---|---|---|
| Description | NO | (N to C) |
| EpCAM | 485 | QVQLVQSGAEVKKPGSSVKVSCKAS |
| | | GYTFTSYGISWVRQAPGQGLEWMGE |
| | | IYPRSDNTYYAQKFQGRVTITADKS |
| | | TSTAYMELSSLRSEDTAVYYCARRA |
| | | GMWFAYWGQGTLVTVSSASTKGPSV |
| | | FPLAPSSKSTSGGTAALGCLVKDYF |
| | | PEPVTVSWNSGALTSGVHTFPAVLQ |
| | | SSGLYSLSSVVTVPSSSLGTQTYIC |
| | | NVNHKPSNTKVDKKVEPKSCDKTHT |
| | | CPPCPAPELLGGPSVFLFPPKPKDT |
| Mab008-1.IgG1 | | LMISRTPEVTCVVVDVSHEDPEVKF |
| HC knob | | NWYVDGVEVHNAKTKPREEQYNSTY |
| | | RVVSVLTVLHQDWLNGKEYKCKVSN |
| | | KALPAPIEKTISKAKGQPREPQVYT |
| | | LPPSRDELTKNQVSLWCLVKGFYPS |
| | | DIAVEWESNGQPENNYKTTPPVLDS |
| | | DGSFFLYSKLTVDKSRWQQGNVFSC |
| | | SVMHEALHNHYTQKSLSLSPGK |
| EpCAM | 486 | DIQMTQSPSSLSASVGDRVTITCRA |
| Mab008-1. | | SSSVSDMHWYQQKPGKAPKLLIYEI |
| Kappa LC | | SKLASGVPSRFSGSGSGTDFTLTIS |
| | | SLQPEDFATYYCQHWSYPLMTFGQG |
| | | TKLEIKRTVAAPSVFIFPPSDEQLK |
| | | SGTASVVCLLNNFYPREAKVQWKVD |
| | | NALQSGNSQESVTEQDSKDSTYSLS |
| | | STLTLSKADYEKHKVYACEVTHQGL |
| | | SSPVTKSFNRGEC |
| CD47.VIR47.V8. | 487 | EVQLLESGGGLVQPGGSLRLSCAAS |
| scFv hole | | GFTFSNFAMSWVRQAPGKGLEWVST |
| | | ISASGGRTFYADSVKGRFTISRDNS |
| | | KNTLYLQMNSLRAEDTAVYYCAKEG |
| | | SFGEGVDPWGQGTLVTVSSGGGGSG |
| | | GGGSGGGGSSYELTQPPSVSVSPGQ |
| | | TARITCSGDALPKKYAYWYQQKSGQ |
| | | APVLVIYEDNKRPSGIPERFSGSSS |
| | | GTMATLTISGAQVEDEADYYCYSTD |
| | | ISGNHWVFGGGTKLTVLGGSGGSGG |
| | | ASKTHTCPPCPAPELLGGPSVFLFP |
| | | PKPKDTLMISRTPEVTCVVVDVSHE |
| | | DPEVKFNWYVDGVEVHNAKTKPREE |
| | | QYNSTYRVVSVLTVLHQDWLNGKEY |
| | | KCKVSNKALPAPIEKTISKAKGQPR |
| | | EPQVYTLPPSRDELTKNQVSLSCAV |
| | | KGFYPSDIAVEWESNGQPENNYKTT |
| | | PPVLDSDGSFFLVSKLTVDKSRWQQ |
| | | GNVFSCSVMHEALHNHYTQKSLSLS |
| | | PGK |

In some embodiments related to the multispecific antibody comprising a CD47-binding domain and an EpCAM binding domain, the multispecific (e.g., bispecific) antibodies, and binding fragments thereof, comprise a heavy chain variable fragment (VH) of the anti-CD47 binding domain fused to a first T-cell receptor (TCR) constant region, and a light chain variable domain (VL) of the anti-CD47 binding domain fused to a second TCR constant region, wherein the first and second TCR constant regions are capable of forming a dimer. In some embodiments, the TCR constant regions are the TCR alpha and TCR beta constant regions. In some embodiments, the anti-CD47 VH-TCR fusion polypeptide is fused to a human IgG (e.g., IgG1) Fc domain. See WO 2019/057122. In some embodiments, the anti-EpCAM binding domain comprises an antigen-binding fragment (Fab) domain. In some embodiments, the anti-EpCAM binding domain comprises a single-chain variable fragment (scFv) domain.

In some embodiments related to the multispecific antibody comprising a CD47-binding domain and an EpCAM binding domain, the anti-CD47 binding domain comprises a VH-CD47 domain and a VL-CD47 domain, wherein the VH-CD47 domain is fused to the TCR beta constant domain; and the VL-CD47 domain is fused to the TCR alpha constant domain. In some embodiments, the anti-CD47 binding domain comprises a VH-CD47 domain and a VL-CD47 domain, wherein the VH-CD47 domain is fused to the CH1 IgG domain; and the VL-CD47 domain is fused to the CL IgG domain. In some embodiments, the anti-EpCAM binding domain comprises an antigen-binding fragment (Fab) domain. In some embodiments, the anti-EpCAM binding domain comprises a single-chain variable fragment (scFv) domain.

In some embodiments related to the multispecific antibody comprising a CD47-binding domain and an EpCAM binding domain, the multispecific (e.g., bispecific) antibodies, and binding fragments thereof, comprise a heavy chain variable fragment (VH) of the anti-EpCAM binding domain fused to a first T-cell receptor (TCR) constant region, and a light chain variable domain (VL) of the anti-EpCAM binding domain fused to a second TCR constant region, wherein the first and second TCR constant regions are capable of forming a dimer. In some embodiments, the TCR constant regions are the TCR alpha and TCR beta constant regions. In some embodiments, the anti-EpCAM VH-TCR fusion polypeptide is fused to a human IgG (e.g., IgG1) Fc domain. See WO 2019/057122. In some embodiments, the anti-CD47 binding domain comprises an antigen-binding fragment (Fab) domain. In some embodiments, the anti-CD47 binding domain comprises a single-chain variable fragment (scFv) domain.

In some embodiments related to the multispecific antibody comprising a CD47-binding domain and an EpCAM binding domain, the anti-EpCAM binding domain comprises a VH-EpCAM domain and a VL-EpCAM domain, wherein the VH-EpCAM domain is fused to the TCR beta constant domain of the anti-EpCAM binding domain; and the VL-EpCAM domain is fused to the TCR alpha constant domain of anti-EpCAM binding domain. In some embodiments, the anti-EpCAM binding domain comprises a VH-EpCAM domain and a VL-EpCAM domain, wherein the VH-EpCAM domain is fused to the CH1 IgG domain of the anti-EpCAM binding domain; and the VL-EpCAM domain is fused to the CL IgG domain of the anti-EpCAM binding domain. In some embodiments, the anti-CD47 binding domain comprises an antigen-binding fragment (Fab) domain. In some embodiments, the anti-CD47 binding domain comprises a single-chain variable fragment (scFv) domain.

In some embodiments related to the multispecific antibody comprising a CD47-binding domain and an EpCAM binding domain, the multispecific antibody comprises one or more sequences (e.g., two or more, or three) each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 28.

In some embodiments related to the multispecific antibody comprising a CD47-binding domain and an EpCAM binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 488; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 489; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 490; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 491.

TABLE 28
Polypeptide sequences of an example four-
chain, anti-CD47 × anti-EpCAM bispecific
antibody “CD47xEpCAM_v7.”

| Construct | SEQ ID | Amino Acid Sequence |
|---|---|---|
| Description | NO | (N to C) |
| Anti-CD47 | 488 | SYELTQPPSVSVSPGQTARITCSGD |
| TCRα LC | | ALPKKYAYWYQQKSGQAPVLVIYED |
| | | NKRPSGIPERFSGSSSGTMATLTIS |
| | | GAQVEDEADYYCYSTDISGNHWVFG |
| | | GGTKLTVLPDIQNPDPAVYQLRDSK |
| | | SSDKSVCLFTDFDSQTQVSQSKDSD |
| | | VYITDKCVLDMRSMDFKSNSAVAWS |
| | | QKSDFACANAFQNSIIPEDTFFPSP |
| | | ESS |
| Anti-CD47 | 489 | EVQLLESGGGLVQPGGSLRLSCAAS |
| TCRβ. HC | | GFTFSNFAMSWVRQAPGKGLEWVST |
| | | ISASGGRTFYADSVKGRFTISRDNS |
| | | KNTLYLQMNSLRAEDTAVYYCAKEG |
| | | SFGEGVDPWGQGTLVTVLEDLKNVF |
| | | PPEVAVFEPSEAEISHTQKATLVCL |
| | | ATGFYPDHVELSWWVNGKEVHSGVC |
| | | TDPQPLKEQPALQDSRYALSSRLRV |
| | | SATFWQNPRNHFRCQVQFYGLSEND |
| | | EWTQDRAKPVTQIVSAEAWGRASDK |
| | | THTCPPCPAPELLGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPCREEMTKNQVSLWCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPG |
| EpCAM | 490 | EVQLVESGGGLVQPGGSLRLSCAAS |
| “Chain 1” | | GFTFNTYAMHWVRQAPGKGLEWVAR |
| | | IRSKTYNYATFYADSVKGRFTISRD |
| | | DSKNTLYLQMNSLRAEDTAVYYCVT |
| | | GSSYAMDYWGQGTTVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKRVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | CTLPPSREEMTKNQVSLSCAVKGFY |
| | | PSDIAVEWESNGQPENNYKTTPPVL |
| | | DSDGSFFLVSKLTVDKSRWQQGNVF |
| | | SCSVMHEALHNHYTQKSLSLSPG |
| EpCAM | 491 | DIQLTQSPSSLSASVGDRVTFTCRA |
| “Chain 2” | | SQSIGTSLHWYQQKPGKAPKLLIKY |
| | | ASESISGIPSRFSGSGSGTDFTLTI |
| | | SSVQPEDFATYYCQQSNSWPYTFGQ |
| | | GTKLEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |

In some embodiments related to the multispecific antibody comprising a CD47-binding domain and an EpCAM binding domain, the multispecific antibody comprises one or more sequences (e.g., two or more, or three) each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 29.

In some embodiments related to the multispecific antibody comprising a CD47-binding domain and an EpCAM binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 492; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 493; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 494; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 495.

TABLE 29
Polypeptide sequences of an example four-
chain, anti-CD47 × anti-EpCAM bispecific
antibody “CD47xEpCAM_v8.”

| Construct | SEQ ID | Amino Acid Sequence |
|---|---|---|
| Description | NO | (N to C) |
| Anti-EpCAM | 492 | DIQLTQSPSSLSASVGDRVTFTCRA |
| TCRα LC | | SQSIGTSLHWYQQKPGKAPKLLIKY |
| | | ASESISGIPSRFSGSGSGTDFTLTI |
| | | SSVQPEDFATYYCQQSNSWPYTFGQ |
| | | GTKLEIKPDIQNPDPAVYQLRDSKS |
| | | SDKSVCLFTDFDSQTQVSQSKDSDV |
| | | YITDKCVLDMRSMDFKSNSAVAWSQ |
| | | KSDFACANAFONSIIPEDTFFPSPE |
| | | SS |
| Anti-EpCAM | 493 | EVQLVESGGGLVQPGGSLRLSCAAS |
| TCRβ. HC | | GFTFNTYAMHWVRQAPGKGLEWVAR |
| | | IRSKTYNYATFYADSVKGRFTISRD |
| | | DSKNTLYLQMNSLRAEDTAVYYCVT |
| | | GSSYAMDYWGQGTTVTVLEDLKNVF |
| | | PPEVAVFEPSEAEISHTQKATLVCL |
| | | ATGFYPDHVELSWWVNGKEVHSGVC |
| | | TDPQPLKEQPALQDSRYALSSRLRV |
| | | SATFWQNPRNHFRCQVQFYGLSEND |
| | | EWTQDRAKPVTQIVSAEAWGRASDK |
| | | THTCPPCPAPELLGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPCREEMTKNQVSLWCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPG |
| CD47 “Chain 1” | 494 | SYELTQPPSVSVSPGQTARITCSGD |
| | | ALPKKYAYWYQQKSGQAPVLVIYED |
| | | NKRPSGIPERFSGSSSGTMATLTIS |
| | | GAQVEDEADYYCYSTDISGNHWVFG |
| | | GGTKLTVLGQPKAAPSVTLFPPSSE |
| | | ELQANKATLVCLISDFYPGAVTVAW |
| | | KADSSPVKAGVETTTPSKQSNNKYA |
| | | ASSYLSLTPEQWKSHKSYSCQVTHE |
| | | GSTVEKTVAPTECS |
| CD47 “Chain 2” | 495 | EVQLLESGGGLVQPGGSLRLSCAAS |
| | | GFTFSNFAMSWVRQAPGKGLEWVST |
| | | ISASGGRTFYADSVKGRFTISRDNS |
| | | KNTLYLQMNSLRAEDTAVYYCAKEG |
| | | SFGEGVDPWGQGTLVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKRVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | CTLPPSREEMTKNQVSLSCAVKGFY |
| | | PSDIAVEWESNGQPENNYKTTPPVL |
| | | DSDGSFFLVSKLTVDKSRWQQGNVF |
| | | SCSVMHEALHNHYTQKSLSLSPG |

In some embodiments related to the multispecific antibody comprising a CD47-binding domain and an EpCAM binding domain, the multispecific antibody comprises one or more sequences (e.g., two or more, or three) each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 30.

In some embodiments related to the multispecific antibody comprising a CD47-binding domain and an EpCAM binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 503; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 504; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 505.

TABLE 30
Polypeptide sequences of an example three-
chain, anti-CD47 × anti-EpCAM bispecific
antibody “CD47xEpCAM_v9.”

| Construct | SEQ ID | Amino Acid Sequence |
|---|---|---|
| Description | NO | (N to C) |
| EpCAM | 503 | EVQLVESGGGLVQPGGSLRLSCAAS |
| Mab002-11. | | GFTFNTYAMHWVRQAPGKGLEWVAR |
| IgG1 HC knob | | IRSKTYNYATFYADSVKGRFTISRD |
| | | DSKNTLYLQMNSLRAEDTAVYYCVT |
| | | GSSYAMDYWGQGTTVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKKVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLWCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGK |
| EpCAM | 504 | DIQLTQSPSSLSASVGDRVTFTCRA |
| Mab002-11. | | SQSIGTSLHWYQQKPGKAPKLLIKY |
| Kappa LC | | ASESISGIPSRFSGSGSGTDFTLTI |
| | | SSVQPEDFATYYCQQSNSWPYTFGQ |
| | | GTKLEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| CD47.VIR47.V8. | 505 | EVQLLESGGGLVQPGGSLRLSCAAS |
| scFv Hole | | GFTFSNFAMSWVRQAPGKGLEWVST |
| | | ISASGGRTFYADSVKGRFTISRDNS |
| | | KNTLYLQMNSLRAEDTAVYYCAKEG |
| | | SFGEGVDPWGQGTLVTVSSGGGGSG |
| | | GGGSGGGGSSYELTQPPSVSVSPGQ |
| | | TARITCSGDALPKKYAYWYQQKSGQ |
| | | APVLVIYEDNKRPSGIPERFSGSSS |
| | | GTMATLTISGAQVEDEADYYCYSTD |
| | | ISGNHWVFGGGTKLTVLGGSGGSGG |
| | | ASKTHTCPPCPAPELLGGPSVFLFP |
| | | PKPKDTLMISRTPEVTCVVVDVSHE |
| | | DPEVKFNWYVDGVEVHNAKTKPREE |
| | | QYNSTYRVVSVLTVLHQDWLNGKEY |
| | | KCKVSNKALPAPIEKTISKAKGQPR |
| | | EPQVYTLPPSRDELTKNQVSLSCAV |
| | | KGFYPSDIAVEWESNGQPENNYKTT |
| | | PPVLDSDGSFFLVSKLTVDKSRWQQ |
| | | GNVFSCSVMHEALHNHYTQKSLSLS |
| | | PGK |

In some embodiments related to the multispecific antibody comprising a CD47-binding domain and an EpCAM binding domain, the multispecific antibody comprises one or more sequences (e.g., two or more, or three) each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 31.

In some embodiments related to the multispecific antibody comprising a CD47-binding domain and an EpCAM binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 482; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 483; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 506.

TABLE 31
Polypeptide sequences of an example three-
chain, anti-CD47 × anti-EpCAM bispecific
antibody “CD47xEpCAM_v10.”

| Construct | SEQ ID | Amino Acid Sequence |
|---|---|---|
| Description | NO | (N to C) |
| CD47. | 482 | EVQLLESGGGLVQPGGSLRLSCAAS |
| VIR47.V8. | | GFTFSNFAMSWVRQAPGKGLEWVST |
| huIgG1.knob | | ISASGGRTFYADSVKGRFTISRDNS |
| | | KNTLYLQMNSLRAEDTAVYYCAKEG |
| | | SFGEGVDPWGQGTLVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKKVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLWCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGK |
| VIR47.V8. | 483 | SYELTQPPSVSVSPGQTARITCSGD |
| Kappa LC | | ALPKKYAYWYQQKSGQAPVLVIYED |
| | | NKRPSGIPERFSGSSSGTMATLTIS |
| | | GAQVEDEADYYCYSTDISGNHWVFG |
| | | GGTKLTVLGQPKAAPSVTLFPPSSE |
| | | ELQANKATLVCLISDFYPGAVTVAW |
| | | KADSSPVKAGVETTTPSKQSNNKYA |
| | | ASSYLSLTPEQWKSHRSYSCQVTHE |
| | | GSTVEKTVAPTECS |
| EpCAM. | 506 | DIQMTQSPSSLSASVGDRVTLTCRA |
| Mab001-12. | | SQDIGNSLNWLQQKPGKAPKRLIYV |
| scFv hole | | TSRLDSGVPSRFSGSRSGTDYTFTI |
| | | SSLQPEDFATYYCLQYASSPFTFGQ |
| | | GTKLEIKGGGGSGGGGSGGGGSQVQ |
| | | LVQSGAEVKKPGASVKVSCKASGYT |
| | | FTSYDINWVRQAPGQGLEWMGWIYP |
| | | RDGSTKYAQKFQGRVTMTVDTSTST |
| | | AYMELSSLRSEDTAVYYCARDPYYY |
| | | GTIYGYFDVWGQGTTVTVSSGGSGG |
| | | SGGASKTHTCPPCPAPELLGGPSVF |
| | | LFPPKPKDTLMISRTPEVTCVVVDV |
| | | SHEDPEVKFNWYVDGVEVHNAKTKP |
| | | REEQYNSTYRVVSVLTVLHQDWLNG |
| | | KEYKCKVSNKALPAPIEKTISKAKG |
| | | QPREPQVYTLPPSRDELTKNQVSLS |
| | | CAVKGFYPSDIAVEWESNGQPENNY |
| | | KTTPPVLDSDGSFFLVSKLTVDKSR |
| | | WQQGNVFSCSVMHEALHNHYTQKSL |
| | | SLSPGK |

In some embodiments related to the multispecific antibody comprising a CD47-binding domain and an EpCAM binding domain, the multispecific antibody comprises one or more sequences (e.g., two or more, or three) each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 32.

In some embodiments related to the multispecific antibody comprising a CD47-binding domain and an EpCAM binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 482; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 483; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 507.

TABLE 32
Polypeptide sequences of an example three-
chain, anti-CD47 × anti-EpCAM bispecific
antibody “CD47xEpCAM_v11.”

| Construct | SEQ ID | Amino Acid Sequence |
|---|---|---|
| Description | NO | (N to C) |
| CD47. | 482 | EVQLLESGGGLVQPGGSLRLSCAAS |
| VIR47.V8. | | GFTFSNFAMSWVRQAPGKGLEWVST |
| huIgG1.knob | | ISASGGRTFYADSVKGRFTISRDNS |
| | | KNTLYLQMNSLRAEDTAVYYCAKEG |
| | | SFGEGVDPWGQGTLVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKKVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLWCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGK |
| VIR47.V8. | 483 | SYELTQPPSVSVSPGQTARITCSGD |
| Kappa LC | | ALPKKYAYWYQQKSGQAPVLVIYED |
| | | NKRPSGIPERFSGSSSGTMATLTIS |
| | | GAQVEDEADYYCYSTDISGNHWVFG |
| | | GGTKLTVLGQPKAAPSVTLFPPSSE |
| | | ELQANKATLVCLISDFYPGAVTVAW |
| | | KADSSPVKAGVETTTPSKQSNNKYA |
| | | ASSYLSLTPEQWKSHRSYSCQVTHE |
| | | GSTVEKTVAPTECS |
| EpCAM. | 507 | DIQLTQSPSSLSASVGDRVTFTCRA |
| Mab002-8. | | SQSIGTSLHWYQQKPGKAPKLLIKY |
| scFv hole | | ASESISGIPSRFSGSGSGTDFTLTI |
| | | SSVQPEDIATYYCQQSNSWPYTFGQ |
| | | GTKLEIKGGGGSGGGGSGGGGSEVQ |
| | | LVESGGGLVQPGGSLRLSCAASGFT |
| | | FNTYAMHWVRQAPGKGLEWVARIRS |
| | | KTYNYATFYADSVKGRFTISRDNSK |
| | | NTLYLQMNSLRAEDTAVYYCVTGSS |
| | | YAMDYWGQGTTVTVSSGGSGGSGGA |
| | | SKTHTCPPCPAPELLGGPSVFLFPP |
| | | KPKDTLMISRTPEVTCVVVDVSHED |
| | | PEVKFNWYVDGVEVHNAKTKPREEQ |
| | | YNSTYRVVSVLTVLHQDWLNGKEYK |
| | | CKVSNKALPAPIEKTISKAKGQPRE |
| | | PQVYTLPPSRDELTKNQVSLSCAVK |
| | | GFYPSDIAVEWESNGQPENNYKTTP |
| | | PVLDSDGSFFLVSKLTVDKSRWQQG |
| | | NVFSCSVMHEALHNHYTQKSLSLSP |
| | | GK |

In some embodiments related to the multispecific antibody comprising the CD47-binding domain and the EGFR-binding domain, the multispecific antibody has a higher affinity for CD47 expressed on the surface of a tumor cell than for CD47 expressed on the surface of a normal cell, such as red blood cell, platelet, T cell B cell or NK cell. In some embodiments, the tumor cell expresses EGFR. In some embodiments, a concentration of the multispecific antibody required for half-maximal binding to a (e.g., human) red blood cell is greater than about 500 nanomolar (nM). In some embodiments, the multispecific antibody binds to (e.g., human) CD47 with a dissociation constant (KD) of less than about 100 nanomolar (nM). In some embodiments, the multispecific antibody binds to (e.g., human) CD47 with a dissociation constant (KD) of from about 1 nanomolar (nM) to about 100 nM, from about 1 nM to about 50 nM, or from about 5 nM to about 50 nM. In some embodiments, the dissociation constant (KD) is determined by surface plasmon resonance. In some embodiments, the multispecific antibody binds to EGFR with a dissociation constant (KD) of less than about 25 nanomolar (nM). In some embodiments, the multispecific antibody binds to EGFR with a dissociation constant (KD) of from about 0.2 nanomolar (nM) to about 25 nM, from about 0.2 nM to about 10 nM, from about 0.2 nM to about 2 nM, or from about 2 nM to about 10 nM. In some embodiments, the dissociation constant (KD) is determined by surface plasmon resonance. In some embodiments, less than about 1 nanomolar (nM) or less than about 0.01 nM of the multispecific antibody increases a percentage of A431 cells engulfed by macrophage by at least 4-fold compared to a nonspecific IgG1 antibody control.

In some embodiments related to the multispecific antibody comprising the CD47-binding domain and the EGFR-binding domain, a concentration of the antibody required to mediate antibody-dependent cellular phagocytosis of an EGFR-positive, CD47-positive tumor cell by a macrophage is from about 0.01 nanomolar (nM) to about 3 nM. In some embodiments, the EGFR-positive, CD47-positive tumor cell is an OVISE cell or an A431 cell. In some embodiments, the EGFR-positive, CD47-positive tumor cell is selected from epidermoid carcinoma cell, colorectal adenocarcinoma cell, head and neck squamous cell carcinoma cell, bladder carcinoma cell, pancreas adenocarcinoma cell, lung squamous cell carcinoma cell, lung adenocarcinoma cell, renal cell carcinoma cell, a esophageal cancer cell, or gastric carcinoma cell. In some embodiments, about 100 nanomolar (nM) of the multispecific antibody inhibits the binding of SIRPα to CD47 on the surface of a cell by at least about 30%. In some embodiments, the cell is a CD47+ EGFR+ tumor cell. In some embodiments, the cell expresses at least as many EGFR proteins on its surface as an HCC-44 cell. In some embodiments, the cell expresses at least 50,000, at least 100,000, at least 300,000, at least 600,000 or at least 1,500,000 EGFR proteins on its surface. In some embodiments, about 400 nanomolar (nM) of the multispecific antibody does not induce hemolysis of red blood cells in a hemagglutination assay.

In some embodiments related to the multispecific antibody comprising an CD47-binding domain and an EGFR binding domain, the multispecific antibody comprises one or more (e.g., two or more, or three) sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 33.

In some embodiments related to the multispecific antibody comprising a CD47-binding domain and an EGFR binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 496; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 497; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 498.

In some embodiments related to the multispecific antibody comprising a CD47-binding domain and an EGFR binding domain, the multispecific (e.g., bispecific) antibodies, and binding fragments thereof, comprise a heavy chain variable fragment (VH) of the anti-CD47 binding domain fused to a first T-cell receptor (TCR) constant region, and a light chain variable domain (VL) of the anti-CD47 binding domain fused to a second TCR constant region, wherein the first and second TCR constant regions are capable of forming a dimer. In some embodiments, the TCR constant regions are the TCR alpha and TCR beta constant regions. In some embodiments, the anti-CD47 VH-TCR fusion polypeptide is fused to a human IgG (e.g., IgG1) Fc domain. See WO 2019/057122. In some embodiments, the anti-EGFR binding domain comprises an antigen-binding fragment (Fab) domain. In some embodiments, the anti-EGFR binding domain comprises a single-chain variable fragment (scFv) domain.

In some embodiments related to the multispecific antibody comprising a CD47-binding domain and an EGFR binding domain, the anti-CD47 binding domain comprises a VH-CD47 domain and a VL-CD47 domain, wherein the VH-CD47 domain is fused to the TCR beta constant domain; and the VL-CD47 domain is fused to the TCR alpha constant domain. In some embodiments, the anti-CD47 binding domain comprises a VH-CD47 domain and a VL-CD47 domain, wherein the VH-CD47 domain is fused to the CH1 IgG domain; and the VL-CD47 domain is fused to the CL IgG domain. In some embodiments, the anti-EGFR binding domain comprises an antigen-binding fragment (Fab) domain. In some embodiments, the anti-EGFR binding domain comprises a single-chain variable fragment (scFv) domain.

In some embodiments related to the multispecific antibody comprising a CD47-binding domain and an EGFR binding domain, the multispecific (e.g., bispecific) antibodies, and binding fragments thereof, comprise a heavy chain variable fragment (VH) of the anti-EGFR binding domain fused to a first T-cell receptor (TCR) constant region, and a light chain variable domain (VL) of the anti-EGFR binding domain fused to a second TCR constant region, wherein the first and second TCR constant regions are capable of forming a dimer. In some embodiments, the TCR constant regions are the TCR alpha and TCR beta constant regions. In some embodiments, the anti-EGFR VH-TCR fusion polypeptide is fused to a human IgG (e.g., IgG1) Fc domain. See WO 2019/057122. In some embodiments, the anti-CD47 binding domain comprises an antigen-binding fragment (Fab) domain. In some embodiments, the anti-CD47 binding domain comprises a single-chain variable fragment (scFv) domain.

In some embodiments related to the multispecific antibody comprising a CD47-binding domain and an EGFR binding domain, the anti-EGFR binding domain comprises a VH-EGFR domain and a VL-EGFR domain, wherein the VH-EGFR domain is fused to the TCR beta constant domain of the anti-EGFR binding domain; and the VL-EGFR domain is fused to the TCR alpha constant domain of anti-EGFR binding domain. In some embodiments, the anti-EGFR binding domain comprises a VH-EGFR domain and a VL-EGFR domain, wherein the VH-EGFR domain is fused to the CH1 IgG domain of the anti-EGFR binding domain; and the VL-EGFR domain is fused to the CL IgG domain of the anti-EGFR binding domain. In some embodiments, the anti-CD47 binding domain comprises an antigen-binding fragment (Fab) domain. In some embodiments, the anti-CD47 binding domain comprises a single-chain variable fragment (scFv) domain.

In some embodiments related to the multispecific antibody comprising an CD47-binding domain and an EGFR binding domain, the multispecific antibody comprises one or more (e.g., two or more, three or more, or four) sequences having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 34.

In some embodiments related to the multispecific antibody comprising a CD47-binding domain and an EGFR binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 499; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 500; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 501; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 502.

In some embodiments related to the multispecific antibody comprising an CD47-binding domain and an EGFR binding domain, the multispecific antibody comprises one or more (e.g., two or more, three or more, or four) sequences having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 35.

In some embodiments related to the multispecific antibody comprising a CD47-binding domain and an EGFR binding domain, the multispecific antibody comprises a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 508; a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 509; and a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 510.

TABLE 33
Polypeptide sequences of the proof-of-
concept CD47 × EGFR bispecific antibody.
Panitumumab × VIR47.V8_v2

| Construct | SEQ ID | Amino Acid Sequence |
|---|---|---|
| Description | NO | (N to C) |
| EGFR. | 496 | QVQLQESGPGLVKPSETLSLTCTVS |
| Panitumumab. | | GGSVSSGDYYWTWIRQSPGKGLEWI |
| IgG1 HC knob | | GHIYYSGNTNYNPSLKSRLTISIDT |
| | | SKTQFSLKLSSVTAADTAIYYCVRD |
| | | RVTGAFDIWGQGTMVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKKVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLWCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGK |
| EGFR. | 497 | DIQMTQSPSSLSASVGDRVTITCQA |
| Panitumumab. | | SQDISNYLNWYQQKPGKAPKLLIYD |
| Kappa LC | | ASNLETGVPSRFSGSGSGTDFTFTI |
| | | SSLQPEDIATYFCQHFDHLPLAFGG |
| | | GTKVEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| CD47.VIR47. | 498 | EVQLLESGGGLVQPGGSLRLSCAAS |
| V8. | | GFTFSNFAMSWVRQAPGKGLEWVST |
| scFv hole | | ISASGGRTFYADSVKGRFTISRDNS |
| | | KNTLYLQMNSLRAEDTAVYYCAKEG |
| | | SFGEGVDPWGQGTLVTVSSGGGGSG |
| | | GGGSGGGGSSYELTQPPSVSVSPGQ |
| | | TARITCSGDALPKKYAYWYQQKSGQ |
| | | APVLVIYEDNKRPSGIPERFSGSSS |
| | | GTMATLTISGAQVEDEADYYCYSTD |
| | | ISGNHWVFGGGTKLTVLGGSGGSGG |
| | | ASKTHTCPPCPAPELLGGPSVFLFP |
| | | PKPKDTLMISRTPEVTCVVVDVSHE |
| | | DPEVKFNWYVDGVEVHNAKTKPREE |
| | | QYNSTYRVVSVLTVLHQDWLNGKEY |
| | | KCKVSNKALPAPIEKTISKAKGQPR |
| | | EPQVYTLPPSRDELTKNQVSLSCAV |
| | | KGFYPSDIAVEWESNGQPENNYKTT |
| | | PPVLDSDGSFFLVSKLTVDKSRWQQ |
| | | GNVFSCSVMHEALHNHYTQKSLSLS |
| | | PGK |

TABLE 34
Polypeptide sequences of the proof-of-concept
CD47 × EGFR bispecific antibody:
Cetuximab × VIR47.V8 v2.

| Construct | SEQ ID | Amino Acid Sequence |
|---|---|---|
| Description | NO | (N to C) |
| Anti-CD47 | 499 | SYELTQPPSVSVSPGQTARITCSGD |
| TCRα LC | | ALPKKYAYWYQQKSGQAPVLVIYED |
| | | NKRPSGIPERFSGSSSGTMATLTIS |
| | | GAQVEDEADYYCYSTDISGNHWVFG |
| | | GGTKLTVLPDIQNPDPAVYQLRDSK |
| | | SSDKSVCLFTDFDSQTQVSQSKDSD |
| | | VYITDKCVLDMRSMDFKSNSAVAWS |
| | | QKSDFACANAFQNSIIPEDTFFCS |
| Anti-CD47 | 500 | EVQLLESGGGLVQPGGSLRLSCAAS |
| TCRβ. HC | | GFTFSNFAMSWVRQAPGKGLEWVST |
| | | ISASGGRTFYADSVKGRFTISRDNS |
| | | KNTLYLQMNSLRAEDTAVYYCAKEG |
| | | SFGEGVDPWGQGTLVTVLEDLKNVF |
| | | PPEVAVFEPSECEISHTQKATLVCL |
| | | ATGFYPDHVELSWWVNGKEVHSGVC |
| | | TDPQPLKEQPALQDSRYALSSRLRV |
| | | SATFWQNPRNHFRCQVQFYGLSEND |
| | | EWTQDRAKPVTQIVSAEAWGRASDK |
| | | THTCPPCPAPELLGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPCREEMTKNQVSLWCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPGK |
| Cetuximab | 501 | QVQLKQSGPGLVQPSQSLSITCTVS |
| “Chain 1” | | GFSLTNYGVHWVRQSPGKGLEWLGV |
| | | IWSGGNTDYNTPFTSRLSINKDNSK |
| | | SQVFFKMNSLQSNDTAIYYCARALT |
| | | YYDYEFAYWGQGTLVTVSAASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKRVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | CTLPPSREEMTKNQVSLSCAVKGFY |
| | | PSDIAVEWESNGQPENNYKTTPPVL |
| | | DSDGSFFLVSKLTVDKSRWQQGNVF |
| | | SCSVMHEALHNHYTQKSLSLSPGK |
| Cetuximab | 502 | DILLTQSPVILSVSPGERVSFSCRA |
| “Chain 2” | | SQSIGTNIHWYQQRTNGSPRLLIKY |
| | | ASESISGIPSRFSGSGSGTDFTLSI |
| | | NSVESEDIADYYCQQNNNWPTTFGA |
| | | GTKLELKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |

TABLE 35
Polypeptide sequences of an example three-
chain, anti-CD47 × anti-EGFR bispecific
antibody Cetuximab × VIR47.V8_v3.

| Construct | SEQ ID | Amino Acid Sequence |
|---|---|---|
| Description | NO | (N to C) |
| Cetuximab. | 508 | QVQLKQSGPGLVQPSQSLSITCTVS |
| huIgG1 | | GFSLTNYGVHWVRQSPGKGLEWLGV |
| HC knob | | IWSGGNTDYNTPFTSRLSINKDNSK |
| | | SQVFFKMNSLQSNDTAIYYCARGAL |
| | | TYYDYEFAYWQGTLVTVSAASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKKVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLWCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGK |
| Cetuximab. | 509 | DILLTQSPVILSVSPGERVSFSCRA |
| Kappa LC | | SQSIGTNIHWYQQRTNGSPRLLIKY |
| | | ASESISGIPSRFSGSGSGTDFTLSI |
| | | NSVESEDIADYYCQQNNNWPTTFGA |
| | | GTKLELKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| CD47.VIR47. | 510 | EVQLLESGGGLVQPGGSLRLSCAAS |
| V8.scFv hole | | GFTFSNFAMSWVRQAPGKGLEWVST |
| | | ISASGGRTFYADSVKGRFTISRDNS |
| | | KNTLYLQMNSLRAEDTAVYYCAKEG |
| | | SFGEGVDPWGQGTLVTVSSGGGGSG |
| | | GGGSGGGGSSYELTQPPSVSVSPGQ |
| | | TARITCSGDALPKKYAYWYQQKSGQ |
| | | APVLVIYEDNKRPSGIPERFSGSSS |
| | | GTMATLTISGAQVEDEADYYCYSTD |
| | | ISGNHWVFGGGTKLTVLGGSGGSGG |
| | | ASKTHTCPPCPAPELLGGPSVFLFP |
| | | PKPKDTLMISRTPEVTCVVVDVSHE |
| | | DPEVKFNWYVDGVEVHNAKTKPREE |
| | | QYNSTYRVVSVLTVLHQDWLNGKEY |
| | | KCKVSNKALPAPIEKTISKAKGQPR |
| | | EPQVYTLPPSRDELTKNQVSLSCAV |
| | | KGFYPSDIAVEWESNGQPENNYKTT |
| | | PPVLDSDGSFFLVSKLTVDKSRWQQ |
| | | GNVFSCSVMHEALHNHYTQKSLSLS |
| | | PGK |

In some embodiments, the multispecific (e.g., bispecific) antibodies, and binding fragments thereof, are derived from non-human (e.g. rabbit or mouse) antibodies. In some instances, the humanized form of the non-human antibody contains a minimal non-human sequence to maintain original antigenic specificity. In some cases, the humanized antibodies are human immunoglobulins (acceptor antibody), wherein the CDRs of the acceptor antibody are replaced by residues of the CDRs of a non-human immunoglobulin (donor antibody), such as rat, rabbit, or mouse donor having the desired specificity, affinity, avidity, binding kinetics, and/or capacity. In some instances, one or more framework region (FR) residues of the human immunoglobulin are replaced by corresponding non-human residues of the donor antibody.

### Multispecific Antibody Binding to Target Cells

In some embodiments, a multispecific (e.g., bispecific) antibody of the present disclosure that comprises a first component that binds CD47 and a second component that binds EpCAM, binds to a cell that expresses on its surface target antigens of the multispecific (e.g., bispecific) protein, with at least 2-50 fold, 10-100 fold, 2-fold, 5-fold, 10-fold, 25-fold, 50-fold or 100-fold, or 20-50%, 50-100%, 20%, 25%, 30%, 40%, 50%, 60%, 70%, 80%, 90% or 100% or more higher affinity (e.g., preferentially binds) compared to the binding affinity of an antibody that is bivalent to only one of CD47 or EpCAM, to the cell.

In some embodiments, a multispecific (e.g., bispecific) protein provided herein binds to a target cell that expresses a higher level of EpCAM than CD47, on its surface. For instance, the ratio of EpCAM to CD47 protein expression on the target cell surface, is from about 1, 1.5, 2.0, 2.5, 5, 10, 15, 20, 25, 50, 100, or greater than 200 as measured by flow cytometry.

In some embodiments, a multispecific (e.g., bispecific) protein provided herein binds to a target cell that expresses a higher level of CD47 than EpCAM, on its surface. For instance, the ratio of CD47 to EpCAM protein expression on the target cell surface, is from about 1, 1.5, 2.0, 2.5, 5, 10, 15, 20, 50, 100, or greater than 200 as measured by flow cytometry.

In some embodiments, a multispecific (e.g., bispecific) protein provided herein binds to a target cell that expresses equal levels of CD47 and EpCAM, on its surface. For instance, the ratio of CD47 to EpCAM protein expression on the target cell surface, is about 1 as measured by flow cytometry.

In some cases, a cell expresses about 35,000 CD47 proteins to about 250,000 CD47 proteins. In some cases, a cell expresses at least about 35,000 CD47 proteins. In some cases, a cell expresses at most about 250,000 CD47 proteins. In some cases, a cell expresses about 35,000 CD47 proteins to about 40,000 CD47 proteins, about 35,000 CD47 proteins to about 45,000 CD47 proteins, about 35,000 CD47 proteins to about 60,000 CD47 proteins, about 35,000 CD47 proteins to about 100,000 CD47 proteins, about 35,000 CD47 proteins to about 150,000 CD47 proteins, about 35,000 CD47 proteins to about 250,000 CD47 proteins, about 40,000 CD47 proteins to about 45,000 CD47 proteins, about 40,000 CD47 proteins to about 60,000 CD47 proteins, about 40,000 CD47 proteins to about 120,000 CD47 proteins, about 40,000 CD47 proteins to about 150,000 CD47 proteins, about 40,000 CD47 proteins to about 250,000 CD47 proteins, about 45,000 CD47 proteins to about 60,000 CD47 proteins, about 45,000 CD47 proteins to about 120,000 CD47 proteins, about 45,000 CD47 proteins to about 150,000 CD47 proteins, about 45,000 CD47 proteins to about 250,000 CD47 proteins, about 60,000 CD47 proteins to about 120,000 CD47 proteins, about 60,000 CD47 proteins to about 150,000 CD47 proteins, about 60,000 CD47 proteins to about 250,000 CD47 proteins, about 120,000 CD47 proteins to about 150,000 CD47 proteins, about 120,000 CD47 proteins to about 250,000 CD47 proteins, or about 150,000 CD47 proteins to about 250,000 CD47 proteins. In some cases, the CD47 proteins are expressed on the surface of a cell. In some cases, the cell is a tumor cell. In some cases, the cell is derived from a ductal pancreatic adenocarcinoma, an ovarian clear cell adenocarcinoma, a colon adenocarcinoma, a lung adenocarcinoma, a lung squamous cell carcinoma, a lung small cell carcinoma, a hepatocellular carcinoma, a gastric adenocarcinoma, an esophageal cancer, a cholangiocarcinoma, a head and neck squamous cell carcinoma, a prostate carcinoma, an endometrial carcinoma, an ovarian adeno carcinoma, or a vulvar squamous cell carcinoma. In some cases, the number of CD47 proteins expressed on a cell is measured by flow cytometry. In some cases, the number of CD47 proteins expressed on a cell is measured by quantitative flow cytometry.

In some cases, a cell expresses about 35,000 EpCAM proteins to about 2×10⁷ EpCAM proteins. In some cases, a cell expresses at least about 35,000 EpCAM proteins. In some cases, a cell expresses at most about 2×10⁷ EpCAM proteins. In some cases, a cell expresses about 35,000 EpCAM proteins to about 85,000 EpCAM proteins, about 35,000 EpCAM proteins to about 170,000 EpCAM proteins, about 35,000 EpCAM proteins to about 300,000 EpCAM proteins, about 35,000 EpCAM proteins to about 400,000 EpCAM proteins, about 35,000 EpCAM proteins to about 650,000 EpCAM proteins, about 35,000 EpCAM proteins to about 750,000 EpCAM proteins, about 35,000 EpCAM proteins to about 1,500,000 EpCAM proteins, about 35,000 EpCAM proteins to about 1,900,000 EpCAM proteins, about 35,000 EpCAM proteins to about 4×10⁶ EpCAM proteins, about 35,000 EpCAM proteins to about 6×10⁶ EpCAM proteins, about 35,000 EpCAM proteins to about 7×10⁶ EpCAM proteins, about 35,000 EpCAM proteins to about 15×10⁶ EpCAM proteins, about 35,000 EpCAM proteins to about 2×10⁷ EpCAM proteins, about 85,000 EpCAM proteins to about 170,000 EpCAM proteins, about 85,000 EpCAM proteins to about 300,000 EpCAM proteins, about 85,000 EpCAM proteins to about 400,000 EpCAM proteins, about 85,000 EpCAM proteins to about 650,000 EpCAM proteins, about 85,000 EpCAM proteins to about 750,000 EpCAM proteins, about 85,000 EpCAM proteins to about 1,500,000 EpCAM proteins, about 85,000 EpCAM proteins to about 1,900,000 EpCAM proteins, about 85,000 EpCAM proteins to about 4×10⁶ EpCAM proteins, about 85,000 EpCAM proteins to about 6×10⁶ EpCAM proteins, about 85,000 EpCAM proteins to about 7×10⁶ EpCAM proteins, about 85,000 EpCAM proteins to about 15×10⁶ EpCAM proteins, about 85,000 EpCAM proteins to about 2×10⁷ EpCAM proteins, about 170,000 EpCAM proteins to about 300,000 EpCAM proteins, about 170,000 EpCAM proteins to about 400,000 EpCAM proteins, about 170,000 EpCAM proteins to about 650,000 EpCAM proteins, about 170,000 EpCAM proteins to about 750,000 EpCAM proteins, about 170,000 EpCAM proteins to about 1,500,000 EpCAM proteins, about 170,000 EpCAM proteins to about 1,900,000 EpCAM proteins, about 170,000 EpCAM proteins to about 4×10⁶ EpCAM proteins, about 170,000 EpCAM proteins to about 6×10⁶ EpCAM proteins, about 170,000 EpCAM proteins to about 7×10⁶ EpCAM proteins, about 170,000 EpCAM proteins to about 15×10⁶ EpCAM proteins, about 170,000 EpCAM proteins to about 2×10⁷ EpCAM proteins, about 300,000 EpCAM proteins to about 400,000 EpCAM proteins, about 300,000 EpCAM proteins to about 650,000 EpCAM proteins, about 300,000 EpCAM proteins to about 750,000 EpCAM proteins, about 300,000 EpCAM proteins to about 1,500,000 EpCAM proteins, about 300,000 EpCAM proteins to about 1,900,000 EpCAM proteins, about 300,000 EpCAM proteins to about 4×10⁶ EpCAM proteins, about 300,000 EpCAM proteins to about 6×10⁶ EpCAM proteins, about 300,000 EpCAM proteins to about 7×10⁶ EpCAM proteins, about 300,000 EpCAM proteins to about 15×10⁶ EpCAM proteins, about 300,000 EpCAM proteins to about 2×10⁷ EpCAM proteins, about 400,000 EpCAM proteins to about 650,000 EpCAM proteins, about 400,000 EpCAM proteins to about 750,000 EpCAM proteins, about 400,000 EpCAM proteins to about 1,500,000 EpCAM proteins, about 400,000 EpCAM proteins to about 1,900,000 EpCAM proteins, about 400,000 EpCAM proteins to about 4×10⁶ EpCAM proteins, about 400,000 EpCAM proteins to about 6×10⁶ EpCAM proteins, about 400,000 EpCAM proteins to about 7×10⁶ EpCAM proteins, about 400,000 EpCAM proteins to about 15×10⁶ EpCAM proteins, about 400,000 EpCAM proteins to about 2×10⁷ EpCAM proteins, about 650,000 EpCAM proteins to about 750,000 EpCAM proteins, about 650,000 EpCAM proteins to about 1,500,000 EpCAM proteins, about 650,000 EpCAM proteins to about 1,900,000 EpCAM proteins, about 650,000 EpCAM proteins to about 4×10⁶ EpCAM proteins, about 650,000 EpCAM proteins to about 6×10⁶ EpCAM proteins, about 650,000 EpCAM proteins to about 7×10⁶ EpCAM proteins, about 650,000 EpCAM proteins to about 15×10⁶ EpCAM proteins, about 650,000 EpCAM proteins to about 2×10⁷ EpCAM proteins, about 750,000 EpCAM proteins to about 1,500,000 EpCAM proteins, about 750,000 EpCAM proteins to about 1,900,000 EpCAM proteins, about 750,000 EpCAM proteins to about 4×10⁶ EpCAM proteins, about 750,000 EpCAM proteins to about 6×10⁶ EpCAM proteins, about 750,000 EpCAM proteins to about 7×10⁶ EpCAM proteins, about 750,000 EpCAM proteins to about 15×10⁶ EpCAM proteins, about 750,000 EpCAM proteins to about 2×10⁷ EpCAM proteins, about 1,500,000 EpCAM proteins to about 1,900,000 EpCAM proteins, about 1,500,000 EpCAM proteins to about 4×10⁶ EpCAM proteins, about 1,500,000 EpCAM proteins to about 6×10⁶ EpCAM proteins, about 1,500,000 EpCAM proteins to about 7×10⁶ EpCAM proteins, about 1,500,000 EpCAM proteins to about 15×10⁶ EpCAM proteins, about 1,500,000 EpCAM proteins to about 2×10⁷ EpCAM proteins, about 1,900,000 EpCAM proteins to about 4×10⁶ EpCAM proteins, about 1,900,000 EpCAM proteins to about 6×10⁶ EpCAM proteins, about 1,900,000 EpCAM proteins to about 7×10⁶ EpCAM proteins, about 1,900,000 EpCAM proteins to about 15×10⁶ EpCAM proteins, about 1,900,000 EpCAM proteins to about 2×10⁷ EpCAM proteins, about 4×10⁶ EpCAM proteins to about 6×10⁶ EpCAM proteins, about 4×10⁶ EpCAM proteins to about 7×10⁶ EpCAM proteins, about 4×10⁶ EpCAM proteins to about 15×10⁶ EpCAM proteins, about 4×10⁶ EpCAM proteins to about 2×10⁷ EpCAM proteins, about 6×10⁶ EpCAM proteins to about 7×10⁶ EpCAM proteins, about 6×10⁶ EpCAM proteins to about 15×10⁶ EpCAM proteins, about 6×10⁶ EpCAM proteins to about 2×10⁷ EpCAM proteins, or about 15×10⁶ EpCAM proteins to about 2×10⁷ EpCAM proteins. In some cases, the EpCAM proteins are expressed on the surface of a cell. In some cases, the cell is a tumor cell. In some cases, the cell is derived from a ductal pancreatic adenocarcinoma, an ovarian clear cell adenocarcinoma, a colon adenocarcinoma, a lung adenocarcinoma, a lung squamous cell carcinoma, a lung small cell carcinoma, a hepatocellular carcinoma, a gastric adenocarcinoma, a cholangiocarcinoma, an esophageal cancer, a head and neck squamous cell carcinoma, a prostate carcinoma, an endometrial carcinoma, an ovarian adeno carcinoma, or a vulvar squamous cell carcinoma. In some cases, the number of EpCAM proteins expressed on a cell is measured by flow cytometry. In some cases, the number of EpCAM proteins expressed on a cell is measured by quantitative flow cytometry.

In some embodiments, a multispecific (e.g., bispecific) protein with a CD47-binding domain and an EpCAM-binding domain has enhanced affinity for a cell that expresses CD47 and EpCAM compared to a bivalent protein with one or more CD47-binding domains and/or a bivalent protein with one or more EpCAM-binding domains. In some instances, the multispecific (e.g., bispecific) protein has 1.5, 2, 3, 4, 5, or 10-fold higher affinity for the CD47-expressing cell than a bivalent protein that binds to CD47 or a bivalent protein that binds to EpCAM. In some embodiments, a multispecific (e.g., bispecific) protein with a CD47-binding domain and an EpCAM-binding domain has enhanced affinity for a cell that expresses higher levels of CD47 than EpCAM compared to a bivalent protein with a CD47-binding domain. In some instances, the multispecific (e.g., bispecific) protein has 1.5, 2, 3, 4, 5, or 10-fold higher affinity for the cell with higher EpCAM expression than CD47 expression compared to a bivalent protein that binds to CD47.

In some embodiments, a multispecific (e.g., bispecific) antibody with a CD47 binding domain and an EpCAM binding domain has decreased red blood cell (RBC) binding compared to CD47 BMK-1 or CD47 BMK-2 (such as described herein). In some cases, the decreased RBC binding is a decrease of at least 10%, 20%, 30%, 40%, 50%, 60%, 70%, 80%, 90%, 95%, or 100% compared to CD47 BMK-1. In some cases, the decreased RBC binding is a decrease of at least 10%, 20%, 30%, 40%, 50%, 60%, 70%, 80%, 90%, 95%, or 100% compared to CD47 BMK-2.

In some embodiments, a multispecific (e.g., bispecific) antibody of the present disclosure that comprises a first component that binds CD47 and a second component that binds EGFR, binds to a cell that expresses on its surface target antigens of the multispecific (e.g., bispecific) protein, with at least 2-50 fold, 10-100 fold, 2-fold, 5-fold, 10-fold, 25-fold, 50-fold or 100-fold, or 20-50%, 50-100%, 20%, 25%, 30%, 40%, 50%, 60%, 70%, 80%, 90% or 100% or more higher affinity (e.g., preferentially binds) compared to the binding affinity of an antibody that is bivalent to only one of CD47 or EGFR, to the cell.

In some embodiments, a multispecific (e.g., bispecific) protein provided herein binds to a target cell that expresses a higher level of EGFR than CD47, on its surface. For instance, the ratio of EGFR to CD47 protein expression on the target cell surface, is from about 1, 1.5, 2.0, 2.5, 5, 10, 15, 20, 25, 50, 100, or greater than 200 as measured by flow cytometry.

In some embodiments, a multispecific (e.g., bispecific) protein provided herein binds to a target cell that expresses a higher level of CD47 than EGFR, on its surface. For instance, the ratio of CD47 to EGFR protein expression on the target cell surface, is from about 1, 1.5, 2.0, 2.5, 5, 10, 15, 20, 50, 100, or greater than 200 as measured by flow cytometry.

In some embodiments, a multispecific (e.g., bispecific) protein provided herein binds to a target cell that expresses equal levels of CD47 and EGFR, on its surface. For instance, the ratio of CD47 to EGFR protein expression on the target cell surface, is about 1 as measured by flow cytometry.

In some cases, a cell expresses about 35,000 CD47 proteins to about 250,000 CD47 proteins. In some cases, a cell expresses at least about 35,000 CD47 proteins. In some cases, a cell expresses at most about 250,000 CD47 proteins. In some cases, a cell expresses about 35,000 CD47 proteins to about 40,000 CD47 proteins, about 35,000 CD47 proteins to about 45,000 CD47 proteins, about 35,000 CD47 proteins to about 60,000 CD47 proteins, about 35,000 CD47 proteins to about 100,000 CD47 proteins, about 35,000 CD47 proteins to about 150,000 CD47 proteins, about 35,000 CD47 proteins to about 250,000 CD47 proteins, about 40,000 CD47 proteins to about 45,000 CD47 proteins, about 40,000 CD47 proteins to about 60,000 CD47 proteins, about 40,000 CD47 proteins to about 120,000 CD47 proteins, about 40,000 CD47 proteins to about 150,000 CD47 proteins, about 40,000 CD47 proteins to about 250,000 CD47 proteins, about 45,000 CD47 proteins to about 60,000 CD47 proteins, about 45,000 CD47 proteins to about 120,000 CD47 proteins, about 45,000 CD47 proteins to about 150,000 CD47 proteins, about 45,000 CD47 proteins to about 250,000 CD47 proteins, about 60,000 CD47 proteins to about 120,000 CD47 proteins, about 60,000 CD47 proteins to about 150,000 CD47 proteins, about 60,000 CD47 proteins to about 250,000 CD47 proteins, about 120,000 CD47 proteins to about 150,000 CD47 proteins, about 120,000 CD47 proteins to about 250,000 CD47 proteins, or about 150,000 CD47 proteins to about 250,000 CD47 proteins. In some cases, the CD47 proteins are expressed on the surface of a cell. In some cases, the cell is a tumor cell. In some cases, the cell is derived from a ductal pancreatic adenocarcinoma, an ovarian clear cell adenocarcinoma, a colon adenocarcinoma, a lung adenocarcinoma, a lung squamous cell carcinoma, a lung small cell carcinoma, a hepatocellular carcinoma, a gastric adenocarcinoma, an esophageal cancer, a cholangiocarcinoma, a head and neck squamous cell carcinoma, a prostate carcinoma, an endometrial carcinoma, an ovarian adeno carcinoma, or a vulvar squamous cell carcinoma. In some cases, the number of CD47 proteins expressed on a cell is measured by flow cytometry. In some cases, the number of CD47 proteins expressed on a cell is measured by quantitative flow cytometry.

In some cases, a cell expresses about 35,000 EGFR proteins to about 2×10⁷ EGFR proteins. In some cases, a cell expresses at least about 35,000 EGFR proteins. In some cases, a cell expresses at most about 2×10⁷ EGFR proteins. In some cases, a cell expresses about 35,000 EGFR proteins to about 85,000 EGFR proteins, about 35,000 EGFR proteins to about 170,000 EGFR proteins, about 35,000 EGFR proteins to about 300,000 EGFR proteins, about 35,000 EGFR proteins to about 400,000 EGFR proteins, about 35,000 EGFR proteins to about 650,000 EGFR proteins, about 35,000 EGFR proteins to about 750,000 EGFR proteins, about 35,000 EGFR proteins to about 1,500,000 EGFR proteins, about 35,000 EGFR proteins to about 1,900,000 EGFR proteins, about 35,000 EGFR proteins to about 4×10⁶ EGFR proteins, about 35,000 EGFR proteins to about 6×10⁶ EGFR proteins, about 35,000 EGFR proteins to about 7×10⁶ EGFR proteins, about 35,000 EGFR proteins to about 15×10⁶ EGFR proteins, about 35,000 EGFR proteins to about 2×10⁷ EGFR proteins, about 85,000 EGFR proteins to about 170,000 EGFR proteins, about 85,000 EGFR proteins to about 300,000 EGFR proteins, about 85,000 EGFR proteins to about 400,000 EGFR proteins, about 85,000 EGFR proteins to about 650,000 EGFR proteins, about 85,000 EGFR proteins to about 750,000 EGFR proteins, about 85,000 EGFR proteins to about 1,500,000 EGFR proteins, about 85,000 EGFR proteins to about 1,900,000 EGFR proteins, about 85,000 EGFR proteins to about 4×10⁶ EGFR proteins, about 85,000 EGFR proteins to about 6×10⁶ EGFR proteins, about 85,000 EGFR proteins to about 7×10⁶ EGFR proteins, about 85,000 EGFR proteins to about 15×10⁶ EGFR proteins, about 85,000 EGFR proteins to about 2×10⁷ EGFR proteins, about 170,000 EGFR proteins to about 300,000 EGFR proteins, about 170,000 EGFR proteins to about 400,000 EGFR proteins, about 170,000 EGFR proteins to about 650,000 EGFR proteins, about 170,000 EGFR proteins to about 750,000 EGFR proteins, about 170,000 EGFR proteins to about 1,500,000 EGFR proteins, about 170,000 EGFR proteins to about 1,900,000 EGFR proteins, about 170,000 EGFR proteins to about 4×10⁶ EGFR proteins, about 170,000 EGFR proteins to about 6×10⁶ EGFR proteins, about 170,000 EGFR proteins to about 7×10⁶ EGFR proteins, about 170,000 EGFR proteins to about 15×10⁶ EGFR proteins, about 170,000 EGFR proteins to about 2×10⁷ EGFR proteins, about 300,000 EGFR proteins to about 400,000 EGFR proteins, about 300,000 EGFR proteins to about 650,000 EGFR proteins, about 300,000 EGFR proteins to about 750,000 EGFR proteins, about 300,000 EGFR proteins to about 1,500,000 EGFR proteins, about 300,000 EGFR proteins to about 1,900,000 EGFR proteins, about 300,000 EGFR proteins to about 4×10⁶ EGFR proteins, about 300,000 EGFR proteins to about 6×10⁶ EGFR proteins, about 300,000 EGFR proteins to about 7×10⁶ EGFR proteins, about 300,000 EGFR proteins to about 15×10⁶ EGFR proteins, about 300,000 EGFR proteins to about 2×10⁷ EGFR proteins, about 400,000 EGFR proteins to about 650,000 EGFR proteins, about 400,000 EGFR proteins to about 750,000 EGFR proteins, about 400,000 EGFR proteins to about 1,500,000 EGFR proteins, about 400,000 EGFR proteins to about 1,900,000 EGFR proteins, about 400,000 EGFR proteins to about 4×10⁶ EGFR proteins, about 400,000 EGFR proteins to about 6×10⁶ EGFR proteins, about 400,000 EGFR proteins to about 7×10⁶ EGFR proteins, about 400,000 EGFR proteins to about 15×10⁶ EGFR proteins, about 400,000 EGFR proteins to about 2×10⁷ EGFR proteins, about 650,000 EGFR proteins to about 750,000 EGFR proteins, about 650,000 EGFR proteins to about 1,500,000 EGFR proteins, about 650,000 EGFR proteins to about 1,900,000 EGFR proteins, about 650,000 EGFR proteins to about 4×10⁶ EGFR proteins, about 650,000 EGFR proteins to about 6×10⁶ EGFR proteins, about 650,000 EGFR proteins to about 7×10⁶ EGFR proteins, about 650,000 EGFR proteins to about 15×10⁶ EGFR proteins, about 650,000 EGFR proteins to about 2×10⁷ EGFR proteins, about 750,000 EGFR proteins to about 1,500,000 EGFR proteins, about 750,000 EGFR proteins to about 1,900,000 EGFR proteins, about 750,000 EGFR proteins to about 4×10⁶ EGFR proteins, about 750,000 EGFR proteins to about 6×10⁶ EGFR proteins, about 750,000 EGFR proteins to about 7×10⁶ EGFR proteins, about 750,000 EGFR proteins to about 15×10⁶ EGFR proteins, about 750,000 EGFR proteins to about 2×10⁷ EGFR proteins, about 1,500,000 EGFR proteins to about 1,900,000 EGFR proteins, about 1,500,000 EGFR proteins to about 4×10⁶ EGFR proteins, about 1,500,000 EGFR proteins to about 6×10⁶ EGFR proteins, about 1,500,000 EGFR proteins to about 7×10⁶ EGFR proteins, about 1,500,000 EGFR proteins to about 15×10⁶ EGFR proteins, about 1,500,000 EGFR proteins to about 2×10⁷ EGFR proteins, about 1,900,000 EGFR proteins to about 4×10⁶ EGFR proteins, about 1,900,000 EGFR proteins to about 6×10⁶ EGFR proteins, about 1,900,000 EGFR proteins to about 7×10⁶ EGFR proteins, about 1,900,000 EGFR proteins to about 15×10⁶ EGFR proteins, about 1,900,000 EGFR proteins to about 2×10⁷ EGFR proteins, about 4×10⁶ EGFR proteins to about 6×10⁶ EGFR proteins, about 4×10⁶ EGFR proteins to about 7×10⁶ EGFR proteins, about 4×10⁶ EGFR proteins to about 15×10⁶ EGFR proteins, about 4×10⁶ EGFR proteins to about 2×10⁷ EGFR proteins, about 6×10⁶ EGFR proteins to about 7×10⁶ EGFR proteins, about 6×10⁶ EGFR proteins to about 15×10⁶ EGFR proteins, about 6×10⁶ EGFR proteins to about 2×10⁷ EGFR proteins, or about 15×10⁶ EGFR proteins to about 2×10⁷ EGFR proteins. In some cases, the EGFR proteins are expressed on the surface of a cell. In some cases, the cell is a tumor cell. In some cases, the cell is derived from a ductal pancreatic adenocarcinoma, an ovarian clear cell adenocarcinoma, a colon adenocarcinoma, a lung adenocarcinoma, a lung squamous cell carcinoma, a lung small cell carcinoma, a hepatocellular carcinoma, a gastric adenocarcinoma, an esophageal cancer, a cholangiocarcinoma, a head and neck squamous cell carcinoma, a prostate carcinoma, an endometrial carcinoma, an ovarian adeno carcinoma, or a vulvar squamous cell carcinoma. In some cases, the number of EGFR proteins expressed on a cell is measured by flow cytometry. In some cases, the number of EGFR proteins expressed on a cell is measured by quantitative flow cytometry.

In some embodiments, a multispecific (e.g., bispecific) protein with a CD47-binding domain and an EGFR-binding domain has enhanced affinity for a cell that expresses CD47 and EGFR compared to a bivalent protein with one or more CD47-binding domains and/or a bivalent protein with one or more EGFR-binding domains. In some instances, the multispecific (e.g., bispecific) protein has 1.5, 2, 3, 4, 5, or 10-fold higher affinity for the CD47-expressing cell than a bivalent protein that binds to CD47 or a bivalent protein that binds to EGFR. In some embodiments, a multispecific (e.g., bispecific) protein with a CD47-binding domain and an EGFR-binding domain has enhanced affinity for a cell that expresses higher levels of CD47 than EGFR compared to a bivalent protein with a CD47-binding domain. In some instances, the multispecific (e.g., bispecific) protein has 1.5, 2, 3, 4, 5, or 10-fold higher affinity for the cell with higher EGFR expression than CD47 expression compared to a bivalent protein that binds to CD47.

In some embodiments, a multispecific (e.g., bispecific) antibody with a CD47 binding domain and an EGFR binding domain has decreased red blood cell (RBC) binding compared to CD47 BMK-1 or CD47 BMK-2 (such as described herein). In some cases, the decreased RBC binding is a decrease of at least 10%, 20%, 30%, 40%, 50%, 60%, 70%, 80%, 90%, 95%, or 100% compared to CD47 BMK-1. In some cases, the decreased RBC binding is a decrease of at least 10%, 20%, 30%, 40%, 50%, 60%, 70%, 80%, 90%, 95%, or 100% compared to CD47 BMK-2.

### Immunological Activity of Multispecific Antibodies on Target Cells

In some embodiments, the multispecific antibody with a CD47-binding domain and an EpCAM-binding domain has a higher immunologic activity against a CD47-expressing cell compared to a bivalent antibody with a CD47-binding domain and/or a bivalent antibody with an EpCAM-binding domain. In some instances, the multispecific antibody has a 1.5, 2, 3, 4, 5, or 10-fold higher immunological activity than a bivalent antibody with a CD47-binding domain and/or a bivalent antibody with an EpCAM-binding domain. Various immunological activities of a multispecific antibody can be measured in in vitro assays such as an ADCC assay and an ADCP assay. In some instances, the multispecific antibody has a 1.5, 2, 3, 4, 5, or 10-fold higher ADCC activity than a bivalent antibody with a CD47-binding domain and/or a bivalent antibody with an EpCAM-binding domain. In some instances, the multispecific antibody has a 1.5, 2, 3, 4, 5, or 10-fold higher ADCP activity than a bivalent antibody with a CD47-binding domain and/or a bivalent antibody with an EpCAM-binding domain.

In some embodiments, the multispecific antibody with a CD47-binding domain and an EpCAM-binding domain has a higher immunologic activity against a CD47-expressing cell compared to a bivalent antibody with a CD47-binding domain and/or a bivalent antibody with an EpCAM-binding domain. In some instances, the multispecific antibody has a 10-20%, 21-30%, 31-40%, 41-50% or at least 51% higher immunological activity than a bivalent antibody with a CD47-binding domain and/or a bivalent antibody with an EpCAM-binding domain. Various immunological activities of a multispecific antibody can be measured in in vitro assays such as an ADCC assay and an ADCP assay. In some instances, the multispecific antibody has a 10-20%, 21-30%, 31-40%, 41-50% or at least 51% higher ADCC activity than a bivalent antibody with a CD47-binding domain and/or a bivalent antibody with an EpCAM-binding domain. In some instances, the multispecific antibody has a 10-20%, 21-30%, 31-40%, 41-50% or at least 51% higher ADCP activity than a bivalent antibody with a CD47-binding domain and/or a bivalent antibody with an EpCAM-binding domain.

In some embodiments, described herein is a multispecific antibody which comprises a first targeting moiety that specifically binds to CD47 and a second targeting moiety that specifically binds to EpCAM. In some instances, the multispecific antibody further comprises an enhanced ADCP effect compared to an ADCP effect by reference antibody CD47 BMK-1 (such as described herein). In some instances, the multispecific antibody further comprises an enhanced ADCC effect compared to an ADCC effect by reference antibody CD47 BMK-2. In some cases, the enhanced ADCP is at least 2-fold, 3-fold, 4-fold, or higher than the ADCP effect of reference antibody CD47 BMK-1. In some cases, the enhanced ADCP is at least 30%, 40%, 50%, 60%, 70%, 80%, 90%, or higher than the CD47 BMK-1 effect of reference antibody CD47 BMK-1. In some cases, the enhanced ADCC is at least 2-fold, 3-fold, 4-fold, 5-fold, or higher than the ADCC effect of reference antibody CD47 BMK-2. In some cases, the enhanced ADCC is at least 30%, 40%, 50%, 60%, 70%, 80%, 90%, or higher than the ADCC effect of reference antibody CD47 BMK-2.

In some embodiments, described herein is a multispecific antibody which comprises a first targeting moiety that specifically binds to CD47 and a second targeting moiety that specifically binds to EpCAM. In some instances, the multispecific antibody further comprises an enhanced ADCP effect compared to an ADCP effect by reference antibody CD47 BMK-1 (such as described herein). In some instances, the multispecific antibody further comprises an enhanced ADCC effect compared to an ADCC effect by reference antibody CD47 BMK-2. In some cases, the enhanced ADCP is at least 2-fold, 3-fold, 4-fold, or higher than the ADCP effect of reference antibody CD47 BMK-2. In some cases, the enhanced ADCP is at least 30%, 40%, 50%, 60%, 70%, 80%, 90%, or higher than the CD47 BMK-2 effect of reference antibody CD47 BMK-2. In some cases, the enhanced ADCC is at least 2-fold, 3-fold, 4-fold, 5-fold, or higher than the ADCC effect of reference antibody CD47 BMK-2. In some cases, the enhanced ADCC is at least 30%, 40%, 50%, 60%, 70%, 80%, 90%, or higher than the ADCC effect of reference antibody CD47 BMK-2.

In some embodiments, described herein is multispecific antibody comprising a first component that binds specifically to CD47 and a second component that binds specifically to EpCAM, wherein the multispecific antibody mediates ADCC more efficiently than a bivalent antibody that comprises either the first component or the second component, wherein the ADCC activity is determined using an in vitro cytotoxicity assay. In some embodiments, the multispecific antibody mediates at least about 5%, at least about 10%, at least about 20%, at least about 30%, at least about 40%, at least about 50%, at least about 75%, at least about 100% higher maximum cytotoxicity in an in vitro ADCC assay than the bivalent antibody that comprises either the first component or the second component. In some embodiments, the multispecific antibody mediates at least 2-fold, at least 3-fold, at least 4-fold, at least 5-fold or at least 10-fold higher maximum cytotoxicity in an in vitro ADCC assay than the bivalent antibody that comprises either the first component or the second component.

In some embodiments, described herein is multispecific antibody comprising a first component that binds specifically to CD47 and a second component that binds specifically to EpCAM, wherein the multispecific antibody mediates antibody dependent cellular phagocytosis (ADCP) more efficiently than a bivalent antibody that comprises either the first component or the second component, wherein the ADCP activity is determined using an in vitro FACS based phagocytosis assay. In some embodiments, the multispecific antibody mediates at least about 5%, at least about 10%, at least about 20%, at least about 30%, at least about 40%, at least about 50%, at least about 75%, at least about 100% higher maximum phagocytosis in an in vitro ADCP assay than the bivalent antibody that comprises either the first component or the second component.

In some instances, the multispecific antibody further comprises improved cancer cell killing by immune cells compared to the killing by immune cells of the reference antibody CD47 BMK-1 (such as described herein). In some cases, the cancer cell killing is improved by about 10%, 20%, 30%, 40%, 50%, 60%, 70%, 80%, 90%, or higher compared to the cancer cell killed in the presence of reference antibody CD47 BMK-1. In some cases, the immune cell is a Natural Killer cell. In some cases, the immune cell is a phagocytic cell. In some cases, the immune cell is a macrophage.

In some instances, the multispecific antibody further comprises improved cancer cell killing by immune cells compared to the killing by immune cells of the reference antibody CD47 BMK-2 (such as described herein). In some cases, the cancer cell killing is improved by about 10%, 20%, 30%, 40%, 50%, 60%, 70%, 80%, 90%, or higher compared to the cancer cell killed in the presence of reference antibody CD47 BMK-2. In some cases, the immune cell is a Natural Killer cell. In some cases, the immune cell is a phagocytic cell. In some cases, the immune cell is a macrophage.

Immunological activity can also be measured in a cell-line derived xenograft assay, wherein transformed cells are injected into mice and form a tumor. In some instances, a multispecific protein with a CD47-binding domain and an EpCAM-binding domain inhibits the growth of a tumor comprising CD47-expressing cells to a greater extent than a bivalent protein with the same CD47-binding domain and/or a bivalent protein with the same EpCAM-binding domain. In some instances, the multispecific protein exhibits 1.5, 2, 3, 4, 5, or 10-fold higher inhibition of xenograft tumor growth compared to a bivalent protein with a CD47-binding domain and/or a bivalent protein with an EpCAM-binding domain.

In some embodiments, a multispecific antibody with a CD47-binding domain and an EGFR-binding domain has a higher immunologic activity against a CD47-expressing cell compared to a bivalent antibody with a CD47-binding domain and/or a bivalent antibody with an EGFR-binding domain. In some instances, the multispecific antibody has a 1.5, 2, 3, 4, 5, or 10-fold higher immunological activity than a bivalent antibody with a CD47-binding domain and/or a bivalent antibody with an EGFR-binding domain. Various immunological activities of a multispecific antibody can be measured in in vitro assays such as an ADCC assay and an ADCP assay. In some instances, the multispecific antibody has a 1.5, 2, 3, 4, 5, or 10-fold higher ADCC activity than a bivalent antibody with a CD47-binding domain and/or a bivalent antibody with an EGFR-binding domain. In some instances, the multispecific antibody has a 1.5, 2, 3, 4, 5, or 10-fold higher ADCP activity than a bivalent antibody with a CD47-binding domain and/or a bivalent antibody with an EGFR-binding domain.

In some embodiments, a multispecific antibody with a CD47-binding domain and an EGFR-binding domain has a higher immunologic activity against a CD47-expressing cell compared to a bivalent antibody with a CD47-binding domain and/or a bivalent antibody with an EGFR-binding domain. In some instances, the multispecific antibody has a 10-20%, 21-30%, 31-40%, 41-50% or at least 51% higher immunological activity than a bivalent antibody with a CD47-binding domain and/or a bivalent antibody with an EGFR-binding domain. Various immunological activities of a multispecific antibody can be measured in in vitro assays such as an ADCC assay and an ADCP assay. In some instances, the multispecific antibody has a 10-20%, 21-30%, 31-40%, 41-50% or at least 51% higher ADCC activity than a bivalent antibody with a CD47-binding domain and/or a bivalent antibody with an EGFR-binding domain. In some instances, the multispecific antibody has a 10-20%, 21-30%, 31-40%, 41-50% or at least 51% higher ADCP activity than a bivalent antibody with a CD47-binding domain and/or a bivalent antibody with an EGFR-binding domain.

In some embodiments, described herein is a multispecific antibody which comprises a first targeting moiety that specifically binds to CD47 and a second targeting moiety that specifically binds to EGFR. In some instances, the multispecific antibody further comprises an enhanced ADCP effect compared to an ADCP effect by reference antibody CD47 BMK-1 (such as described herein). In some instances, the multispecific antibody further comprises an enhanced ADCC effect compared to an ADCC effect by reference antibody CD47 BMK-1. In some cases, the enhanced ADCP is at least 2-fold, 3-fold, 4-fold, or higher than the ADCP effect of reference antibody CD47 BMK-1. In some cases, the enhanced ADCP is at least 30%, 40%, 50%, 60%, 70%, 80%, 90%, or higher than the ADCP effect of reference antibody CD47 BMK-1. In some cases, the enhanced ADCC is at least 2-fold, 3-fold, 4-fold, 5-fold, or higher than the ADCC effect of reference antibody CD47 BMK-1. In some cases, the enhanced ADCC is at least 30%, 40%, 50%, 60%, 70%, 80%, 90%, or higher than the ADCC effect of reference antibody CD47 BMK-1.

In some embodiments, described herein is a multispecific antibody which comprises a first targeting moiety that specifically binds to CD47 and a second targeting moiety that specifically binds to EGFR. In some instances, the multispecific antibody further comprises an enhanced ADCP effect compared to an ADCP effect by reference antibody CD47 BMK-2 (such as described herein). In some instances, the multispecific antibody further comprises an enhanced ADCC effect compared to an ADCC effect by reference antibody CD47 BMK-2. In some cases, the enhanced ADCP is at least 2-fold, 3-fold, 4-fold, or higher than the ADCP effect of reference antibody CD47 BMK-2. In some cases, the enhanced ADCP is at least 30%, 40%, 50%, 60%, 70%, 80%, 90%, or higher than the ADCP effect of reference antibody CD47 BMK-2. In some cases, the enhanced ADCC is at least 2-fold, 3-fold, 4-fold, 5-fold, or higher than the ADCC effect of reference antibody CD47 BMK-2. In some cases, the enhanced ADCC is at least 30%, 40%, 50%, 60%, 70%, 80%, 90%, or higher than the ADCC effect of reference antibody CD47 BMK-2.

In some embodiments, described herein is multispecific antibody comprising a first component that binds specifically to CD47 and a second component that binds specifically to EGFR, wherein the multispecific antibody mediates ADCC more efficiently than a bivalent antibody that comprises either the first component or the second component, wherein the ADCC activity is determined using an in vitro cytotoxicity assay. In some embodiments, the multispecific antibody mediates at least about 5%, at least about 10%, at least about 20%, at least about 30%, at least about 40%, at least about 50%, at least about 75%, at least about 100% higher maximum cytotoxicity in an in vitro ADCC assay than the bivalent antibody that comprises either the first component or the second component. In some embodiments, the multispecific antibody mediates at least 2-fold, at least 3-fold, at least 4-fold, at least 5-fold or at least 10-fold higher maximum cytotoxicity in an in vitro ADCC assay than the bivalent antibody that comprises either the first component or the second component.

In some embodiments, described herein is multispecific antibody comprising a first component that binds specifically to CD47 and a second component that binds specifically to EGFR, wherein the multispecific antibody mediates antibody dependent cellular phagocytosis (ADCP) more efficiently than a bivalent antibody that comprises either the first component or the second component, wherein the ADCP activity is determined using an in vitro FACS based phagocytosis assay. In some embodiments, the multispecific antibody mediates at least about 5%, at least about 10%, at least about 20%, at least about 30%, at least about 40%, at least about 50%, at least about 75%, at least about 100% higher maximum phagocytosis in an in vitro ADCP assay than the bivalent antibody that comprises either the first component or the second component.

In some instances, the multispecific antibody further comprises improved cancer cell killing by immune cells compared to the killing by immune cells of the reference antibody CD47 BMK-1 (such as described herein). In some cases, the cancer cell killing is improved by about 10%, 20%, 30%, 40%, 50%, 60%, 70%, 80%, 90%, or higher compared to the cancer cell killed in the presence of reference antibody CD47 BMK-1. In some cases, the immune cell is a Natural Killer cell. In some cases, the immune cell is a phagocytic cell. In some cases, the immune cell is a macrophage.

In some instances, the multispecific antibody further comprises improved cancer cell killing by immune cells compared to the killing by immune cells of the reference antibody CD47 BMK-2 (such as described herein). In some cases, the cancer cell killing is improved by about 10%, 20%, 30%, 40%, 50%, 60%, 70%, 80%, 90%, or higher compared to the cancer cell killed in the presence of reference antibody CD47 BMK-2. In some cases, the immune cell is a Natural Killer cell. In some cases, the immune cell is a phagocytic cell. In some cases, the immune cell is a macrophage.

Immunological activity can also be measured in a cell-line derived xenograft assay, wherein transformed cells are injected into mice and form a tumor. In some instances, a multispecific protein with a CD47-binding domain and an EGFR-binding domain inhibits the growth of a tumor comprising CD47-expressing cells to a greater extent than a bivalent protein with the same CD47-binding domain and/or a bivalent protein with the same EGFR-binding domain. In some instances, the multispecific protein exhibits 1.5, 2, 3, 4, 5, or 10-fold higher inhibition of xenograft tumor growth compared to a bivalent protein with a CD47-binding domain and/or a bivalent protein with an EGFR-binding domain.

As used herein, “Antibody-dependent cell-mediated cytotoxicity” and “ADCC” refers to a cell-mediated reaction in which non-specific cytotoxic cells (e.g., Natural Killer (NK) cells, neutrophils, and macrophages) recognize bound antibody on a target cell and subsequently cause lysis of the target cell. In one embodiment, the target cell is a human cell, such as a tumor (or cancer) cell (e.g., a myeloma cell). In some embodiments the tumor cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, an esophageal cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell. In some embodiments, the tumor cell is a derived from an adenocarcinoma, a lymphoma, a carcinoma. In some cases, the adenocarcinoma is a ductal pancreatic adenocarcinoma, an ovarian clear cell adenocarcinoma, a colon adenocarcinoma, a lung adenocarcinoma, a gastric adenocarcinoma cell, or an ovarian adenocarcinoma. In other cases, the carcinoma is a lung squamous cell carcinoma cell, a lung small cell carcinoma cell, a hepatocellular carcinoma cell, a prostate carcinoma cell, an endometrial carcinoma cell, a cholangiocarcinoma cell, or a vulvar squamous cell carcinoma. While not wishing to be bound by any particular mechanism of action, the cytotoxic cells that mediate ADCC generally express Fc receptors (FcRs). Cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991).

To assess ADCC activity of a multispecific protein as described herein, an in vitro ADCC assay, such as a cytotoxic assay using cancer cell lines, is carried out in some embodiments. Useful effector cells for such assays include, but are not limited to, peripheral blood mononuclear cells (PBMC) and Natural Killer (NK) cells. Alternatively, or additionally, ADCC activity of the multispecific proteins of interest is assessed, in some embodiments, in vivo, e.g., in an animal.

“Antibody dependent cellular phagocytosis” or “ADCP” refers to the ability of a phagocytic cell (e.g. macrophage) to eliminate an antibody-coated target cell. In some embodiments, a phagocytosis assay is used to measure the ADCP effect.

In some embodiments, a multispecific protein as described herein, that binds CD47 and EpCAM mediates antibody-dependent cellular phagocytosis of at least 50% of the cells in an exponentially growing population of CD47+/EpCAM+ cancer cell. In some embodiments, the tumor (or cancer) cell is a derived from an adenocarcinoma, a lymphoma, a carcinoma. In some cases, the adenocarcinoma is a ductal pancreatic adenocarcinoma, an ovarian clear cell adenocarcinoma, a colon adenocarcinoma, a lung adenocarcinoma, a gastric adenocarcinoma cell, or an ovarian adenocarcinoma.

In some embodiments, a multispecific protein as described herein binds CD47 and EpCAM mediates inhibition of a CD47-SIRPα interaction. In some embodiments the multispecific antibody inhibits binding of SIRPα to CD47 by at least 50%. In some embodiments, binding inhibition is measured by ELISA. In some embodiments binding inhibition is measured with CD47+ cells. In some embodiments, the CD47+ cells are CD47+ EpCAM+ tumor (or cancer) cells.

In some embodiments, the multispecific antibody has reduced binding to CD47+ non-tumor cells such as red blood cells and platelets. In some cases, binding to CD47+ non-tumor cells is reduced by 10%, 20%, 30%, 40%, 50%, 60%, 70%, 80%, 90%, 100% compared to the reference antibody, CD47 BMK-1 (such as described herein). In some cases, the multispecific antibody binding to CD47+ non-tumor cells is measured by a red blood cells (RBCs) binding assay or a flow cytometry assay.

In some embodiments, the multispecific antibody has reduced binding to CD47+ non-tumor cells such as red blood cells and platelets. In some cases, binding to CD47+ non-tumor cells is reduced by 10%, 20%, 30%, 40%, 50%, 60%, 70%, 80%, 90%, 100% compared to the reference antibody, CD47 BMK-2 (such as described herein). In some cases, the multispecific antibody binding to CD47+ non-tumor cells is measured by a red blood cells (RBCs) binding assay or a flow cytometry assay.

In some embodiments, the concentration of multispecific antibody required to induce RBC lysis is at least 2-fold, 3-fold, 4-fold, or higher than the hemolysis effect of reference antibody CD47 BMK-1 (such as described herein).

In some embodiments, the concentration of multispecific antibody required to induce RBC lysis is at least 2-fold, 3-fold, 4-fold, or higher than the hemolysis effect of reference antibody CD47 BMK-2 (such as described herein).

### Production of Antibodies or Binding Fragments Thereof

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11 (5): 155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLDI Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stb12™, Stbl3™, or Stbl4™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomyces (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293 FT cell line, 293F cells, 293 H cells, CHO DG44 cells, CHO-S cells, CHO-KI cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per. C6 cells, T-REX™-293 cell line, T-REX™-CHO cell line, and T-REX™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Complexes

In certain embodiments, disclosed herein is a complex comprising a multispecific antibody that comprises a CD47-binding domain and an EpCAM binding domain as described herein as described herein, and a CD47+ EpCAM+ target cell. In some embodiments, the target cell expresses at least as many EpCAM proteins on its surface as an HCC-44 cell. In some embodiments, the target cell expresses at least 50,000, at least 100,000, at least 300,000, at least 600,000 or at least 1,500,000 EpCAM proteins on its surface. In some embodiments, the CD47+ EpCAM+ target cell is a cancer cell. In some embodiments, the cancer cell is a ductal pancreatic adenocarcinoma cell, an ovarian clear cell adenocarcinoma cell, a colon adenocarcinoma cell, a lung adenocarcinoma cell, an ovarian adenocarcinoma cell, or a vulvar squamous cell carcinoma cell. In some embodiments the cancer cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

In certain embodiments, disclosed herein is a complex comprising a multispecific antibody that comprises a CD47-binding domain and an EGFR binding domain as described herein, and a CD47+ EGFR+ target cell. In some embodiments, the target cell expresses at least as many EGFR proteins on its surface as an HCC-44 cell. In some embodiments, the target cell expresses at least 50,000, at least 100,000, at least 300,000, at least 600,000 or at least 1,500,000 EGFR proteins on its surface. In some embodiments, the CD47+ EGFR+ target cell is a cancer cell. In some embodiments, the cancer cell is selected from an epidermoid carcinoma cell, a colorectal adenocarcinoma cell, a head and neck squamous cell carcinoma cell, a bladder carcinoma cell, a pancreas adenocarcinoma cell, a lung squamous cell carcinoma cell, a lung adenocarcinoma cell, a gastric carcinoma cell, a ductal pancreatic adenocarcinoma cell, an ovarian clear cell adenocarcinoma cell, an ovarian adenocarcinoma cell, a colon adenocarcinoma cell, a rectum adenocarcinoma cell, a esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, and a vulvar squamous cell carcinoma cell.

### Pharmaceutical Compositions

In certain embodiments, disclosed herein is a pharmaceutical composition comprising a multispecific (e.g., bispecific) antibody that comprises a CD47-binding domain and an EpCAM binding domain as described herein.

In certain embodiments, disclosed herein is a pharmaceutical composition comprising a multispecific (e.g., bispecific) antibody that comprises a CD47-binding domain and an EGFR binding domain as described herein.

### Methods or Uses

In certain embodiments, disclosed herein is a method of inducing phagocytosis of a CD47+ EpCAM+ target cell, the method comprising administering to a subject in need thereof a multispecific antibody that comprises a CD47-binding domain and an EpCAM binding domain as described herein, or a pharmaceutical composition thereof as described herein. In some embodiments, the multispecific antibody is administered at a concentration of less than about 5 nanomolar (nM), less than about 2 nM, less than about 1 nM, less than about 0.5 nM, less than about 0.2 nM, less than about 0.1 nM, or less than about 0.05 nM.

In certain embodiments, disclosed herein is a method of inducing phagocytosis of a CD47+ EGFR+ target cell, the method comprising administering to a subject in need thereof a multispecific antibody that comprises a CD47-binding domain and an EGFR binding domain as described herein, or a pharmaceutical composition thereof as described herein. In some embodiments, the multispecific antibody is administered at a concentration of less than about 5 nanomolar (nM), less than about 2 nM, less than about 1 nM, less than about 0.5 nM, less than about 0.2 nM, less than about 0.1 nM, or less than about 0.05 nM.

In certain embodiments, disclosed herein is a method of killing a CD47+ EpCAM+ target cell, the method comprising administering a multispecific antibody that comprises a CD47-binding domain and an EpCAM binding domain as described herein, or a pharmaceutical composition thereof as described herein, wherein the multispecific antibody inhibits the binding of SIRPα to CD47 on the surface of the CD47+ EpCAM+ target cell. In some embodiments, the multispecific antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 to about 0.05 nM, or less than about 0.01 nM. In some embodiments, the CD47+ EpCAM+ target cell is selected from an HCT-15 cell, an A431 cell, an HCC-44 cell, a SKOV-3 cell, an OVISE cell, or a CFPAC-1 cell. In some embodiments, the CD47+ EpCAM+ target cell is selected from a ductal pancreatic adenocarcinoma cell, an ovarian clear cell adenocarcinoma cell, a colon adenocarcinoma cell, a lung adenocarcinoma cell, or a vulvar squamous cell carcinoma cell. In some embodiments the target cancer cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

In certain embodiments, disclosed herein is a method of killing a CD47+ EGFR+ target cell, the method comprising administering a multispecific antibody that comprises a CD47-binding domain and an EGFR binding domain as described herein, or a pharmaceutical composition thereof as described herein, wherein the multispecific antibody inhibits the binding of SIRPα to CD47 on the surface of the CD47+ EGFR+ target cell. In some embodiments, the multispecific antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 nM to about 0.05 nM, or less than about 0.01 nM. In some embodiments, the CD47+ EGFR+ target cell is selected from an A431 cell, a NCI-H747 cell, an ASPC-1 cell, an EBC-1 cell, or an SNU-5 cell. In some embodiments, the CD47+ EGFR+ target cell is selected from an epidermoid carcinoma cell, a colorectal adenocarcinoma cell, a head and neck squamous cell carcinoma cell, a bladder carcinoma cell, a pancreas adenocarcinoma cell, a lung squamous cell carcinoma cell, a lung adenocarcinoma cell, a gastric carcinoma cell, a ductal pancreatic adenocarcinoma cell, an ovarian clear cell adenocarcinoma cell, an ovarian adenocarcinoma cell, a colon adenocarcinoma cell, a rectum adenocarcinoma cell, a esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, and a vulvar squamous cell carcinoma cell.

In certain embodiments, disclosed herein is a method of killing a CD47+ EpCAM+ target cell comprising administering a multispecific antibody that comprises a CD47-binding domain and an EpCAM binding domain as described herein, or a pharmaceutical composition thereof as described herein, wherein the multispecific antibody induces antibody-dependent cellular cytotoxicity that kills the CD47+ EpCAM+ target cell. In some embodiments, the multispecific antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 to about 0.05 nM, or less than about 0.01 nM. In some embodiments, the CD47+ EpCAM+ target cell is selected from an HCT-15 cell, an A431 cell, an HCC-44 cell, a SKOV-3 cell, an OVISE cell, or a CFPAC-1 cell. In some embodiments, the CD47+ EpCAM+ target cell is selected from a ductal pancreatic adenocarcinoma cell, an ovarian clear cell adenocarcinoma cell, a colon adenocarcinoma cell, a lung adenocarcinoma cell, or a vulvar squamous cell carcinoma cell. In some embodiments the target cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

In certain embodiments, disclosed herein is a method of killing a CD47+ EGFR+ target cell, the method comprising administering to a subject in need thereof a multispecific antibody that comprises a CD47-binding domain and an EGFR binding domain as described herein, or a pharmaceutical composition thereof as described herein, wherein the multispecific antibody induces antibody-dependent cellular cytotoxicity (ADCC) that kills the CD47+ EGFR+ target cell. In some embodiments, the multispecific antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 nM to about 0.05 nM, or less than about 0.01 nM. In some embodiments, the CD47+ EGFR+ target cell is selected from an A431 cell, a NCI-H747 cell, an ASPC-1 cell, an EBC-1 cell, or an SNU-5 cell. In some embodiments, the CD47+ EGFR+ target cell is selected from an epidermoid carcinoma cell, a colorectal adenocarcinoma cell, a head and neck squamous cell carcinoma cell, a bladder carcinoma cell, a pancreas adenocarcinoma cell, a lung squamous cell carcinoma cell, a lung adenocarcinoma cell, a gastric carcinoma cell, a ductal pancreatic adenocarcinoma cell, an ovarian clear cell adenocarcinoma cell, an ovarian adenocarcinoma cell, a colon adenocarcinoma cell, a rectum adenocarcinoma cell, a esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, and a vulvar squamous cell carcinoma cell.

In certain embodiments, disclosed herein is a method of treating a subject with cancer, the method comprising administering to the subject (e.g., an effective amount of) a multispecific antibody that comprises a CD47-binding domain and an EpCAM binding domain as described herein, or (e.g., an effective amount of) a pharmaceutical composition thereof as described herein. In some embodiments, the cancer is ductal pancreatic adenocarcinoma cell, ovarian clear cell adenocarcinoma cell, ovarian adenocarcinoma cell, colon adenocarcinoma cell, lung adenocarcinoma, rectum adenocarcinoma, esophageal cancer, gastric cancer, non-small cell lung cancer, small cell lung cancer, endometrial cancer, a hepatocellular carcinoma cell, a cholangiocarcinoma cell, prostate adenocarcinoma, thyroid carcinoma, breast cancer, cervical carcinoma, renal cell carcinoma, testis cancer, or vulvar squamous cell carcinoma.

In certain embodiments, disclosed herein is a method of treating a subject with cancer, the method comprising administering to the subject (e.g., an effective amount of) a multispecific antibody that comprises a CD47-binding domain and an EGFR binding domain as described herein, or (e.g., an effective amount of) a pharmaceutical composition thereof as described herein. In some embodiments, the cancer is epidermoid carcinoma, colorectal adenocarcinoma, head and neck squamous cell carcinoma, bladder carcinoma, pancreas adenocarcinom, lung squamous cell carcinoma, lung adenocarcinoma, gastric carcinoma, ductal pancreatic adenocarcinoma, ovarian clear cell adenocarcinoma, ovarian adenocarcinoma, colon adenocarcinoma, rectum adenocarcinoma, esophageal cancer, gastric cancer, non-small cell lung cancer, small cell lung cancer, endometrial cancer, a hepatocellular carcinoma cell, a cholangiocarcinoma cell, prostate adenocarcinoma, thyroid carcinoma, breast cancer, cervical carcinoma, renal cell carcinoma, testis cancer, or vulvar squamous cell carcinoma.

### EMBODIMENTS

Embodiment 1. A multispecific antibody comprising a CD47-binding domain and an EpCAM binding domain.

Embodiment 2. The multispecific antibody of embodiment 1, wherein the multispecific antibody is bispecific, trispecific, or tetraspecific.

Embodiment 3. The multispecific antibody of embodiment 2, wherein the multispecific antibody is bispecific.

Embodiment 4. The multispecific antibody of embodiment 1, wherein the multispecific antibody is bivalent, trivalent, or tetravalent.

Embodiment 5. The multispecific antibody of embodiment 4, wherein the multispecific antibody is bivalent.

Embodiment 6. The multispecific antibody of any of embodiments 1-5, wherein the multispecific antibody has a higher affinity for CD47 expressed on the surface of a tumor cell than for CD47 expressed on the surface of a normal cell, such as red blood cell, platelet, T cell, B cell or NK cell.

Embodiment 7. The multispecific antibody of embodiment 6, wherein the tumor cell expresses EpCAM.

Embodiment 8. The multispecific antibody of any of embodiments 1-7, wherein a concentration of the multispecific antibody required for half-maximal binding to a (e.g., human) red blood cell is greater than about 200 nanomolar (nM).

Embodiment 9. The multispecific antibody of any of embodiments 1-8, wherein the multispecific antibody binds to (e.g., human) CD47 with a dissociation constant (KD) of less than about 100 nanomolar (nM).

Embodiment 10. The multispecific antibody of embodiment 9, wherein the multispecific antibody binds to (e.g., human) CD47 with a dissociation constant (KD) of from about 1 nanomolar (nM) to about 100 nM, from about 1 nM to about 50 nM, or from about 5 nM to about 50 nM.

Embodiment 11. The multispecific antibody of any of embodiments 1-10, wherein the multispecific antibody binds to EpCAM with a dissociation constant (KD) of less than about 500 nanomolar (nM).

Embodiment 12. The multispecific antibody of embodiment 11, wherein the multispecific antibody binds to EpCAM with a dissociation constant (KD) of from about 0.2 nanomolar (nM) to about 500 nM, from about 1 nM to about 300 nM, from about 5 nM to about 200 nM, or from about 10 nM to about 150 nM.

Embodiment 13. The multispecific antibody of any of embodiments 9-12, wherein the dissociation constant (KD) is determined by surface plasmon resonance.

Embodiment 14. The multispecific antibody of any of embodiments 1-13, wherein the EpCAM-binding domain is a human or engineered human EpCAM-binding domain.

Embodiment 15. The multispecific antibody of any of embodiments 1-14, wherein the EpCAM binding domain comprises an antibody, or functional fragment or functional variant thereof, that binds specifically to EpCAM.

Embodiment 16. The multispecific antibody of embodiment 15, wherein the antibody, or functional fragment or functional variant thereof that binds specifically to EpCAM comprises an anti-EpCAM heavy chain and an anti-EpCAM light chain.

Embodiment 17. The multispecific antibody of embodiment 16, wherein the anti-EpCAM heavy chain comprises an anti-EpCAM heavy chain variable domain.

Embodiment 18. The multispecific antibody of embodiment 16, wherein the anti-EpCAM heavy chain variable domain comprises a variable domain of an IgG1, IgG2, IgG3, or IgG4 heavy chain.

Embodiment 19. The multispecific antibody of any one of embodiments 16-18, wherein the anti-EpCAM light chain comprises an anti-EpCAM light chain variable domain.

Embodiment 20. The multispecific antibody of embodiment 19, wherein the anti-EpCAM light chain variable domain comprises a Kappa or Lambda light chain.

Embodiment 21. The multispecific antibody of any one of embodiments 16-20, wherein the EpCAM binding domain comprises a single-chain variable fragment (scFv).

Embodiment 22. The multispecific antibody of any one of embodiments 16-20, wherein the EpCAM binding domain comprises an antigen-binding fragment (Fab).

Embodiment 23. The multispecific antibody of embodiment 16, wherein the EpCAM binding domain comprises a T cell receptor (TCR) constant region.

Embodiment 24. The multispecific antibody of embodiment 23, wherein the TCR constant region of the EpCAM binding domain comprises a TCR alpha constant domain and a TCR beta constant domain.

Embodiment 25. The multispecific antibody of embodiment 23, wherein the EpCAM binding domain comprises a VH-EpCAM domain and a VL-EpCAM domain; wherein the VH-EpCAM domain is fused to the TCR alpha constant domain; and the VL-EpCAM domain is fused to the TCR beta constant domain.

Embodiment 26. The multispecific antibody of embodiment 23, wherein the EpCAM binding domain comprises a VH-EpCAM domain and a VL-EpCAM domain; wherein the VH-EpCAM domain is fused to the TCR beta constant domain; and the VL-EpCAM domain is fused to the TCR alpha constant domain.

Embodiment 27. The multispecific antibody of any one of embodiments 23-26, wherein the EpCAM binding domain comprises a VH-EpCAM domain and a VL-EpCAM domain; wherein the VH-EpCAM domain is fused to the CH1 IgG domain; and the VL-EpCAM domain is fused to the CL IgG domain.

Embodiment 28. The multispecific antibody of any of embodiments 1-27, wherein the CD47-binding domain is a human or engineered human CD47-binding domain.

Embodiment 29. The multispecific antibody of any of embodiments 1-28, wherein the CD47 binding domain comprises an antibody, or functional fragment or functional variant thereof, that binds specifically to CD47.

Embodiment 30. The multispecific antibody of embodiment 29, wherein the CD47 binding domain comprises an anti-CD47 heavy chain and an anti-CD47 light chain.

Embodiment 31. The multispecific antibody of embodiment 30, wherein the anti-CD47 heavy chain comprises an anti-CD47 heavy chain variable domain.

Embodiment 32. The multispecific antibody of embodiment 31, wherein the anti-CD47 heavy chain variable domain comprises a variable domain of an IgG1, IgG2, IgG3, or IgG4 heavy chain.

Embodiment 33. The multispecific antibody of any one of embodiments 30-32, wherein the anti-CD47 light chain comprises an anti-CD47 light chain variable domain.

Embodiment 34. The multispecific antibody of embodiment 33, wherein the anti-CD47 light chain variable domain comprises a variable domain of a Kappa or Lambda light chain.

Embodiment 35. The multispecific antibody of embodiment 30, wherein the anti-CD47 heavy chain variable domain comprises the variable domain of an IgG1 heavy chain and the anti-CD47 light chain variable domain comprises a Kappa light chain.

Embodiment 36. The multispecific antibody of embodiment 30, wherein the anti-CD47 heavy chain variable domain comprises the variable domain of an IgG1 heavy chain and the anti-CD47 light chain variable domain comprises a Lambda light chain.

Embodiment 37. The multispecific antibody of embodiment 30, wherein the CD47 binding domain comprises a single-chain variable fragment (scFv).

Embodiment 38. The multispecific antibody of embodiment 30, wherein the CD47 binding domain comprises an antigen-binding fragment (Fab).

Embodiment 39. The multispecific antibody of embodiment 30, wherein the CD47 binding domain comprises a T cell receptor (TCR) constant region.

Embodiment 40. The multispecific antibody of embodiment 39, wherein the TCR constant region of the CD47 binding domain comprises a TCR alpha constant domain and a TCR beta constant domain.

Embodiment 41. The multispecific antibody of embodiment 39, wherein the CD47 binding domain comprises a VH-CD47 domain and a VL-CD47 domain; wherein the VH-CD47 domain is fused to the TCR alpha constant domain; and the VL-CD47 domain is fused to the TCR beta constant domain.

Embodiment 42. The multispecific antibody of embodiment 39, wherein the CD47 binding domain comprises a VH-CD47 domain and a VL-CD47 domain; wherein the VH-CD47 domain is fused to the TCR beta constant domain; and the VL-CD47 domain is fused to the TCR alpha constant domain.

Embodiment 43. The multispecific antibody of any one of embodiments 39-42, wherein the CD47 binding domain comprises a VH-CD47 domain and a VL-CD47 domain; wherein the VH-CD47 domain is fused to the CH1 IgG domain; and the VL-CD47 domain is fused to the CL IgG domain.

Embodiment 44. The multispecific antibody of any of embodiments 1-43, further comprising an Fc domain.

Embodiment 45. The multispecific antibody of embodiment 44, wherein the Fc domain is a human Fc domain.

Embodiment 46. The multispecific antibody of embodiment 44 or 45, wherein the isotype of the Fc domain is IgG1, IgG2, IgG3, or IgG4.

Embodiment 47. The multispecific antibody of any of embodiments 1-46, wherein the Fc domain is a heterodimeric Fc domain, wherein the heterodimeric Fc region comprises a knob chain and a hole chain, forming a knob-into-hole (KiH) structure.

Embodiment 48. The multispecific antibody of embodiment 47, wherein the knob chain comprises an IgG1, IgG2, IgG3, or IgG4 domain.

Embodiment 49. The multispecific antibody of embodiment 47 or 48, wherein the knob chain comprises the mutation T366W and the hole chain comprises the mutations T366S, L368A, and Y407V, wherein amino acid position numbering is according to the EU index of Kabat et al.

Embodiment 50. The multispecific antibody of any of embodiments 47-49, wherein the hole chain comprises an IgG1, IgG2, IgG3, or IgG4 domain.

Embodiment 51. The multispecific antibody of any of embodiments 1-50, wherein the multispecific antibody has an asymmetric three-chain knob-into-hole structure.

Embodiment 52. The multispecific antibody of any of embodiments 1-51, wherein the EpCAM-binding domain comprises three heavy chain (HC) complementarity determining regions (CDRs): HC-CDR1, HC-CDR2, and HC-CDR3; wherein the HC-CDR1, the HC-CDR2, and the HC-CDR3 of the EpCAM binding domain comprises amino acid sequences selected from those set forth in Table 9.

Embodiment 53. The multispecific antibody of any of embodiments 1-52, wherein the HC-CDR1 of the EpCAM binding domain comprises an amino acid sequence selected from SEQ ID NOs: 222, 225, 231, 234, 243, and 246.

Embodiment 54. The multispecific antibody of any of embodiments 1-53, wherein the HC-CDR2 of the EpCAM binding domain comprises an amino acid sequence selected from SEQ ID NOs: 223, 226, 232, 235, 244, and 247.

Embodiment 55. The multispecific antibody of any of embodiments 1-54, wherein the HC-CDR3 of the EpCAM binding domain comprises an amino acid sequence selected from SEQ ID NOs: 224, 227, 233, 236, 245, and 248.

Embodiment 56. The multispecific antibody of any of embodiments 1-55, wherein the EpCAM-binding domain comprises three light chain (LC) complementarity determining regions (CDRs): the LC-CDR1, the LC-CDR2, and the LC-CDR3; wherein the LC-CDR1, the LC-CDR2, and the LC-CDR3 of the EpCAM binding domain comprises amino acid sequences selected from those set forth in Table 10.

Embodiment 57. The multispecific antibody of any of embodiments 1-56, wherein the LC-CDR1 of the EpCAM binding domain comprises an amino acid sequence selected from SEQ ID NOs: 276, 279, 285, 288, 297, and 300.

Embodiment 58. The multispecific antibody of any of embodiments 1-57, wherein the LC-CDR2 of the EpCAM binding domain comprises an amino acid sequence selected from SEQ ID NOs: 277, 280, 286, 289, 298, and 301.

Embodiment 59. The multispecific antibody of any of embodiments 1-58, wherein the LC-CDR3 of the EpCAM binding domain comprises an amino acid sequence selected from SEQ ID NOs: 278, 281, 287, 290, 299, and 302.

Embodiment 60. The multispecific antibody of any of embodiments 1-59, wherein the EpCAM-binding domain comprises a heavy chain framework region (HFR) comprising an amino acid sequence selected from those set forth in Table 11; and wherein the HFR comprises from 0-3 amino acid modification(s).

Embodiment 61. The multispecific antibody of any of embodiments 1-59, wherein the EpCAM-binding domain comprises a heavy chain framework region (HFR) comprising an amino acid sequence selected from SEQ ID NOs: 331-334, 335-338, 343-346, 347-350, 355-358, and 359-362; and wherein the HFR comprises from 0-3 amino acid modification(s).

Embodiment 62. The multispecific antibody of embodiment 60 or 61, wherein the HFR of the EpCAM-binding domain comprises from 0-2 amino acid modification(s).

Embodiment 63. The multispecific antibody of embodiment 60 or 61, wherein the HFR of the EpCAM-binding domain comprises from 0-1 amino acid modification(s).

Embodiment 64. The multispecific antibody of any of embodiments 1-63, wherein the EpCAM-binding domain comprises a light chain framework region (LFR) comprising an amino acid sequence selected from those set forth in Table 12; and wherein the LFR comprises from 0-3 amino acid modification(s).

Embodiment 65. The multispecific antibody of any of embodiments 1-63, wherein the EpCAM-binding domain comprises a light chain framework region (LFR) comprising an amino acid sequence selected from SEQ ID NOs: 367-370, 371-374, 379-382, 383-386, 391-394, and 395-398; and wherein the HFR comprises from 0-3 amino acid modification(s).

Embodiment 66. The multispecific antibody of embodiment 64 or 65, wherein the LFR of the EpCAM-binding domain comprises from 0-2 amino acid modification(s).

Embodiment 67. The multispecific antibody of embodiment 64 or 65, wherein the LFR of the EpCAM-binding domain comprises from 0-1 amino acid modification(s).

Embodiment 68. The multispecific antibody of any of embodiments 1-67, wherein the EpCAM-binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 13.

Embodiment 69. The multispecific antibody of any of embodiments 1-68, wherein the EpCAM-binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 14.

Embodiment 70. The multispecific antibody of any of embodiments 1-69, wherein the EpCAM-binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 15.

Embodiment 71. The multispecific antibody of any of embodiments 1-70, wherein the EpCAM-binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 16.

Embodiment 72. The multispecific antibody of any of embodiments 1-71, wherein the EpCAM binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 17

Embodiment 73. The multispecific antibody of any of embodiments 1-71, wherein the EpCAM binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18.

Embodiment 74. The multispecific antibody of any of embodiments 1-71, wherein the EpCAM binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18.

Embodiment 75. The multispecific antibody of any of embodiments 1-71, wherein the EpCAM binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 18.

Embodiment 76. The multispecific antibody of any of embodiments 1-75, wherein the CD47-binding domain comprises three heavy chain (HC) complementarity determining regions (CDRs), HC-CDR1, HC-CDR2, and HC-CDR3; wherein the HC-CDR1, the HC-CDR2, and the HC-CDR3 of the CD47 binding domain comprises amino acid sequences selected from those set forth in Table 1; and wherein at least one of the HC-CDR1, the HC-CDR2, and the HC-CDR3 comprise from 0-3 amino acid modification(s).

Embodiment 77. The multispecific antibody of embodiment 76, wherein at least one of the HC-CDR1, the HC-CDR2, and the HC-CDR3 of the CD47-binding domain comprise from 0-2 amino acid modification(s).

Embodiment 78. The multispecific antibody of embodiment 76, wherein at least one of the HC-CDR1, the HC-CDR2, and the HC-CDR3 of the CD47-binding domain comprise from 0-1 amino acid modification(s).

Embodiment 79. The multispecific antibody of any of embodiments 1-78, wherein the CD47 binding domain comprises three light chain (LC) complementarity determining regions (CDRs), the LC-CDR1, the LC-CDR2, and the LC-CDR3; wherein the LC-CDR1, the LC-CDR2, and the LC-CDR3 of the CD47 binding domain comprises amino acid sequences selected from those set forth in Table 2; and wherein at least one of the LC-CDR1, the LC-CDR2, and the LC-CDR3 comprise from 0-3 amino acid modification(s).

Embodiment 80. The multispecific antibody of embodiment 79, wherein at least one of the LC-CDR1, the LC-CDR2, and the LC-CDR3 of the CD47-binding domain comprise from 0-2 amino acid modification(s).

Embodiment 81. The multispecific antibody of embodiment 79, wherein at least one of the LC-CDR1, the LC-CDR2, and the LC-CDR3 of the CD47-binding domain comprise from 0-1 amino acid modification(s).

Embodiment 82. The multispecific antibody of any of embodiments 1-81, wherein the CD47 binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 3.

Embodiment 83. The multispecific antibody of any of embodiments 1-82, wherein the CD47 binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 4.

Embodiment 84. The multispecific antibody of any of embodiments 1-83, wherein the CD47 binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 5.

Embodiment 85. The multispecific antibody of any of embodiments 1-84, wherein the CD47 binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 6.

Embodiment 86. The multispecific antibody of any of embodiments 1-84, wherein the CD47 binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 7.

Embodiment 87. The multispecific antibody of any of embodiments 1-84, wherein the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8.

Embodiment 88. The multispecific antibody of any of embodiments 1-84, wherein the CD47 binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8.

Embodiment 89. The multispecific antibody of any of embodiments 1-84, wherein the CD47 binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8.

Embodiment 90. The multispecific antibody of any of embodiments 1-86, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 26.

Embodiment 91. The multispecific antibody of any of embodiments 1-86, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 27.

Embodiment 92. The multispecific antibody of any of embodiments 1-86, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 28.

Embodiment 93. The multispecific antibody of any of embodiments 1-86, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 29.

Embodiment 94. The multispecific antibody of any of embodiments 1-86, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 30.

Embodiment 95. The multispecific antibody of any of embodiments 1-86, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 31.

Embodiment 96. The multispecific antibody of any of embodiments 1-86, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 32

Embodiment 97. The multispecific antibody of any of embodiments 1-96, wherein the EpCAM-binding domain comprises three heavy chain (HC) complementarity determining regions (CDRs): HC-CDR1, HC-CDR2, and HC-CDR3 and three light chain (LC) complementarity determining regions (CDRs): LC-CDR1, LC-CDR2, and LC-CDR3 and wherein the CD47-binding domain comprises three heavy chain (HC) complementarity determining regions (CDRs), HC-CDR1, HC-CDR2, and HC-CDR3 and the CD47 binding domain comprises three light chain (LC) complementarity determining regions (CDRs), LC-CDR1, LC-CDR2, and the LC-CDR3.

Embodiment 98. The multispecific antibody of any of embodiments 1-97, wherein the HC-CDR2 of the EpCAM-binding domain comprises the amino acid sequence according to SEQ ID NO: 235.

Embodiment 99. The multispecific antibody of any of embodiments 1-98, wherein the HC-CDR1, HC-CDR2, and HC-CDR3 of the EpCAM-binding domain comprises the amino acid sequences according to HC-CDR1: SEQ ID NO: 234; HC-CDR2: SEQ ID NO: 235, and HC-CDR3: SEQ ID NO: 236.

Embodiment 100. The multispecific antibody of any of embodiments 1-99, wherein the HC-CDR1, HC-CDR2, and HC-CDR3 of the EpCAM-binding domain comprises the amino acid sequences according to HC-CDR1: SEQ ID NO: 234; HC-CDR2: SEQ ID NO: 235, and HC-CDR3: SEQ ID NO: 236 and the LC-CDR1, LC-CDR2, and LC-CDR3 of the EpCAM-binding domain comprises the amino acid sequences according to LC-CDR1: SEQ ID NO: 288; LC-CDR2: SEQ ID NO: 289, and LC-CDR3: SEQ ID NO: 290.

Embodiment 101. The multispecific antibody of any of embodiments 1-100, wherein the HC-CDR1, HC-CDR2, and HC-CDR3 of the EpCAM-binding domain comprises the amino acid sequences according to HC-CDR1: SEQ ID NO: 234; HC-CDR2: SEQ ID NO: 235, and HC-CDR3: SEQ ID NO: 236 and the LC-CDR1, LC-CDR2, and LC-CDR3 of the EpCAM-binding domain comprises the amino acid sequences according to LC-CDR1: SEQ ID NO: 288; LC-CDR2: SEQ ID NO: 289, and LC-CDR3: SEQ ID NO: 290, and the HC-CDR1, HC-CDR2, and HC-CDR3 of the CD47-binding domain comprises the amino acid sequences according to HC-CDR1: SEQ ID NO: 1; HC-CDR2: SEQ ID NO: 2, and HC-CDR3: SEQ ID NO: 3.

Embodiment 102. The multispecific antibody of any of embodiments 1-101, wherein the HC-CDR1, HC-CDR2, and HC-CDR3 of the EpCAM-binding domain comprises the amino acid sequences according to HC-CDR1: SEQ ID NO: 234; HC-CDR2: SEQ ID NO: 235, and HC-CDR3: SEQ ID NO: 236 and the LC-CDR1, LC-CDR2, and LC-CDR3 of the EpCAM-binding domain comprises the amino acid sequences according to LC-CDR1: SEQ ID NO: 288; LC-CDR2: SEQ ID NO: 289, and LC-CDR3: SEQ ID NO: 290, and the HC-CDR1, HC-CDR2, and HC-CDR3 of the CD47-binding domain comprises the amino acid sequences according to HC-CDR1: SEQ ID NO: 1; HC-CDR2: SEQ ID NO: 2, and HC-CDR3: SEQ ID NO: 3 and the LC-CDR1, LC-CDR2, and LC-CDR3 of the CD47-binding domain comprises the amino acid sequences according to LC-CDR1: SEQ ID NO: 61; LC-CDR2: SEQ ID NO: 62, and LC-CDR3: SEQ ID NO: 63.

Embodiment 103. The multispecific antibody of any of embodiments 1-102, wherein the multispecific antibody comprises an asymmetric three-chain knob-into-hole structure and the EpCAM-binding domain comprises a single chain variable fragment (scFv) and the CD47-binding domain comprises an antigen-binding fragment (Fab).

Embodiment 104. The multispecific antibody of any of embodiments 1-97, wherein the multispecific antibody comprises amino acid sequences with at least 95% sequence identity to SEQ ID NOs: 482, 483, and 484.

Embodiment 105. The multispecific antibody of any of embodiments 1-97 or 104, wherein the multispecific antibody comprises the amino acid sequences according to SEQ ID NOs: 482, 483, and 484.

Embodiment 106. The multispecific antibody of any of embodiments 1-97 or 104-105, wherein the multispecific antibody comprises amino acid sequences with at least 95% sequence identity to SEQ ID NOs: 482, 483, and 507.

Embodiment 107. The multispecific antibody of any of embodiments 1-97 or 104-106, wherein the multispecific antibody comprises the amino acid sequences according to SEQ ID NOs: 482, 483, and 507.

Embodiment 108. The multispecific antibody of any of embodiments 1-97 or 104-107, wherein the multispecific antibody comprises an asymmetric three-chain knob-into-hole structure and the CD47-binding domain comprises a single chain variable fragment (scFv) and the EpCAM-binding domain comprises an antigen-binding fragment (Fab).

Embodiment 109. The multispecific antibody of any of embodiments 1-97 or 104-108, wherein the multispecific antibody comprises amino acid sequences with at least 95% sequence identity to SEQ ID NOs: 503, 504, and 505.

Embodiment 110. The multispecific antibody of any of embodiments 1-97 or 104-109, wherein the multispecific antibody comprises the amino acid sequences according to SEQ ID NOs: 503, 504, and 505.

Embodiment 111. The multispecific antibody of any of embodiments 1-97, wherein the HC-CDR2 of the EpCAM-binding domain comprises the amino acid sequence according to SEQ ID NO: 244.

Embodiment 112. The multispecific antibody of any of embodiments 1-97 or 111, wherein the HC-CDR1, HC-CDR2, and HC-CDR3 of the EpCAM-binding domain comprises the amino acid sequences according to HC-CDR1: SEQ ID NO: 243; HC-CDR2: SEQ ID NO: 244, and HC-CDR3: SEQ ID NO: 245.

Embodiment 113. The multispecific antibody of any of embodiments 1-97 or 111-112, wherein the HC-CDR1, HC-CDR2, and HC-CDR3 of the EpCAM-binding domain comprises the amino acid sequences according to HC-CDR1: SEQ ID NO: 243; HC-CDR2: SEQ ID NO: 244, and HC-CDR3: SEQ ID NO: 245 and the LC-CDR1, LC-CDR2, and LC-CDR3 of the EpCAM-binding domain comprises the amino acid sequences according to LC-CDR1: SEQ ID NO: 297; LC-CDR2: SEQ ID NO: 298, and LC-CDR3: SEQ ID NO: 299.

Embodiment 114. The multispecific antibody of any of embodiments 1-97 or 111-113, wherein the HC-CDR1, HC-CDR2, and HC-CDR3 of the EpCAM-binding domain comprises the amino acid sequences according to HC-CDR1: SEQ ID NO: 243; HC-CDR2: SEQ ID NO: 244, and HC-CDR3: SEQ ID NO: 245 and the LC-CDR1, LC-CDR2, and LC-CDR3 of the EpCAM-binding domain comprises the amino acid sequences according to LC-CDR1: SEQ ID NO: 297; LC-CDR2: SEQ ID NO: 298, and LC-CDR3: SEQ ID NO: 299, and the HC-CDR1, HC-CDR2, and HC-CDR3 of the CD47-binding domain comprises the amino acid sequences according to HC-CDR1: SEQ ID NO: 1; HC-CDR2: SEQ ID NO: 2, and HC-CDR3: SEQ ID NO: 3.

Embodiment 115. The multispecific antibody of any of embodiments 1-97 or 111-114, wherein the HC-CDR1, HC-CDR2, and HC-CDR3 of the EpCAM-binding domain comprises the amino acid sequences according to HC-CDR1: SEQ ID NO: 243; HC-CDR2: SEQ ID NO: 244, and HC-CDR3: SEQ ID NO: 245 and the LC-CDR1, LC-CDR2, and LC-CDR3 of the EpCAM-binding domain comprises the amino acid sequences according to LC-CDR1: SEQ ID NO: 297; LC-CDR2: SEQ ID NO: 298, and LC-CDR3: SEQ ID NO: 299, and the HC-CDR1, HC-CDR2, and HC-CDR3 of the CD47-binding domain comprises the amino acid sequences according to HC-CDR1: SEQ ID NO: 1; HC-CDR2: SEQ ID NO: 2, and HC-CDR3: SEQ ID NO: 3 and the LC-CDR1, LC-CDR2, and LC-CDR3 of the CD47-binding domain comprises the amino acid sequences according to LC-CDR1: SEQ ID NO: 61; LC-CDR2: SEQ ID NO: 62, and LC-CDR3: SEQ ID NO: 63.

Embodiment 116. The multispecific antibody of any of embodiments 1-97 or 111-115, wherein the multispecific antibody comprises an asymmetric three-chain knob-into-hole structure and the EpCAM-binding domain comprises a single chain variable fragment (scFv) and the CD47-binding domain comprises an antigen-binding fragment (Fab).

Embodiment 117. The multispecific antibody of any of embodiments 1-97 or 111-116, wherein the multispecific antibody comprises an asymmetric three-chain knob-into-hole structure and the CD47-binding domain comprises a single chain variable fragment (scFv) and the EpCAM-binding domain comprises an antigen-binding fragment (Fab).

Embodiment 118. The multispecific antibody of any of embodiments 1-97 or 111-117, wherein the multispecific antibody comprises amino acid sequences with at least 95% sequence identity to SEQ ID NOs: 485, 486, and 487.

Embodiment 119. The multispecific antibody of any of embodiments 1-97 or 111-118, wherein the multispecific antibody comprises the amino acid sequences according to SEQ ID NOs: 485, 486, and 487.

Embodiment 120. The multispecific antibody of any of embodiments 1-97, wherein the HC-CDR1, HC-CDR2, and HC-CDR3 of the EpCAM-binding domain comprises the amino acid sequences according to HC-CDR1: SEQ ID NO: 222; HC-CDR2: SEQ ID NO: 223, and HC-CDR3: SEQ ID NO: 224.

Embodiment 121. The multispecific antibody of any of embodiments 1-97 or 120, wherein the HC-CDR1, HC-CDR2, and HC-CDR3 of the EpCAM-binding domain comprises the amino acid sequences according to HC-CDR1: SEQ ID NO: 222; HC-CDR2: SEQ ID NO: 223, and HC-CDR3: SEQ ID NO: 224 and the LC-CDR1, LC-CDR2, and LC-CDR3 of the EpCAM-binding domain comprises the amino acid sequences according to LC-CDR1: SEQ ID NO: 276; LC-CDR2: SEQ ID NO: 277, and LC-CDR3: SEQ ID NO: 275.

Embodiment 122. The multispecific antibody of any of embodiments 1-97 or 120-121, wherein the HC-CDR1, HC-CDR2, and HC-CDR3 of the EpCAM-binding domain comprises the amino acid sequences according to HC-CDR1: SEQ ID NO: 222; HC-CDR2: SEQ ID NO: 223, and HC-CDR3: SEQ ID NO: 224 and the LC-CDR1, LC-CDR2, and LC-CDR3 of the EpCAM-binding domain comprises the amino acid sequences according to LC-CDR1: SEQ ID NO: 276; LC-CDR2: SEQ ID NO: 277, and LC-CDR3: SEQ ID NO: 275, and the HC-CDR1, HC-CDR2, and HC-CDR3 of the CD47-binding domain comprises the amino acid sequences according to HC-CDR1: SEQ ID NO: 1; HC-CDR2: SEQ ID NO: 2, and HC-CDR3: SEQ ID NO: 3.

Embodiment 123. The multispecific antibody of any of embodiments 1-97 or 120-122, wherein the HC-CDR1, HC-CDR2, and HC-CDR3 of the EpCAM-binding domain comprises the amino acid sequences according to HC-CDR1: SEQ ID NO: 222; HC-CDR2: SEQ ID NO: 223, and HC-CDR3: SEQ ID NO: 224 and the LC-CDR1, LC-CDR2, and LC-CDR3 of the EpCAM-binding domain comprises the amino acid sequences according to LC-CDR1: SEQ ID NO: 276; LC-CDR2: SEQ ID NO: 277, and LC-CDR3: SEQ ID NO: 275, and the HC-CDR1, HC-CDR2, and HC-CDR3 of the CD47-binding domain comprises the amino acid sequences according to HC-CDR1: SEQ ID NO: 1; HC-CDR2: SEQ ID NO: 2, and HC-CDR3: SEQ ID NO: 3 and the LC-CDR1, LC-CDR2, and LC-CDR3 of the CD47-binding domain comprises the amino acid sequences according to LC-CDR1: SEQ ID NO: 61; LC-CDR2: SEQ ID NO: 62, and LC-CDR3: SEQ ID NO: 63.

Embodiment 124. The multispecific antibody of any of embodiments 1-97 or 120-123, wherein the multispecific antibody comprises an asymmetric three-chain knob-into-hole structure and the EpCAM-binding domain comprises a single chain variable fragment (scFv) and the CD47-binding domain comprises an antigen-binding fragment (Fab).

Embodiment 125. The multispecific antibody of any of embodiments 1-97 or 120-124, wherein the multispecific antibody comprises an asymmetric three-chain knob-into-hole structure and the CD47-binding domain comprises a single chain variable fragment (scFv) and the EpCAM-binding domain comprises an antigen-binding fragment (Fab).

Embodiment 126. The multispecific antibody of any of embodiments 1-97 or 120-125, wherein the multispecific antibody comprises amino acid sequences with at least 95% sequence identity to SEQ ID NOs: 482, 483, and 506.

Embodiment 127. The multispecific antibody of any of embodiments 1-97 or 120-127, wherein the multispecific antibody comprises the amino acid sequences according to SEQ ID NOs: 482, 483, and 506.

Embodiment 128. The multispecific antibody of any of embodiments 1-93, wherein less than about 1 nanomolar (nM) or less than about 0.01 nM of the multispecific antibody increases a percentage of HCT-15 cells engulfed by macrophage by at least 4-fold compared to a nonspecific IgG1 antibody control.

Embodiment 129. The multispecific antibody of embodiment 94, wherein a concentration of the antibody required to mediate antibody-dependent cellular phagocytosis of an EpCAM-positive, CD47-positive tumor cell by a macrophage is from about 0.001 nanomolar (nM) to about 3 nM.

Embodiment 130. The multispecific antibody of embodiment 129, wherein the EpCAM-positive, CD47-positive tumor cell is an HCT-15 cell.

Embodiment 131. The multispecific antibody of embodiment 129, wherein the EpCAM-positive, CD47-positive tumor cell is selected from a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

Embodiment 132. The multispecific antibody of any of embodiments 1-131, wherein about 100 nanomolar (nM) of the multispecific antibody inhibits the binding of SIRPα to CD47 on the surface of a cell by at least about 30%.

Embodiment 133. The multispecific antibody of embodiment 132, wherein the cell is a CD47+ EpCAM+ tumor cell.

Embodiment 134. The multispecific antibody of embodiment 133, wherein the cell expresses at least as many EpCAM proteins on its surface as an HCC-44 cell.

Embodiment 135. The multispecific antibody of embodiment 133, wherein the cell expresses at least 50,000, at least 100,000, at least 300,000, at least 600,000 or at least 1,500,000 EpCAM proteins on its surface.

Embodiment 136. The multispecific antibody of any of embodiments 1-133, wherein about 1000 nanomolar (nM) of the multispecific antibody does not induce hemolysis of red blood cells in a hemagglutination assay.

Embodiment 137. A complex comprising the multispecific antibody of any of embodiments 1-136 and a CD47+ EpCAM+ target cell.

Embodiment 138. The complex of embodiment 137, wherein the target cell expresses at least as many EpCAM proteins on its surface as an HCC-44 cell.

Embodiment 139. The complex of embodiment 137, wherein the target cell expresses at least 50,000, at least 100,000, at least 300,000, at least 600,000 or at least 1,500,000 EpCAM proteins on its surface.

Embodiment 140. The complex of any of embodiments 137-139, wherein the CD47+ EpCAM+ target cell is a cancer cell.

Embodiment 141. The complex of embodiment 140, wherein the cancer cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

Embodiment 142. A method of inducing phagocytosis of a CD47+ EpCAM+ target cell comprising administering the multispecific antibody of any of embodiments 1-136.

Embodiment 143. The method of embodiment 142, wherein the multispecific antibody is administered at a concentration of less than about 5 nanomolar (nM), less than about 2 nM, less than about 1 nM, less than about 0.5 nM, less than about 0.2 nM, less than about 0.1 nM, or less than about 0.05 nM.

Embodiment 144. The method of embodiment 142 or 143, wherein the CD47+ EpCAM+ target cell is selected from an HCT-15 cell, an A431 cell, an HCC-44 cell, a SKOV-3 cell, an OVISE cell, or a CFPAC-1 cell.

Embodiment 145. The method of embodiment 142 or 143, wherein the CD47+ EpCAM+ target cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

Embodiment 146. A method of killing a CD47+ EpCAM+ target cell comprising administering the multispecific antibody of any of embodiments 1-136, wherein the multispecific antibody inhibits the binding of SIRPα to CD47 on the surface of the CD47+ EpCAM+ target cell.

Embodiment 147. A method of killing a CD47+ EpCAM+ target cell comprising administering the multispecific antibody of any of embodiments 1-136, wherein the multispecific antibody induces antibody-dependent cellular cytotoxicity that kills the CD47+ EpCAM+ target cell.

Embodiment 148. The method of embodiment 147, wherein the antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 to about 0.05 nM, or less than about 0.01 nM.

Embodiment 149. The method of embodiment 147 or 148, wherein the CD47+ EpCAM+ target cell is selected from an HCT-15 cell, an A431 cell, an HCC-44 cell, a SKOV-3 cell, an OVISE cell, or a CFPAC-1 cell.

Embodiment 150. The method of embodiment 147 or 148, wherein the CD47+ EpCAM+ target cell is a ductal pancreatic adenocarcinoma cell, an ovarian clear cell adenocarcinoma cell, an ovarian adenocarcinoma cell, a colon adenocarcinoma cell, a rectum adenocarcinoma cell, an esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, or a vulvar squamous cell carcinoma cell.

Embodiment 151. A pharmaceutical composition comprising the multispecific antibody of any of embodiments 1-136.

Embodiment 152. The pharmaceutical composition of embodiment 151, further comprising an anti-cancer agent.

Embodiment 153. The pharmaceutical composition of embodiment 152, wherein the anti-cancer agent is a chemotherapeutic agent or a biologic agent.

Embodiment 154. The pharmaceutical composition of embodiment 153, wherein the chemotherapeutic agent comprises a topoisomerase inhibitor.

Embodiment 155. The pharmaceutical composition of embodiment 154, wherein the topoisomerase inhibitor comprises camptothecin or a camptothecin derivative.

Embodiment 156. The pharmaceutical composition of embodiment 155, wherein the camptothecin derivative comprises irinotecan.

Embodiment 157. The pharmaceutical composition of embodiment 156, wherein the chemotherapeutic agent comprises oxaliplatin.

Embodiment 158. The pharmaceutical composition of embodiment 157, wherein the chemotherapeutic agent comprises 5-Fluorouracil (5-FU).

Embodiment 159. The pharmaceutical composition of embodiment 158, wherein the chemotherapeutic agent comprises irinotecan, oxaliplatin, or 5-FU.

Embodiment 160. A method of treating a subject with cancer comprising administering to the subject the pharmaceutical composition of embodiment 151.

Embodiment 161. The method of embodiment 160, wherein the cancer is ductal pancreatic adenocarcinoma, ovarian clear cell adenocarcinoma, ovarian adenocarcinoma, colon adenocarcinoma, lung adenocarcinoma, rectum adenocarcinoma, esophageal cancer, gastric cancer, non-small cell lung cancer, small cell lung cancer, endometrial cancer, hepatocellular carcinoma, cholangiocarcinoma, prostate adenocarcinoma, thyroid carcinoma, breast cancer, cervical carcinoma, renal cell carcinoma, testis cancer, or vulvar squamous cell carcinoma.

Embodiment 162. The method of embodiment 161, further comprising administering to the subject an anti-cancer agent.

Embodiment 163. The method of embodiment 162, wherein the anti-cancer agent is a chemotherapeutic agent or a biologic agent.

Embodiment 164. The method of any one of embodiments 162-163, wherein the anti-cancer agent is administered to the subject before the multispecific antibody is administered to the subject.

Embodiment 165. The method of any one of embodiments 162-163, wherein the anti-cancer agent is administered to the subject after the multispecific antibody is administered to the subject.

Embodiment 166. The method of any one of embodiments 162-163, wherein the anti-cancer agent is administered to the subject at the same time as the multispecific antibody is administered to the subject.

Embodiment 167. The method of any one of embodiments 162-166, wherein the chemotherapeutic agent comprises a topoisomerase inhibitor.

Embodiment 168. The method of embodiment 167, wherein the topoisomerase inhibitor comprises camptothecin or a camptothecin derivative.

Embodiment 169. The method of embodiment 168, wherein the camptothecin derivative comprises irinotecan.

Embodiment 170. The method of any one of embodiments 162-166, wherein the chemotherapeutic agent comprises oxaliplatin.

Embodiment 171. The method of any one of embodiments 162-166, wherein the chemotherapeutic agent comprises 5-Fluorouracil (5-FU).

Embodiment 172. The method of any one of embodiments 162-166, wherein the chemotherapeutic agent comprises irinotecan, oxaliplatin, or 5-FU.

Embodiment 173. A multispecific antibody comprising a CD47-binding domain and an EGFR binding domain.

Embodiment 174. The multispecific antibody of embodiment 173, wherein the multispecific antibody is bispecific, trispecific, or tetraspecific.

Embodiment 175. The multispecific antibody of embodiment 174, wherein the multispecific antibody is bispecific.

Embodiment 176. The multispecific antibody of embodiment 173, wherein the multispecific antibody is bivalent, trivalent, or tetravalent.

Embodiment 177. The multispecific antibody of embodiment 176, wherein the multispecific antibody is bivalent.

Embodiment 178. The multispecific antibody of embodiment 176 or 177, wherein the multispecific antibody has a higher affinity for CD47 expressed on the surface of a tumor cell than for CD47 expressed on the surface of a normal cell, such as red blood cell, platelet, T cell or NK cell.

Embodiment 179. The multispecific antibody of embodiment 178, wherein the tumor cell expresses EGFR.

Embodiment 180. The multispecific antibody of any of embodiments 176-179, wherein a concentration of the multispecific antibody required for half-maximal binding to a (e.g., human) red blood cell is greater than about 200 nanomolar (nM).

Embodiment 181. The multispecific antibody of any of embodiments 176-180, wherein multispecific antibody binds to (e.g., human) CD47 with a dissociation constant (KD) of less than about 100 nanomolar (nM).

Embodiment 182. The multispecific antibody of embodiment 181, wherein the multispecific antibody binds to (e.g., human) CD47 with a dissociation constant (KD) of from about 1 nanomolar (nM) to about 100 nM, from about 1 nM to about 50 nM, or from about 5 nM to about 50 nM.

Embodiment 183. The multispecific antibody of any of embodiments 176-182, wherein the multispecific antibody binds to EGFR with a dissociation constant (KD) of less than about 25 nanomolar (nM).

Embodiment 184. The multispecific antibody of embodiment 183, wherein the multispecific antibody binds to EGFR with a dissociation constant (KD) of from about 0.2 nanomolar (nM) to about 25 nM, from about 0.2 nM to about 10 nM, from about 0.2 nM to about 2 nM, or from about 2 nM to about 10 nM.

Embodiment 185. The multispecific antibody of any of embodiments 181-184, wherein the dissociation constant (KD) is determined by surface plasmon resonance.

Embodiment 186. The multispecific antibody of any of embodiments 176-185, wherein the CD47-binding domain is a human or engineered human CD47-binding domain.

Embodiment 187. The multispecific antibody of any of embodiments 176-186, wherein the CD47 binding domain comprises an antibody, or functional fragment or functional variant thereof, that binds specifically to CD47.

Embodiment 188. The multispecific antibody of embodiment 187, wherein the CD47 binding domain comprises an anti-CD47 heavy chain and an anti-CD47 light chain.

Embodiment 189. The multispecific antibody of embodiment 188, wherein the anti-CD47 heavy chain comprises an anti-CD47 heavy chain variable domain.

Embodiment 190. The multispecific antibody of embodiment 189, wherein the anti-CD47 heavy chain variable domain comprises a variable domain of an IgG1, IgG2, IgG3, or IgG4 heavy chain.

Embodiment 191. The multispecific antibody of any one of embodiments 187-190, wherein the anti-CD47 light chain comprises an anti-CD47 light chain variable domain.

Embodiment 192. The multispecific antibody of embodiment 191, wherein the anti-CD47 light chain variable domain comprises a Kappa or Lambda light chain.

Embodiment 193. The multispecific antibody of embodiment 188, wherein the anti-CD47 heavy chain variable domain comprises the variable domain of an IgG1 heavy chain and the anti-CD47 light chain variable domain comprises a Kappa light chain.

Embodiment 194. The multispecific antibody of embodiment 188, wherein the anti-CD47 heavy chain variable domain comprises the variable domain of an IgG1 heavy chain and the anti-CD47 light chain variable domain comprises a Lambda light chain.

Embodiment 195. The multispecific antibody of embodiment 187, wherein the CD47 binding domain comprises a single-chain variable fragment (scFv).

Embodiment 196. The multispecific antibody of embodiment 187, wherein the CD47 binding domain comprises an antigen-binding fragment (Fab).

Embodiment 197. The multispecific antibody of embodiment 187, wherein the CD47 binding domain comprises a T cell receptor (TCR) constant region.

Embodiment 198. The multispecific antibody of embodiment 197, wherein the TCR constant region of the CD47 binding domain comprises a TCR alpha constant domain and a TCR beta constant domain.

Embodiment 199. The multispecific antibody of embodiment 197, wherein the CD47 binding domain comprises a VH-CD47 domain and a VL-CD47 domain; wherein the VH-CD47 domain is fused to the TCR alpha constant domain; and the VL-CD47 domain is fused to the TCR beta constant domain.

Embodiment 200. The multispecific antibody of embodiment 197, wherein the CD47 binding domain comprises a VH-CD47 domain and a VL-CD47 domain; wherein the VH-CD47 domain is fused to the TCR beta constant domain; and the VL-CD47 domain is fused to the TCR alpha constant domain.

Embodiment 201. The multispecific antibody of any one of embodiments 197-200, wherein the CD47 binding domain comprises a VH-CD47 domain and a VL-CD47 domain; wherein the VH-CD47 domain is fused to the CH1 IgG domain; and the VL-CD47 domain is fused to the CL IgG domain.

Embodiment 202. The multispecific antibody of any of embodiments 176-201, wherein the EGFR-binding domain comprises a heavy chain variable domain and a light chain variable domain.

Embodiment 203. The multispecific antibody of embodiment 202, wherein the EGFR binding domain comprises a single-chain variable fragment (scFv).

Embodiment 204. The multispecific antibody of embodiment 202, wherein the EGFR binding domain comprises an antigen-binding fragment (Fab).

Embodiment 205. The multispecific antibody of embodiment 202, wherein the EGFR binding domain comprises a T cell receptor (TCR) constant region.

Embodiment 206. The multispecific antibody of embodiment 205, wherein the TCR constant region of the EGFR binding domain comprises a TCR alpha constant domain and a TCR beta constant domain.

Embodiment 207. The multispecific antibody of embodiment 205, wherein the EGFR binding domain comprises a VH-EGFR domain and a VL-EGFR domain; wherein the VH-EGFR domain is fused to the TCR alpha constant domain; and the VL-EGFR domain is fused to the TCR beta constant domain.

Embodiment 208. The multispecific antibody of embodiment 205, wherein the EGFR binding domain comprises a VH-EGFR domain and a VL-EGFR domain; wherein the VH-EGFR domain is fused to the TCR beta constant domain; and the VL-EGFR domain is fused to the TCR alpha constant domain.

Embodiment 209. The multispecific antibody of any one of embodiments 205-208, wherein the EGFR binding domain comprises a VH-EGFR domain and a VL-EGFR domain; wherein the VH-EGFR domain is fused to the CH1 IgG domain; and the VL-EGFR domain is fused to the CL IgG domain.

Embodiment 210. The multispecific antibody of any of embodiments 176-209, further comprising an Fc domain.

Embodiment 211. The multispecific antibody of embodiment 203, wherein the Fc domain is a human Fc domain.

Embodiment 212. The multispecific antibody of embodiment 203 or 211, wherein the isotype of the Fc domain is IgG1, IgG2, IgG3, or IgG4.

Embodiment 213. The multispecific antibody of any of embodiments 176-212, wherein the Fc domain is a heterodimeric Fc domain, wherein the heterodimeric Fc region comprises a knob chain and a hole chain, forming a knob-into-hole (KiH) structure.

Embodiment 214. The multispecific antibody of embodiment 213, wherein the knob chain comprises an IgG1, IgG2, IgG3, or IgG4 domain.

Embodiment 215. The multispecific antibody of embodiment 213 or 214, wherein the knob chain comprises the mutation T366W and the hole chain comprises the mutations T366S, L368A, and Y407V, wherein amino acid position numbering is according to the EU index of Kabat et al.

Embodiment 216. The multispecific antibody of any of embodiments 213-215, wherein the hole chain comprises an IgG1, IgG2, IgG3, or IgG4 domain.

Embodiment 217. The multispecific antibody of any of embodiments 176-216, wherein the multispecific antibody has an asymmetric three-chain knob-into-hole structure.

Embodiment 218. The multispecific antibody of any of embodiments 176-217, wherein the CD47-binding domain comprises three heavy chain (HC) complementarity determining regions (CDRs), HC-CDR1, HC-CDR2, and HC-CDR3; wherein the HC-CDR1, the HC-CDR2, and the HC-CDR3 of the CD47 binding domain comprises amino acid sequences selected from those set forth in Table 1; and wherein at least one of the HC-CDR1, the HC-CDR2, and the HC-CDR3 comprise from 0-3 amino acid modification(s).

Embodiment 219. The multispecific antibody of embodiment 218, wherein at least one of the HC-CDR1, the HC-CDR2, and the HC-CDR3 of the CD47-binding domain comprise from 0-2 amino acid modification(s).

Embodiment 220. The multispecific antibody of embodiment 218, wherein at least one of the HC-CDR1, the HC-CDR2, and the HC-CDR3 of the CD47-binding domain comprise from 0-1 amino acid modification(s).

Embodiment 221. The multispecific antibody of any of embodiments 176-220, wherein the CD47 binding domain comprises three light chain (LC) complementarity determining regions (CDRs), the LC-CDR1, the LC-CDR2, and the LC-CDR3; wherein the LC-CDR1, the LC-CDR2, and the LC-CDR3 of the CD47 binding domain comprises amino acid sequences selected from those set forth in Table 2; and wherein at least one of the LC-CDR1, the LC-CDR2, and the LC-CDR3 comprise from 0-3 amino acid modification(s).

Embodiment 222. The multispecific antibody of embodiment 221, wherein at least one of the LC-CDR1, the LC-CDR2, and the LC-CDR3 of the CD47-binding domain comprise from 0-2 amino acid modification(s).

Embodiment 223. The multispecific antibody of embodiment 221, wherein at least one of the LC-CDR1, the LC-CDR2, and the LC-CDR3 of the CD47-binding domain comprise from 0-1 amino acid modification(s).

Embodiment 224. The multispecific antibody of any of embodiments 176-223, wherein the CD47 binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 3.

Embodiment 225. The multispecific antibody of any of embodiments 176-224, wherein the CD47 binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 4.

Embodiment 226. The multispecific antibody of any of embodiments 176-225, wherein the CD47 binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 5.

Embodiment 227. The multispecific antibody of any of embodiments 176-226, wherein the CD47 binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 6.

Embodiment 228. The multispecific antibody of any of embodiments 176-227, wherein the CD47 binding domain comprises a single-chain variable fragment (scFv) comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 7.

Embodiment 229. The multispecific antibody of any of embodiments 176-228, wherein the CD47 binding domain comprises a TCR constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8.

Embodiment 230. The multispecific antibody of any of embodiments 176-229, wherein the CD47 binding domain comprises a TCR alpha constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8.

Embodiment 231. The multispecific antibody of any of embodiments 176-229, wherein the CD47 binding domain comprises a TCR beta constant domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 8.

Embodiment 232. The multispecific antibody of any of embodiments 176-231, wherein the EGFR-binding domain comprises three heavy chain (HC) complementarity determining regions (CDRs), HC-CDR1, HC-CDR2, and HC-CDR3; wherein the HC-CDR1, the HC-CDR2, and the HC-CDR3 of the EGFR binding domain comprises amino acid sequences selected from those set forth in Table 19; and wherein at least one of the HC-CDR1, the HC-CDR2, and the HC-CDR3 comprise from 0-3 amino acid modification(s).

Embodiment 233. The multispecific antibody of embodiment 232, wherein at least one of the HC-CDR1, the HC-CDR2, and the HC-CDR3 of the EGFR-binding domain comprise from 0-2 amino acid modification(s).

Embodiment 234. The multispecific antibody of embodiment 232, wherein at least one of the HC-CDR1, the HC-CDR2, and the HC-CDR3 of the EGFR-binding domain comprise from 0-1 amino acid modification(s).

Embodiment 235. The multispecific antibody of any of embodiments 176-233, wherein the EGFR binding domain comprises three light chain (LC) complementarity determining regions (CDRs), the LC-CDR1, the LC-CDR2, and the LC-CDR3; wherein the LC-CDR1, the LC-CDR2, and the LC-CDR3 of the EGFR binding domain comprises amino acid sequences selected from those set forth in Table 20; and wherein at least one of the LC-CDR1, the LC-CDR2, and the LC-CDR3 comprise from 0-3 amino acid modification(s).

Embodiment 236. The multispecific antibody of embodiment 235, wherein at least one of the LC-CDR1, the LC-CDR2, and the LC-CDR3 of the EGFR-binding domain comprise from 0-2 amino acid modification(s).

Embodiment 237. The multispecific antibody of embodiment 235, wherein at least one of the LC-CDR1, the LC-CDR2, and the LC-CDR3 of the EGFR-binding domain comprise from 0-1 amino acid modification(s).

Embodiment 238. The multispecific antibody of any of embodiments 176-237, wherein the EGFR binding domain comprises a heavy chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 21.

Embodiment 239. The multispecific antibody of any of embodiments 176-238, wherein the EGFR binding domain comprises a light chain variable domain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 22.

Embodiment 240. The multispecific antibody of any of embodiments 176-239, wherein the EGFR binding domain comprises a full-length heavy chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 23.

Embodiment 241. The multispecific antibody of any of embodiments 176-240, wherein the EGFR binding domain comprises a full-length light chain comprising a sequence having at least about 80%, 81%, 82%, 83%, 84%, 85%, 86%, 87%, 88%, 89%, 90%, 91%, 92%, 93%, 94%, 95%, 96%, 97%, 98%, or 99% identity to a sequence set forth in Table 24.

Embodiment 242. The multispecific antibody of any of embodiment 176-241, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 33.

Embodiment 243. The multispecific antibody of any of embodiment 176-241, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 34.

Embodiment 244. The multispecific antibody of any of embodiment 176-241, wherein the multispecific antibody comprises one or more sequences each having at least about 80%, 81%, 82%, 83%, 84%, 85%, 86%, 87%, 88%, 89%, 90%, 91%, 92%, 93%, 94%, 95%, 96%, 97%, 98%, or 99% identity to a corresponding sequence set forth in Table 35.

Embodiment 245. The multispecific antibody of any of embodiments 173-244, wherein the EGFR-binding domain comprises three heavy chain (HC) complementarity determining regions (CDRs): HC-CDR1, HC-CDR2, and HC-CDR3 and three light chain (LC) complementarity determining regions (CDRs): LC-CDR1, LC-CDR2, and LC-CDR3 and wherein the CD47-binding domain comprises three heavy chain (HC) complementarity determining regions (CDRs), HC-CDR1, HC-CDR2, and HC-CDR3 and the CD47 binding domain comprises three light chain (LC) complementarity determining regions (CDRs), LC-CDR1, LC-CDR2, and the LC-CDR3.

Embodiment 246. The multispecific antibody of embodiment 245, wherein the HC-CDR1, HC-CDR2, and HC-CDR3 of the EGFR-binding domain comprises the amino acid sequences according to HC-CDR1: SEQ ID NO: 460; HC-CDR2: SEQ ID NO: 461, and HC-CDR3: SEQ ID NO: 462.

Embodiment 247. The multispecific antibody of any of embodiments 245-246, wherein the HC-CDR1, HC-CDR2, and HC-CDR3 of the EGFR-binding domain comprises the amino acid sequences according to HC-CDR1: SEQ ID NO: 460; HC-CDR2: SEQ ID NO: 461, and HC-CDR3: SEQ ID NO: 462 and the LC-CDR1, LC-CDR2, and LC-CDR3 of the EGFR-binding domain comprises the amino acid sequences according to LC-CDR1: SEQ ID NO: 466; LC-CDR2: SEQ ID NO: 467, and LC-CDR3: SEQ ID NO: 468.

Embodiment 248. The multispecific antibody of any of embodiments 245-247, wherein the HC-CDR1, HC-CDR2, and HC-CDR3 of the EGFR-binding domain comprises the amino acid sequences according to HC-CDR1: SEQ ID NO: 460; HC-CDR2: SEQ ID NO: 461, and HC-CDR3: SEQ ID NO: 462 and the LC-CDR1, LC-CDR2, and LC-CDR3 of the EGFR-binding domain comprises the amino acid sequences according to LC-CDR1: SEQ ID NO: 466; LC-CDR2: SEQ ID NO: 467, and LC-CDR3: SEQ ID NO: 468, and the HC-CDR1, HC-CDR2, and HC-CDR3 of the CD47-binding domain comprises the amino acid sequences according to HC-CDR1: SEQ ID NO: 1; HC-CDR2: SEQ ID NO: 2, and HC-CDR3: SEQ ID NO: 3.

Embodiment 249. The multispecific antibody of any of embodiments 245-248, wherein the HC-CDR1, HC-CDR2, and HC-CDR3 of the EGFR-binding domain comprises the amino acid sequences according to HC-CDR1: SEQ ID NO: 460; HC-CDR2: SEQ ID NO: 461, and HC-CDR3: SEQ ID NO: 462 and the LC-CDR1, LC-CDR2, and LC-CDR3 of the EGFR-binding domain comprises the amino acid sequences according to LC-CDR1: SEQ ID NO: 466; LC-CDR2: SEQ ID NO: 467, and LC-CDR3: SEQ ID NO: 468, and the HC-CDR1, HC-CDR2, and HC-CDR3 of the CD47-binding domain comprises the amino acid sequences according to HC-CDR1: SEQ ID NO: 1; HC-CDR2: SEQ ID NO: 2, and HC-CDR3: SEQ ID NO: 3 and the LC-CDR1, LC-CDR2, and LC-CDR3 of the CD47-binding domain comprises the amino acid sequences according to LC-CDR1: SEQ ID NO: 61; LC-CDR2: SEQ ID NO: 62, and LC-CDR3: SEQ ID NO: 63.

Embodiment 250. The multispecific antibody of any of embodiments 245-249, wherein the multispecific antibody comprises an asymmetric three-chain knob-into-hole structure and the EGFR-binding domain comprises a single chain variable fragment (scFv) and the CD47-binding domain comprises an antigen-binding fragment (Fab).

Embodiment 251. The multispecific antibody of any of embodiments 245-250, wherein the multispecific antibody comprises an asymmetric three-chain knob-into-hole structure and the CD47-binding domain comprises a single chain variable fragment (scFv) and the EGFR-binding domain comprises an antigen-binding fragment (Fab).

Embodiment 252. The multispecific antibody of any of embodiments 245-251, wherein the multispecific antibody comprises amino acid sequences with at least 95% sequence identity to SEQ ID NOs: 508, 509, and 510.

Embodiment 253. The multispecific antibody of any of embodiments 245-252, wherein the multispecific antibody comprises the amino acid sequences according to SEQ ID NOs: 508, 509, and 510.

Embodiment 254. The multispecific antibody of any of embodiment 176-243, wherein less than about 1 nanomolar (nM) or less than about 0.01 nM of the multispecific antibody increases a percentage of A431 cells engulfed by macrophage by at least 4-fold compared to a nonspecific IgG1 antibody control.

Embodiment 255. The multispecific antibody of embodiment 244, wherein a concentration of the antibody required to mediate antibody-dependent cellular phagocytosis of an EGFR-positive, CD47-positive tumor cell by a macrophage is from about 0.01 nanomolar (nM) to about 3 nM.

Embodiment 256. The multispecific antibody of embodiment 255, wherein the EGFR-positive, CD47-positive tumor cell is an OVISE cell or an A431 cell.

Embodiment 257. The multispecific antibody of embodiment 255, wherein the EGFR-positive, CD47-positive tumor cell is selected from an epidermoid carcinoma cell, a colorectal adenocarcinoma cell, a head and neck squamous cell carcinoma cell, a bladder carcinoma cell, a pancreas adenocarcinoma cell, a lung squamous cell carcinoma cell, a lung adenocarcinoma cell, a gastric carcinoma cell, a ductal pancreatic adenocarcinoma cell, an ovarian clear cell adenocarcinoma cell, an ovarian adenocarcinoma cell, a colon adenocarcinoma cell, a rectum adenocarcinoma cell, a esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, and a vulvar squamous cell carcinoma cell.

Embodiment 258. The multispecific antibody of any of embodiments 176-243, wherein about 100 nanomolar (nM) of the multispecific antibody inhibits the binding of SIRPα to CD47 on the surface of a cell by at least about 30%.

Embodiment 259. The multispecific antibody of embodiment 258, wherein the cell is a CD47+ EGFR+ tumor cell.

Embodiment 260. The multispecific antibody of embodiment 259, wherein the cell expresses at least as many EGFR proteins on its surface as an HCC-44 cell.

Embodiment 261. The multispecific antibody of embodiment 259, wherein the cell expresses at least 50,000, at least 100,000, at least 300,000, at least 600,000 or at least 1,500,000 EGFR proteins on its surface.

Embodiment 262. The multispecific antibody of any of embodiments 176-259, wherein about 400 nanomolar (nM) of the multispecific antibody does not induce hemolysis of red blood cells in a hemagglutination assay.

Embodiment 263. A complex comprising the multispecific antibody of any of embodiments 176-262 and a CD47+ EGFR+ target cell.

Embodiment 264. The complex of embodiment 263, wherein the target cell expresses at least as many EGFR proteins on its surface as an HCC-44 cell.

Embodiment 265. The complex of embodiment 263, wherein the target cell expresses at least 50,000, at least 100,000, at least 300,000, at least 600,000 or at least 1,500,000 EGFR proteins on its surface.

Embodiment 266. The complex of any of embodiments 263-265, wherein the CD47+ EGFR+ target cell is a cancer cell.

Embodiment 267. The complex of embodiment 266, wherein the cancer cell is epidermoid carcinoma cell, colorectal adenocarcinoma cell, head and neck squamous cell carcinoma cell, bladder adenocarcinoma cell, pancreas adenocarcinoma cell, lung squamous cell carcinoma, gastric carcinoma cell, ductal pancreatic adenocarcinoma cell, ovarian clear cell adenocarcinoma cell, ovarian adenocarcinoma cell, colon adenocarcinoma cell, lung adenocarcinoma, rectum adenocarcinoma, esophageal cancer, gastric cancer, non-small cell lung cancer, small cell lung cancer, endometrial cancer, hepatocellular carcinoma, cholangiocarcinoma, prostate adenocarcinoma, thyroid carcinoma, breast cancer, cervical carcinoma, renal cell carcinoma, testis cancer, or vulvar squamous cell carcinoma.

Embodiment 268. A method of inducing phagocytosis of a CD47+ EGFR+ target cell comprising administering the multispecific antibody of any of embodiments 176-262.

Embodiment 269. The method of embodiment 268, wherein the multispecific antibody is administered at a concentration of less than about 5 nanomolar (nM), less than about 2 nM, less than about 1 nM, less than about 0.5 nM, less than about 0.2 nM, less than about 0.1 nM, or less than about 0.05 nM.

Embodiment 270. A method of killing a CD47+ EGFR+ target cell comprising administering the multispecific antibody of any of embodiments 176-262, wherein the multispecific antibody inhibits the binding of SIRPα to CD47 on the surface of the CD47+ EGFR+ target cell.

Embodiment 271. The method of embodiment 270, wherein the antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 nM to about 0.05 nM, or less than about 0.01 nM.

Embodiment 272. The method of embodiment 270 or 271, wherein the CD47+ EGFR+ target cell is selected from an A431 cell, a NCI-H747 cell, an ASPC-1 cell, an EBC-1 cell, or a SNU-5 cell.

Embodiment 273. The method of embodiment 270 or 271, wherein the CD47+ EGFR+ target cell is selected from an epidermoid carcinoma cell, a colorectal adenocarcinoma cell, a head and neck squamous cell carcinoma cell, a bladder carcinoma cell, a pancreas adenocarcinoma cell, a lung squamous cell carcinoma cell, a lung adenocarcinoma cell, a gastric carcinoma cell, a ductal pancreatic adenocarcinoma cell, an ovarian clear cell adenocarcinoma cell, an ovarian adenocarcinoma cell, a colon adenocarcinoma cell, a rectum adenocarcinoma cell, a esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, and a vulvar squamous cell carcinoma cell.

Embodiment 274. A method of killing a CD47+ EGFR+ target cell comprising administering the multispecific antibody of any of embodiments 176-262, wherein the multispecific antibody induces antibody-dependent cellular cytotoxicity that kills the CD47+ EGFR+ target cell.

Embodiment 275. The method of embodiment 274, wherein the antibody is administered at a concentration of from about 0.01 nanomolar (nM) to about 1 nM, from about 0.01 nM to about 0.5 nM, from about 0.01 nM to about 0.25 nM, from about 0.01 nM to about 0.1 nM, from about 0.01 nM to about 0.05 nM, or less than about 0.01 nM.

Embodiment 276. The method of embodiment 274 or 275, wherein the CD47+ EGFR+ target cell is selected from an A431 cell, a NCI-H747 cell, an ASPC-1 cell, an EBC-1 cell, or a SNU-5 cell.

Embodiment 277. The method of embodiment 274 or 275, wherein the CD47+ EGFR+ target cell is selected from an epidermoid carcinoma cell, a colorectal adenocarcinoma cell, a head and neck squamous cell carcinoma cell, a bladder carcinoma cell, a pancreas adenocarcinoma cell, a lung squamous cell carcinoma cell, a lung adenocarcinoma cell, a gastric carcinoma cell, a ductal pancreatic adenocarcinoma cell, an ovarian clear cell adenocarcinoma cell, an ovarian adenocarcinoma cell, a colon adenocarcinoma cell, a rectum adenocarcinoma cell, a esophageal cancer cell, a gastric cancer cell, a non-small cell lung cancer cell, a small cell lung cancer cell, an endometrial cancer cell, a hepatocellular carcinoma cell, a cholangiocarcinoma cell, a prostate adenocarcinoma cell, a thyroid carcinoma cell, a breast cancer cell, a cervical carcinoma cell, a renal cell carcinoma cell, a testis cancer cell, and a vulvar squamous cell carcinoma cell.

Embodiment 278. A pharmaceutical composition comprising the multispecific antibody of any of embodiments 176-262.

Embodiment 279. A method of treating a subject with cancer comprising administering to the subject the pharmaceutical composition of embodiment 278.

Embodiment 280. The method of embodiment 279, wherein the cancer is epidermoid carcinoma, colorectal adenocarcinoma, head and neck squamous cell carcinoma, bladder carcinoma, pancreas adenocarcinom, lung squamous cell carcinoma, lung adenocarcinoma, gastric carcinoma, ductal pancreatic adenocarcinoma, ovarian clear cell adenocarcinoma, ovarian adenocarcinoma, colon adenocarcinoma, rectum adenocarcinoma, esophageal cancer, gastric cancer, non-small cell lung cancer, small cell lung cancer, endometrial cancer, hepatocellular carcinoma, cholangiocarcinoma, prostate adenocarcinoma, thyroid carcinoma, breast cancer, cervical carcinoma, renal cell carcinoma, testis cancer, or vulvar squamous cell carcinoma.

### EXAMPLES

### Example 1: Benchmark Antibodies and Reagents

Sequences of the benchmark antibodies and reagents used in this study were parsed from patent literature and cloned into a human IgG backbone as indicated in Table 36.

TABLE 36
Benchmark reagents.

| | | | Source of |
|---|---|---|---|
| Name | Description | Parent molecule | parent molecule |
| CD47 BMK-1 | αCD47 IgG4 | 5F9 | Gilead |
| CD47 BMK-2 | SIRPα- Fc fusion | TTI-621 | Pfizer/Trillium |
| | | | Therapeutics |

### Example 2. Humanization of Anti-EpCAM Binding Domains

The anti-EpCAM antibodies Mab-001, Mab-002 and Mab-008 from mouse hybridoma were humanized by grafting their CDR sequences into a human framework. The CDR graft variants had reduced affinity for their antigens compared to the chimeric antibodies, and they contained sequences that might reduce their stability. Thus, they were further engineered by replacing human framework residues with the corresponding mouse framework residues at positions that contribute to binding and replacing potential chemical hot spots with residues that do not have chemical liabilities.

The sequences of Mab-001 variable domains were aligned to sequences from the international ImMunoGeneTics Information System® (IMGT®) library to identify human germline VH and VL-kappa sequences with homology to the HV and VL chains of Mab-001. IGKV1-39*01 was chosen as the VL human germline acceptor sequence. IGHV1-46*01 was chosen as the VH human germline acceptor sequence.

The sequences of Mab-002 variable domains were aligned to sequences from the international ImMunoGeneTics Information System® (IMGT®) library to identify human germline VH and VL-kappa sequences with homology to the HV and VL chains of Mab-002. IGKV1-39*01 was chosen as the VL human germline acceptor sequence. IGHV3-23*04 was chosen as the VH human germline acceptor sequence.

The sequences of Mab-008 variable domains were aligned to sequences from the international ImMunoGeneTics Information System® (IMGT®) library to identify human germline VH and VL-kappa sequences with homology to the HV and VL chains of Mab-008. IGKV1-39*01 was chosen as the VL human germline acceptor sequence. IGHV1-69*04 was chosen as the VH human germline acceptor sequence.

### Example 3: Antibody Binding to Antigen Measured by ELISA

A 96-well plate was coated overnight at 4° C. with 1 μg/ml recombinant human CD47 or human EpCAM. After washing 3 times, the plate was blocked with 300 μl 1% BSA in PBST at 37° C. for 1 hour. Serially diluted antibodies were added and incubated at 37° C. for 1 hour. The plate was then washed 4 times with PBST and incubated with 1:5000 diluted peroxidase labeled goat anti-human IgG (Fab specific) secondary antibody (Sigma, Cat #A0293) for 1 hour at 37° C. The plate was washed again 4 times with PBST, incubated with 3,3′,5,5′-tetramethylbenzidine (TMB) substrate for 15 min at room temperature, terminated with 1N HCl, and then read at 450 nm. The binding on EpCAM of humanized version of Mab-001, Mab-002 and Mab-008 had been confirmed by ELISA, all the humanized anti-EpCAM variants showed robust binding on human EpCAM recombinant protein.

### Example 4. Generation of Anti-CD47/Anti-EpCAM Bispecific Antibodies

CD47×EpCAM bispecific antibodies with different binding stoichiometry and geometry were designed and generated using VH and VL sequences from the anti-CD47 antibody VIR47.V8 and anti-EpCAM antibodies sequences. The corresponding heavy chain (HC) and light chain (LC) DNAs were synthesized and cloned into the pRK5 mammalian expression vector (ATCC). Each HC and LC pair were then co-transfected in CHO cells. The conditioned medium was harvested by centrifugation (4° C., 4000 rpm for 40 min), then filtered to remove cell debris. The clarified medium was loaded onto MabSelect SuRe column (GE, 17-5438) which was pre-equilibrated with Buffer A (25 mM Tris, 150 mM NaCl, pH 8.0). The column was washed sequentially with 5 column volume of Buffer A, then 30 column volume of Buffer B (Buffer A+0.1% Triton X100+0.1% Triton X114), then 15 column volume of Buffer A. The antibodies were eluted with Buffer C (100 mM sodium citrate, 150 mM NaCl, pH 3.0) and neutralized immediately with Buffer D (200 mM Arginine, 137 mM Succinic acid, pH 5.0). The final product was dialyzed against the buffer PBS, pH 7.4, concentrated and filtrated through MILLEX-MP 0.22 um (MILLIPORE).

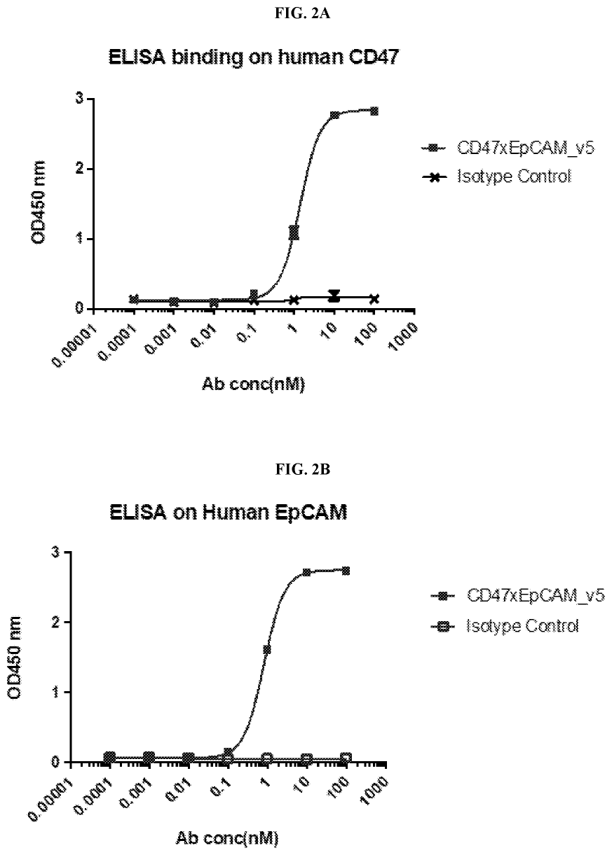

FIGS. 2A-2B showed the ELISA binding results for anti-CD47×EpCAM bispecific antibody CD47×EpCAM_v5 on human CD47 and human EpCAM, respectively.

### Example 5. Inhibition of SIRPα Binding to CD47+ Cells

Tumor cells were harvested, centrifuged, and then resuspended in FACS buffer (PBS plus 2% FBS) at a concentration of 2×10⁶ cells/mL. 100 μL of the cell suspension was dispensed into each well of a 96-well plate. The plate was centrifuged for 5 min and 300×g, and the supernatants were discarded. The cells were incubated with 50 μL per well of serially-diluted bispecific or bivalent anti-CD47 antibodies and a constant amount of SIRPα-mIgG2a fusion protein (0.2 μg/mL for Raji cells; 1 μg/mL for HCT-15 cells) in FACS buffer for 1 h at 4° C. Then, the plates were washed twice with FACS buffer and incubated for 1 hour at 4° C. in the dark with 100 μL of Alexa Fluor 488 donkey anti-Mouse IgG (H+L) secondary antibody (Invitrogen, Cat #A21202, 1:1000). After washing twice with FACS buffer, the plates were resuspended with 300 μL FACS buffer and analyzed by flow cytometry.

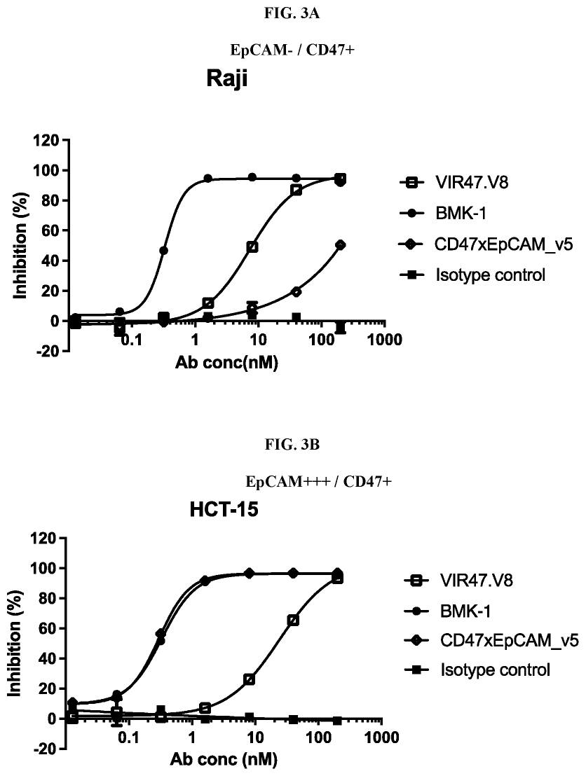

In FIG. 3A, CD47×EpCAM_v5 showed weak SIRPα blocking activity on EpCAM negative Raji cells with an IC₅₀ greater than 100 nM. In FIG. 3B, on EpCAM high HCT-15 cells CD47×EpCAM_v5 showed potent SIRPα blocking activity with subnanomolar IC₅₀, nearly 500-fold lower than that of Raji cells. On the other hand, the BMK-1 and parental VIR47.V8 showed similar SIRPα blocking activity on these two tumor cells lines without discrimination. Therefore, CD47×EpCAM_v5 shows strong selectivity on EpCAM+/CD47+ tumor cells over CD47+ only cells, whereas the anti-CD47 bivalent antibodies have no selectivity.

### Example 6. Bispecific CD47×EpCAM Antibody Induces Phagocytosis of CD47+ EpCAM+ Cells

The bispecific and bivalent antibodies were tested in an antibody-dependent cellular phagocytosis (ADCP) assay. Peripheral blood mononuclear cells (PBMCs) were isolated from human donors. Monocytes were enriched using a Human Monocyte Enrichment Kit without CD16 depletion (STEMCELL, Cat #19058). Isolated monocytes were differentiated into macrophages by culturing monocytes in complete culture media (RPMI 1640+10% FBS) with 20 ng/ml of human Macrophage Colony-Stimulating Factor (M-CSF, Peprotech, Cat #: 3-25-10). The media was changed every three days. After 7 days of culturing in M-CSF containing culture media, macrophages were collected and counted. Target tumor cells were collected and washed with D-PBS two times to remove remaining FBS. The washed tumor cells were resuspended in PBS at a cell concentration of 5-10×10⁶/mL. Cancer cells were stained with CFSE (ebiosciences, Cat #: 65-0580-84) at a final concentration of 3 μM and mixed immediately. The cells were stained in the dark at room temperature for 10 minutes. The staining was terminated by adding 4-5 volumes of cold complete media and incubating on ice for 5 minutes. Stained cells were washed three times with RPMI 1640+10% FBS. Cells were resuspended in 1 mL RPMI 1640+10% FBS and counted, then adjusted the cell numbers to 3×10⁵ cells/mL. 50 μL cells were seeded into a 96 well deep U-plate (Axygen, Cat #: P-DW-20-C) wherein each well contained 1.5×10⁴ cells. 50 μL of diluted antibodies were added to each well. 100 μL of macrophages (1.5×10⁴ cells) were added to each well and incubated at 37° C., 5% CO2 for 1.5 hours. After incubation, cells were washed with 2 mL of 2% FBS in D-PBS once. 100 μL of diluted Fc blocker (Human TruStain FcX (Fc Receptor Blocking Solution), Biolegend Cat #: 422302)) was added and the cells were incubated at room temperature for 10 minutes. 20 μL of diluted anti-human CD11b antibody was added to each well and incubated for 30 minutes at 4° C. in the dark. Cells were washed with 2% FBS-D-PBS once. Phagocytosis was detected in a flow cytometer by the appearance of CFSE/CD11b double positive cells indicative of macrophages that engulfed the tumor cells.

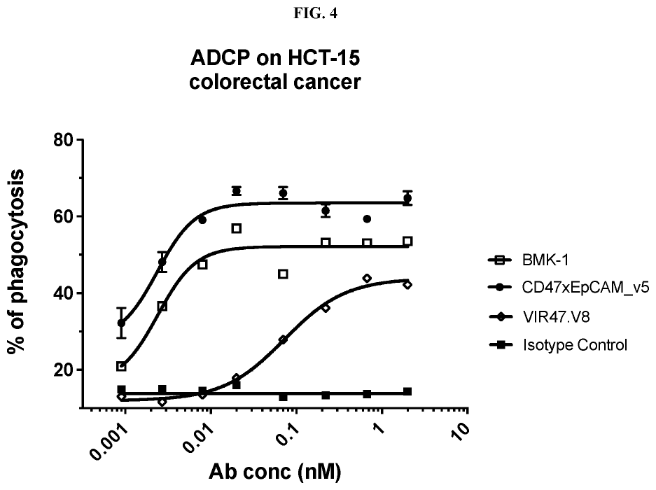

In FIG. 4. CD47×EpCAM_v5 showed robust ADCP on CD47+/EpCAM+ HCT-15 cells, more potent than its parental CD47 antibody VIR47.V8 and BMK-1.

### Example 7. Antibody-Dependent Cellular Cytotoxicity

The ability of anti-CD47 antibodies to mediate antibody-dependent cellular cytotoxicity (ADCC) was tested on KYSE-150, esophageal cancer cells, which express high levels of CD47 and EpCAM. The cells were washed once with balanced salt solution or culture medium and cell numbers were adjusted to 1×10⁶ cells/mL. 2 μL of BATDA fluorescence enhancing ligand (Perkin Elmer, Cat #C136-100) was then added to each mL of cells and incubated for 20 min at 37° C. in a cell incubator. After incubation, cells were centrifuged, culture medium was aspirated. The labeled cells were washed 4 times with PBS. After the final wash, cells were re-suspended in culture medium and adjusted to 5×10⁴ cell/mL. 200 μL cell suspension was then added to each well of the 96-well plate to make the cell number per well to 1×10⁴. Background release was determined by withdrawing an aliquot of the labeled target cells, centrifuge and supernatant was transferred into an empty well. The reading was background release. 1×10⁴ labeled target cells were transferred to sterile 96-well assay plate. Antibodies were serially diluted with RPMI-1640 containing 10% FBS. 50 μL of serially-diluted antibodies were added to assay plate containing target cell and incubated at 37° C., 5% CO2 for 5-10 min. Effector cells PBMCs were harvested and suspended in RPMI-1640 containing 10% FBS. 50 μL/well effector cells were added to each well of assay plate at different effector to target ratios. Set up controls: target spontaneous (target cell+100 μL medium); target maximum (target cell+100 μL medium+10 μL lysis buffer); background (100 μL the labeled target cell supernatant and 100 μL dilution medium). The plates were incubated in a humidified 5% CO2 atmosphere at 37° C. for 2 hours. At the end of incubation, 10 μL of Lysis Buffer (Perkin Elmer, Cat #4005-0010) was added to the maximum release well. The plates were centrifuged for 5 min at 500×g. 20 μL of the supernatant from each well was transferred to a flat-bottom detection plate. 200 μL of Europium Solution (Perkin Elmer, Cat #C135-100) was then added to each well of the detection plate. The plate was shaken at 250 rpm for 15 min at room temperature and the fluorescence was then measured in a time-resolved fluorometer within 5 h.

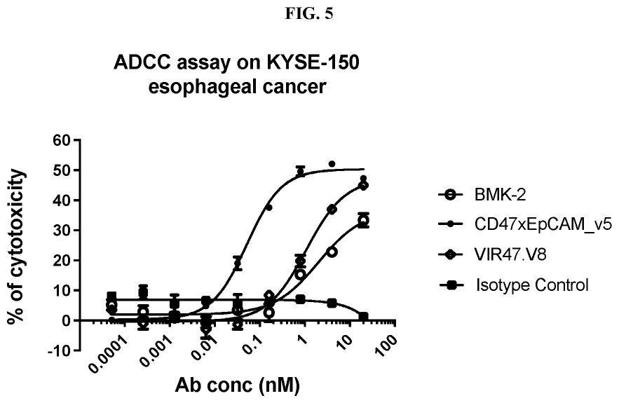

In FIG. 5 CD47×EpCAM_v5 showed robust ADCC activity on CD47+/EpCAM+ tumor cells KYSE-150, more potent than BMK-2 and its bivalent CD47 parental antibody VIR47.V8.

### Example 8. Binding To Red Blood Cells and Hemagglutination

A CD47 binding domain that preferentially binds to the forms of CD47 expressed on tumor cells compared to the forms of CD47 expressed on red blood cells or platelets is desirable to enhance the safety profile of a therapeutic agent.

### Binding of Antibodies on RBCs

The RBC binding assay was performed by spinning down fresh human whole blood at 200×g for 10 minutes. Collected RBCs were washed twice with PBS and counted using flow cytometry. 1×10⁶ cells were dispensed into each well of a 96 well culture plate. Serially diluted anti-CD47 antibodies were added and incubated for 1 hour at 4° C. Cells were washed with FACS buffer (PBS+2% FBS) twice. Secondary antibody (Alexa Fluor® 488 Goat Anti-Human IgG (H+L)) was added and incubated for 1 hour at 4° C. Cells were washed twice and resuspended in 200 μL of FACS buffer and analyzed by flow cytometry.

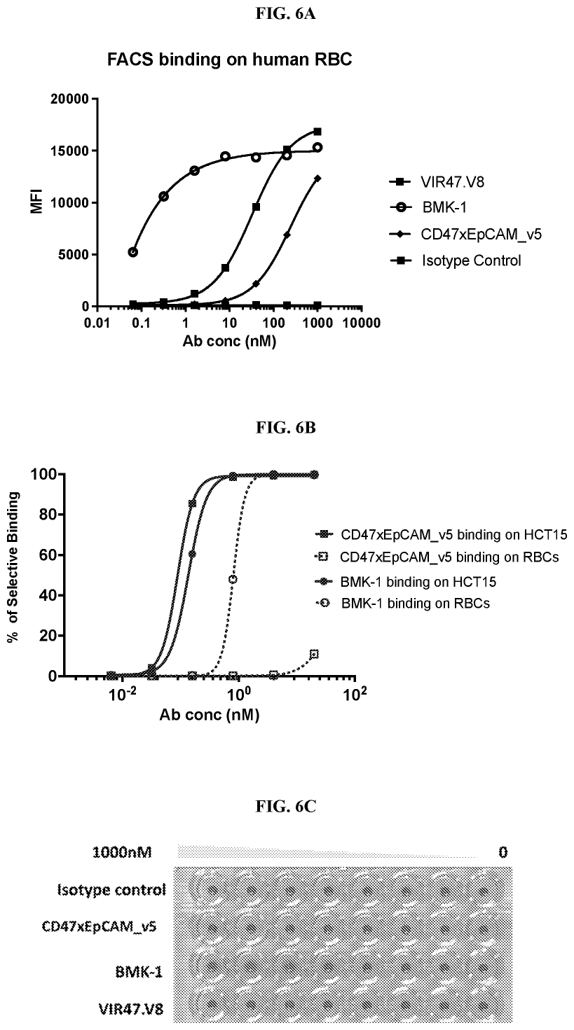

In FIG. 6A, CD47×EpCAM_v5 showed much weaker RBC binding compared with BMK-1 and VIR47.V8.

### Binding of Antibodies on Mixed Tumor Cells and RBCs

Binding of antibodies on mixed tumor cells and RBCs was performed by FACS. In brief, antibodies were incubated with a mixture of HCT-15 tumor cells and RBCs (in 20-fold excess of tumor) for 1 h at 4° C. The binding of antibodies on HCT-15 cells and RBCs were identified by using different FACS markers and detected by flow cytometry. In FIG. 6B, both CD47×EpCAM_v5 and BMK-1 showed robust binding on HCT-15 tumor cells with similar binding EC₅₀. On the RBCs, BMK-1 binding remained strong whereas binding of CD47×EpCAM_v5 is barely detectable even at the highest concentration tested. The binding EC₅₀ of CD47×EpCAM_v5 on tumor cells is >200 fold higher than its binding EC₅₀ on RBCs.

### Hemagglutination Assay

The hemagglutination assay was performed by diluting human red blood cells (RBCs) and incubating RBCs at 37° C. for 2 hours with a titration of CD47 antibodies (from 100 μg/mL) in a round bottom 96 well plate. Hemagglutination is demonstrated by the presence of crosslinked RBCs, which appear as a haze because they do not settle to the bottom of the well, in contrast to non-hemagglutinated RBCs.

CD47×EpCAM_v5 didn't cause hemagglutination even at 1 μM treatment as shown in FIG. 6C.

### Example 9. Binding to Human T Cells from PBMC Isolation

Human PBMCs were plated in 96 well deep well plate at 1×10⁶ per well. The cells were stained with live-dead dye by incubating at room temperature for 15 minutes and then washed one time. Fc blocker (5 μL/test) was added to all samples and incubated for 10 minutes at room temperature. Then, the samples were incubated with serially diluted test antibodies at the top concentration of 100 nM for 30 minutes at 4° C. After that anti-CD3 antibody was added to each sample and further incubated for 30 min at 4° C. following with 30 min incubation of secondary antibody at 4° C. Samples were washed twice before analyzed by flow cytometry.

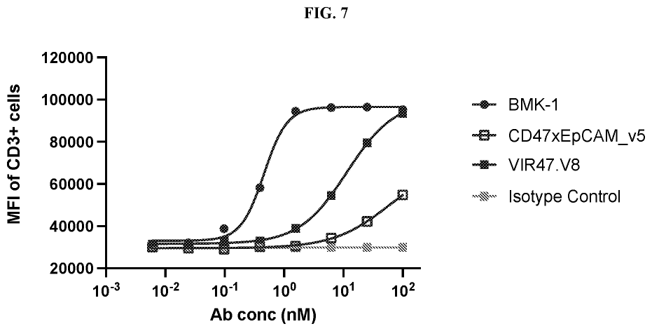

In FIG. 7 CD47×EpCAM_v5 showed weak binding on human T cells compared to BMK-1 and its parental anti-CD47 bivalent antibody VIR47.V8.

### Example 10. In Vivo Anti-Tumor Activities of CD47×EpCAM Bispecific and CD47 or EpCAM Bivalent Antibodies were Investigated in KATOIII Gastric Cancer Xenograft Model and OE-19 Esophageal Cancer Model

The KATOIII tumor cells or OE-19 tumor cells were cultured and harvested for tumor inoculation. Female NCG or CB17/SCID mice of 6-7 weeks in age (Shanghai Lingchang Biotechnology) were inoculated subcutaneously with 10⁷ cells in 0.2 mL of PBS supplemented with Matrigel (1:1) for tumor development. On day 6-8 post tumor inoculation when the mean tumor volume reached approximately 175 mm³, animals were randomized by tumor volumes into 6 animals per group. Test antibodies were administered through bolus tail vein injection at doses mentioned in the graphs. Tumor volume was measured twice weekly. Animals were humanely euthanized if their tumor volumes exceeded 3000 mm³ or if they experienced over 20% of body weight loss. Black arrows indicated the dosing day. α CD47 and a EpCAM antibodies represent the parental bivalent anti-CD47 and anti-EpCAM antibodies for CD47×EpCAM_v5.

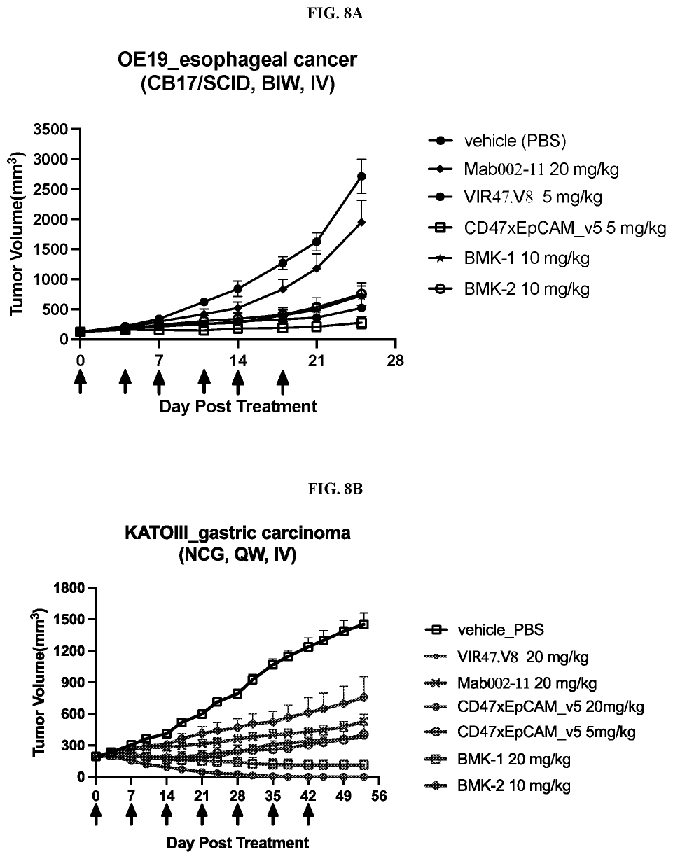

In FIG. 8A, CD47×EpCAM_v5 completely inhibited tumor growth in OE-19, an esophageal cancer model, superior to BMK-1 and BMK-2. In FIG. 8B, CD47×EpCAM_v5 drove complete tumor regression in KATOIII and all animals remained tumor free even 10 days after treatment ended. In comparison, CD47×EPCAM_v5 is superior to its parental bivalent antibodies, BMK-1 and BMK-2.

Our findings suggest that CD47×EpCAM_v5, a novel CD47×EpCAM bispecific antibody selectively binds to CD47 and blocks CD47/SIRPα binding on EpCAM overexpressing tumor cells. Its human IgG1 backbone allows engagement of both ADCP and ADCC for potent single agent anti-tumor activity. CD47×EpCAM_v5 has reduced RBC binding compared to the bivalent CD47 monospecific antibodies and thus is expected to induce less hematological toxicities. In conclusion, CD47×EpCAM_v5 shows specificity toward EpCAM positive tumor cells and robust single agent in vitro and in vivo activity. The CD47×EpCAM bispecific antibody represents a novel approach for treating EpCAM positive tumors.

### Example 11. Generation of Anti-CD47/Anti-EGFR Bispecific Antibodies

Anti-CD47/EGFR bispecific antibodies with different binding stoichiometry and geometry were designed and generated using VH and VL sequences from the anti-CD47 antibody VIR47. V8 and anti-EGFR antibody panitumumab or cetuximab sequences. The corresponding heavy chain (HC) and light chain (LC) DNAs were synthesized and cloned into the pRK5 mammalian expression vector (ATCC). Each HC and LC pair were then co-transfected in CHO cells. The conditioned medium was harvested by centrifugation (4° C., 4000 rpm for 40 min), then filtered to remove cell debris. The clarified medium was loaded onto MabSelect SuRe column (GE, 17-5438) which was pre-equilibrated with Buffer A (25 mM Tris, 150 mM NaCl, pH 8.0). The column was washed sequentially with 5 column volume of Buffer A, then 30 column volume of Buffer B (Buffer A+0.1% Triton X100+0.1% Triton X114), then 15 column volume of Buffer A. The antibodies were eluted with Buffer C (100 mM sodium citrate, 150 mM NaCl, pH 3.0) and neutralized immediately with Buffer D (200 mM Arginine, 137 mM Succinic acid, pH 5.0). The final product was dialyzed against the buffer PBS, pH 7.4, concentrated and filtrated through MILLEX-MP 0.22 um (MILLIPORE).

### Example 12: Antibody Binding to Antigen Measured by ELISA

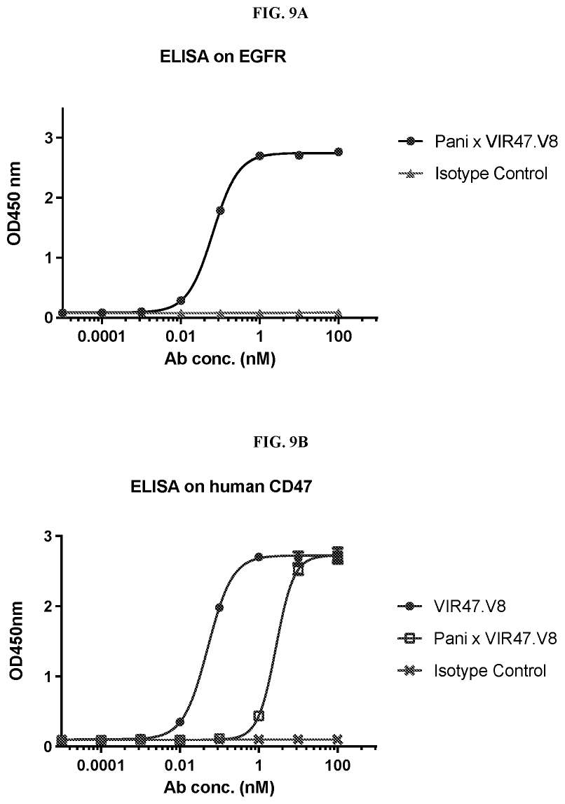

A 96-well plate was coated overnight at 4° C. with 1 μg/mL recombinant human CD47 or human EGFR. After washing 3 times, the plate was blocked with 300 μL 1% BSA in PBST at 37° C. for 1 hour. Serially diluted antibodies were added and incubated at 37° C. for 1 hour. The plate was then washed 4 times with PBST and incubated with 1:5000 diluted peroxidase labeled goat anti-human IgG (Fab specific) secondary antibody (Sigma, Cat #A0293) for 1 hour at 37° C. The plate was washed again 4 times with PBST, incubated with 3,3′,5,5′-tetramethylbenzidine (TMB) substrate for 15 min at room temperature, terminated with 1N HCl, and then read at 450 nm. FIGS. 9A-9B showed the ELISA binding results for anti-CD47/EGFR bispecific antibody Panitumumab xVIR47.V8 on human CD47 and human EGFR, respectively.

### Example 13: Combination of CD47×EpCAM Bispecific Antibody with Standard of Care Chemotherapy Agents in DLD-1 Colorectal Cancer Xenograft Model

DLD-1 tumor cells were cultured in RPMI 1640 medium supplemented with 10% FBS at 37° C. in an atmosphere of 5% CO₂ in air. Female Balb/c nude mice (Shanghai Lingchang Laboratory Animal Technology Co., Ltd.) of 6-8 weeks in age were inoculated subcutaneously with 5×10⁶ cells in 0.1 mL PBS for tumor development. Once the average tumor volume reached approximately 150-200 mm³, the animals were randomized to 6 animals per group and started treatment. Oxaliplatin and 5-fluorouracil (5-FU) were administered through intraperitoneal injection three times per week at a dose of 8 mg/kg and 35 mg/kg, respectively. A total of 4 doses of oxaliplatin and 5 doses of 5-FU were given. Irinotecan was administered through intraperitoneal injection twice per week at a dose of 30 mg/kg for a total of 6 doses. CD47×EpCAM_v5 bispecific antibody was administered through bolus tail vein injection at 20 mg/kg weekly for a total of 5 doses. For the combination groups, the route and dosing schedule were the same as in the single agent treatment groups. Tumor volume was measured twice weekly.

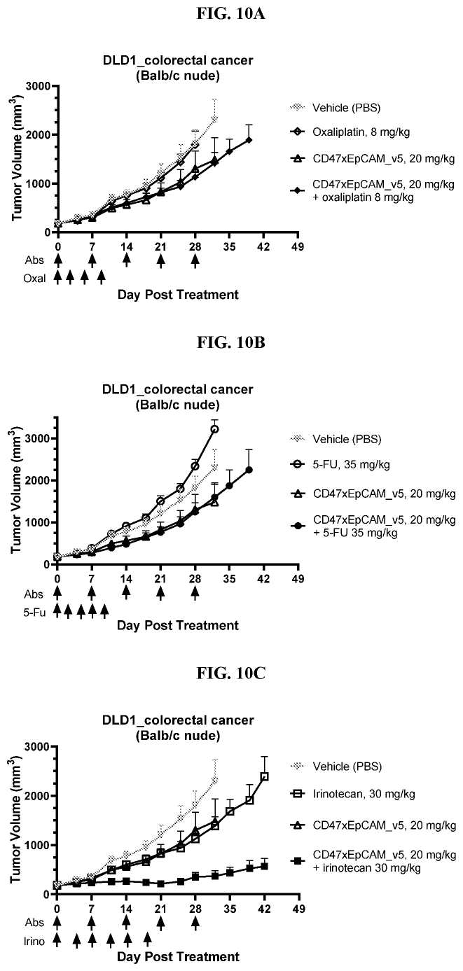

As shown in FIGS. 10A and 10B, treatment with CD47×EpCAM_v5 bispecific antibody alone only marginally slowed tumor growth. Combining CD47×EpCAM_v5 with either oxaliplatin or 5-FU treatment did not improve the anti-tumor efficacy. Surprisingly, combining irinotecan and CD47×EpCAM_v5 synergistically and effectively inhibited tumor progression although irinotecan or CD47×EpCAM alone was not very effective (FIG. 10C).

### Example 14: Combination of CD47×EpCAM Bispecific Antibodies, Benchmark or Bivalent Monospecific Antibodies with Irinotecan in DLD-1 Colorectal Cancer Xenograft Model

DLD-1 xenograft model was established as described in Example 13. Treatment was initiated once the average tumor volume reached approximately 150-200 mm³. Irinotecan was administered through intraperitoneal injection twice per week at a dose of 30 mg/kg for a total of 5 doses. All tested antibodies were administered through bolus tail vein injection at 20 mg/kg weekly for a total of 5 doses. Combination groups followed the same dosing schedule and route of administration as their corresponding monotherapy groups.

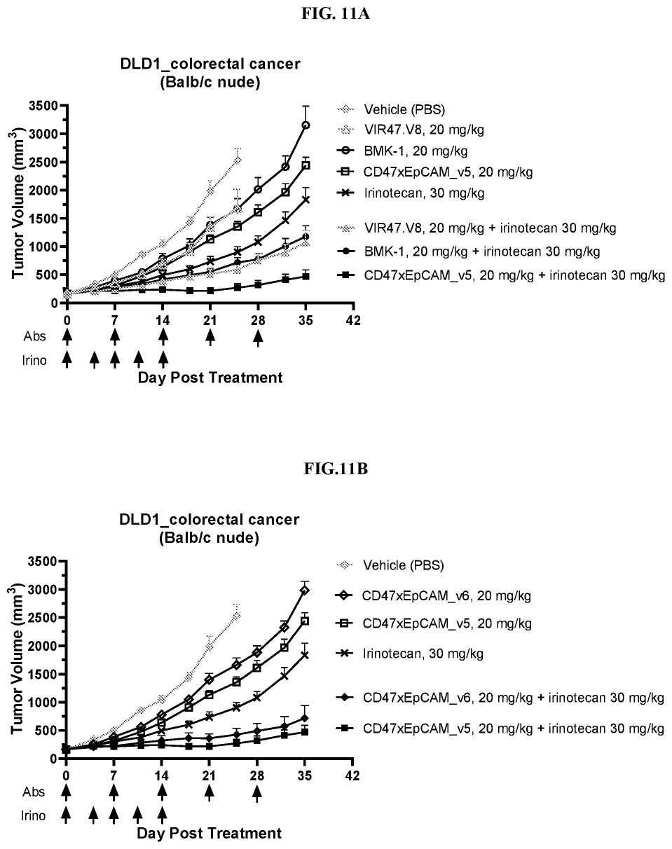

As shown in FIG. 11A, single agent treatment with irinotecan, BMK-1, CD47×EpCAM_v5 bispecific antibody or its parental bivalent anti-CD47 antibody VIR47.V8 only resulted in partial tumor growth inhibition. Combining irinotecan treatment with BMK-1 or anti-CD47 bivalent antibody VIR47.V8 only slightly improved anti-tumor activities over single agent treatments. In contrast, combination of irinotecan with CD47×EpCAM_v5 bispecific antibody again resulted in the largest anti-tumor effect. FIG. 11B shows that combining irinotecan with CD47×EpCAM_v6 resulted in similar anti-tumor efficacy as combining with CD47×EPCAM_v5. Taken together, these results demonstrate that the synergistic effect of combining irinotecan and CD47×EpCAM bispecific antibody could not be reproduced by combining with a benchmark (BMK-1) or the parental CD47 (VIR47.V8) bivalent antibodies. The irinotecan combination requires a CD47×EpCAM bispecific antibody such as CD47×EpCAM_v5 or CD47×EpCAM_6.

### Example 15: Combination of CD47×EpCAM Bispecific Antibody with Irinotecan in Hep3B2.1-7 Hepatocellular Carcinoma Xenograft Model

Hep3B2.1-7 tumor cells were cultured in EMEM medium supplemented with 10% FBS at 37° C. in an atmosphere containing 5% CO₂ in air. Female Balb/c nude mice (Shanghai Lingchang Laboratory Animal Technology Co., Ltd.) of 6-8 weeks in age were inoculated subcutaneously with 8×10⁶ cells in 0.1 mL of PBS for tumor development. Once the average tumor volume reached approximately 150-200 mm³, the animals were randomized to 6 animals per group and started treatment. Irinotecan was administered through intraperitoneal injection twice per week at a dose of 15 mg/kg for a total of 4 doses. CD47×EpCAM_v5 bispecific antibody was administered through bolus tail vein injection at 20 mg/kg weekly for a total of 4 doses. Combination groups followed the same dosing schedule and route of administration as their corresponding monotherapy groups. Tumor volume was measured twice weekly.

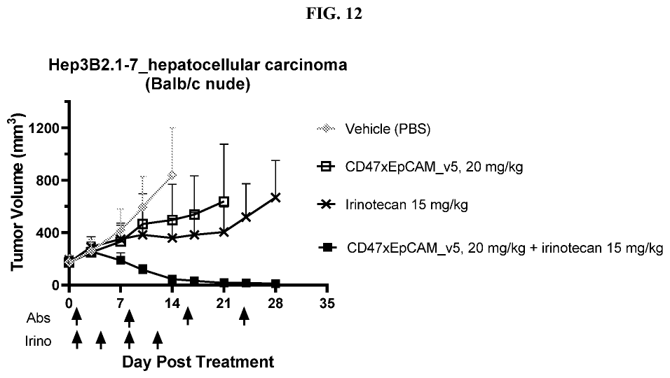

As shown in FIG. 12, single agent treatments with either irinotecan or CD47×EpCAM_v5 bispecific antibody slowed tumor growth but did not inhibit it. Interestingly, combining irinotecan and CD47×EpCAM_v5 treatments led to effective tumor regression.

### Example 16: Antibody Binding to Antigen Measured by ELISA

ELISA assay was performed as described in EXAMPLE 3. The bindings of CD47×EpCAM_v9, v10 and v11 on human EpCAM and human CD47 were tested.

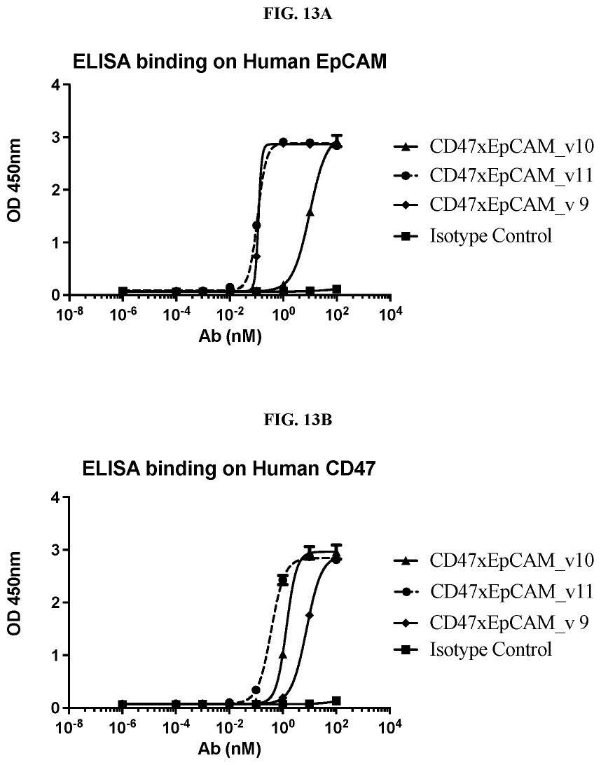

FIGS. 13A-13B showed the ELISA binding results for anti-CD47×EpCAM bispecific antibodies CD47×EpCAM_v9, v10 and v11 on human EpCAM and human CD47, respectively.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-150. (canceled)

151. A multispecific antibody comprising an EpCAM binding domain, wherein the EpCAM binding domain comprises three heavy chain (HC) complementarity determining regions (CDRs): HC-CDR1, HC-CDR2, and HC-CDR3, and three light chain (LC) CDRs: LC-CDR1, LC-CDR2, and LC-CDR3, wherein the HC-CDR1 comprises the amino acid sequence according to SEQ ID NO: 243, wherein the HC-CDR2 comprises the amino acid sequence according to SEQ ID NO: 244, the HC-CDR3 comprises the amino acid sequence according to SEQ ID NO: 245, wherein the LC-CDR1 comprises the amino acid sequence according to SEQ ID NO: 297, the LC-CDR2 comprises the amino acid sequence according to SEQ ID NO: 298, and the LC-CDR3 comprises the amino acid sequence according to SEQ ID NO: 299.

152. The multispecific antibody of claim 151, wherein the EpCAM binding domain comprises a heavy chain variable domain (VH) comprising a sequence having at least 90% identity to SEQ ID NO: 407 or a light chain variable domain (VL) a sequence having at least 90% identity to SEQ ID NO: 425.

153. The multispecific antibody of claim 151, wherein the EpCAM binding domain comprises a heavy chain (HC) comprising a sequence having at least 90% identity to SEQ ID NO: 442 or a light chain (LC) comprising a sequence having at least 90% identity to SEQ ID NO: 451.

154. The multispecific antibody of claim 151, further comprising a CD47-binding domain.

155. The multispecific antibody of claim 154, wherein the CD47-binding domain comprises an antigen-binding fragment (Fab), and the EpCAM-binding domain comprises a single-chain variable fragment (scFv); or wherein the CD47-binding domain comprises a single-chain variable fragment (scFv), and the EpCAM-binding domain comprises an antigen-binding fragment (Fab).

156. The multispecific antibody of claim 154, wherein the CD47-binding domain comprises three heavy chain (HC) complementarity determining regions (CDRs): HC-CDR1, HC-CDR2, and HC-CDR3, and three light chain (LC) CDRs: LC-CDR1, LC-CDR2, and LC-CDR3, wherein the HC-CDR1 comprises the amino acid sequence according to SEQ ID NO: 1, wherein the HC-CDR2 comprises the amino acid sequence according to SEQ ID NO: 2, the HC-CDR3 comprises the amino acid sequence according to SEQ ID NO: 3, wherein the LC-CDR1 comprises the amino acid sequence according to SEQ ID NO: 61, the LC-CDR2 comprises the amino acid sequence according to SEQ ID NO: 62, and the LC-CDR3 comprises the amino acid sequence according to SEQ ID NO: 63.

157. The multispecific antibody of claim 154, wherein the CD47-binding domain comprises a heavy chain variable domain (VH) comprising a sequence having at least 90% identity to SEQ ID NO: 123 or a light chain variable domain (VL) a sequence having at least 90% identity to SEQ ID NO: 144.

158. The multispecific antibody of claim 151, further comprising an Fc domain.

159. The multispecific antibody of claim 158, wherein the Fc domain is a heterodimeric Fc domain, wherein the heterodimeric Fc region comprises a knob chain and a hole chain, forming a knob-into-hole (KiH) structure.

160. The multispecific antibody of claim 159, wherein the knob chain comprises the mutation T366W and the hole chain comprises the mutations T366S, L368A, and Y407V, wherein amino acid position numbering is according to the EU index of Kabat et al.

161. The multispecific antibody of claim 158, wherein the isotype of the Fc domain is IgG1, IgG2, IgG3, or IgG4.

162. A pharmaceutical composition comprising the multispecific antibody of claim 151 and an anti-cancer agent.

163. The pharmaceutical composition of claim 162, wherein the anti-cancer agent is a chemotherapeutic agent or a biologic agent.

164. The pharmaceutical composition of claim 163, wherein the chemotherapeutic agent comprises irinotecan, oxaliplatin, or 5-FU.

165. A method of treating cancer in a subject in need thereof comprising administering to the subject the multispecific antibody of claim 151.

166. The method of claim 165, further comprising administering an anti-cancer agent.

167. The method of claim 166, wherein the anti-cancer agent is a chemotherapeutic agent or a biologic agent.

168. The method of claim 167, wherein the chemotherapeutic agent comprises irinotecan, oxaliplatin, or 5-FU.

169. The method of claim 165, wherein the cancer comprises epidermoid carcinoma, colorectal adenocarcinoma, head and neck squamous cell carcinoma, bladder carcinoma, pancreas adenocarcinoma, lung squamous cell carcinoma, lung adenocarcinoma, gastric carcinoma, ductal pancreatic adenocarcinoma, ovarian clear cell adenocarcinoma, ovarian adenocarcinoma, colon adenocarcinoma, rectum adenocarcinoma, esophageal cancer, gastric cancer, non-small cell lung cancer, small cell lung cancer, endometrial cancer, hepatocellular carcinoma, cholangiocarcinoma, prostate adenocarcinoma, thyroid carcinoma, breast cancer, cervical carcinoma, renal cell carcinoma, testis cancer, or vulvar squamous cell carcinoma.

170. The method of claim 165, wherein the cancer comprises small cell lung cancer, lung adenocarcinoma, gastric cancer, colorectal cancer, ovarian cancer, or any combination thereof.


SEQUENCE LISTING (510 sequences)

SEQ ID NO: 1 - protein, 5 aa, synthetic construct
NFAMS

SEQ ID NO: 2 - protein, 17 aa, synthetic construct
TISASGGRTFYADSVKG

SEQ ID NO: 3 - protein, 10 aa, synthetic construct
EGSFGEGVDP

SEQ ID NO: 4 - protein, 5 aa, synthetic construct
SYVMH

SEQ ID NO: 5 - protein, 17 aa, synthetic construct
YIDPYNDGTKYNEKFKG

SEQ ID NO: 6 - protein, 10 aa, synthetic construct
GGMARRSFDY

SEQ ID NO: 7 - protein, 5 aa, synthetic construct
DTYMH

SEQ ID NO: 8 - protein, 17 aa, synthetic construct
RIDPANGKTKSDPKFQG

SEQ ID NO: 9 - protein, 11 aa, synthetic construct
GDGYYVGGLDY

SEQ ID NO: 10 - protein, 5 aa, synthetic construct
EYTIH

SEQ ID NO: 11 - protein, 17 aa, synthetic construct
SINPNNGLFRYNQKFKG

SEQ ID NO: 12 - protein, 9 aa, synthetic construct
DYGFGYSDY

SEQ ID NO: 13 - protein, 5 aa, synthetic construct
DTYMH

SEQ ID NO: 14 - protein, 17 aa, synthetic construct
RIDPANGNTKYDPKFQG

SEQ ID NO: 15 - protein, 11 aa, synthetic construct
GDGHYVGGLNY

SEQ ID NO: 16 - protein, 5 aa, synthetic construct
SYWMN

SEQ ID NO: 17 - protein, 17 aa, synthetic construct
SIDPYDSETHYNQKFKD

SEQ ID NO: 18 - protein, 8 aa, synthetic construct
GRGYAMDY

SEQ ID NO: 19 - protein, 7 aa, synthetic construct
SSGYYMC

SEQ ID NO: 20 - protein, 17 aa, synthetic construct
CIYMRSGDTYLASWAKG

SEQ ID NO: 21 - protein, 9 aa, synthetic construct
GGTSYALNL

SEQ ID NO: 22 - protein, 5 aa, synthetic construct
SYWMA

SEQ ID NO: 23 - protein, 18 aa, synthetic construct
CIYIGSGSGTTYYASWAK

SEQ ID NO: 24 - protein, 10 aa, synthetic construct
WAGSDGHFTL

SEQ ID NO: 25 - protein, 6 aa, synthetic construct
SYYYVC

SEQ ID NO: 26 - protein, 18 aa, synthetic construct
CIYAGSSGDTYYASWAKG

SEQ ID NO: 27 - protein, 13 aa, synthetic construct
ARGYTGYSYQFNF

SEQ ID NO: 28 - protein, 5 aa, synthetic construct
SYHMC

SEQ ID NO: 29 - protein, 17 aa, synthetic construct
CIYPNNGFTWYASWAKG

SEQ ID NO: 30 - protein, 11 aa, synthetic construct
GSGNSYNQLDL

SEQ ID NO: 31 - protein, 5 aa, synthetic construct
SYHMN

SEQ ID NO: 32 - protein, 17 aa, synthetic construct
IIYVGSGNADYASWAKG

SEQ ID NO: 33 - protein, 12 aa, synthetic construct
GPTNTYPTYFNL

SEQ ID NO: 34 - protein, 5 aa, synthetic construct
SGYMC

SEQ ID NO: 35 - protein, 18 aa, synthetic construct
CIYAGSSGKTYYASWATG

SEQ ID NO: 36 - protein, 13 aa, synthetic construct
DDGGTYYTDYLYL

SEQ ID NO: 37 - protein, 5 aa, synthetic construct
SYHMC

SEQ ID NO: 38 - protein, 17 aa, synthetic construct
CIYPNNDFTWYATWAKG

SEQ ID NO: 39 - protein, 11 aa, synthetic construct
ASGNAYNLLDL

SEQ ID NO: 40 - protein, 6 aa, synthetic construct
SGYYMC

SEQ ID NO: 41 - protein, 17 aa, synthetic construct
CIYVRSDSTYYASWAKG

SEQ ID NO: 42 - protein, 11 aa, synthetic construct
GVTGDSYRFNF

SEQ ID NO: 43 - protein, 5 aa, synthetic construct
SYHMS

SEQ ID NO: 44 - protein, 17 aa, synthetic construct
IIYADTGNADYASWTKG

SEQ ID NO: 45 - protein, 12 aa, synthetic construct
GPTNTYPTYFAL

SEQ ID NO: 46 - protein, 5 aa, synthetic construct
SYVMH

SEQ ID NO: 47 - protein, 17 aa, synthetic construct
YIDPYNEGTKYNEKFKG

SEQ ID NO: 48 - protein, 10 aa, synthetic construct
GGMARRSFDY

SEQ ID NO: 49 - protein, 5 aa, synthetic construct
SYVMH

SEQ ID NO: 50 - protein, 17 aa, synthetic construct
YIDPYNEGTKYNEKFKG

SEQ ID NO: 51 - protein, 10 aa, synthetic construct
GGMARRSFDY

SEQ ID NO: 52 - protein, 5 aa, synthetic construct
SGYMS

SEQ ID NO: 53 - protein, 18 aa, synthetic construct
SIYAGSSGKTYYADWAKG

SEQ ID NO: 54 - protein, 13 aa, synthetic construct
DSGGTYYTDYLYA

SEQ ID NO: 55 - protein, 5 aa, synthetic construct
SGYMS

SEQ ID NO: 56 - protein, 18 aa, synthetic construct
SIYAGSSGKTYYADWAKG

SEQ ID NO: 57 - protein, 13 aa, synthetic construct
DSGGTYYTDYLYL

SEQ ID NO: 58 - protein, 5 aa, synthetic construct
SGYMS

SEQ ID NO: 59 - protein, 18 aa, synthetic construct
SIYAGSSGKTYYADWAKG

SEQ ID NO: 60 - protein, 13 aa, synthetic construct
DSGGTYYTDYLYL

SEQ ID NO: 61 - protein, 11 aa, synthetic construct
SGDALPKKYAY

SEQ ID NO: 62 - protein, 7 aa, synthetic construct
EDNKRPS

SEQ ID NO: 63 - protein, 11 aa, synthetic construct
YSTDISGNHWV

SEQ ID NO: 64 - protein, 11 aa, synthetic construct
LASQTIGTWLT

SEQ ID NO: 65 - protein, 7 aa, synthetic construct
AATTLAD

SEQ ID NO: 66 - protein, 9 aa, synthetic construct
QQLYSTPWT

SEQ ID NO: 67 - protein, 11 aa, synthetic construct
HASQNIHVWLS

SEQ ID NO: 68 - protein, 7 aa, synthetic construct
KASNLHT

SEQ ID NO: 69 - protein, 9 aa, synthetic construct
QQGQSYPYT

SEQ ID NO: 70 - protein, 11 aa, synthetic construct
RASQGIRGNLD

SEQ ID NO: 71 - protein, 7 aa, synthetic construct
STSNLNS

SEQ ID NO: 72 - protein, 9 aa, synthetic construct
LQRNAFPWT

SEQ ID NO: 73 - protein, 11 aa, synthetic construct
HASQNINVWLS

SEQ ID NO: 74 - protein, 7 aa, synthetic construct
KASNLHT

SEQ ID NO: 75 - protein, 9 aa, synthetic construct
QQGQNYPYT

SEQ ID NO: 76 - protein, 11 aa, synthetic construct
RASGNIHNYLA

SEQ ID NO: 77 - protein, 7 aa, synthetic construct
NAKTLAD

SEQ ID NO: 78 - protein, 9 aa, synthetic construct
QHFWSTPPT

SEQ ID NO: 79 - protein, 11 aa, synthetic construct
QASQSISNECS

SEQ ID NO: 80 - protein, 7 aa, synthetic construct
RASTLES

SEQ ID NO: 81 - protein, 11 aa, synthetic construct
QSTYYGTTGNT

SEQ ID NO: 82 - protein, 11 aa, synthetic construct
QASQSINNLLA

SEQ ID NO: 83 - protein, 7 aa, synthetic construct
DASDLAS

SEQ ID NO: 84 - protein, 12 aa, synthetic construct
QGYFYGSSVDLV

SEQ ID NO: 85 - protein, 7 aa, synthetic construct
RASTLES

SEQ ID NO: 86 - protein, 11 aa, synthetic construct
QASQSISNELS

SEQ ID NO: 87 - protein, 10 aa, synthetic construct
QCTYITYGNA

SEQ ID NO: 88 - protein, 13 aa, synthetic construct
QSSESVYRDNCLG

SEQ ID NO: 89 - protein, 7 aa, synthetic construct
KASTLAS

SEQ ID NO: 90 - protein, 12 aa, synthetic construct
AGYKSTITDGTA

SEQ ID NO: 91 - protein, 11 aa, synthetic construct
QASRSITSYLA

SEQ ID NO: 92 - protein, 7 aa, synthetic construct
AASNVAS

SEQ ID NO: 93 - protein, 10 aa, synthetic construct
QSYYAISTNT

SEQ ID NO: 94 - protein, 11 aa, synthetic construct
QASEGISSYLS

SEQ ID NO: 95 - protein, 7 aa, synthetic construct
KASTLTS

SEQ ID NO: 96 - protein, 15 aa, synthetic construct
QCTYGSSGSSGYGAA

SEQ ID NO: 97 - protein, 13 aa, synthetic construct
QSSESVYRDNCLG

SEQ ID NO: 98 - protein, 7 aa, synthetic construct
RASTLAS

SEQ ID NO: 99 - protein, 12 aa, synthetic construct
AGYKSTITDGTA

SEQ ID NO: 100 - protein, 11 aa, synthetic construct
QASQSISNELS

SEQ ID NO: 101 - protein, 7 aa, synthetic construct
RASTLES

SEQ ID NO: 102 - protein, 12 aa, synthetic construct
QSTYYGTSYGNA

SEQ ID NO: 103 - protein, 11 aa, synthetic construct
QASQSITTYLA

SEQ ID NO: 104 - protein, 7 aa, synthetic construct
GASNLES

SEQ ID NO: 105 - protein, 10 aa, synthetic construct
QSYYAISKSA

SEQ ID NO: 106 - protein, 11 aa, synthetic construct
LASQTIGTWLT

SEQ ID NO: 107 - protein, 7 aa, synthetic construct
AATTLAE

SEQ ID NO: 108 - protein, 9 aa, synthetic construct
QQLYSTPWT

SEQ ID NO: 109 - protein, 11 aa, synthetic construct
LASQTIGTWLT

SEQ ID NO: 110 - protein, 7 aa, synthetic construct
AATTLAE

SEQ ID NO: 111 - protein, 9 aa, synthetic construct
QQLYSTPWT

SEQ ID NO: 112 - protein, 11 aa, synthetic construct
RASEGISSYLS

SEQ ID NO: 113 - protein, 7 aa, synthetic construct
KASTLTS

SEQ ID NO: 114 - protein, 15 aa, synthetic construct
QSTYGSSGSSGYGAA

SEQ ID NO: 115 - protein, 11 aa, synthetic construct
RASEGISSYLS

SEQ ID NO: 116 - protein, 7 aa, synthetic construct
KASTLTS

SEQ ID NO: 117 - protein, 15 aa, synthetic construct
QSAYGSSGSSGYGAA

SEQ ID NO: 118 - protein, 11 aa, synthetic construct
RASEGISSYLS

SEQ ID NO: 119 - protein, 7 aa, synthetic construct
KASTLTS

SEQ ID NO: 120 - protein, 15 aa, synthetic construct
QSTYGSSGSSGYAAA

SEQ ID NO: 121 - protein, 119 aa, synthetic construct
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEGVDPWGQGTLVTVSS

SEQ ID NO: 122 - protein, 119 aa, synthetic construct
EVQLLESGGGLVQPGGSLRLSCAASGFTFSNFAMSWVRQAPGKGLEWVSTISASGGRTFYADSVKGRITISRDNSKNTLYLQMNSLRAEDTAVYYCAKEGSFGEGVDPWGQGTLVTVSS

SEQ ID NO: 123 - protein, 119 aa, synthetic construct
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSS

SEQ ID NO: 124 - protein, 119 aa, synthetic construct
EVQLQQSGPELVKPGAPVKMSCKASGYTFTSYVMHWVKQKPGQGLEWIGYIDPYNDGTKYNEKFKGKATLTSDKSSSTAYMELSSLTSEDSAVYYCSRGGMARRSFDYWGQGTTLTVSS

SEQ ID NO: 125 - protein, 120 aa, synthetic construct
EVQLQQSGAELVRPGASVKLSCTASGFNIEDTYMHWVKQRPEQGLEWIGRIDPANGKTKSDPKFQGKATITTDTSSNTAYLQLSSLTSEDTAVYYCAKGDGYYVGGLDYWGQGTTLTVSS

SEQ ID NO: 126 - protein, 118 aa, synthetic construct
EVQLQQSGPELVKPGASVKMSCKTSGYTFTEYTIHWVKQSHEKSLEWIGSINPNNGLFRYNQKFKGKATLTVDKSSSTAYMELRSLTSEDSAVYYCARDYGFGYSDYWGQGTTLTVSS

SEQ ID NO: 127 - protein, 120 aa, synthetic construct
EVQLQQSGAELVKPGASVKLSCTASGFNIEDTYMHWVKQRPEQGLEWIGRIDPANGNTKYDPKFQGKATVTADTSSNTAYLQLSSLTSEDTAVYCCARGDGHYVGGLNYWGQGTSLTVSS

SEQ ID NO: 128 - protein, 117 aa, synthetic construct
QVQLQQPGAELVRPGASVKLSCKASGYTFTSYWMNWVKQRPEQGLEWIGSIDPYDSETHYNQKFKDKAILTVDKSSSTAYMQLSSLTSEDSAVYYCARGRGYAMDYWGQGTSVTVSS

SEQ ID NO: 129 - protein, 117 aa, synthetic construct
QSLEESGGDLVKPGASLTLTCTASGFSFSSGYYMCWVRQAPGKGLEWIACIYMRSGDTYLASWAKGQFTISKTSSTTVTLQMTSLTAADTATYFCGRGGTSYALNLWGQGTLVTVSS

SEQ ID NO: 130 - protein, 119 aa, synthetic construct
QEQLEESGGDLVKPEGSLTLTCTASGFSFSSYWMAWVRQAPGKGLEWIACIYIGSGSGTTYYASWAKGRFTISKTSSTTVTLQMTSLTAADTAIYFCARWAGSDGHFTLWGPGTLVTVS

SEQ ID NO: 131 - protein, 120 aa, synthetic construct
QSLEESGGDLVKPGASLTVTCTASGFSFSSYYYVCWVRQAPGKGLEWIACIYAGSSGDTYYASWAKGRFTISKTSSTTVTLQMTSLTAADTATYFCARGYTGYSYQFNFWGPGTLVTVSS

SEQ ID NO: 132 - protein, 119 aa, synthetic construct
QEQLKESGGGLVTPGGTLTLTCTASGFTITSYHMCWVRQAPGKGLEWIGCIYPNNGFTWYASWAKGRFTISKTSSTTVDLKMTSPTTEEMATYFCARGSGNSYNQLDLWGQGTLVTVSS

SEQ ID NO: 133 - protein, 118 aa, synthetic construct
QSLEESGGRLVTPGTPLTLTCTASGFTISSYHMNWVRQAPGEGLEWIGIIYVGSGNADYASWAKGRFTISKASTTVDLKITSPTTEDTATYFCARGPTNTYPTYFNLWGPGTLVTVSS

SEQ ID NO: 134 - protein, 121 aa, synthetic construct
QSLEESGGDLVKPGASLTLTCKASGFSFNSGYMCWVRQAPGKGLEWIACIYAGSSGKTYYASWATGRFTISKTSSTTVTLQMTSLTVADTATYFCARDDGGTYYTDYLYLWGPGTLVTVSS

SEQ ID NO: 135 - protein, 119 aa, synthetic construct
QEQLKESGGGLVTPGGTLTLTCTASGFTISSYHMCWVRQAPGKGLEWIGCIYPNNDFTWYATWAKGRFTISKTSSTTVDLRMTTPTTEETATYFCARASGNAYNLLDLWGQGTLVTVSS

SEQ ID NO: 136 - protein, 120 aa, synthetic construct
QEQLVESGGGLVKPGASLTLTCKASGFSFSSGYYMCWVRQAPGKGLEWIGCIYVRSDSTYYASWAKGRFTISKASSTTVTLQMTSLTAADTATYFCARGVTGDSYRFNFWGPGTLVTVSS

SEQ ID NO: 137 - protein, 118 aa, synthetic construct
QSLEESGGRLVTPGTPLTLTCTASGFSLNSYHMSWVRQAPGEGLEWIGIIYADTGNADYASWTKGRFTISKASTTVELRITSPTSEDTATYFCARGPTNTYPTYFALWGPGTLVTVSS

SEQ ID NO: 138 - protein, 119 aa, synthetic construct
EVQLVQSGAEVKKPGASVKVSCKASGYTFTSYVMHWVRQAPGQRLEWMGYIDPYNEGTKYNEKFKGRVTITRDTSASTAYMELSSLRSEDTAVYYCSRGGMARRSFDYWGQGTLVTVSS

SEQ ID NO: 139 - protein, 119 aa, synthetic construct
EVQLVQSGAEVKKPGASVKVSCKASGYTFTSYVMHWVRQAPGQRLEWMGYIDPYNEGTKYNEKFKGRVTITSDTSASTAYMELSSLRSEDTAVYYCSRGGMARRSFDYWGQGTLVTVSS

SEQ ID NO: 140 - protein, 123 aa, synthetic construct
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYLYAWGQGTLVTVSS

SEQ ID NO: 141 - protein, 123 aa, synthetic construct
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYLYLWGQGTLVTVSS

SEQ ID NO: 142 - protein, 123 aa, synthetic construct; identical to SEQ ID NO: 141

SEQ ID NO: 143 - protein, 118 aa, synthetic construct
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVL

SEQ ID NO: 144 - protein, 108 aa, synthetic construct
SYELTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVL

SEQ ID NO: 145 - protein, 107 aa, synthetic construct
DIQMTQSPASQSASLGESVTITCLASQTIGTWLTWYQQKPGKSPQLLIYAATTLADGVPSRFSGSGSGTKFSFKISSLQAEDFVSYYCQQLYSTPWTFGGGTKLEIK

SEQ ID NO: 146 - protein, 107 aa, synthetic construct
DIQMNQSPSSLSASLGDTITITCHASQNIHVWLSWYQQKPGNIPKLLIYKASNLHTGVPSRFSGSGSGTGFTLTISSLQPEDIATYYCQQGQSYPYTFGGGTKLEIK

SEQ ID NO: 147 - protein, 107 aa, synthetic construct
DIQMIQSPSSMFASLGDRVSLSCRASQGIRGNLDWYQQKPGGTIKLLIYSTSNLNSGVPSRFSGSGSGSDYSLTISSLESEDFADYYCLQRNAFPWTFGGGTKLEIK

SEQ ID NO: 148 - protein, 107 aa, synthetic construct
DIQMNQSPSSLSASLGDTITITCHASQNINVWLSWYQQKPGNIPKLLIYKASNLHTGVPSRFSGSGSGTGFTLTISSLQPEDIATYYCQQGQNYPYTFGGGTKLEIK

SEQ ID NO: 149 - protein, 107 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPPTFGGGTKLEIK

SEQ ID NO: 150 - protein, 109 aa, synthetic construct
DVVMTQTPASVSEPVGGTVTINCQASQSISNECSWYQQKPGQPPKLLIYRASTLESGVPSRFSGSGSGTEFTLTISDLESADAATYYCQSTYYGTTGNTFGGGTEVVVK

SEQ ID NO: 151 - protein, 110 aa, synthetic construct
ALVMTQTPASVEAAVGGTVTINCQASQSINNLLAWYQQKPGQRPKLLIYDASDLASGVSSRFKGSGSGTEYTLTISGVQSADAATYYCQGYFYGSSVDLVFGGGTEVVVK

SEQ ID NO: 152 - protein, 108 aa, synthetic construct
DVVMTQTPASVEAAVGGTVTIKCQASQSISNELSWYQQKPGQPPKLLIYRASTLESGVPSRFKGSGSGTEFTLTISDLESADAATYYCQCTYITYGNAFGGGTEVVVK

SEQ ID NO: 153 - protein, 112 aa, synthetic construct
AIVMTQTPASVSATLGGSVTINCQSSESVYRDNCLGWFQQKPGQPPKLLIYKASTLASGVPSRFKGSGSGTQFTLTISDVVSDDAATYYCAGYKSTITDGTAFGGGTEVVVK

SEQ ID NO: 154 - protein, 108 aa, synthetic construct
AFELTQTPSSVEAAVGGTVTIKCQASRSITSYLAWYQHKPGQPPTLLIYAASNVASGVSSRFKGSRSGTEYTLTISDLESADAATYYCQSYYAISTNTFGGGTEVVVK

SEQ ID NO: 155 - protein, 113 aa, synthetic construct
DVVMTQTPASVEAAVGGTVTIKCQASEGISSYLSWYQQKPGQPPKLLIYKASTLTSGVPSRFKGSGSGTDFTLTISDLESADAATYYCQCTYGSSGSSGYGAAFGGGTEVVVK

SEQ ID NO: 156 - protein, 112 aa, synthetic construct
AIVMTQTPASVSVAVGGSVTINCQSSESVYRDNCLGWFQQKPGQPPKLLIYRASTLASGVPSRFKGSGSGTQFTLTISDVVSDDAATYYCAGYKSTITDGTAFGGGTEVVVK

SEQ ID NO: 157 - protein, 110 aa, synthetic construct
DVVMTQTPASVEAAVGGTVTIKCQASQSISNELSWYQQKSGQPPKLLIYRASTLESGVPSRFKGSGSGTEFTLTISDLESADAATYYCQSTYYGTSYGNAFGGGTEVVVK

SEQ ID NO: 158 - protein, 108 aa, synthetic construct
AFELTQTPSSVEAAVGGTVTIKCQASQSITTYLAWYQQKPGQPPKLLIYGASNLESGVPSRFKGSRSGTEYTLTISDLESADAATYYCQSYYAISKSAFGGGTEVVVK

SEQ ID NO: 159 - protein, 107 aa, synthetic construct
DIQMTQSPSSLSASVGDRVTITCLASQTIGTWLTWYQQKPGKAPKLLIYAATTLAEGVPSRFSGSGSGTDFTLTISSLQPEDFATYYCQQLYSTPWTFGGGTKVEIK

SEQ ID NO: 160 - protein, 107 aa, synthetic construct; identical to SEQ ID NO: 159

SEQ ID NO: 161 - protein, 113 aa, synthetic construct
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSGYGAAFGGGTKVEIK

SEQ ID NO: 162 - protein, 113 aa, synthetic construct
DIQMTQSPSSLSASVGDRVTITCRASEGISSYLSWYQQKPGKAPKLLIYKASTLTSGVPSRFSGSGSGTDFTLTISSLQPEDFATYYCQSAYGSSGSSGYGAAFGGGTKVEIK

SEQ ID NO: 163 - protein, 113 aa, synthetic construct
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSGYAAAFGGGTKVEIK

SEQ ID NO: 164 - protein, 108 aa, synthetic construct; identical to SEQ ID NO: 144

SEQ ID NO: 165 - protein, 446 aa, synthetic construct
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEGVDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 166 - protein, 449 aa, synthetic construct
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 449 aa, synthetic construct
EVQLLESGGGLVQPGGSLRLSCAASGFTFSNFAMSWVRQAPGKGLEWVSTISASGGRTFYADSVKGRITISRDNSKNTLYLQMNSLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 449 aa, synthetic construct
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 449 aa, synthetic construct
EVQLQQSGPELVKPGAPVKMSCKASGYTFTSYVMHWVKQKPGQGLEWIGYIDPYNDGTKYNEKFKGKATLTSDKSSSTAYMELSSLTSEDSAVYYCSRGGMARRSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 450 aa, synthetic construct
EVQLQQSGAELVRPGASVKLSCTASGFNIEDTYMHWVKQRPEQGLEWIGRIDPANGKTKSDPKFQGKATITTDTSSNTAYLQLSSLTSEDTAVYYCAKGDGYYVGGLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 448 aa, synthetic construct
EVQLQQSGPELVKPGASVKMSCKTSGYTFTEYTIHWVKQSHEKSLEWIGSINPNNGLFRYNQKFKGKATLTVDKSSSTAYMELRSLTSEDSAVYYCARDYGFGYSDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 450 aa, synthetic construct
EVQLQQSGAELVKPGASVKLSCTASGFNIEDTYMHWVKQRPEQGLEWIGRIDPANGNTKYDPKFQGKATVTADTSSNTAYLQLSSLTSEDTAVYCCARGDGHYVGGLNYWGQGTSLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 447 aa, synthetic construct
QVQLQQPGAELVRPGASVKLSCKASGYTFTSYWMNWVKQRPEQGLEWIGSIDPYDSETHYNQKFKDKAILTVDKSSSTAYMQLSSLTSEDSAVYYCARGRGYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 447 aa, synthetic construct
QSLEESGGDLVKPGASLTLTCTASGFSFSSGYYMCWVRQAPGKGLEWIACIYMRSGDTYLASWAKGQFTISKTSSTTVTLQMTSLTAADTATYFCGRGGTSYALNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - protein, 450 aa, synthetic construct
QEQLEESGGDLVKPEGSLTLTCTASGFSFSSYWMAWVRQAPGKGLEWIACIYIGSGSGTTYYASWAKGRFTISKTSSTTVTLQMTSLTAADTAIYFCARWAGSDGHFTLWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 450 aa, synthetic construct
QSLEESGGDLVKPGASLTVTCTASGFSFSSYYYVCWVRQAPGKGLEWIACIYAGSSGDTYYASWAKGRFTISKTSSTTVTLQMTSLTAADTATYFCARGYTGYSYQFNFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 449 aa, synthetic construct
QEQLKESGGGLVTPGGTLTLTCTASGFTITSYHMCWVRQAPGKGLEWIGCIYPNNGFTWYASWAKGRFTISKTSSTTVDLKMTSPTTEEMATYFCARGSGNSYNQLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 448 aa, synthetic construct
QSLEESGGRLVTPGTPLTLTCTASGFTISSYHMNWVRQAPGEGLEWIGIIYVGSGNADYASWAKGRFTISKASTTVDLKITSPTTEDTATYFCARGPTNTYPTYFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 451 aa, synthetic construct
QSLEESGGDLVKPGASLTLTCKASGFSFNSGYMCWVRQAPGKGLEWIACIYAGSSGKTYYASWATGRFTISKTSSTTVTLQMTSLTVADTATYFCARDDGGTYYTDYLYLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 449 aa, synthetic construct
QEQLKESGGGLVTPGGTLTLTCTASGFTISSYHMCWVRQAPGKGLEWIGCIYPNNDFTWYATWAKGRFTISKTSSTTVDLRMTTPTTEETATYFCARASGNAYNLLDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 450 aa, synthetic construct
QEQLVESGGGLVKPGASLTLTCKASGFSFSSGYYMCWVRQAPGKGLEWIGCIYVRSDSTYYASWAKGRFTISKASSTTVTLQMTSLTAADTATYFCARGVTGDSYRFNFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 448 aa, synthetic construct
QSLEESGGRLVTPGTPLTLTCTASGFSLNSYHMSWVRQAPGEGLEWIGIIYADTGNADYASWTKGRFTISKASTTVELRITSPTSEDTATYFCARGPTNTYPTYFALWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 183 - protein, 449 aa, synthetic construct
EVQLVQSGAEVKKPGASVKVSCKASGYTFTSYVMHWVRQAPGQRLEWMGYIDPYNEGTKYNEKFKGRVTITRDTSASTAYMELSSLRSEDTAVYYCSRGGMARRSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 449 aa, synthetic construct
EVQLVQSGAEVKKPGASVKVSCKASGYTFTSYVMHWVRQAPGQRLEWMGYIDPYNEGTKYNEKFKGRVTITSDTSASTAYMELSSLRSEDTAVYYCSRGGMARRSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 453 aa, synthetic construct
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYLYAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 453 aa, synthetic construct
EVQLVESGGGLVQPGGSLRLSCAASGFSFNSGYMSWVRQAPGKGLEWIASIYAGSSGKTYYADWAKGRFTISKTSSKTTVYLQMNSLRAEDTAVYYCARDSGGTYYTDYLYLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 453 aa, synthetic construct; identical to SEQ ID NO: 186

SEQ ID NO: 188 - protein, 460 aa, synthetic construct
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSDYKDDDDK

SEQ ID NO: 189 - protein, 214 aa, synthetic construct
SYEMTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 190 - protein, 214 aa, synthetic construct
SYEMTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 191 - protein, 214 aa, synthetic construct
SYELTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 192 - protein, 214 aa, synthetic construct
DIQMTQSPASQSASLGESVTITCLASQTIGTWLTWYQQKPGKSPQLLIYAATTLADGVPSRFSGSGSGTKFSFKISSLQAEDFVSYYCQQLYSTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 193 - protein, 214 aa, synthetic construct
DIQMNQSPSSLSASLGDTITITCHASQNIHVWLSWYQQKPGNIPKLLIYKASNLHTGVPSRFSGSGSGTGFTLTISSLQPEDIATYYCQQGQSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 214 aa, synthetic construct
DIQMIQSPSSMFASLGDRVSLSCRASQGIRGNLDWYQQKPGGTIKLLIYSTSNLNSGVPSRFSGSGSGSDYSLTISSLESEDFADYYCLQRNAFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - protein, 214 aa, synthetic construct
DIQMNQSPSSLSASLGDTITITCHASQNINVWLSWYQQKPGNIPKLLIYKASNLHTGVPSRFSGSGSGTGFTLTISSLQPEDIATYYCQQGQNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 214 aa, synthetic construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 197 - protein, 216 aa, synthetic construct
DVVMTQTPASVSEPVGGTVTINCQASQSISNECSWYQQKPGQPPKLLIYRASTLESGVPSRFSGSGSGTEFTLTISDLESADAATYYCQSTYYGTTGNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 217 aa, synthetic construct
ALVMTQTPASVEAAVGGTVTINCQASQSINNLLAWYQQKPGQRPKLLIYDASDLASGVSSRFKGSGSGTEYTLTISGVQSADAATYYCQGYFYGSSVDLVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - protein, 215 aa, synthetic construct
DVVMTQTPASVEAAVGGTVTIKCQASQSISNELSWYQQKPGQPPKLLIYRASTLESGVPSRFKGSGSGTEFTLTISDLESADAATYYCQCTYITYGNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 219 aa, synthetic construct
AIVMTQTPASVSATLGGSVTINCQSSESVYRDNCLGWFQQKPGQPPKLLIYKASTLASGVPSRFKGSGSGTQFTLTISDVVSDDAATYYCAGYKSTITDGTAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 201 - protein, 215 aa, synthetic construct
AFELTQTPSSVEAAVGGTVTIKCQASRSITSYLAWYQHKPGQPPTLLIYAASNVASGVSSRFKGSRSGTEYTLTISDLESADAATYYCQSYYAISTNTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 220 aa, synthetic construct
DVVMTQTPASVEAAVGGTVTIKCQASEGISSYLSWYQQKPGQPPKLLIYKASTLTSGVPSRFKGSGSGTDFTLTISDLESADAATYYCQCTYGSSGSSGYGAAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 219 aa, synthetic construct
AIVMTQTPASVSVAVGGSVTINCQSSESVYRDNCLGWFQQKPGQPPKLLIYRASTLASGVPSRFKGSGSGTQFTLTISDVVSDDAATYYCAGYKSTITDGTAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 217 aa, synthetic construct
DVVMTQTPASVEAAVGGTVTIKCQASQSISNELSWYQQKSGQPPKLLIYRASTLESGVPSRFKGSGSGTEFTLTISDLESADAATYYCQSTYYGTSYGNAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 205 - protein, 215 aa, synthetic construct
AFELTQTPSSVEAAVGGTVTIKCQASQSITTYLAWYQQKPGQPPKLLIYGASNLESGVPSRFKGSRSGTEYTLTISDLESADAATYYCQSYYAISKSAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 214 aa, synthetic construct
DIQMTQSPSSLSASVGDRVTITCLASQTIGTWLTWYQQKPGKAPKLLIYAATTLAEGVPSRFSGSGSGTDFTLTISSLQPEDFATYYCQQLYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - protein, 214 aa, synthetic construct; identical to SEQ ID NO: 206

SEQ ID NO: 208 - protein, 220 aa, synthetic construct
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSGYGAAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 209 - protein, 220 aa, synthetic construct
DIQMTQSPSSLSASVGDRVTITCRASEGISSYLSWYQQKPGKAPKLLIYKASTLTSGVPSRFSGSGSGTDFTLTISSLQPEDFATYYCQSAYGSSGSSGYGAAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 220 aa, synthetic construct
DIQMTQSPSSLSASVGDRVTITCRASEGISSYLSWYQQKPGKAPKLLIYKASTLTSGVPSRFSGSGSGTDFTLTISSLQPEDFATYYCQSTYGSSGSSGYAAAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - protein, 214 aa, synthetic construct; identical to SEQ ID NO: 191

SEQ ID NO: 212 - protein, 199 aa, synthetic construct
SYELTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLPDIQNPDPAVYQLRDSKSSDKSVCLFTDFDSQTQVSQSKDSDVYITDKCVLDMRSMDFKSNSAVAWSQKSDFACANAFQNSIIPEDTFFCS

SEQ ID NO: 213 - protein, 478 aa, synthetic construct
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSSGGGGSGGGGSGGGGSSYELTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 214 - protein, 91 aa, synthetic construct
PDIQNPDPAVYQLRDSKSSDKSVCLFTDFDSQTQVSQSKDSDVYITDKCVLDMRSMDFKSNSAVAWSQKSDFACANAFQNSIIPEDTFFCS

SEQ ID NO: 215 - protein, 131 aa, synthetic construct
LEDLKNVFPPEVAVFEPSECEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRAS

SEQ ID NO: 216 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 475 aa, synthetic construct
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVLEDLKNVFPPEVAVFEPSECEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 199 aa, synthetic construct; identical to SEQ ID NO: 212

SEQ ID NO: 219 - protein, 5 aa, synthetic construct
SYDIN

SEQ ID NO: 220 - protein, 17 aa, synthetic construct
WIYPRDGSTKYNERFKG

SEQ ID NO: 221 - protein, 14 aa, synthetic construct
DPYYYGTIYGYFDV

SEQ ID NO: 222 - protein, 5 aa, synthetic construct
SYDIN

SEQ ID NO: 223 - protein, 17 aa, synthetic construct
WIYPRDGSTKYAQKFQG

SEQ ID NO: 224 - protein, 14 aa, synthetic construct
DPYYYGTIYGYFDV

SEQ ID NO: 225 - protein, 5 aa, synthetic construct
SYDIN

SEQ ID NO: 226 - protein, 17 aa, synthetic construct
WIYPREGSTKYAQKFQG

SEQ ID NO: 227 - protein, 14 aa, synthetic construct
DPYYYGTIYGYFDV

SEQ ID NO: 228 - protein, 5 aa, synthetic construct
TYAMH

SEQ ID NO: 229 - protein, 19 aa, synthetic construct
RIRSKTYNYATFYADSVKD

SEQ ID NO: 230 - protein, 8 aa, synthetic construct
GSSYAMDY

SEQ ID NO: 231 - protein, 5 aa, synthetic construct
TYAMH

SEQ ID NO: 232 - protein, 19 aa, synthetic construct
RIRSKTYNYATFYADSVKG

SEQ ID NO: 233 - protein, 8 aa, synthetic construct
GSSYAMDY

SEQ ID NO: 234 - protein, 5 aa, synthetic construct
TYAMH

SEQ ID NO: 235 - protein, 19 aa, synthetic construct
RIRSKTYNYATFYADSVKG

SEQ ID NO: 236 - protein, 8 aa, synthetic construct
GSSYAMDY

SEQ ID NO: 237 - protein, 5 aa, synthetic construct
SYWMH

SEQ ID NO: 238 - protein, 17 aa, synthetic construct
VIDPSNSYSNYNQKFKG

SEQ ID NO: 239 - protein, 11 aa, synthetic construct
PYNHGRDAMDY

SEQ ID NO: 240 - protein, 5 aa, synthetic construct
SYGIS

SEQ ID NO: 241 - protein, 17 aa, synthetic construct
EIYPRSDNTYYNEKFKG

SEQ ID NO: 242 - protein, 8 aa, synthetic construct
RAGMWFAY

SEQ ID NO: 243 - protein, 5 aa, synthetic construct
SYGIS

SEQ ID NO: 244 - protein, 17 aa, synthetic construct
EIYPRSDNTYYAQKFQG

SEQ ID NO: 245 - protein, 8 aa, synthetic construct
RAGMWFAY

SEQ ID NO: 246 - protein, 5 aa, synthetic construct
SYGIS

SEQ ID NO: 247 - protein, 17 aa, synthetic construct
EIYPRSDNTYYAQKFQG

SEQ ID NO: 248 - protein, 8 aa, synthetic construct
RAGMWFAY

SEQ ID NO: 249 - protein, 6 aa, synthetic construct
SDFAWN

SEQ ID NO: 250 - protein, 16 aa, synthetic construct
YITYSGTTSYNPSLKS

SEQ ID NO: 251 - protein, 11 aa, synthetic construct
SFHFFAYGFAY

SEQ ID NO: 252 - protein, 5 aa, synthetic construct
SYGIS

SEQ ID NO: 253 - protein, 17 aa, synthetic construct
EIYPTRDYTYYNENFKG

SEQ ID NO: 254 - protein, 7 aa, synthetic construct
DGPPFAY

SEQ ID NO: 255 - protein, 5 aa, synthetic construct
DYYMS

SEQ ID NO: 256 - protein, 17 aa, synthetic construct
EINPDNGDTSHNQKFKG

SEQ ID NO: 257 - protein, 4 aa, synthetic construct
YFSY

SEQ ID NO: 258 - protein, 5 aa, synthetic construct
NTYIH

SEQ ID NO: 259 - protein, 17 aa, synthetic construct
RIGPANGNTKYAPKFQG

SEQ ID NO: 260 - protein, 6 aa, synthetic construct
AYYRIY

SEQ ID NO: 261 - protein, 5 aa, synthetic construct
NTYMH

SEQ ID NO: 262 - protein, 17 aa, synthetic construct
RIAPANGNTKYAPKFQD

SEQ ID NO: 263 - protein, 6 aa, synthetic construct
IYQLPY

SEQ ID NO: 264 - protein, 5 aa, synthetic construct
DYYIT

SEQ ID NO: 265 - protein, 17 aa, synthetic construct
WIFPGRGISFYNENFKD

SEQ ID NO: 266 - protein, 8 aa, synthetic construct
WGGTWFAY

SEQ ID NO: 267 - protein, 5 aa, synthetic construct
NTYMH

SEQ ID NO: 268 - protein, 17 aa, synthetic construct
RIAPANGNTKYDPKFQD

SEQ ID NO: 269 - protein, 6 aa, synthetic construct
IYQLPY

SEQ ID NO: 270 - protein, 5 aa, synthetic construct
SYGVT

SEQ ID NO: 271 - protein, 17 aa, synthetic construct
WINTYSGVPTYADDFKG

SEQ ID NO: 272 - protein, 7 aa, synthetic construct
SHSNYDF

SEQ ID NO: 273 - protein, 11 aa, synthetic construct
RASQDIGNSLN

SEQ ID NO: 274 - protein, 7 aa, synthetic construct
VTSRLDS

SEQ ID NO: 275 - protein, 9 aa, synthetic construct
LQYASSPFT

SEQ ID NO: 276 - protein, 11 aa, synthetic construct
RASQDIGNSLN

SEQ ID NO: 277 - protein, 7 aa, synthetic construct
VTSRLDS

SEQ ID NO: 278 - protein, 14 aa, synthetic construct
LQYASSPFTSYDIN

SEQ ID NO: 279 - protein, 11 aa, synthetic construct
RASQDIGNSLN

SEQ ID NO: 280 - protein, 7 aa, synthetic construct
VTSRLDS

SEQ ID NO: 281 - protein, 9 aa, synthetic construct
LQYASSPFT

SEQ ID NO: 282 - protein, 11 aa, synthetic construct
RASQSIGTSLH

SEQ ID NO: 283 - protein, 7 aa, synthetic construct
YASESIS

SEQ ID NO: 284 - protein, 9 aa, synthetic construct
QQSNSWPYT

SEQ ID NO: 285 - protein, 11 aa, synthetic construct
RASQSIGTSLH

SEQ ID NO: 286 - protein, 7 aa, synthetic construct
YASESIS

SEQ ID NO: 287 - protein, 9 aa, synthetic construct
QQSNSWPYT

SEQ ID NO: 288 - protein, 11 aa, synthetic construct
RASQSIGTSLH

SEQ ID NO: 289 - protein, 7 aa, synthetic construct
YASESIS

SEQ ID NO: 290 - protein, 9 aa, synthetic construct
QQSNSWPYT

SEQ ID NO: 291 - protein, 16 aa, synthetic construct
RSSQSFVHINGNTYLE

SEQ ID NO: 292 - protein, 7 aa, synthetic construct
KVSNRFS

SEQ ID NO: 293 - protein, 5 aa, synthetic construct
FQGSH

SEQ ID NO: 294 - protein, 10 aa, synthetic construct
SASSSVSDMH

SEQ ID NO: 295 - protein, 7 aa, synthetic construct
EISKLAS

SEQ ID NO: 296 - protein, 9 aa, synthetic construct
QHWSYPLMT

SEQ ID NO: 297 - protein, 10 aa, synthetic construct
RASSSVSDMH

SEQ ID NO: 298 - protein, 7 aa, synthetic construct
EISKLAS

SEQ ID NO: 299 - protein, 9 aa, synthetic construct
QHWSYPLMT

SEQ ID NO: 300 - protein, 10 aa, synthetic construct
RASSSVSDMH

SEQ ID NO: 301 - protein, 7 aa, synthetic construct
EISKLAS

SEQ ID NO: 302 - protein, 9 aa, synthetic construct
QHWSYPLMT

SEQ ID NO: 303 - protein, 16 aa, synthetic construct
KSSQSLLDSDGKTYLN

SEQ ID NO: 304 - protein, 7 aa, synthetic construct
LVSKLDS

SEQ ID NO: 305 - protein, 5 aa, synthetic construct
WQGTH

SEQ ID NO: 306 - protein, 16 aa, synthetic construct
RSSKSLLHSNGFTYLH

SEQ ID NO: 307 - protein, 7 aa, synthetic construct
QMSSLAS

SEQ ID NO: 308 - protein, 9 aa, synthetic construct
AQNLELPRT

SEQ ID NO: 309 - protein, 16 aa, synthetic construct
RSSQSIVDSDGSTYLE

SEQ ID NO: 310 - protein, 7 aa, synthetic construct
KVSNRFS

SEQ ID NO: 311 - protein, 5 aa, synthetic construct
FQGSH

SEQ ID NO: 312 - protein, 15 aa, synthetic construct
RASESVDNSGISFMH

SEQ ID NO: 313 - protein, 7 aa, synthetic construct
RASNLES

SEQ ID NO: 314 - protein, 9 aa, synthetic construct
QQSYENPWT

SEQ ID NO: 315 - protein, 15 aa, synthetic construct
RASESVDNYGISFMH

SEQ ID NO: 316 - protein, 7 aa, synthetic construct
RASSLES

SEQ ID NO: 317 - protein, 6 aa, synthetic construct
QQSYKD

SEQ ID NO: 318 - protein, 10 aa, synthetic construct
SASSSVSYIH

SEQ ID NO: 319 - protein, 7 aa, synthetic construct
EISKLAS

SEQ ID NO: 320 - protein, 9 aa, synthetic construct
QQWNYPLIT

SEQ ID NO: 321 - protein, 15 aa, synthetic construct
RASESVDNYGISFMH

SEQ ID NO: 322 - protein, 7 aa, synthetic construct
RASSLES

SEQ ID NO: 323 - protein, 6 aa, synthetic construct
QQSYKD

SEQ ID NO: 324 - protein, 11 aa, synthetic construct
QASQNVGTDVG

SEQ ID NO: 325 - protein, 7 aa, synthetic construct
SASNRYT

SEQ ID NO: 326 - protein, 9 aa, synthetic construct
QQYSSYPRT

SEQ ID NO: 327 - protein, 30 aa, synthetic construct
QVQLQQSGPELVKPGTSVKLSCRASGYTFT

SEQ ID NO: 328 - protein, 11 aa, synthetic construct
WVKQRPGQGLE

SEQ ID NO: 329 - protein, 32 aa, synthetic construct
KATLTVDTSSSTAYMEFHTLTSEDSAVYFCAR

SEQ ID NO: 330 - protein, 11 aa, synthetic construct
WGTGTTVTVSS

SEQ ID NO: 331 - protein, 30 aa, synthetic construct
QVQLVQSGAEVKKPGASVKVSCKASGYTFT

SEQ ID NO: 332 - protein, 14 aa, synthetic construct
WVRQAPGQGLEWMG

SEQ ID NO: 333 - protein, 32 aa, synthetic construct
RVTMTVDTSTSTAYMELSSLRSEDTAVYYCAR

SEQ ID NO: 334 - protein, 11 aa, synthetic construct
WGQGTTVTVSS

SEQ ID NO: 335 - protein, 30 aa, synthetic construct; identical to SEQ ID NO: 331

SEQ ID NO: 336 - protein, 14 aa, synthetic construct
WVRQAPGQGLEWMG

SEQ ID NO: 337 - protein, 32 aa, synthetic construct; identical to SEQ ID NO: 333

SEQ ID NO: 338 - protein, 11 aa, synthetic construct
WGQGTTVTVSS

SEQ ID NO: 339 - protein, 30 aa, synthetic construct
EVQLVESGGGLVQPKGSLKLSCAASGFTFN

SEQ ID NO: 340 - protein, 14 aa, synthetic construct
WVRQAPGKGLEWVA

SEQ ID NO: 341 - protein, 32 aa, synthetic construct
RFTISRDDSQSMLYLQMNNLKTEDTAMFYCVT

SEQ ID NO: 342 - protein, 11 aa, synthetic construct
WGQGTSVTVSS

SEQ ID NO: 343 - protein, 30 aa, synthetic construct
EVQLVESGGGLVQPGGSLRLSCAASGFTFN

SEQ ID NO: 344 - protein, 14 aa, synthetic construct
WVRQAPGKGLEWVA

SEQ ID NO: 345 - protein, 32 aa, synthetic construct
RFTISRDNSKNTLYLQMNSLRAEDTAVYYCVT

SEQ ID NO: 346 - protein, 11 aa, synthetic construct
WGQGTTVTVSS

SEQ ID NO: 347 - protein, 25 aa, synthetic construct [sequence omitted]

SEQ ID NO: 348 - protein, 14 aa, synthetic construct
WVRQAPGKGLEWVA

SEQ ID NO: 349 - protein, 39 aa, synthetic construct
YADSVKGRFTISRDDSKNTLYLQMNSLRAEDTAVYYCVT

SEQ ID NO: 350 - protein, 11 aa, synthetic construct
WGQGTTVTVSS

SEQ ID NO: 351 - protein, 30 aa, synthetic construct
QVQLQQSGAELARPGASVQLSCKASGYTFT

SEQ ID NO: 352 - protein, 14 aa, synthetic construct
WLKQRAGQGLEWIG

SEQ ID NO: 353 - protein, 32 aa, synthetic construct
KATLTADKSSSTAYMELRSLTSEDSAVYFCAR

SEQ ID NO: 354 - protein, 11 aa, synthetic construct
WGQGTLVTVSA

SEQ ID NO: 355 - protein, 30 aa, synthetic construct
QVQLVQSGAEVKKPGSSVKVSCKASGYTFT

SEQ ID NO: 356 - protein, 14 aa, synthetic construct
WVRQAPGQGLEWMG

SEQ ID NO: 357 - protein, 32 aa, synthetic construct
RVTITADKSTSTAYMELSSLRSEDTAVYYCAR

SEQ ID NO: 358 - protein, 11 aa, synthetic construct
WGQGTLVTVSS

SEQ ID NO: 359 - protein, 30 aa, synthetic construct; identical to SEQ ID NO: 355

SEQ ID NO: 360 - protein, 14 aa, synthetic construct
WVRQAPGQGLEWMG

SEQ ID NO: 361 - protein, 32 aa, synthetic construct; identical to SEQ ID NO: 357

SEQ ID NO: 362 - protein, 11 aa, synthetic construct
WGQGTLVTVSS

SEQ ID NO: 363 - protein, 23 aa, synthetic construct
DIQMTQSPSSLSASLGERVSLTC

SEQ ID NO: 364 - protein, 15 aa, synthetic construct
WLQQKPDGTIKRLIY

SEQ ID NO: 365 - protein, 32 aa, synthetic construct
GVPKRFSGSRSGSDYFFTISSLESEDFADYYC

SEQ ID NO: 366 - protein, 10 aa, synthetic construct
FGSGTKLEIK

SEQ ID NO: 367 - protein, 23 aa, synthetic construct
DIQMTQSPSSLSASVGDRVTLTC

SEQ ID NO: 368 - protein, 15 aa, synthetic construct
WLQQKPGKAPKRLIY

SEQ ID NO: 369 - protein, 32 aa, synthetic construct
GVPSRFSGSGSGTDYTFTISSLQPEDFATYYC

SEQ ID NO: 370 - protein, 10 aa, synthetic construct
FGQGTKLEIK

SEQ ID NO: 371 - protein, 23 aa, synthetic construct
DIQMTQSPSSLSASVGDRVTLTC

SEQ ID NO: 372 - protein, 15 aa, synthetic construct
WLQQKPGKAPKRLIY

SEQ ID NO: 373 - protein, 32 aa, synthetic construct
GVPSRFSGSRSGTDYTFTISSLQPEDFATYYC

SEQ ID NO: 374 - protein, 10 aa, synthetic construct
FGQGTKLEIK

SEQ ID NO: 375 - protein, 23 aa, synthetic construct
DILLTQSPAILSVSPGERVSFSC

SEQ ID NO: 376 - protein, 15 aa, synthetic construct
WYQQRTNGSPRLLIK

SEQ ID NO: 377 - protein, 32 aa, synthetic construct
GIPSRFSGSGSGTDFTLSINSVESEDIADYYC

SEQ ID NO: 378 - protein, 10 aa, synthetic construct
FGGGTKLEIK

SEQ ID NO: 379 - protein, 23 aa, synthetic construct
DIQLTQSPSSLSASVGDRVTFTC

SEQ ID NO: 380 - protein, 15 aa, synthetic construct
WYQQKPGKAPKLLIK

SEQ ID NO: 381 - protein, 32 aa, synthetic construct
GIPSRFSGSGSGTDFTLTISSVQPEDIATYYC

SEQ ID NO: 382 - protein, 10 aa, synthetic construct
FGQGTKLEIK

SEQ ID NO: 383 - protein, 23 aa, synthetic construct
DIQLTQSPSSLSASVGDRVTFTC

SEQ ID NO: 384 - protein, 15 aa, synthetic construct
WYQQKPGKAPKLLIK

SEQ ID NO: 385 - protein, 32 aa, synthetic construct
GIPSRFSGSGSGTDFTLTISSVQPEDFATYYC

SEQ ID NO: 386 - protein, 10 aa, synthetic construct
FGQGTKLEIK

SEQ ID NO: 387 - protein, 23 aa, synthetic construct
EIVLTQSPAITAASLGQKVTITC

SEQ ID NO: 388 - protein, 15 aa, synthetic construct
WYQQKSGTSPKPWIY

SEQ ID NO: 389 - protein, 32 aa, synthetic construct
GVPTRFSGSGSGTSYSLTISSMEAEDAAIYYC

SEQ ID NO: 390 - protein, 10 aa, synthetic construct
FGGGTKLEIK

SEQ ID NO: 391 - protein, 23 aa, synthetic construct
DIQMTQSPSSLSASVGDRVTITC

SEQ ID NO: 392 - protein, 15 aa, synthetic construct
WYQQKPGKAPKLLIY

SEQ ID NO: 393 - protein, 32 aa, synthetic construct
GVPSRFSGSGSGTDFTLTISSLQPEDFATYYC

SEQ ID NO: 394 - protein, 10 aa, synthetic construct
FGQGTKLEIK

SEQ ID NO: 395 - protein, 23 aa, synthetic construct
DIQMTQSPSSLSASVGDRVTITC

SEQ ID NO: 396 - protein, 15 aa, synthetic construct
WYQQKPGKAPKLLIY

SEQ ID NO: 397 - protein, 32 aa, synthetic construct; identical to SEQ ID NO: 393

SEQ ID NO: 398 - protein, 10 aa, synthetic construct
FGQGTKLEIK

SEQ ID NO: 399 - protein, 123 aa, synthetic construct
QVQLQQSGPELVKPGTSVKLSCRASGYTFTSYDINWVKQRPGQGLEWIGWIYPRDGSTKYNERFKGKATLTVDTSSSTAYMEFHTLTSEDSAVYFCARDPYYYGTIYGYFDVWGTGTTVTVSS

SEQ ID NO: 400 - protein, 123 aa, synthetic construct
QVQLVQSGAEVKKPGASVKVSCKASGYTFTSYDINWVRQAPGQGLEWMGWIYPRDGSTKYAQKFQGRVTMTVDTSTSTAYMELSSLRSEDTAVYYCARDPYYYGTIYGYFDVWGQGTTVTVSS

SEQ ID NO: 401 - protein, 123 aa, synthetic construct
QVQLVQSGAEVKKPGASVKVSCKASGYTFTSYDINWVRQAPGQGLEWMGWIYPREGSTKYAQKFQGRVTMTVDTSTSTAYMELSSLRSEDTAVYYCARDPYYYGTIYGYFDVWGQGTTVTVSS

SEQ ID NO: 402 - protein, 119 aa, synthetic construct
EVQLVESGGGLVQPKGSLKLSCAASGFTFNTYAMHWVRQAPGKGLEWVARIRSKTYNYATFYADSVKDRFTISRDDSQSMLYLQMNNLKTEDTAMFYCVTGSSYAMDYWGQGTSVTVSS

SEQ ID NO: 403 - protein, 119 aa, synthetic construct
EVQLVESGGGLVQPGGSLRLSCAASGFTFNTYAMHWVRQAPGKGLEWVARIRSKTYNYATFYADSVKGRFTISRDNSKNTLYLQMNSLRAEDTAVYYCVTGSSYAMDYWGQGTTVTVSS

SEQ ID NO: 404 - protein, 119 aa, synthetic construct
EVQLVESGGGLVQPGGSLRLSCAASGFTFNTYAMHWVRQAPGKGLEWVARIRSKTYNYATFYADSVKGRFTISRDDSKNTLYLQMNSLRAEDTAVYYCVTGSSYAMDYWGQGTTVTVSS

SEQ ID NO: 405 - protein, 120 aa, synthetic construct
QVQLQQPGAELVRPGTSVKLSCKASGYTFTSYWMHWIKQRPGQGLEWIGVIDPSNSYSNYNQKFKGRATLTVDTYSSTAYMQLSSLTSEDSAVYYCARPYNHGRDAMDYWGQGTSVTVSS

SEQ ID NO: 406 - protein, 117 aa, synthetic construct
QVQLQQSGAELARPGASVQLSCKASGYTFTSYGISWLKQRAGQGLEWIGEIYPRSDNTYYNEKFKGKATLTADKSSSTAYMELRSLTSEDSAVYFCARRAGMWFAYWGQGTLVTVSA

SEQ ID NO: 407 - protein, 117 aa, synthetic construct
QVQLVQSGAEVKKPGSSVKVSCKASGYTFTSYGISWVRQAPGQGLEWMGEIYPRSDNTYYAQKFQGRVTITADKSTSTAYMELSSLRSEDTAVYYCARRAGMWFAYWGQGTLVTVSS

SEQ ID NO: 408 - protein, 117 aa, synthetic construct; identical to SEQ ID NO: 407

SEQ ID NO: 409 - protein, 120 aa, synthetic construct
DVQLQESGPGLVKPSQSLSLTCTVTGYSITSDFAWNWIRQFPGNKLEWVGYITYSGTTSYNPSLKSRISITRDTSKNQFFLQLSSVTTEDTATYYCARSFHFFAYGFAYWGQGTLVTVSA

SEQ ID NO: 410 - protein, 116 aa, synthetic construct
QVQLQQSGAELARPGASVKVSCKASGYTFTSYGISWVKQRTGQGLEWIGEIYPTRDYTYYNENFKGKATLTADKSSTTAYMELRSLTSEDSAVYFCARDGPPFAYWGQGTLVTVSA

SEQ ID NO: 411 - protein, 113 aa, synthetic construct
EVQLQQSGPELVKPGASVKISCKASGYTFTDYYMSWVKQSHGRSLEWIGEINPDNGDTSHNQKFKGKATMTVDKSSGTGNMEIRSLTSEDAGIYYCTSYFSYWGQGTLVTVSA

SEQ ID NO: 412 - protein, 115 aa, synthetic construct
EVLLQQSVADLVRPGASVKLSCTASGLNIKNTYIHWVKQMPEQGLEWIGRIGPANGNTKYAPKFQGKATITADTSSDTAYLQLSSLTSEDTAIYYCALAYYRIYWGQGTTLTVSS

SEQ ID NO: 413 - protein, 115 aa, synthetic construct
EVQLQQSVAELVRPGASVKLSCTASGFNIKNTYMHWVKQRPEQGLEWIGRIAPANGNTKYAPKFQDKATITANTSSNTAYLQLGSLTSEDTAIYYCAFIYQLPYWGQGTTLTVSS

SEQ ID NO: 414 - protein, 117 aa, synthetic construct
QVQVQQSGPELVKPGTSVKISCKTSGYTFTDYYITWVKQRPGQGLEWIGWIFPGRGISFYNENFKDKATLTVDKSSNTAFMLLSSLTSEDSAVYFCARWGGTWFAYWGQGTLITVSA

SEQ ID NO: 415 - protein, 115 aa, synthetic construct
EVQLQQSVAELVRPGASVKLSCTASGFNIKNTYMHWVKQRPEQGLEWIGRIAPANGNTKYDPKFQDKATITADSSSNTAYLHLSSLTSEDTAIYYCAFIYQLPYWGQGSTLTVSS

SEQ ID NO: 416 - protein, 116 aa, synthetic construct
QIQLVQSGPELKKPGETVKISCKASGYTFTSYGVTWVKQAPGKGLKWMGWINTYSGVPTYADDFKGRFAFSLETSASTAYLQINNLKNEDTATYFCARSHSNYDFWGQGTTLTVSS

SEQ ID NO: 417 - protein, 107 aa, synthetic construct
DIQMTQSPSSLSASLGERVSLTCRASQDIGNSLNWLQQKPDGTIKRLIYVTSRLDSGVPKRFSGSRSGSDYFFTISSLESEDFADYYCLQYASSPFTFGSGTKLEIK

SEQ ID NO: 418 - protein, 107 aa, synthetic construct
DIQMTQSPSSLSASVGDRVTLTCRASQDIGNSLNWLQQKPGKAPKRLIYVTSRLDSGVPSRFSGSGSGTDYTFTISSLQPEDFATYYCLQYASSPFTFGQGTKLEIK

SEQ ID NO: 419 - protein, 107 aa, synthetic construct
DIQMTQSPSSLSASVGDRVTLTCRASQDIGNSLNWLQQKPGKAPKRLIYVTSRLDSGVPSRFSGSRSGTDYTFTISSLQPEDFATYYCLQYASSPFTFGQGTKLEIK

SEQ ID NO: 420 - protein, 107 aa, synthetic construct
DILLTQSPAILSVSPGERVSFSCRASQSIGTSLHWYQQRTNGSPRLLIKYASESISGIPSRFSGSGSGTDFTLSINSVESEDIADYYCQQSNSWPYTFGGGTKLEIK

SEQ ID NO: 421 - protein, 107 aa, synthetic construct
DIQLTQSPSSLSASVGDRVTFTCRASQSIGTSLHWYQQKPGKAPKLLIKYASESISGIPSRFSGSGSGTDFTLTISSVQPEDIATYYCQQSNSWPYTFGQGTKLEIK

SEQ ID NO: 422 - protein, 107 aa, synthetic construct
DIQLTQSPSSLSASVGDRVTFTCRASQSIGTSLHWYQQKPGKAPKLLIKYASESISGIPSRFSGSGSGTDFTLTISSVQPEDFATYYCQQSNSWPYTFGQGTKLEIK

SEQ ID NO: 423 - protein, 112 aa, synthetic construct
DVLLTQTPLSLSVSLGDQASISCRSSQSFVHINGNTYLEWFLQKPGLSPKLLIYKVSNRFSGVPDRFSGSGSGTDFTLKISRVEAEDLGIYYCFQGSHVPLTFGAGTKLELK

SEQ ID NO: 424 - protein, 106 aa, synthetic construct
EIVLTQSPAITAASLGQKVTITCSASSSVSDMHWYQQKSGTSPKPWIYEISKLASGVPTRFSGSGSGTSYSLTISSMEAEDAAIYYCQHWSYPLMTFGGGTKLEIK

SEQ ID NO: 425 - protein, 106 aa, synthetic construct
DIQMTQSPSSLSASVGDRVTITCRASSSVSDMHWYQQKPGKAPKLLIYEISKLASGVPSRFSGSGSGTDFTLTISSLQPEDFATYYCQHWSYPLMTFGQGTKLEIK

SEQ ID NO: 426 - protein, 106 aa, synthetic construct; identical to SEQ ID NO: 425

SEQ ID NO: 427 - protein, 112 aa, synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 428 - protein, 112 aa, synthetic construct
DIVMTQAAFSNPVTLGTSASISCRSSKSLLHSNGFTYLHWYLQKPGQSPQLLIYQMSSLASGVPDRFSSSGSGTDFTLRISRVEAEDVGVYYCAQNLELPRTFGGGTKLEIK

SEQ ID NO: 429 - protein, 112 aa, synthetic construct
DVLMTQSPLSLPVSLGDQASISCRSSQSIVDSDGSTYLEWYLQKPGQSPKLLIYKVSNRFSGVPDRFSGSGSGTDFTLKISGVEAEDLGVYYCFQGSHVPYTFGGGTKLEIK

SEQ ID NO: 430 - protein, 111 aa, synthetic construct
DIVLTQSPASLAVSLGQRATISCRASESVDNSGISFMHWYQQKPGQSPKLLIYRASNLESGIPARFSGSGSRTDFTLTINPVETDDVATYYCQQSYENPWTFGGGTKLEIK

SEQ ID NO: 431 - protein, 111 aa, synthetic construct
DIVLTQSPASLAVSLGQRATISCRASESVDNYGISFMHWFQQKPGQPPKLLIFRASSLESGIPARFSGSGSRTDFTLTINPVETDDVATYYCQQSYKDPWTFGGATKLEIK

SEQ ID NO: 432 - protein, 106 aa, synthetic construct
EIVLTQSPAITAAFLGQKVTITCSASSSVSYIHWYQQKSDTSPKPWIYEISKLASGVPARFSGSGSGTSYSLTISSMEAEDAAIYYCQQWNYPLITFGGGTKLEIK

SEQ ID NO: 433 - protein, 111 aa, synthetic construct
DIVLTQSPTSLAVSLGQRATISCRASESVDNYGISFMHWYQQKPGQPPKLLIYRASSLESGIPARFSGSGSRTDFTLTINPVESDDVATYYCQQSYKDPWTFGGATKLEIK

SEQ ID NO: 434 - protein, 107 aa, synthetic construct
DIVMTQSQKFMSTTVGDRVSITCQASQNVGTDVGWYQQKPGQSPKLLIYSASNRYTGVPDRFTGSGSGTDFTLTISNMQSEDLADYFCQQYSSYPRTFGGGTKLEIK

SEQ ID NO: 435 - protein, 453 aa, synthetic construct
QVQLQQSGPELVKPGTSVKLSCRASGYTFTSYDINWVKQRPGQGLEWIGWIYPRDGSTKYNERFKGKATLTVDTSSSTAYMEFHTLTSEDSAVYFCARDPYYYGTIYGYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 436 - protein, 453 aa, synthetic construct
QVQLVQSGAEVKKPGASVKVSCKASGYTFTSYDINWVRQAPGQGLEWMGWIYPRDGSTKYAQKFQGRVTMTVDTSTSTAYMELSSLRSEDTAVYYCARDPYYYGTIYGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 437 - protein, 453 aa, synthetic construct
QVQLVQSGAEVKKPGASVKVSCKASGYTFTSYDINWVRQAPGQGLEWMGWIYPREGSTKYAQKFQGRVTMTVDTSTSTAYMELSSLRSEDTAVYYCARDPYYYGTIYGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 438 - protein, 449 aa, synthetic construct
EVQLVESGGGLVQPKGSLKLSCAASGFTFNTYAMHWVRQAPGKGLEWVARIRSKTYNYATFYADSVKDRFTISRDDSQSMLYLQMNNLKTEDTAMFYCVTGSSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 439 - protein, 449 aa, synthetic construct
EVQLVESGGGLVQPGGSLRLSCAASGFTFNTYAMHWVRQAPGKGLEWVARIRSKTYNYATFYADSVKGRFTISRDNSKNTLYLQMNSLRAEDTAVYYCVTGSSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 440 - protein, 449 aa, synthetic construct
EVQLVESGGGLVQPGGSLRLSCAASGFTFNTYAMHWVRQAPGKGLEWVARIRSKTYNYATFYADSVKGRFTISRDDSKNTLYLQMNSLRAEDTAVYYCVTGSSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 441 - protein, 447 aa, synthetic construct
QVQLQQSGAELARPGASVQLSCKASGYTFTSYGISWLKQRAGQGLEWIGEIYPRSDNTYYNEKFKGKATLTADKSSSTAYMELRSLTSEDSAVYFCARRAGMWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 442 - protein, 447 aa, synthetic construct
QVQLVQSGAEVKKPGSSVKVSCKASGYTFTSYGISWVRQAPGQGLEWMGEIYPRSDNTYYAQKFQGRVTITADKSTSTAYMELSSLRSEDTAVYYCARRAGMWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 443 - protein, 447 aa, synthetic construct
QVQLVQSGAEVKKPGSSVKVSCKASGYTFTSYGISWVRQAPGQGLEWMGEIYPRSDNTYYAQKFQGRVTITADKSTSTAYMELSSLRSEDTAVYYCARRAGMWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 444 - protein, 214 aa, synthetic construct
DIQMTQSPSSLSASLGERVSLTCRASQDIGNSLNWLQQKPDGTIKRLIYVTSRLDSGVPKRFSGSRSGSDYFFTISSLESEDFADYYCLQYASSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 445 - protein, 214 aa, synthetic construct
DIQMTQSPSSLSASVGDRVTLTCRASQDIGNSLNWLQQKPGKAPKRLIYVTSRLDSGVPSRFSGSGSGTDYTFTISSLQPEDFATYYCLQYASSPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 446 - protein, 214 aa, synthetic construct
DIQMTQSPSSLSASVGDRVTLTCRASQDIGNSLNWLQQKPGKAPKRLIYVTSRLDSGVPSRFSGSRSGTDYTFTISSLQPEDFATYYCLQYASSPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 447 - protein, 214 aa, synthetic construct
DILLTQSPAILSVSPGERVSFSCRASQSIGTSLHWYQQRTNGSPRLLIKYASESISGIPSRFSGSGSGTDFTLSINSVESEDIADYYCQQSNSWPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 448 - protein, 214 aa, synthetic construct
DIQLTQSPSSLSASVGDRVTFTCRASQSIGTSLHWYQQKPGKAPKLLIKYASESISGIPSRFSGSGSGTDFTLTISSVQPEDIATYYCQQSNSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 449 - protein, 214 aa, synthetic construct
DIQLTQSPSSLSASVGDRVTFTCRASQSIGTSLHWYQQKPGKAPKLLIKYASESISGIPSRFSGSGSGTDFTLTISSVQPEDFATYYCQQSNSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 450 - protein, 213 aa, synthetic construct
EIVLTQSPAITAASLGQKVTITCSASSSVSDMHWYQQKSGTSPKPWIYEISKLASGVPTRFSGSGSGTSYSLTISSMEAEDAAIYYCQHWSYPLMTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 451 - protein, 213 aa, synthetic construct
DIQMTQSPSSLSASVGDRVTITCRASSSVSDMHWYQQKPGKAPKLLIYEISKLASGVPSRFSGSGSGTDFTLTISSLQPEDFATYYCQHWSYPLMTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 452 - protein, 213 aa, synthetic construct; identical to SEQ ID NO: 451

SEQ ID NO: 453 - protein, 485 aa, synthetic construct
DIQMTQSPSSLSASVGDRVTITCRASSSVSDMHWYQQKPGKAPKLLIYEISKLASGVPSRFSGSGSGTDFTLTISSLQPEDFATYYCQHWSYPLMTFGQGTKLEIKGGGGSGGGGSGGGGSQVQLVQSGAEVKKPGSSVKVSCKASGYTFTSYGISWVRQAPGQGLEWMGEIYPRSDNTYYAQKFQGRVTITADKSTSTAYMELSSLRSEDTAVYYCARRAGMWFAYWGQGTLVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSHHHHHHHH

SEQ ID NO: 454 - protein, 492 aa, synthetic construct
DIQMTQSPSSLSASVGDRVTLTCRASQDIGNSLNWLQQKPGKAPKRLIYVTSRLDSGVPSRFSGSGSGTDYTFTISSLQPEDFATYYCLQYASSPFTFGQGTKLEIKGGGGSGGGGSGGGGSQVQLVQSGAEVKKPGASVKVSCKASGYTFTSYDINWVRQAPGQGLEWMGWIYPRDGSTKYAQKFQGRVTMTVDTSTSTAYMELSSLRSEDTAVYYCARDPYYYGTIYGYFDVWGQGTTVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSHHHHHHHH

SEQ ID NO: 455 - protein, 488 aa, synthetic construct
DIQLTQSPSSLSASVGDRVTFTCRASQSIGTSLHWYQQKPGKAPKLLIKYASESISGIPSRFSGSGSGTDFTLTISSVQPEDIATYYCQQSNSWPYTFGQGTKLEIKGGGGSGGGGSGGGGSEVQLVESGGGLVQPGGSLRLSCAASGFTFNTYAMHWVRQAPGKGLEWVARIRSKTYNYATFYADSVKGRFTISRDNSKNTLYLQMNSLRAEDTAVYYCVTGSSYAMDYWGQGTTVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSHHHHHHHH

SEQ ID NO: 456 - protein, 492 aa, synthetic construct
DIQMTQSPSSLSASVGDRVTLTCRASQDIGNSLNWLQQKPGKAPKRLIYVTSRLDSGVPSRFSGSRSGTDYTFTISSLQPEDFATYYCLQYASSPFTFGQGTKLEIKGGGGSGGGGSGGGGSQVQLVQSGAEVKKPGASVKVSCKASGYTFTSYDINWVRQAPGQGLEWMGWIYPREGSTKYAQKFQGRVTMTVDTSTSTAYMELSSLRSEDTAVYYCARDPYYYGTIYGYFDVWGQGTTVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSHHHHHHHH

SEQ ID NO: 457 - protein, 477 aa, synthetic construct
DIQLTQSPSSLSASVGDRVTFTCRASQSIGTSLHWYQQKPGKAPKLLIKYASESISGIPSRFSGSGSGTDFTLTISSVQPEDFATYYCQQSNSWPYTFGQGTKLEIKGGGGSGGGGSGGGGSEVQLVESGGGLVQPGGSLRLSCAASGFTFNTYAMHWVRQAPGKGLEWVARIRSKTYNYATFYADSVKGRFTISRDDSKNTLYLQMNSLRAEDTAVYYCVTGSSYAMDYWGQGTTVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 458 - protein, 202 aa, synthetic construct
DIQLTQSPSSLSASVGDRVTFTCRASQSIGTSLHWYQQKPGKAPKLLIKYASESISGIPSRFSGSGSGTDFTLTISSVQPEDFATYYCQQSNSWPYTFGQGTKLEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 459 - protein, 474 aa, synthetic construct
EVQLVESGGGLVQPGGSLRLSCAASGFTFNTYAMHWVRQAPGKGLEWVARIRSKTYNYATFYADSVKGRFTISRDDSKNTLYLQMNSLRAEDTAVYYCVTGSSYAMDYWGQGTTVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 460 - protein, 5 aa, synthetic construct
NYGVH

SEQ ID NO: 461 - protein, 16 aa, synthetic construct
VIWSGGNTDYNTPFTS

SEQ ID NO: 462 - protein, 11 aa, synthetic construct
ALTYYDYEFAY

SEQ ID NO: 463 - protein, 7 aa, synthetic construct
SGDYYWT

SEQ ID NO: 464 - protein, 16 aa, synthetic construct
HIYYSGNTNYNPSLKS

SEQ ID NO: 465 - protein, 9 aa, synthetic construct
DRVTGAFDI

SEQ ID NO: 466 - protein, 11 aa, synthetic construct
RASQSIGTNIH

SEQ ID NO: 467 - protein, 7 aa, synthetic construct
YASESIS

SEQ ID NO: 468 - protein, 9 aa, synthetic construct
QQNNNWPTT

SEQ ID NO: 469 - protein, 11 aa, synthetic construct
QASQDISNYLN

SEQ ID NO: 470 - protein, 7 aa, synthetic construct
DASNLET

SEQ ID NO: 471 - protein, 9 aa, synthetic construct
QHFDHLPLA

SEQ ID NO: 472 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 473 - protein, 119 aa, synthetic construct [sequence omitted]

SEQ ID NO: 474 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 475 - protein, 107 aa, synthetic construct [sequence omitted]

SEQ ID NO: 476 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 477 - protein, 449 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 478 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 479 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 480 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 481 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 482 - protein, 449 aa, synthetic construct
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 483 - protein, 214 aa, synthetic construct; identical to SEQ ID NO: 191

SEQ ID NO: 484 - protein, 477 aa, synthetic construct; identical to SEQ ID NO: 457

SEQ ID NO: 485 - protein, 447 aa, synthetic construct; identical to SEQ ID NO: 443

SEQ ID NO: 486 - protein, 213 aa, synthetic construct; identical to SEQ ID NO: 451

SEQ ID NO: 487 - protein, 478 aa, synthetic construct; identical to SEQ ID NO: 213

SEQ ID NO: 488 - protein, 203 aa, synthetic construct
SYELTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 489 - protein, 474 aa, synthetic construct
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGRASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 490 - protein, 448 aa, synthetic construct
EVQLVESGGGLVQPGGSLRLSCAASGFTFNTYAMHWVRQAPGKGLEWVARIRSKTYNYATFYADSVKGRFTISRDDSKNTLYLQMNSLRAEDTAVYYCVTGSSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 491 - protein, 214 aa, synthetic construct; identical to SEQ ID NO: 449

SEQ ID NO: 492 - protein, 202 aa, synthetic construct; identical to SEQ ID NO: 458

SEQ ID NO: 493 - protein, 474 aa, synthetic construct; identical to SEQ ID NO: 459

SEQ ID NO: 494 - protein, 214 aa, synthetic construct
SYELTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 495 - protein, 448 aa, synthetic construct
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 496 - protein, 449 aa, synthetic construct; identical to SEQ ID NO: 477

SEQ ID NO: 497 - protein, 214 aa, synthetic construct; identical to SEQ ID NO: 479

SEQ ID NO: 498 - protein, 478 aa, synthetic construct; identical to SEQ ID NO: 213

SEQ ID NO: 499 - protein, 199 aa, synthetic construct; identical to SEQ ID NO: 212

SEQ ID NO: 500 - protein, 475 aa, synthetic construct; identical to SEQ ID NO: 217

SEQ ID NO: 501 - protein, 449 aa, synthetic construct; identical to SEQ ID NO: 481

SEQ ID NO: 502 - protein, 214 aa, synthetic construct; identical to SEQ ID NO: 478

SEQ ID NO: 503 - protein, 449 aa, synthetic construct
EVQLVESGGGLVQPGGSLRLSCAASGFTFNTYAMHWVRQAPGKGLEWVARIRSKTYNYATFYADSVKGRFTISRDDSKNTLYLQMNSLRAEDTAVYYCVTGSSYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 504 - protein, 214 aa, synthetic construct; identical to SEQ ID NO: 449

SEQ ID NO: 505 - protein, 478 aa, synthetic construct; identical to SEQ ID NO: 213

SEQ ID NO: 506 - protein, 481 aa, synthetic construct
DIQMTQSPSSLSASVGDRVTLTCRASQDIGNSLNWLQQKPGKAPKRLIYVTSRLDSGVPSRFSGSRSGTDYTFTISSLQPEDFATYYCLQYASSPFTFGQGTKLEIKGGGGSGGGGSGGGGSQVQLVQSGAEVKKPGASVKVSCKASGYTFTSYDINWVRQAPGQGLEWMGWIYPRDGSTKYAQKFQGRVTMTVDTSTSTAYMELSSLRSEDTAVYYCARDPYYYGTIYGYFDVWGQGTTVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 507 - protein, 477 aa, synthetic construct
DIQLTQSPSSLSASVGDRVTFTCRASQSIGTSLHWYQQKPGKAPKLLIKYASESISGIPSRFSGSGSGTDFTLTISSVQPEDIATYYCQQSNSWPYTFGQGTKLEIKGGGGSGGGGSGGGGSEVQLVESGGGLVQPGGSLRLSCAASGFTFNTYAMHWVRQAPGKGLEWVARIRSKTYNYATFYADSVKGRFTISRDNSKNTLYLQMNSLRAEDTAVYYCVTGSSYAMDYWGQGTTVTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 508 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARGALTYYDYEFAYWQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 509 - protein, 214 aa, synthetic construct; identical to SEQ ID NO: 478

SEQ ID NO: 510 - protein, 478 aa, synthetic construct; identical to SEQ ID NO: 213